(19) United States
(12) Patent Application Publication
(10) Pub. No.: US 2024/0123073 A1
(43) Pub. Date: Apr. 18, 2024

(54) NOVEL RIPK1 KINASE TARGETING PROTACS AND METHODS OF USE THEREOF

(71) Applicant: Baylor College of Medicine, Houston, TX (US)

(72) Inventors: Jin Wang, Houston, TX (US); Dong Lu, Houston, TX (US); Xin Yu, Houston, TX (US)

(73) Assignee: Baylor College of Medicine, Houston, TX (US)

(21) Appl. No.: 18/039,661

(22) PCT Filed: Dec. 3, 2021
(86) PCT No.: PCT/US2021/061720

§ 371 Date: May 31, 2023

Related U.S. Application Data
(60) Provisional application No. 63/121,058, filed on Dec. 3, 2020.

(51) Int. Cl. A61K 47/55; A61K 47/54; A61K 47/60

(52) U.S. Cl. CPC A61K 47/55; A61K 47/545; A61K 47/60

(57) ABSTRACT

Novel small molecule proteolysis-targeting chimeras (PROTACs) are provided, along with methods for their use as RIPK1 kinase degraders. The small molecule PROTACs described herein are useful in treating and/or preventing RIPK1 kinase-related diseases, such as cancer, neurodegenerative disorders, and inflammatory diseases. Also provided are methods for promoting RIPK1 kinase degradation in a cell using the compounds and compositions described herein.

### CROSS-REFERENCE TO PRIORITY APPLICATION

This application claims priority to U.S. Provisional Application No. 63/121,058, filed Dec. 3, 2020, which is incorporated herein by reference in its entirety.

### BACKGROUND

Receptor-interacting serine/threonine-protein kinase 1 (RIPK1) plays a critical role in cell death and has been linked to cell pathways that mediate apoptotic and necrotic cell death. In addition, RIPK1 is understood to be a key mediator in inflammatory pathways. Thus, the RIPK1 kinase is an important therapeutic target for autoimmune, inflammatory, and neurodegenerative diseases.

### SUMMARY

Described herein are novel small molecule proteolysis-targeting chimeras (PROTACs) and methods for their use as receptor-interacting serine/threonine-protein kinase 1 (RIPK1) degraders. Specifically, the present disclosure describes bifunctional compounds, including compositions comprising the same, which function to recruit endogenous protein to an E3 ubiquitin ligase for ubiquitination and subsequent degradation, and methods of using the same. In particular, the present disclosure provides bifunctional or proteolysis targeting chimeric compounds, which find utility as modulators of ubiquitination and degradation of RIPK1 kinase. Thus, in one aspect, the disclosure provides compounds which function to recruit endogenous protein, e.g., RIPK1 kinase, to E3 ubiquitin ligase for ubiquitination and degradation.

In addition, the description provides methods of using an effective amount of the compounds as described herein for the treatment or amelioration of disease conditions due to RIPK1 kinase activity. The compounds described herein are useful in treating and/or preventing RIPK1 kinase-related diseases, such as cancer, neurodegenerative disorders, inflammatory diseases, metabolic disorders, and other indications as described herein.

A class of small molecule RIPK1 kinase degraders as described herein includes compounds of the following formula:

TPM-L-ELM

and pharmaceutically acceptable salts, co-crystals, tautomers, stereoisomers, solvates, hydrates, polymorphs, isotopically enriched derivatives, and prodrugs thereof, wherein TPM is a targeting protein binding moiety, L is a bond or a chemical linker group, and ELM is an E3 ubiquitin ligase binding moiety, wherein L is covalently bonded to the TPM and the ELM.

In some cases, a class of small molecule RIPK1 kinase degraders includes compounds of the following formula:

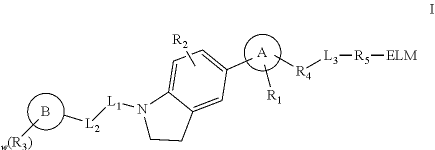

and pharmaceutically acceptable salts, co-crystals, tautomers, stereoisomers, solvates, hydrates, polymorphs, isotopically enriched derivatives, and prodrugs thereof, wherein ELM is an E3 ubiquitin ligase binding moiety; Ring A and Ring B are each independently selected from a 5-10 membered aryl ring or a heterocycle ring; L₁ is —S(O)—, —S(O)₂—, or —C(O)—; L₂ is a bond, —N(R₁)— or —CH(R₁)—; L₃ is —(CH₂)ₙ— or —(OCH₂CH₂)ₙ—; R₁, R₂, R₃ are each independently hydrogen, halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, R₄, tetrazole, aryl, aryl substituted with from one to three substituents independently selected from halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, R₄, heterocycle, heterocycle substituted with from one to three substituents independently selected from halogen, —OH, —OH₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, and R₄; R₄ and R₅ are independently a bond or C₁₋₁₂ alkyl, wherein one or more carbons are optionally replaced with halo, CO, CONH, O, S, SO, SO₂, N, NHCO, heterocycle; n is independently an integer from 0 to 15; and w is independently an integer from 0 to 4.

In some cases, a class of small molecule RIPK1 kinase degraders includes compounds of the following formula:

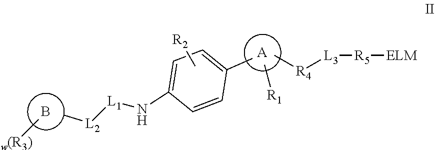

and pharmaceutically acceptable salts, co-crystals, tautomers, stereoisomers, solvates, hydrates, polymorphs, isotopically enriched derivatives, and prodrugs thereof, wherein ELM is an E3 ubiquitin ligase binding moiety; Ring A and Ring B are independently selected from 5-10 membered aryl ring or heterocycle ring; L₁ is —S(O)—, —S(O)₂—, or —C(O)—, L₂ is a bond, —N(R₁)— or —CH(R₁)—; L₃ is —(CH₂)ₙ— or —(OCH₂CH₂)ₙ—; each of R₁, R₂, R₃ are independently hydrogen, halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, R₄, tetrazole, aryl, aryl substituted with from one to three substituents independently selected from halogen. —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, R₄, heterocycle, heterocycle substituted with from one to three substituents independently selected from halogen, —OH, —NH₂, —NR₄R₅, —NR₄COR₅, —CN, —COOH, —COOR₃, —CONH₂, —CONR₄R₅, —OCF₃, —SO₃H, —SO₃R₄, R₄; R₄ and R₅ are independently a bond or C₁₋₁₂ alkyl, wherein one or more carbon is optionally replaced with halo, CO, CONH, O, S, SO, SO₂, N, NHCO, or heterocycle; n is independently an integer from 0 to 15; and w is independently an integer from 0 to 4.

In some cases, a class of small molecule RIPK1 kinase degraders includes compounds of the following formula:

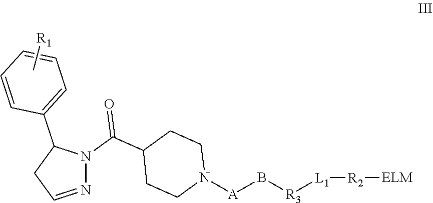

and pharmaceutically acceptable salts, co-crystals, tautomers, stereoisomers, solvates, hydrates, polymorphs, isotopically enriched derivatives, and prodrugs thereof, wherein ELM is an E3 ubiquitin ligase binding moiety; A is —CO—, one or two R₁ substituted 5-10 membered aryl rings or one or two R₁ substituted heteroaryl rings; B is a bond, one or two R₁ substituted 5-10 membered aryl rings, or one or two R₁ substituted heteroaryl rings; R₁ is independently hydrogen, halogen, —OH, —OR₂, —NH₃, —NR₂R₃, —NR₂COR₃, —CN, —COOH, —COOR₃, —CONH₂, —CONR₂R₃, —OCF₃, —SO₃R₃, R₃; R₂ and R₃ are independently a bond or C₁₋₆ alkyl, wherein one or more carbon is optionally replaced with halo, CO, CONH, O, S, SO, SO₂, N, NHCO, L₁ is —(CH₂)ₙ— or —(OCH₂CH₂)ₙ—; and n is independently an integer from 0 to 20.

Optionally, the compound is selected from the group consisting of:

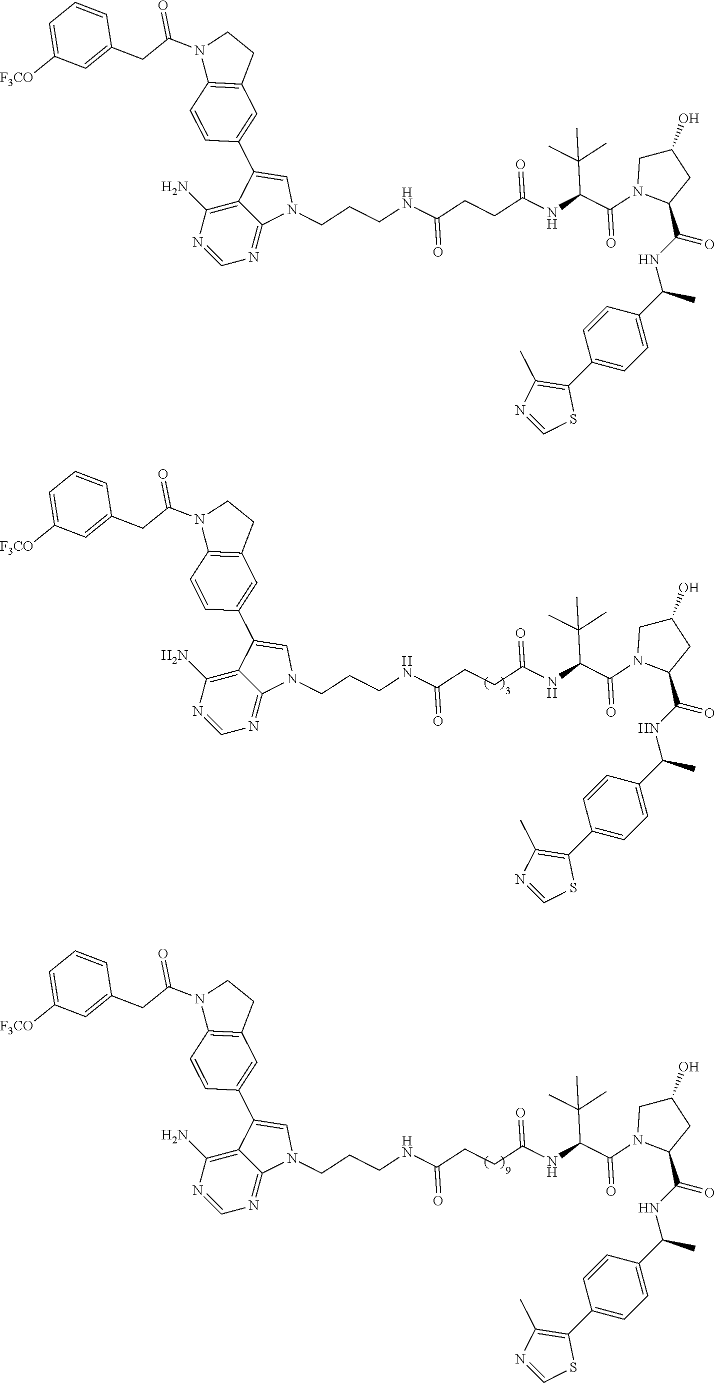

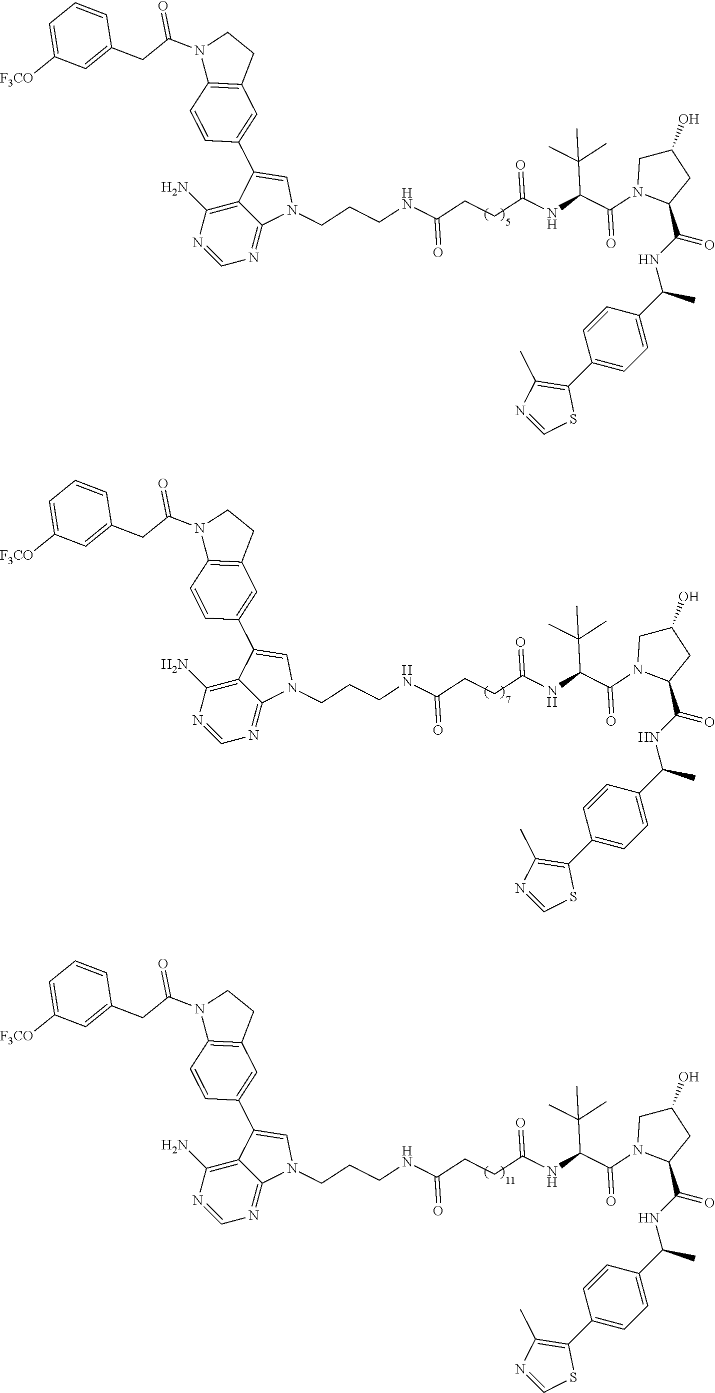

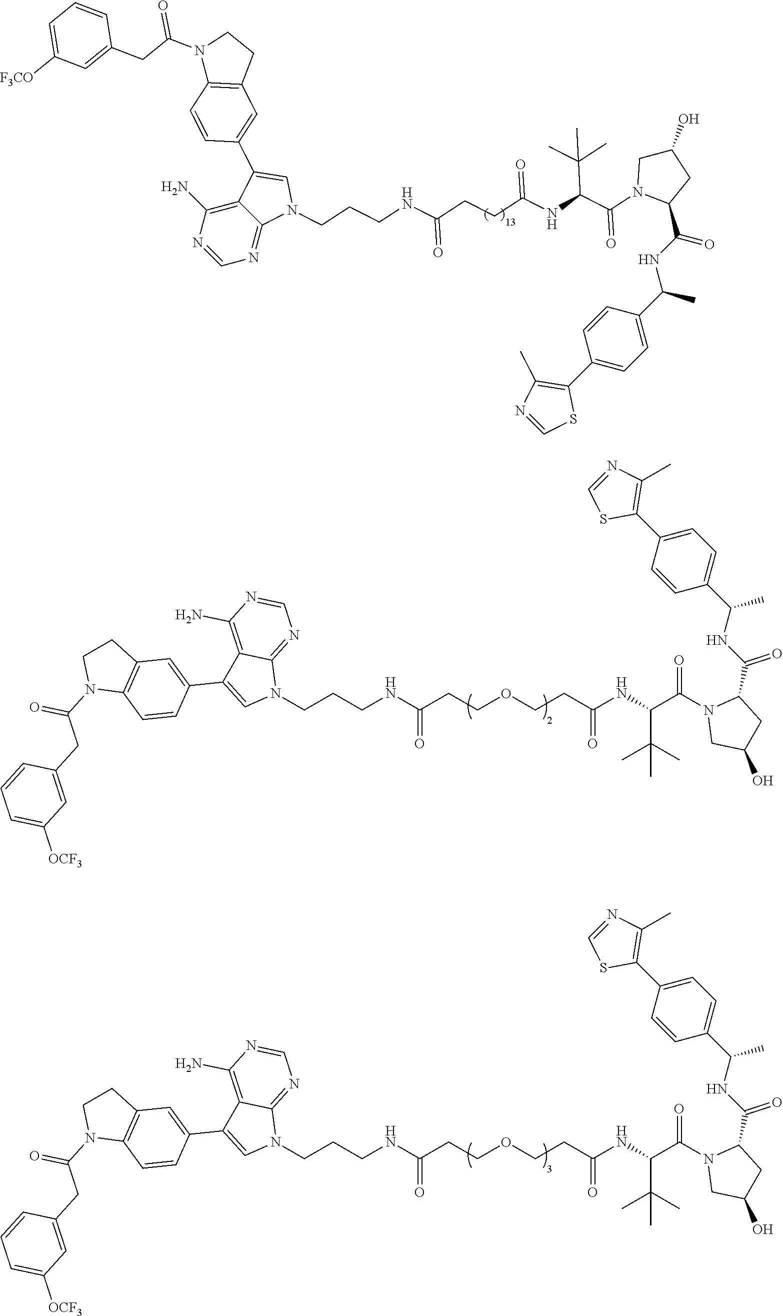

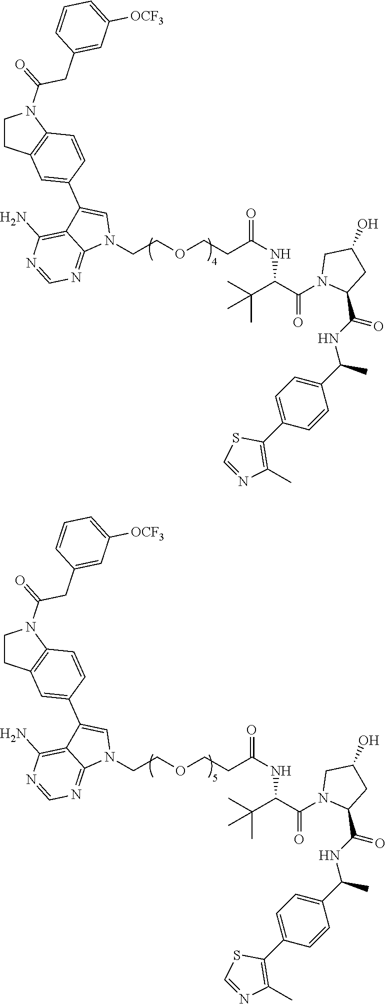

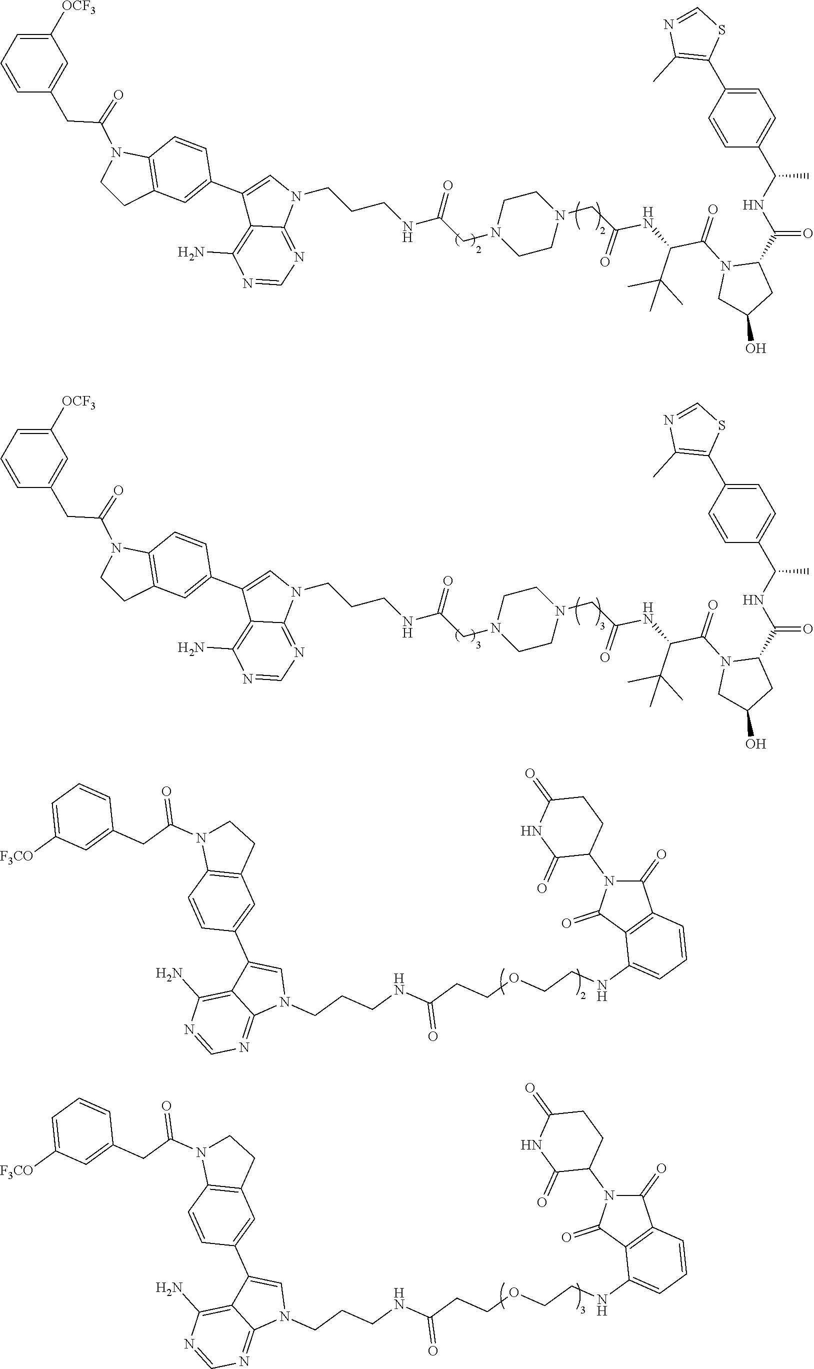

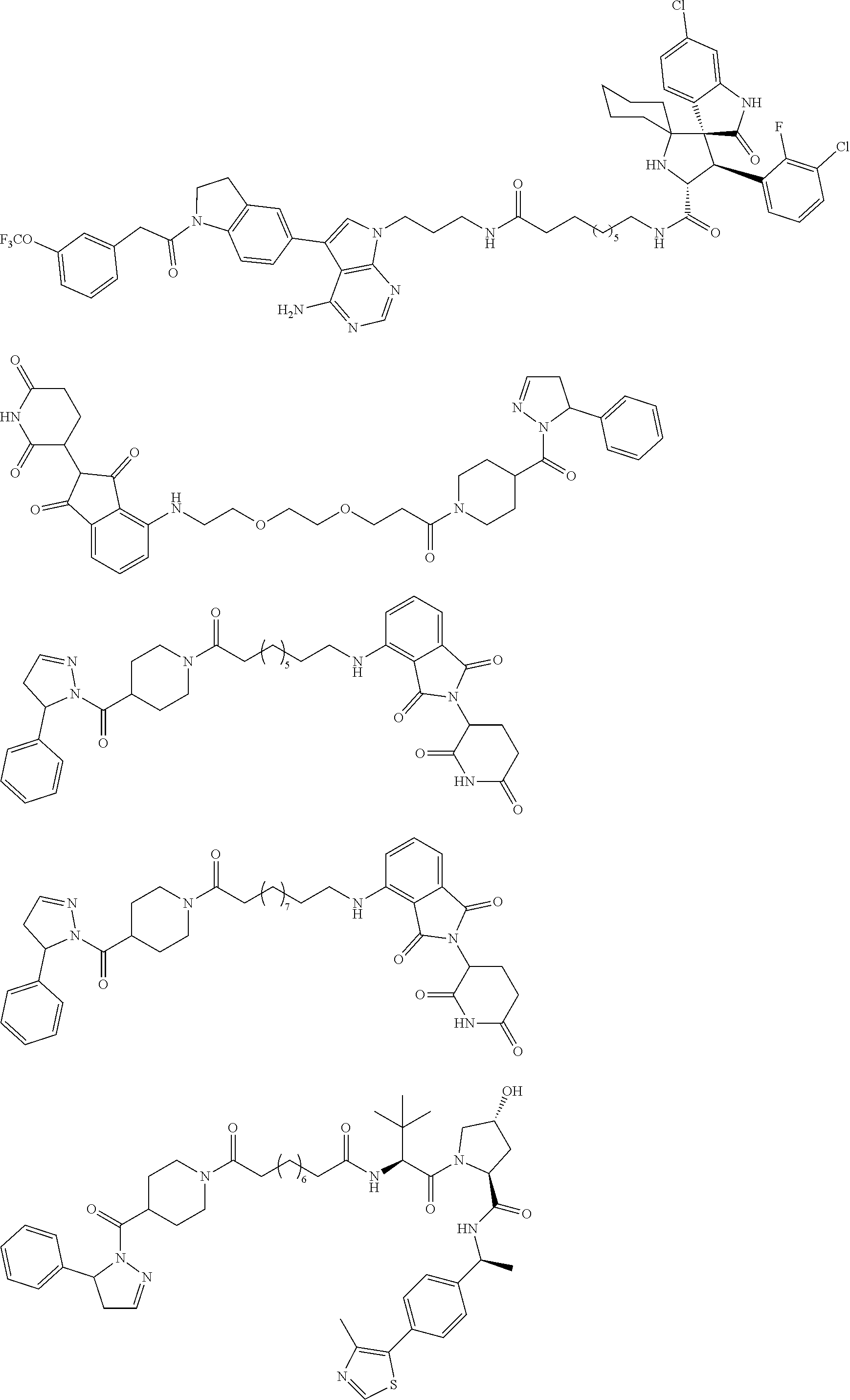

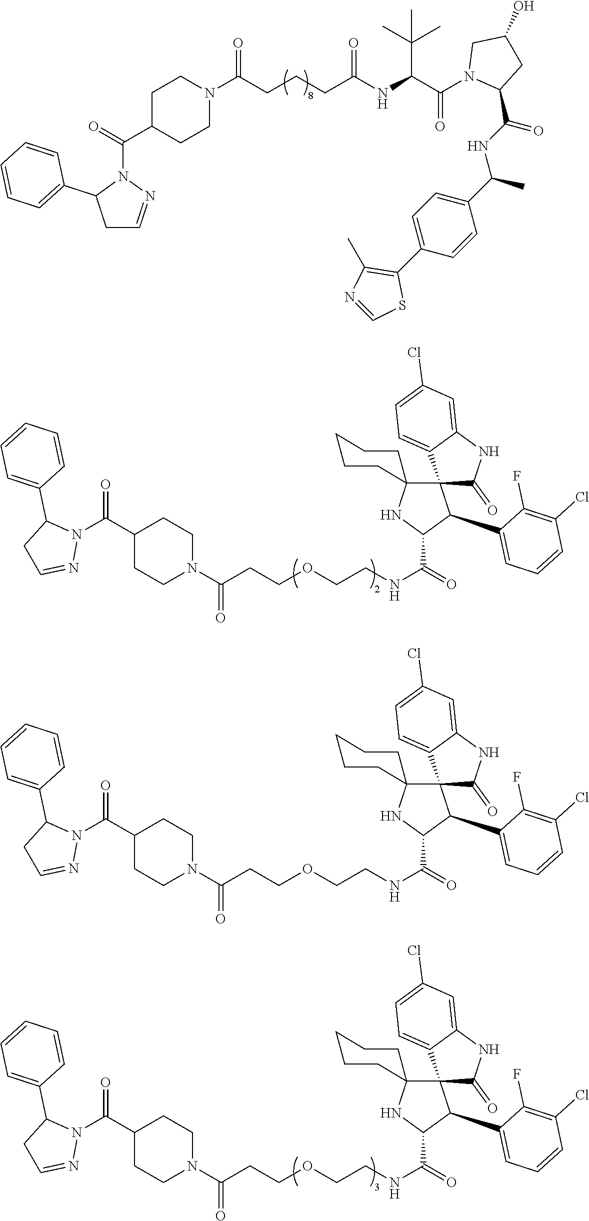

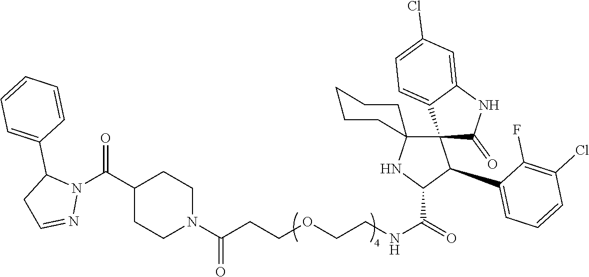

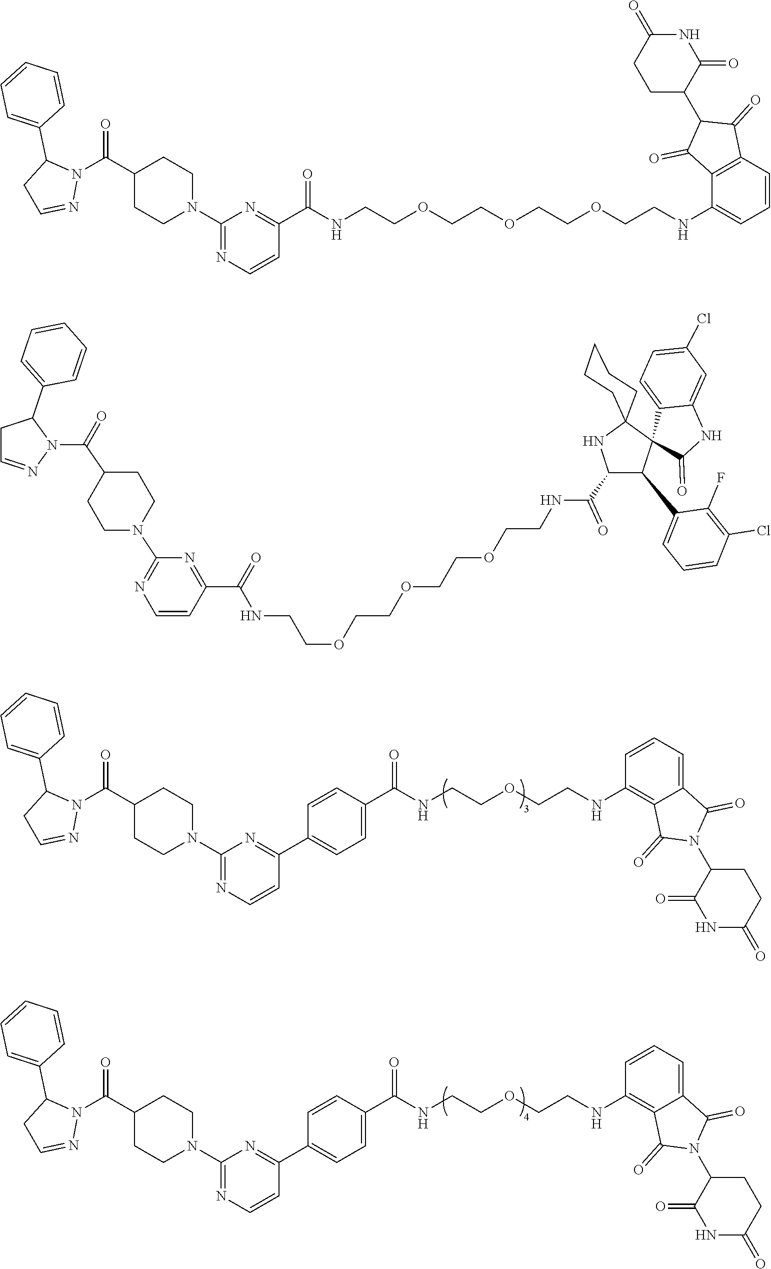

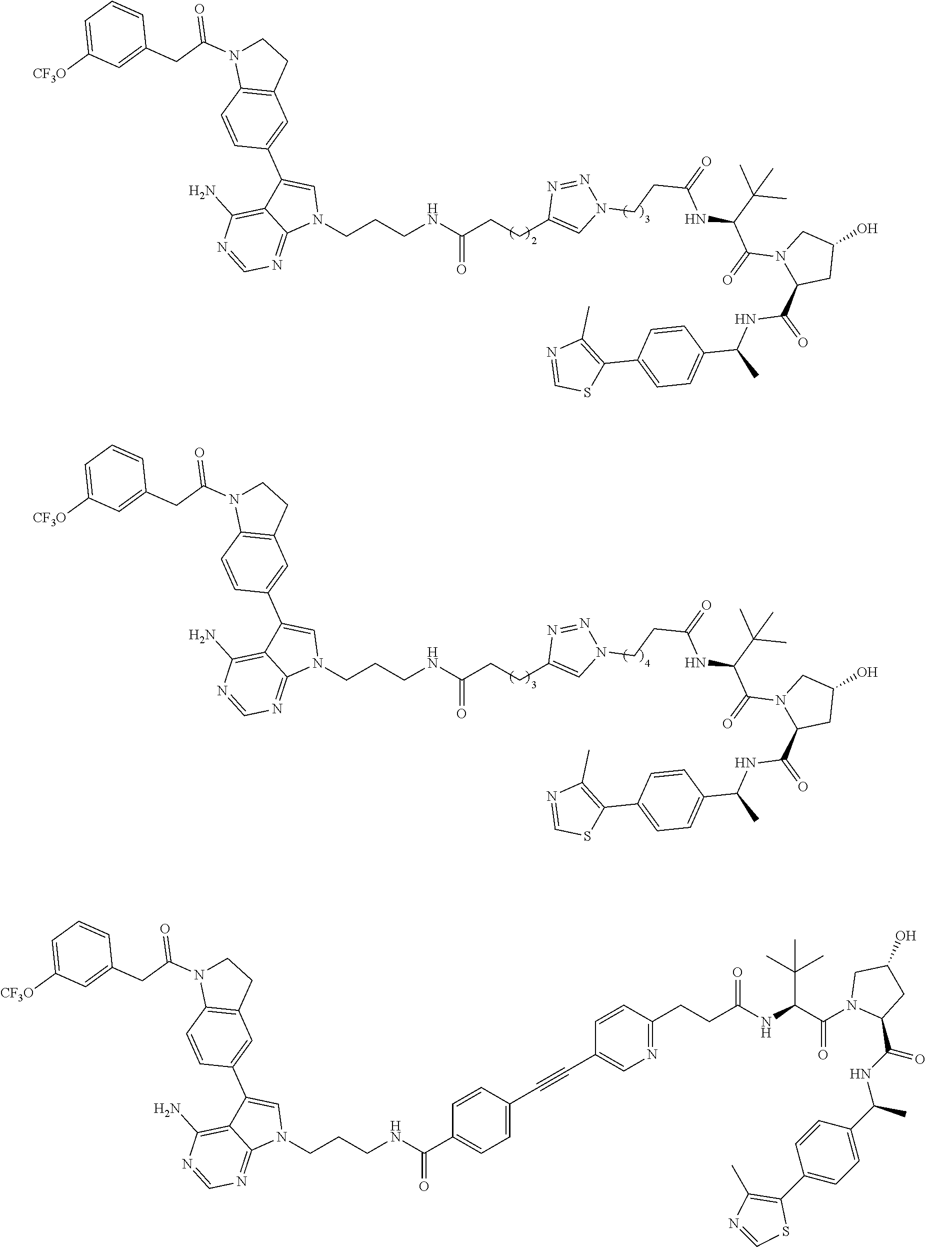

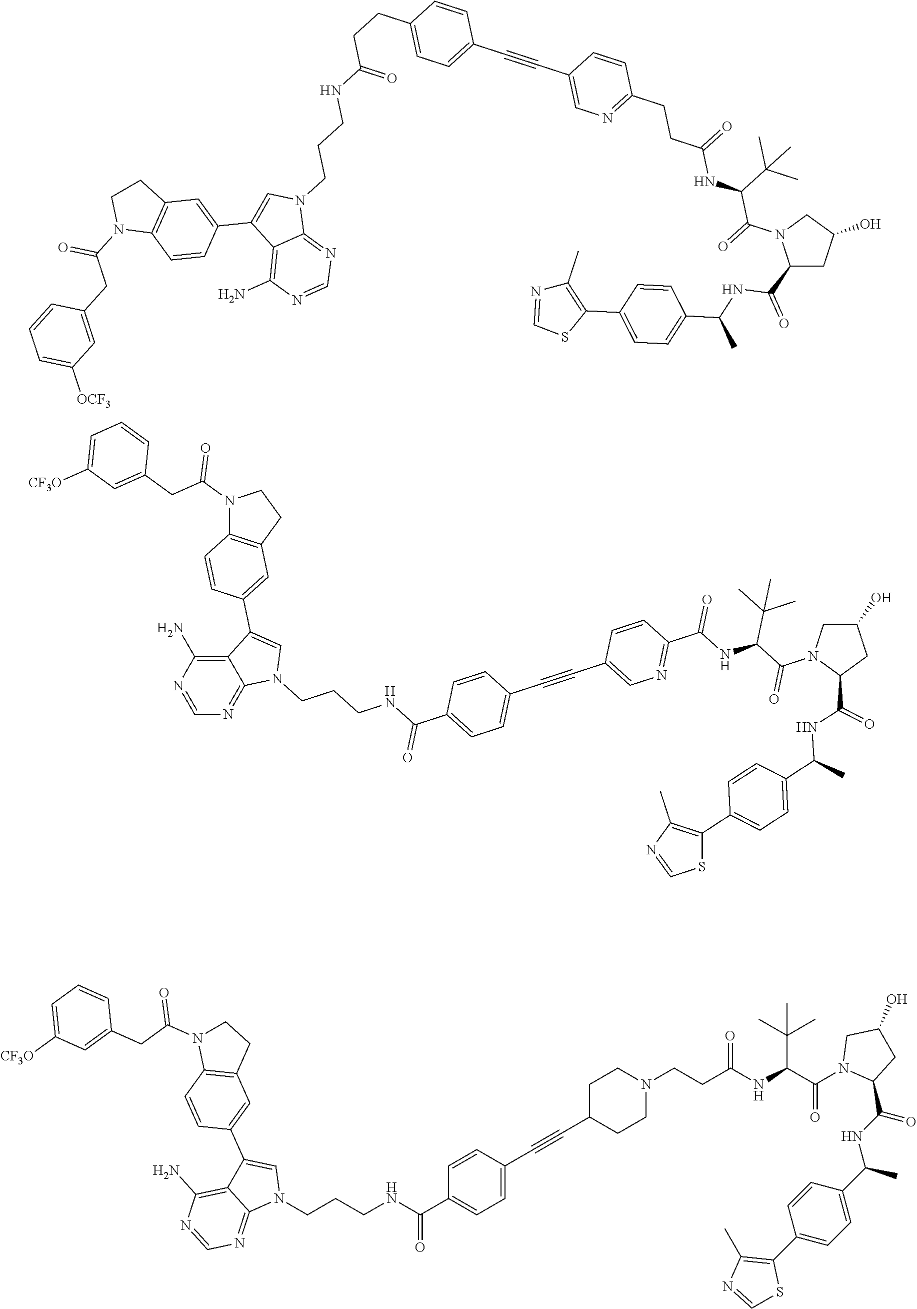

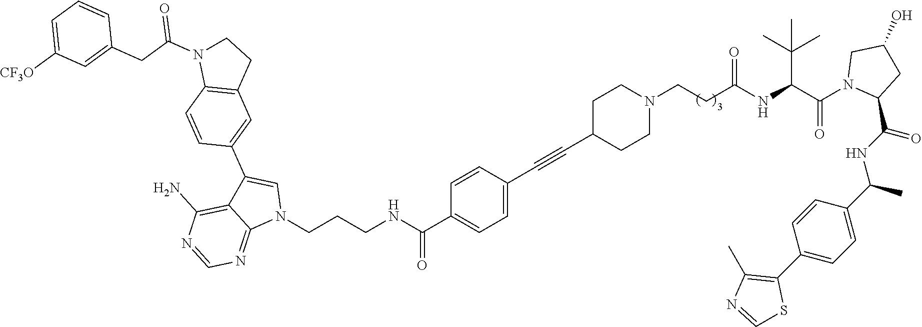

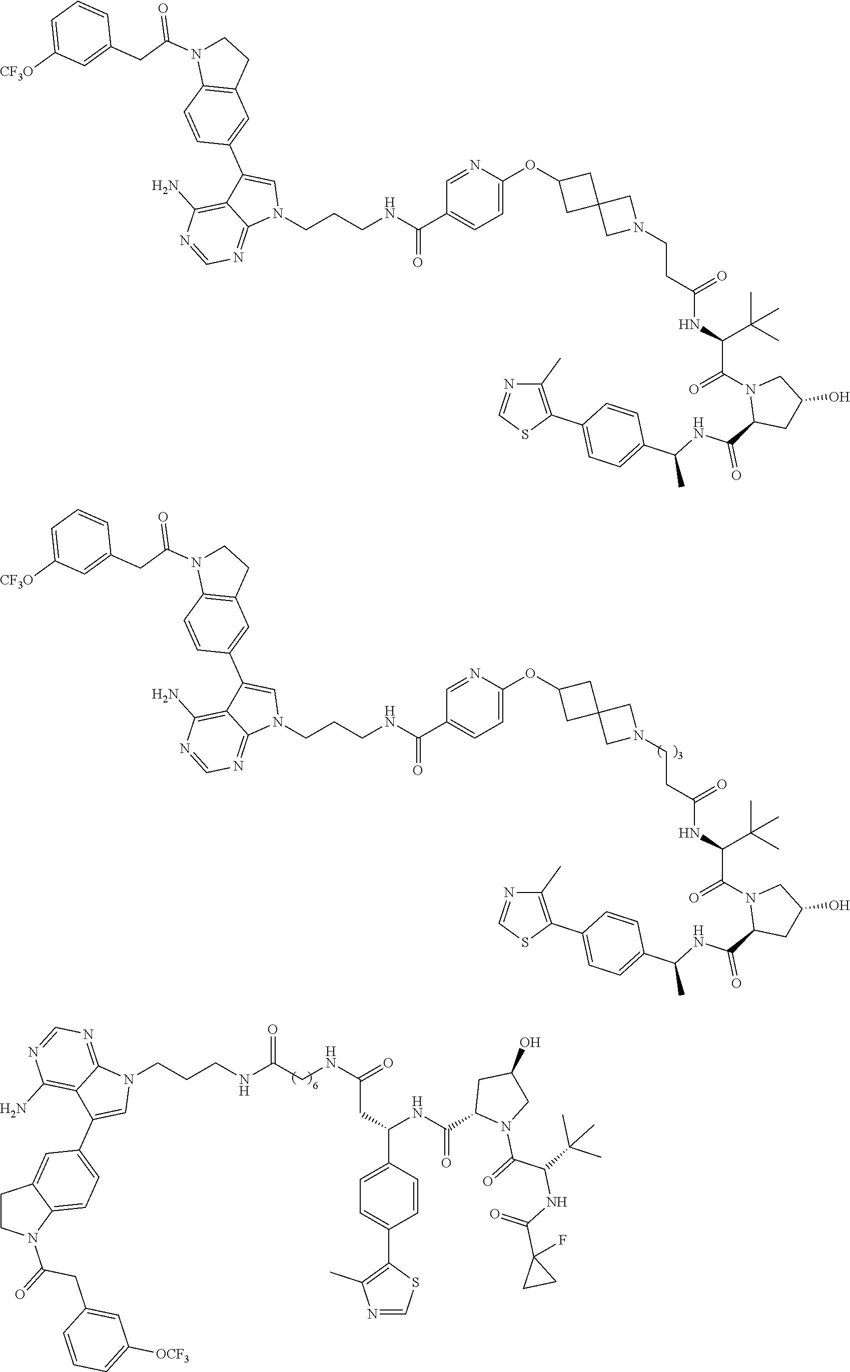

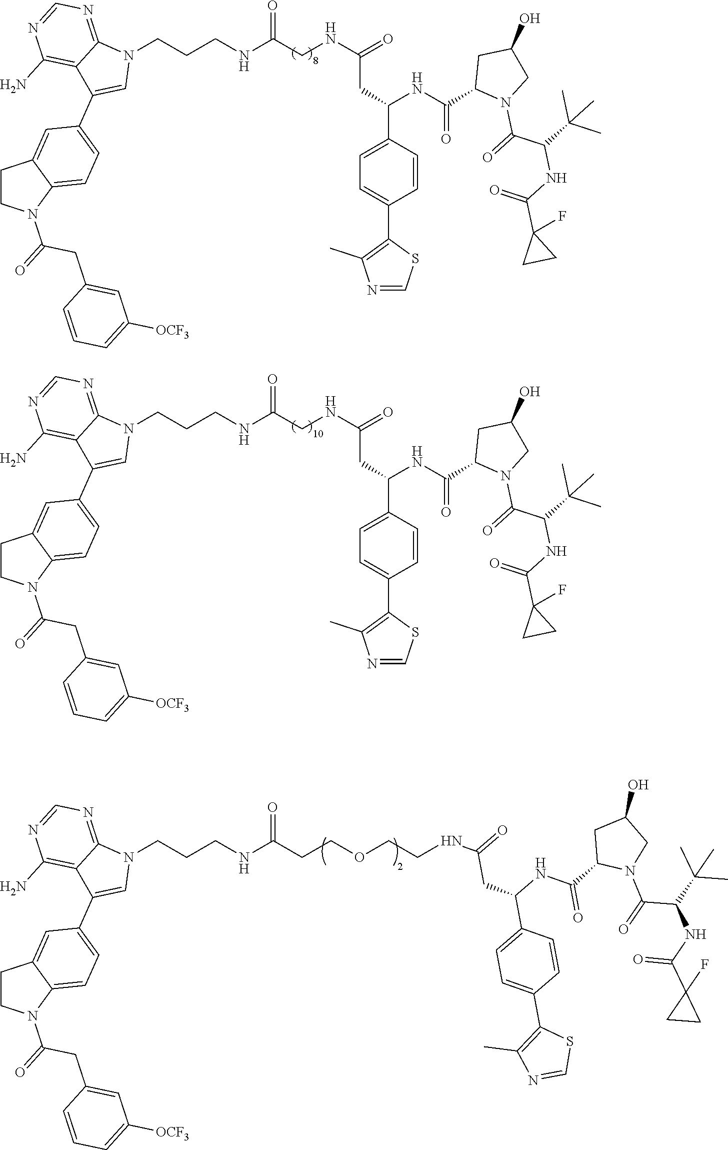

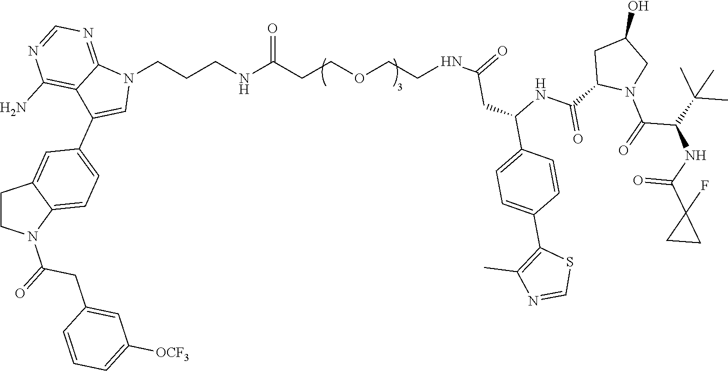

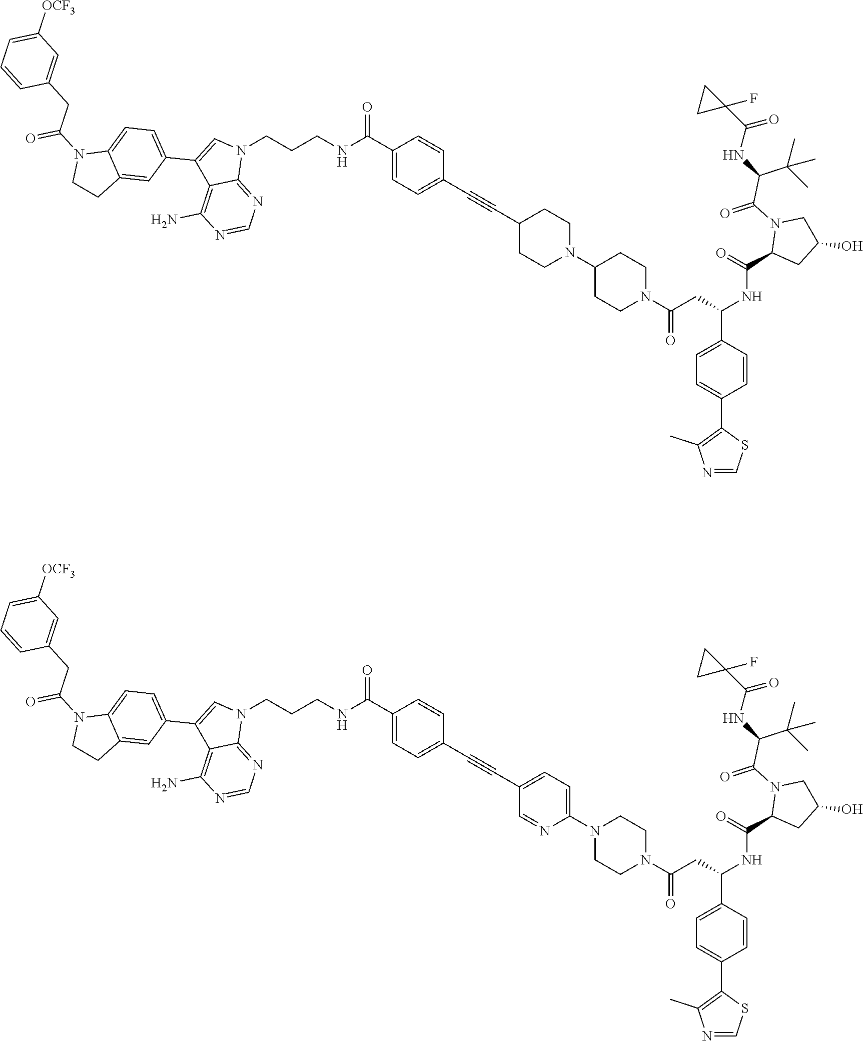 [SMILES: Cc1ncsc1-c1ccc([C@H](CC(=O)N2CCC(N3CCC(C#Cc4ccc(C(=O)NCCCn5cc(-c6ccc7c(c6)CCN7C(=O)Cc6cccc(OC(F)(F)F)c6)c6c(N)ncnc65)cc4)CC3)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)N2CCN(c3ccc(C#Cc4ccc(C(=O)NCCCn5cc(-c6ccc7c(c6)CCN7C(=O)Cc6cccc(OC(F)(F)F)c6)c6c(N)ncnc65)cc4)cn3)CC2)NC(=O)[C@@H]2C[C@@H](O)CN2C(=O)[C@@H](NC(=O)C2(F)CC2)C(C)(C)C)cc1]

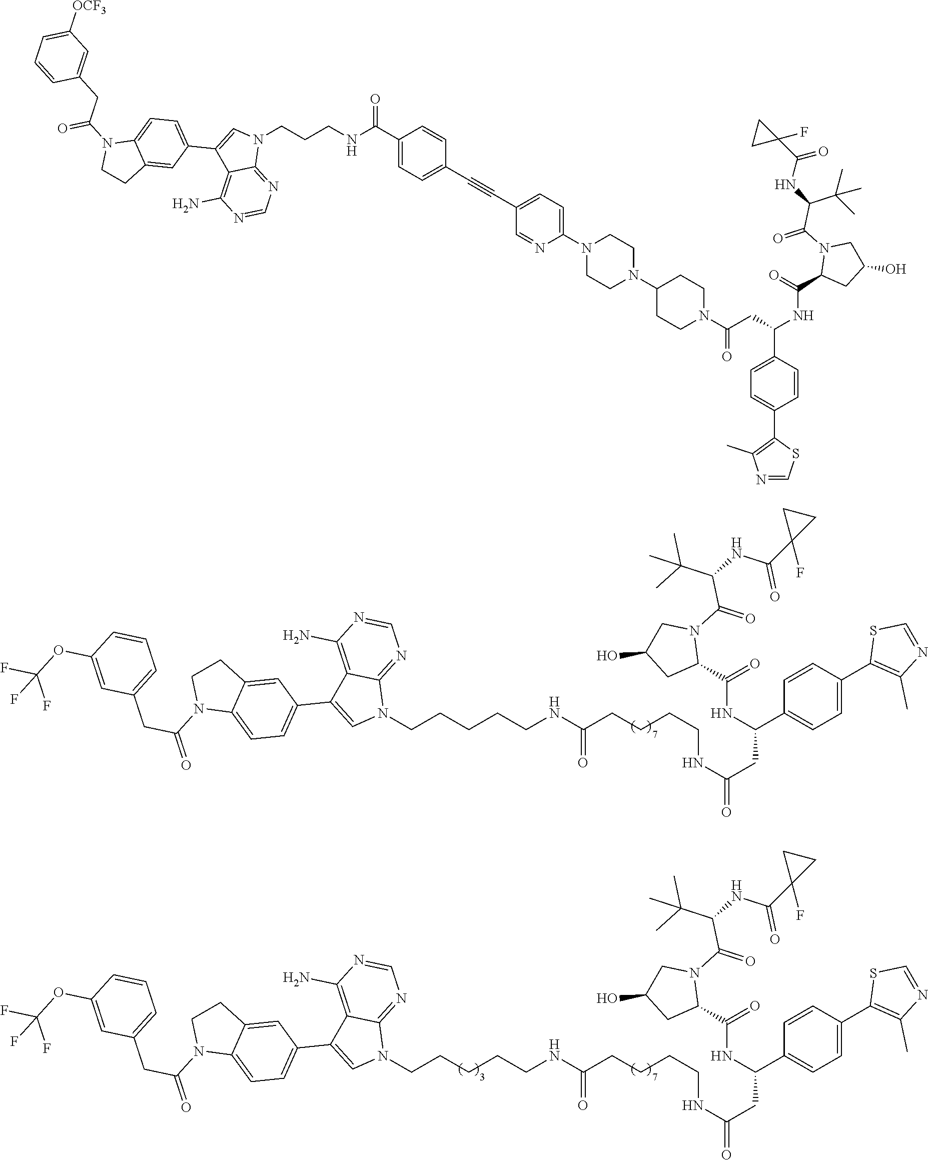

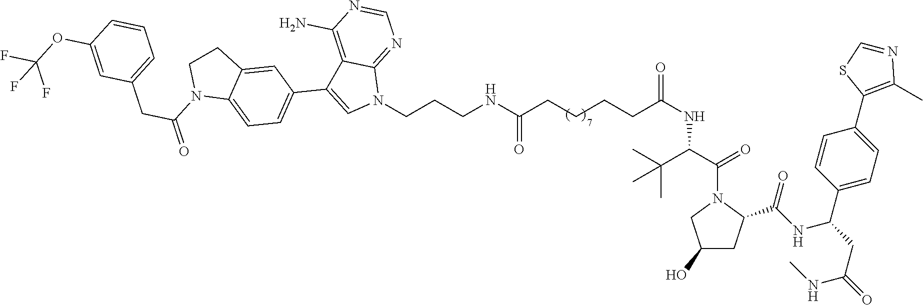

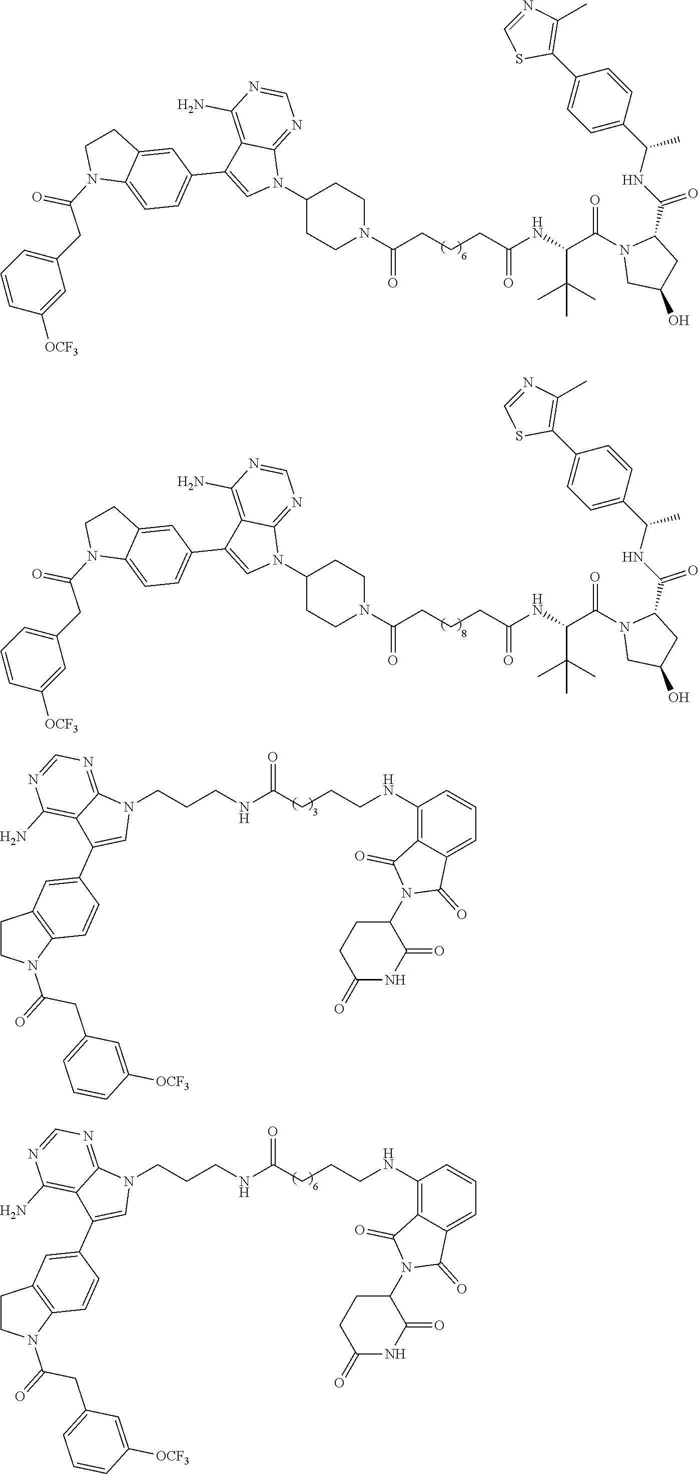

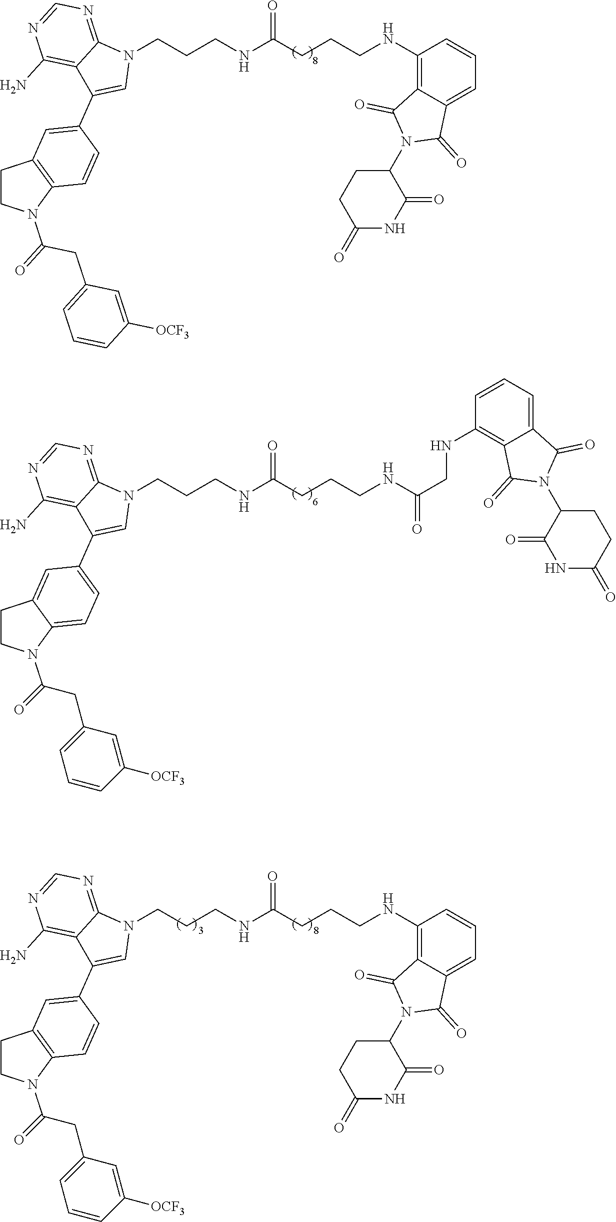

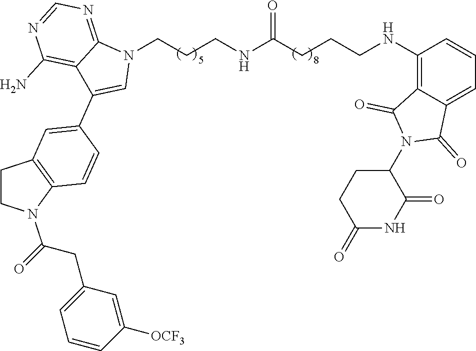

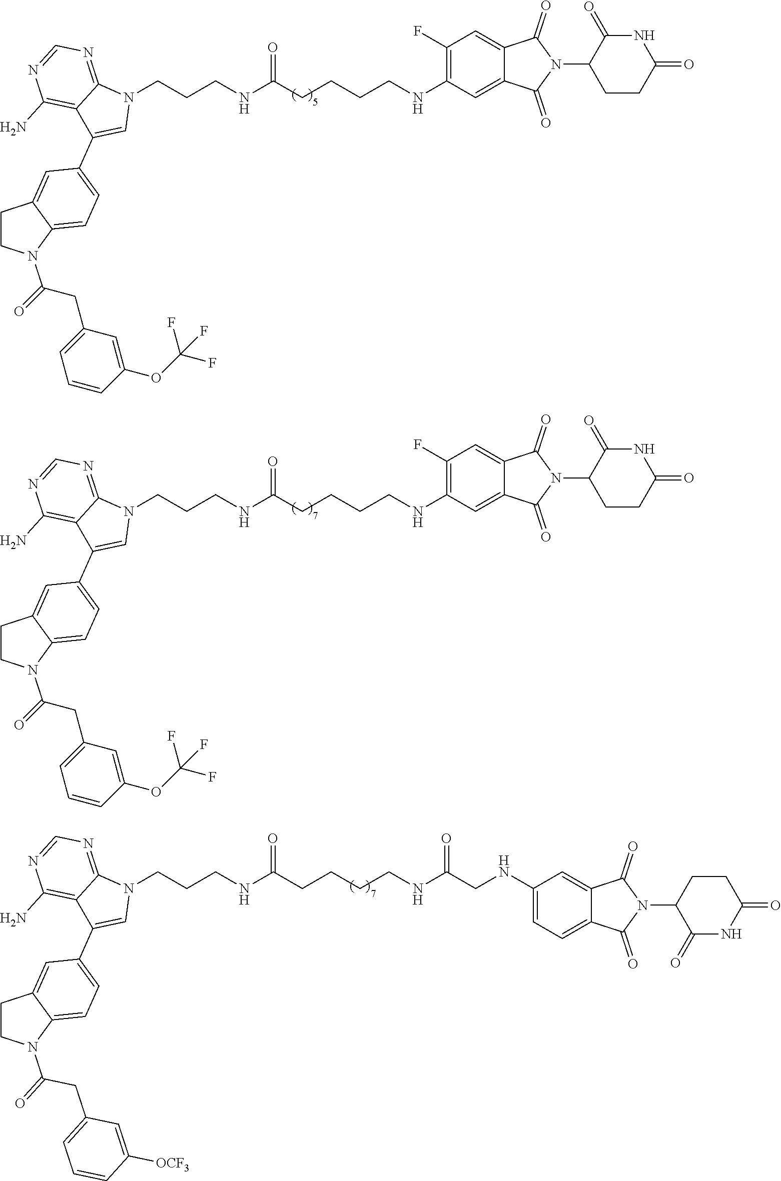

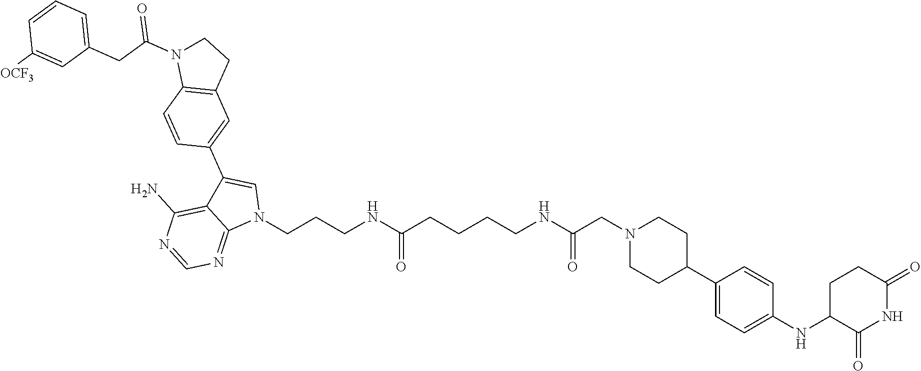 [SMILES: Nc1ncnc2c1c(-c1ccc3c(c1)CCN3C(=O)Cc1cccc(OC(F)(F)F)c1)cn2CCCNC(=O)CCCCNC(=O)CN1CCC(c2ccc(NC3CCC(=O)NC3=O)cc2)CC1]

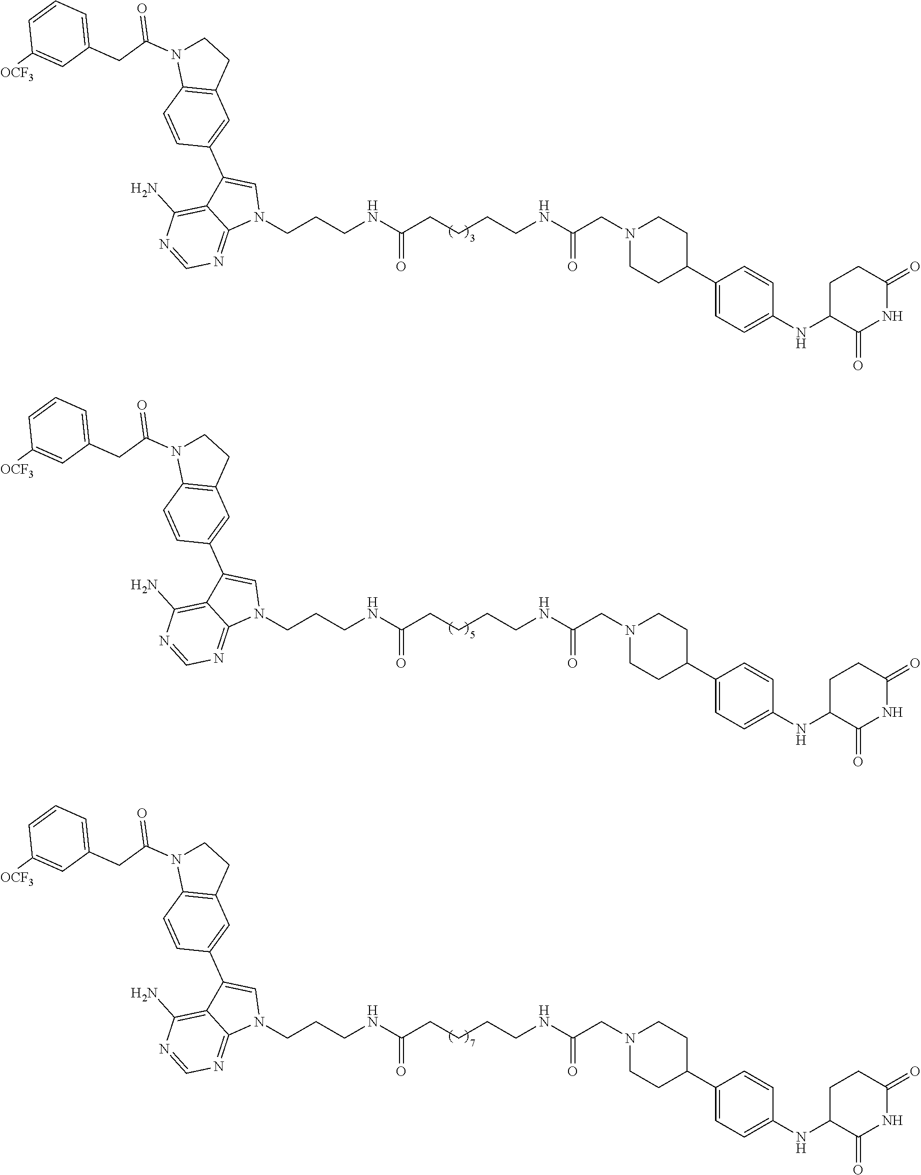

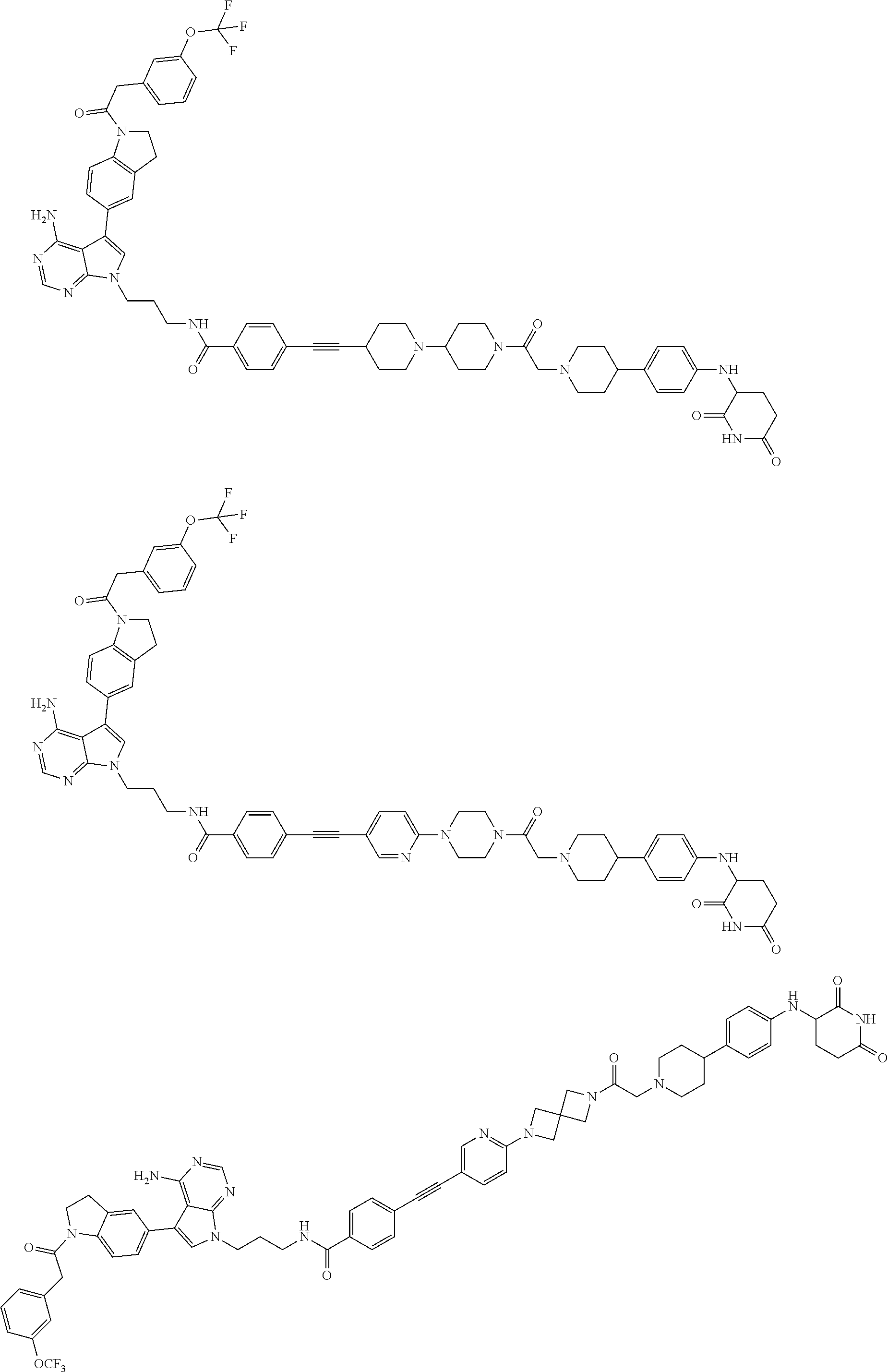 [SMILES: Nc1ncnc2c1c(-c1ccc3c(c1)CCN3C(=O)Cc1cccc(OC(F)(F)F)c1)cn2CCCNC(=O)c1ccc(C#CC2CCN(C3CCN(C(=O)CN4CCC(c5ccc(NC6CCC(=O)NC6=O)cc5)CC4)CC3)CC2)cc1 ; Nc1ncnc2c1c(-c1ccc3c(c1)CCN3C(=O)Cc1cccc(OC(F)(F)F)c1)cn2CCCNC(=O)c1ccc(C#Cc2ccc(N3CCN(C(=O)CN4CCC(c5ccc(NC6CCC(=O)NC6=O)cc5)CC4)CC3)nc2)cc1 ; Nc1ncnc2c1c(-c1ccc3c(c1)CCN3C(=O)Cc1cccc(OC(F)(F)F)c1)cn2CCCNC(=O)c1ccc(C#Cc2ccc(N3CC4(CN(C(=O)CN5CCC(c6ccc(NC7CCC(=O)NC7=O)cc6)CC5)C4)C3)nc2)cc1]

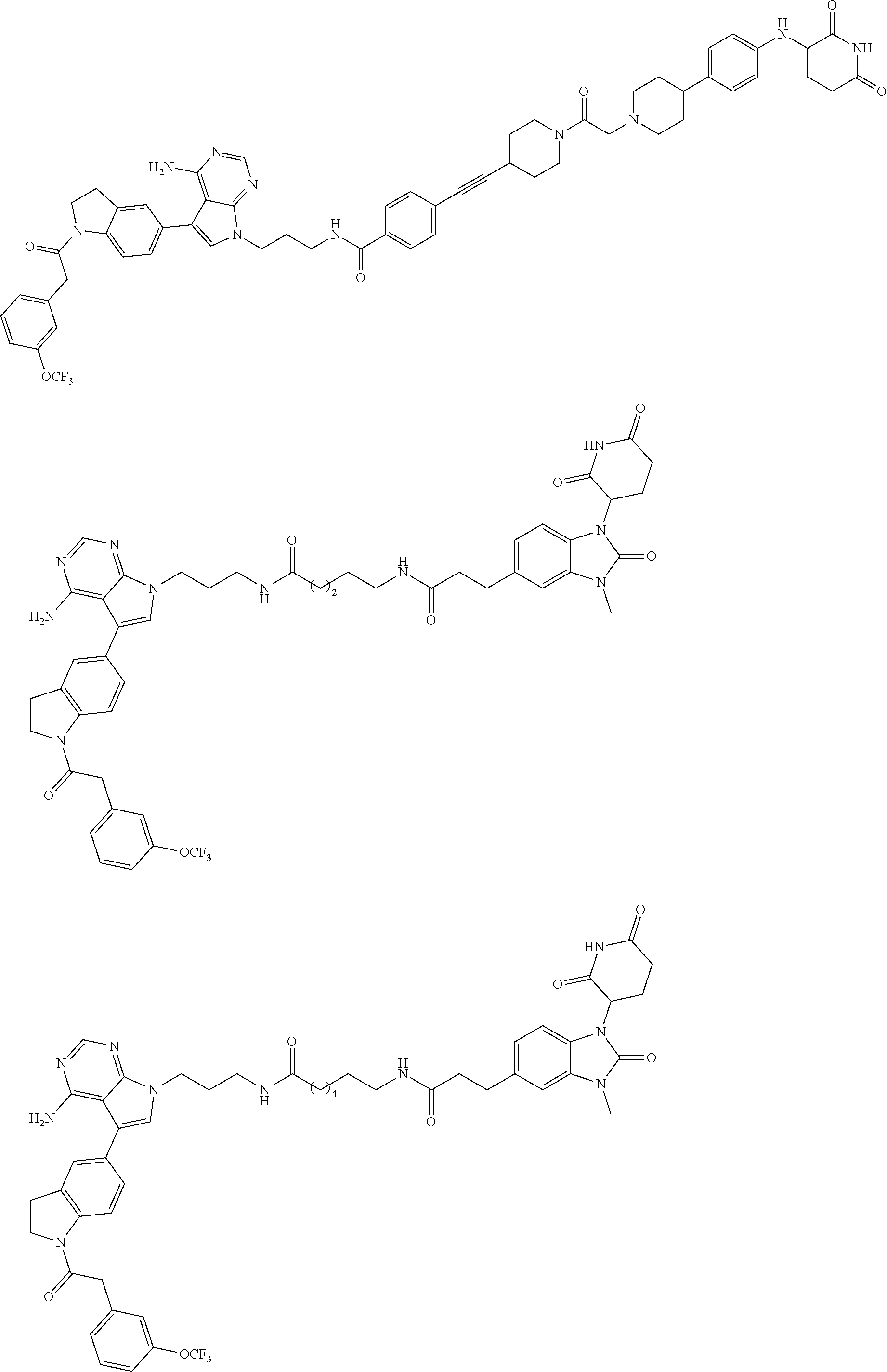

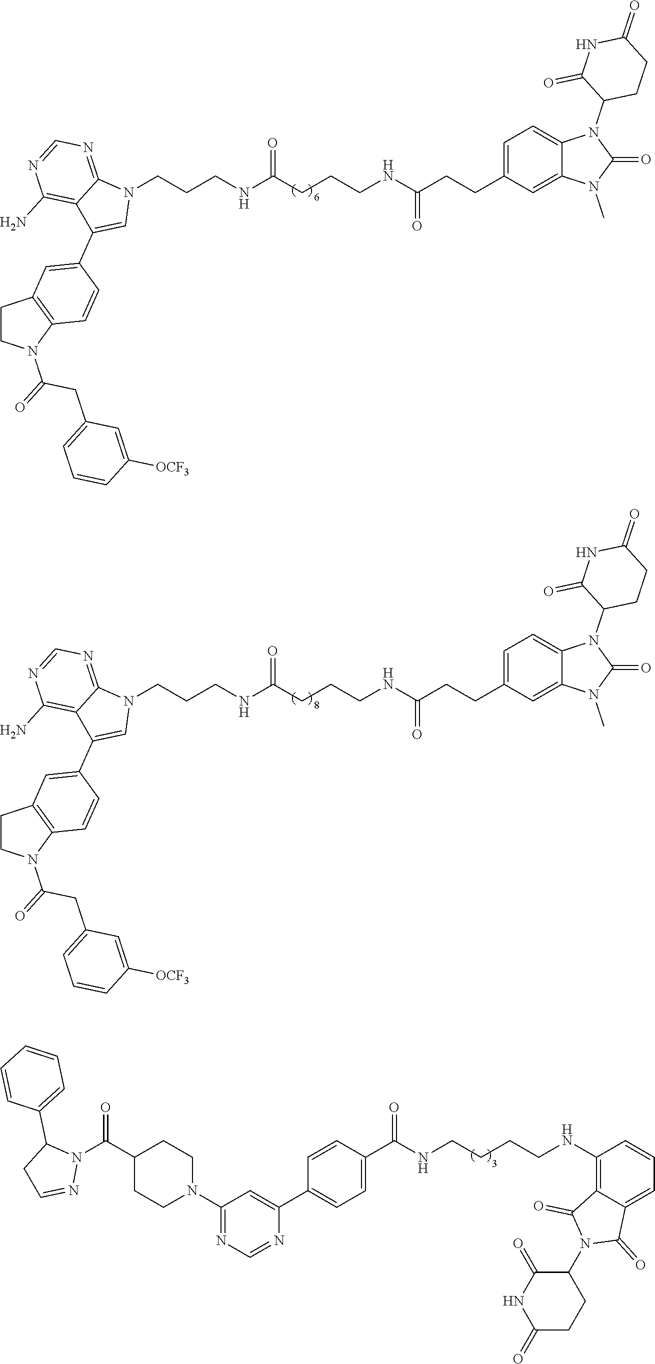

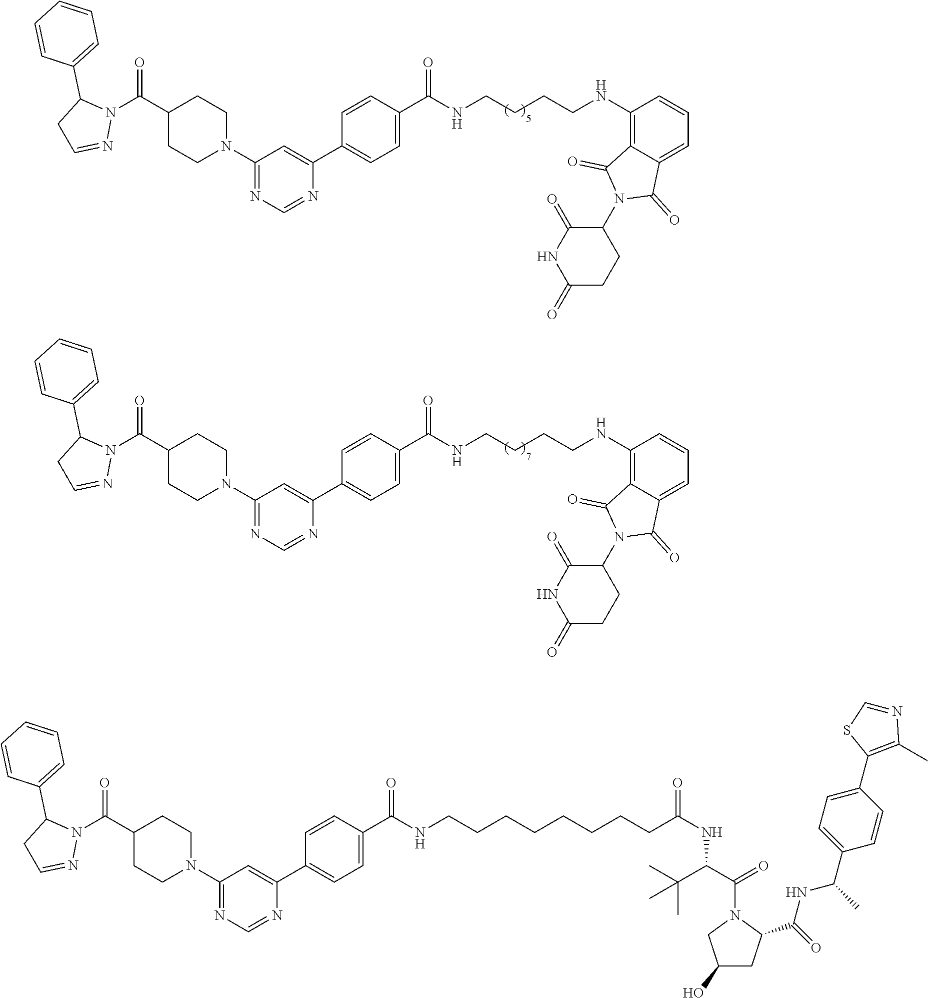

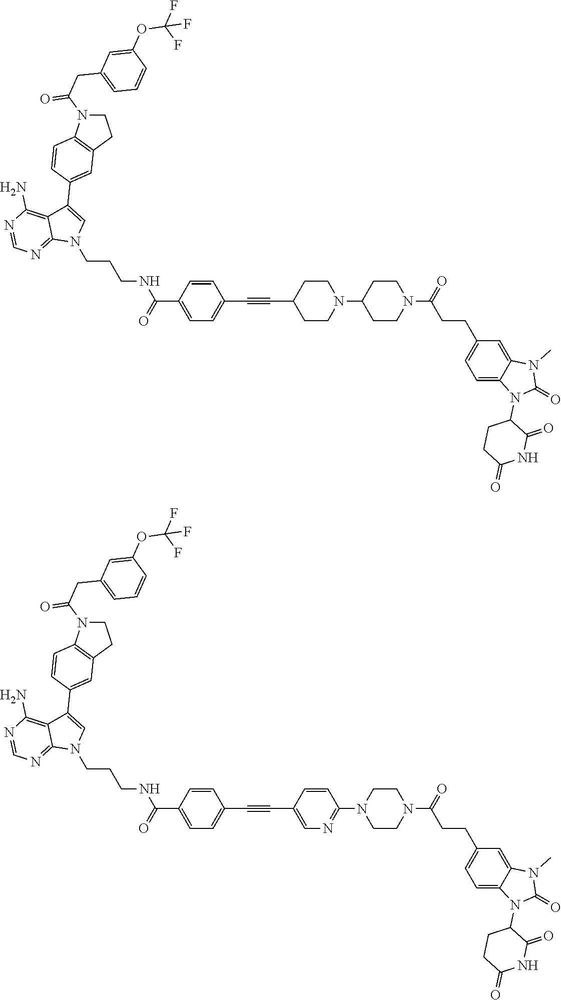 [SMILES: Cn1c(=O)n(C2CCC(=O)NC2=O)c2ccc(CCC(=O)N3CCC(N4CCC(C#Cc5ccc(C(=O)NCCCn6cc(-c7ccc8c(c7)CCN8C(=O)Cc7cccc(OC(F)(F)F)c7)c7c(N)ncnc76)cc5)CC4)CC3)cc21 ; Cn1c(=O)n(C2CCC(=O)NC2=O)c2ccc(CCC(=O)N3CCN(c4ccc(C#Cc5ccc(C(=O)NCCCn6cc(-c7ccc8c(c7)CCN8C(=O)Cc7cccc(OC(F)(F)F)c7)c7c(N)ncnc76)cc5)cn4)CC3)cc21]

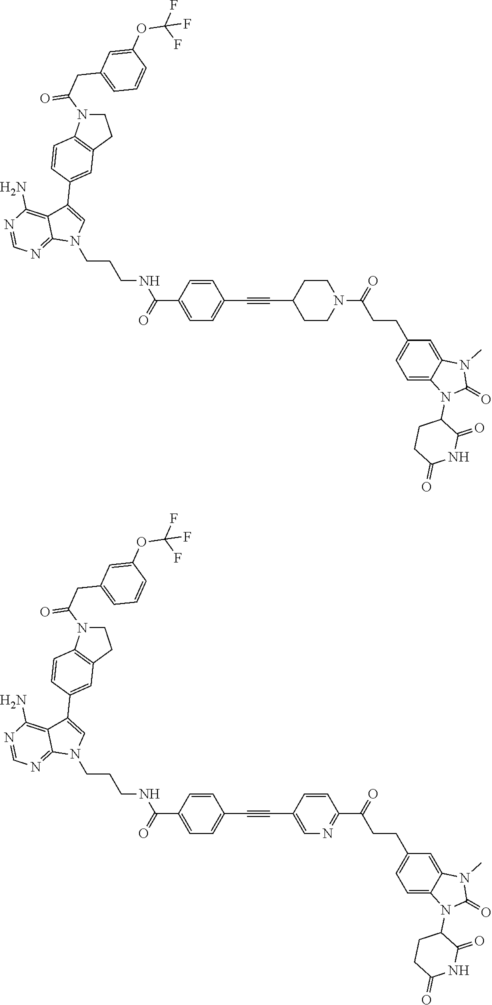 [SMILES: Cn1c(=O)n(C2CCC(=O)NC2=O)c2ccc(CCC(=O)N3CCC(C#Cc4ccc(C(=O)NCCCn5cc(-c6ccc7c(c6)CCN7C(=O)Cc6cccc(OC(F)(F)F)c6)c6c(N)ncnc65)cc4)CC3)cc21 ; Cn1c(=O)n(C2CCC(=O)NC2=O)c2ccc(CCC(=O)c3ccc(C#Cc4ccc(C(=O)NCCCn5cc(-c6ccc7c(c6)CCN7C(=O)Cc6cccc(OC(F)(F)F)c6)c6c(N)ncnc65)cc4)cn3)cc21]

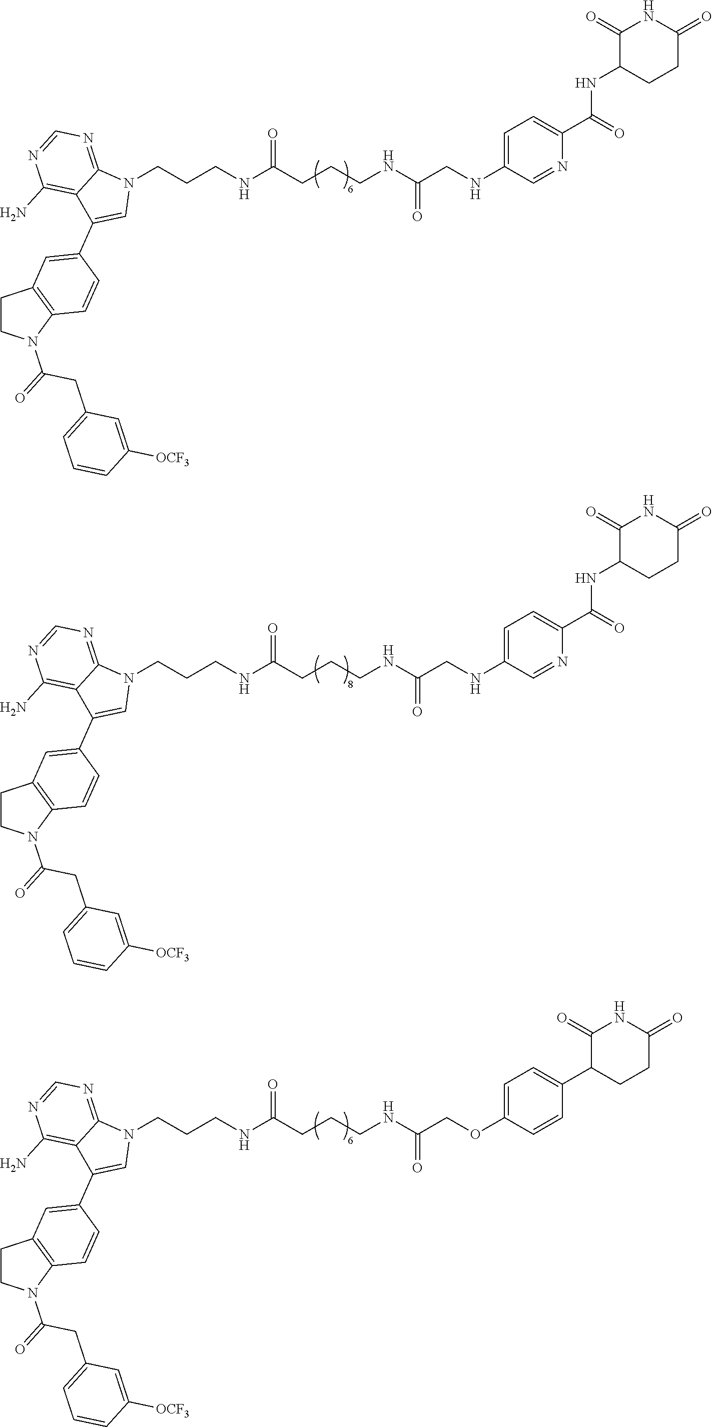

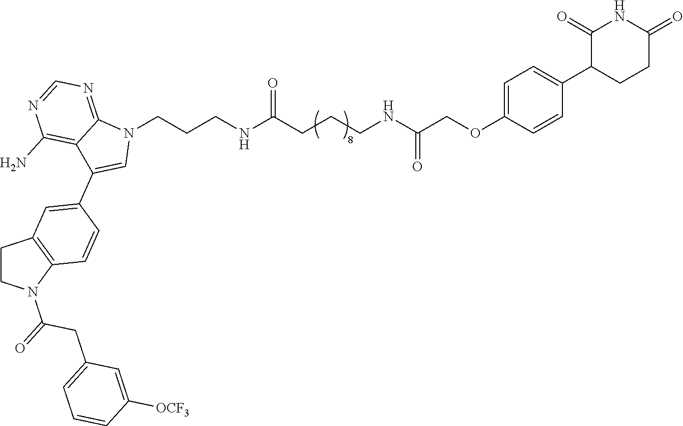

Also described herein is a pharmaceutical composition comprising a compound as described herein and a pharmaceutically acceptable carrier.

Further described herein is a kit comprising a compound or composition as described herein.

Methods of treating or preventing a RIPK1 kinase-related disease in a subject are also provided herein. A method of treating or preventing a RIPK1 kinase-related disease in a subject comprises administering to the subject an effective amount of a compound or pharmaceutical composition as described above. Optionally, the RIPK1 kinase-related disease is cancer (e.g., bladder cancer, blood cancer, a bone marrow cancer, brain cancer, breast cancer, bronchus cancer, colorectal cancer, cervical cancer, chondrosarcoma, endometrial cancer, gastrointestinal cancer, gastric cancer, genitourinary cancer, head and, neck cancer, hepatic cancer, hepatocellular carcinoma, leukemia, liver cancer, lung cancer, lymphoma, melanoma, of the skin, ovarian cancer, pancreatic cancer, prostate cancer, renal cancer, skin cancer, testicular cancer, thyroid cancer, or uterine cancer).

Optionally, the RIPK1 kinase-related disease is an inflammatory disease (e.g., neuroinflammation, asthma, chronic obstructive pulmonary disorder (COPD), chronic bronchitis, cystic fibrosis, atherosclerosis, post-angioplasty, restenosis, coronary artery diseases, angina, rheumatoid arthritis, osteoarthritis, dermatitis, eczematous dermatitis, psoriasis, post transplantation late and chronic solid organ rejection, systemic lupus erythematosis, dermatomyositis, polymyositis, Sjogren's syndrome, polymyalgia rheumatica, temporal arteritis, Behcet's disease, Guillain Barre syndrome, Wegener's granulomatosus, polyarteritis nodosa, an inflammatory neuropathy, vasculitis, an inflammatory disorder of adipose tissue, Kaposi's sarcoma, or a smooth muscle cell proliferative disorder).

In some cases, the RIPK1 kinase-related disease is a neurodegenerative disorder (e.g., Alexander disease, Alper's disease, Alzheimer's disease, amyotrophic lateral sclerosis, ataxia telangiectasia, Batten disease, Canavan disease, Cockayne syndrome, corticobasal degeneration, Creutzfeldt-Jakob disease, Huntington's disease, Kennedy's disease, Krabbe disease, Lewy body dementia, Machado-Joseph disease, spinocerebellar ataxia type 3, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinocerebellar ataxia, spinal muscular atrophy, Steele-Richardson-Olszewski disease, Tay-Sachs, transmissible spongiform encephalopathies (TSE), or tabes dorsalis).

Optionally, the methods further comprise administering to the subject a second compound, biomolecule, or composition. Optionally, the second compound, biomolecule, or composition is an anti-inflammatory agent. The methods can further comprise administering the compound or the pharmaceutical composition as described herein orally, intraperitoneally, sublingually, subcutaneously, intravenously, or any clinically acceptable administration route.

Methods of degrading or inhibiting an RIPK1 kinase in a cell are also provided herein. A method of degrading or inhibiting an RIPK1 kinase in a cell includes contacting a cell with an effective amount of a compound or pharmaceutical composition as described herein. Optionally, the contacting can be performed in vitro or in vivo.

The details of one or more embodiments are set forth in the description below. Other features, objects, and advantages will be apparent from the description and from the claims.

### DETAILED DESCRIPTION

The present disclosure relates to bifunctional compounds that bind RIPK1 kinase and promote targeted ubiquitination for the degradation of RIPK1 kinase. In particular, the present disclosure is directed to bifunctional compounds that can bind RIPK1 kinase and promote its degradation by recruiting an E3 ubiquitin ligase (e.g., VHL), which can ubiquitinate RIPK1 protein, marking it for proteasomal degradation. The present disclosure also provides radiolabeled forms of the bifunctional compounds and pharmaceutical compositions comprising the bifunctional compounds, methods of detecting and/or diagnosing a broad range of pharmacological activities associated with degradation/inhibition of RIPK1 kinase.

Diseases or disorders that result from RIPK1 kinase are treated or prevented with compounds and compositions of the present disclosure. In particular, RIPK1 knockout in tumor cells can synergize with checkpoint inhibitors in cancer immunotherapy. The RIPK1 degraders described herein can phenocopy RIPK1 knockout and improve current cancer immunotherapy. Additionally, patients with non-cleavable RIPK1 mutants have constant fevers and inflammation. The RIPK1 degraders described herein can degrade non-cleavable RIPK1 mutants to reverse the disease phenotype.

I. Compounds

The RIPK1 kinase degraders described herein are represented by the following formula:

TPM-L-ELM

and pharmaceutically acceptable salts, co-crystals, tautomers, stereoisomers, solvates, hydrates, polymorphs, isotopically enriched derivatives, and prodrugs thereof.

In the formula shown above, TPM is a targeting protein binder moiety; L is a bond or a chemical linker group; and ELM is an E3 ubiquitin ligase binding moiety. The L group is covalently bonded to the TPM group and the ELM group. The bifunctional compounds as described herein can be synthesized such that the number and position of the respective functional moieties can be varied.

Exemplary TPM groups are represented by the TPM-1 structure shown below:

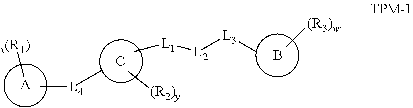

In TPM-1, Ring A, Ring B, and Ring C are each independently selected from aryl, heteroaryl, cycloalkyl, and heterocycloalkyl. For example, Ring A, Ring B, and/or Ring C can be a 5-10 membered aryl ring or a heterocyclic ring. In some embodiments, Ring A is selected from the following:

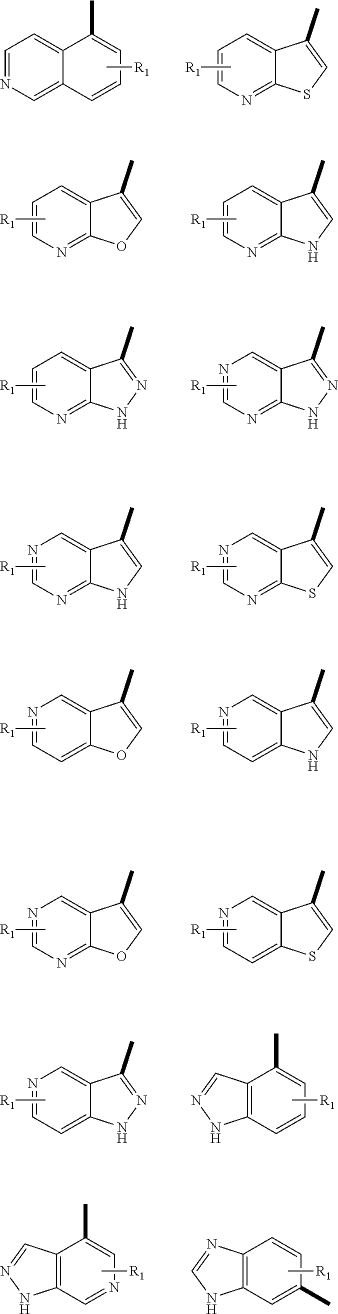

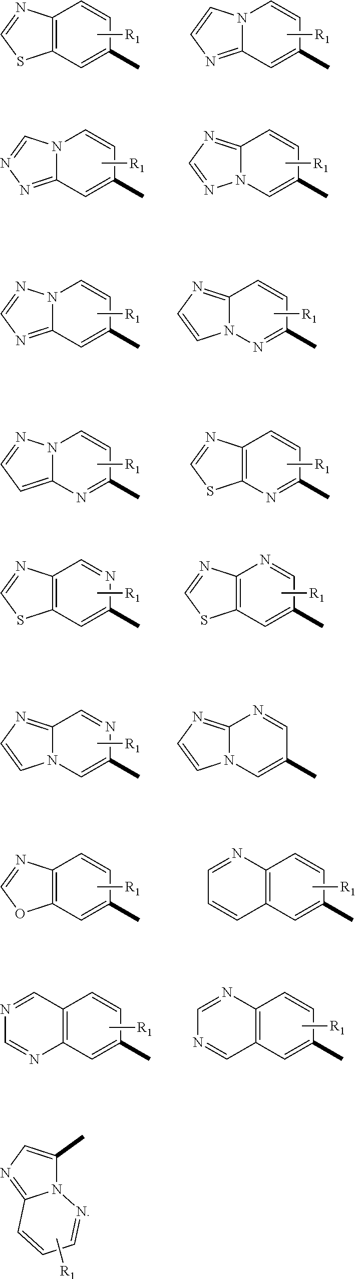

In some embodiments, Ring B and Ring C are each independently phenyl or a 6-10 membered heterocycle ring.

Also in TPM-1, L₁ and L₃ are each independently a bond, —N(R₁)—, —CH(R₁)—, or

Additionally in TPM-1, L₂ is —S(O)—, —S(O)₂—, or —C(O)—.

Further in TPM-1, L₄ is a bond or a C₁₋₃ alkyl.

In TPM-1, R₁, R₂, and R₃ are each independently hydrogen, halogen, —OH, —OR₄, —NH₂, —NR₄R₅, —NR₄COR₅, —CN, —COOH, —COOR₄, —CONR₄R₅, —CF₃, —OCF₃, —SO₃H, —SO₃R₄, tetrazole, aryl, aryl substituted with from one to three substituents independently selected from halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, and —R₄, heterocycle, heterocycle substituted with from one to three substituents independently selected from halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, and —R₄. Optionally, R₂ and R₃ are each independently hydrogen, halogen, —OH, —OR₄, —NH₂, —NR₄R₅, —NR₄COR₅, —CN, —COOH, —COOR₄, —CONH₂, —CONR₄R₅, —CF₃, OCF₃, —SO₃H, —SO₃H₄, and —R₄.

Also in TPM-1, R₄ and R₅ are each independently C₁₋₆ alkyl, wherein one or more carbon is optionally replaced with halo, —CO—, —CONH—, —O—, —S—, —SO—, —SO₂—, —N—, —NHCO—, or a heterocycle.

Additionally in TPM-1, x, y, and w are each independently an integer from 0 to 4. In some cases, x, y, and/or w are each independently an integer from 0 to 3.

Optionally, the TPM-1 group is selected from the group consisting of:

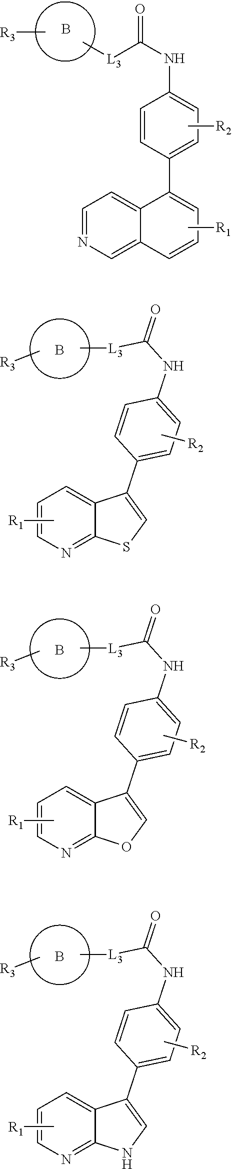

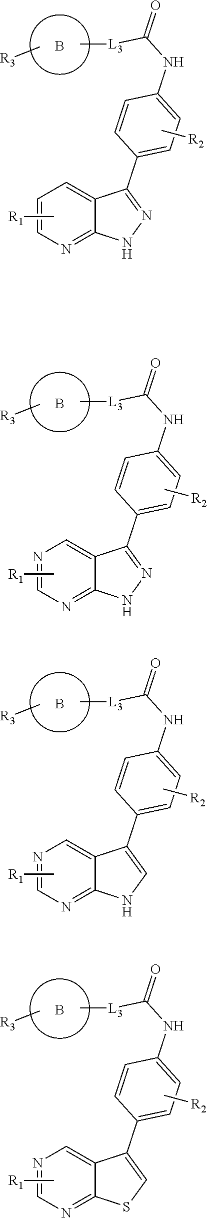

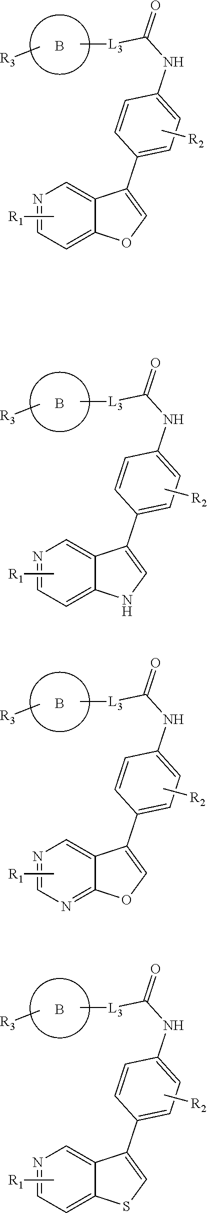

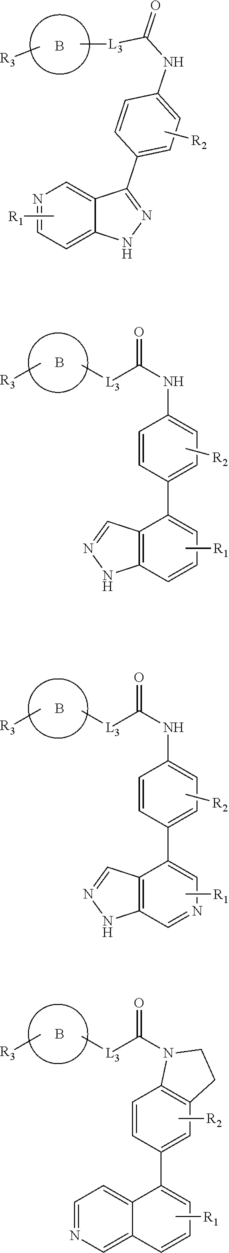

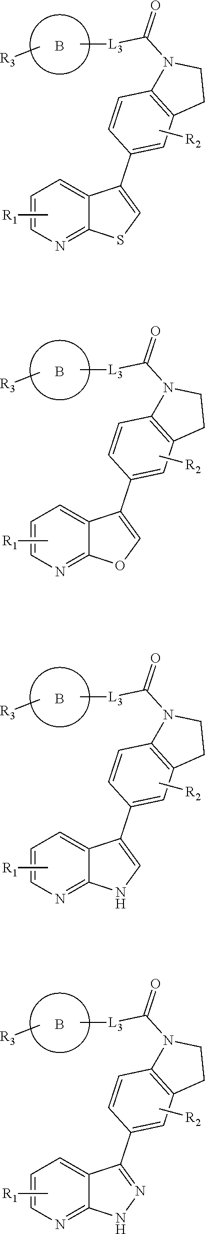

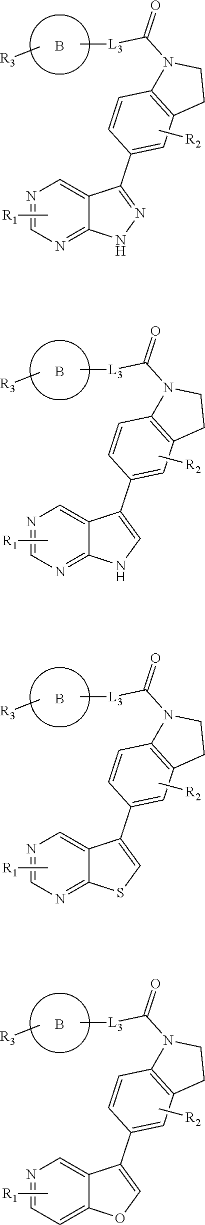

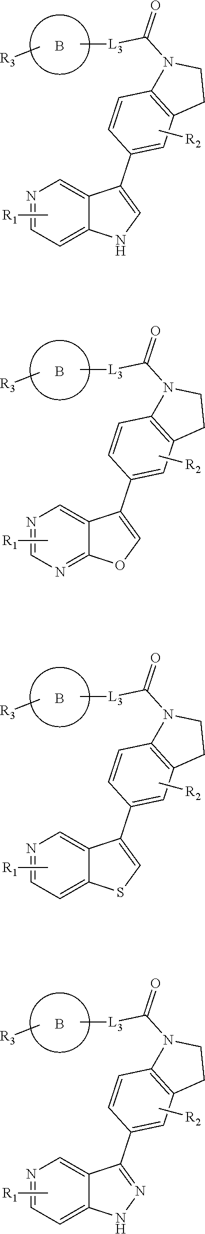

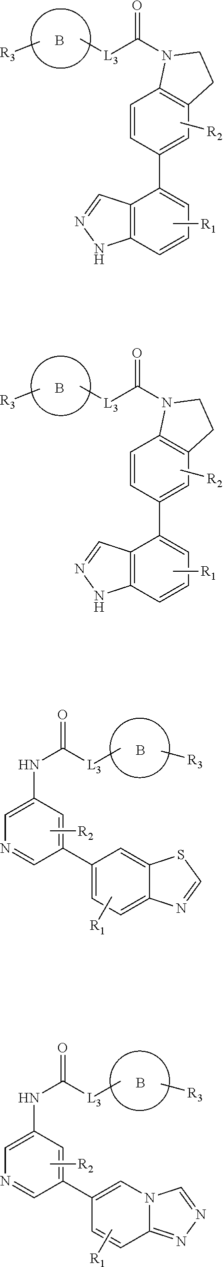

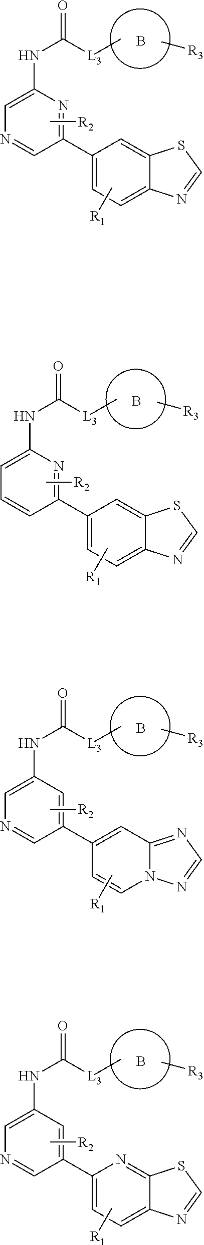

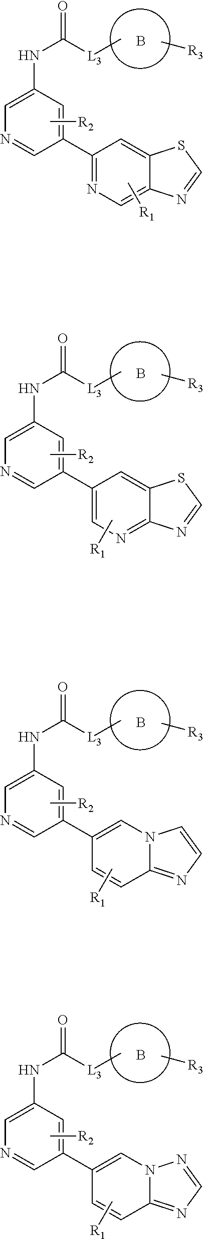

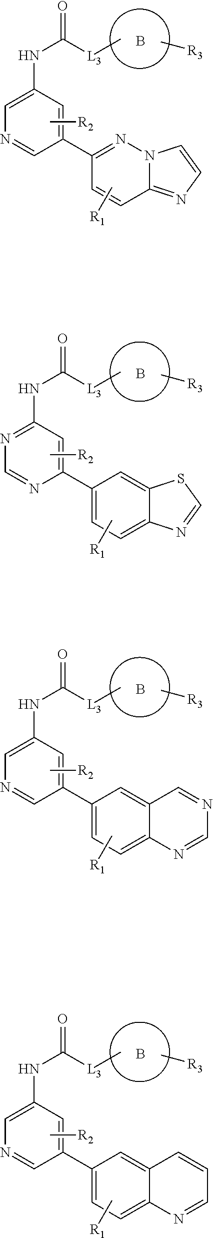

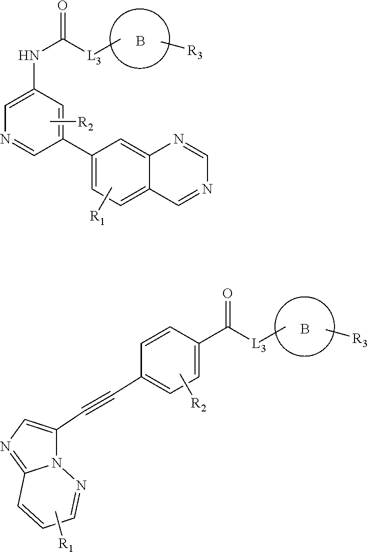

Exemplary TPM groups are represented by the TPM-2 structure shown below:

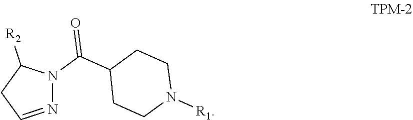

In TPM-2, R₁ is selected from R₃, 6-10 membered aryl, 6-10 membered aryl substituted by one or two R₃, 5-10 membered heteroaryl, or 5-10 membered heteroaryl substituted by one or two R₃. In some examples, R₁ is selected from R₃, phenyl, phenyl substituted by one or two R₃, 5-6 membered heteroaryl, 5-6 membered heteroaryl substituted by one or two substituents R₃, 5-6 membered saturated ring, 5-6 membered saturated ring substituted by one to three substituents selected from halogen, —CN, and R₄.

Also in TPM-2, R₂ is selected from phenyl, phenyl substituted by one to three substituents selected from halogen, —CN, and R₄, 5-6 membered heteroaryl, 5-6 membered heteroaryl substituted by one to three substituents selected from halogen; —CN, and R₄, 5-6 membered saturated ring, and 5-6 membered saturated ring substituted by one to three substituents selected from halogen, —CN, and R₄. In some examples, R₂ is selected from phenyl, phenyl substituted by one to three substituents selected from halogen, —CN, and 5-6 membered heteroaryl, or 5-6 membered heteroaryl substituted by one to three substituents selected from halogen, and —CN.

Additionally in TPM-2, each R₃ is independently hydrogen, halogen, —OH, —OR₄, —NH₂, —NR₄R₅, —NR₄COR₅, —CN, —COOH, —COOR₄, —CONH₂, —CONR₄R₅, —CF₃, OCF₃, —SO₃H, —SO₃R₄, R₄, tetrazole, aryl, aryl substituted with from one to three substituents independently selected from halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, and R₄, heterocycle, and heterocycle substituted with from one to three substituents independently selected from halogen, —OH, —OR₄, —NH₂, —NR₄R₅, —NR₄COR₅, —CN, —COOH, —COOR₄, —CONH₂, —CONR₄R₅, —CF₃, —SO₃H, —SO₃R₄, and R₄.

Optionally, each R₃ is independently selected from the group consisting of hydrogen, halogen, —OH, —NH₂, —NHCOR₄, —CN, —COOH, —COOR₄, —CONH₂, —CONH₂R₄, —CF₃, —OCF₃, —SO₃H, —R₄, tetrazole, aryl, aryl substituted with from one to three substituents independently selected from halo, —OH, —CN, —COOH, —CONH₂, —CONH₂R₄, —CF₃, —OCF₃, —SO₃H, R₄, heterocycle, and heterocycle substituted with from one to three substituents independently selected from halo, —OH, —NH₂, —CN, —COOH, —CONH₂, —CONH₂R₄, —CF₃, —OCF₃, —SO₃H, and —R₄.

Further in TPM-2, R₄ and R₅ are each independently C₁-6 alkyl, wherein one or more carbons are optionally replaced with halo, CO, CONH, O, S, SO, SO₂, N, NHCO, or heterocycle.

In some cases, the TPM-2 group is selected from the group consisting of:

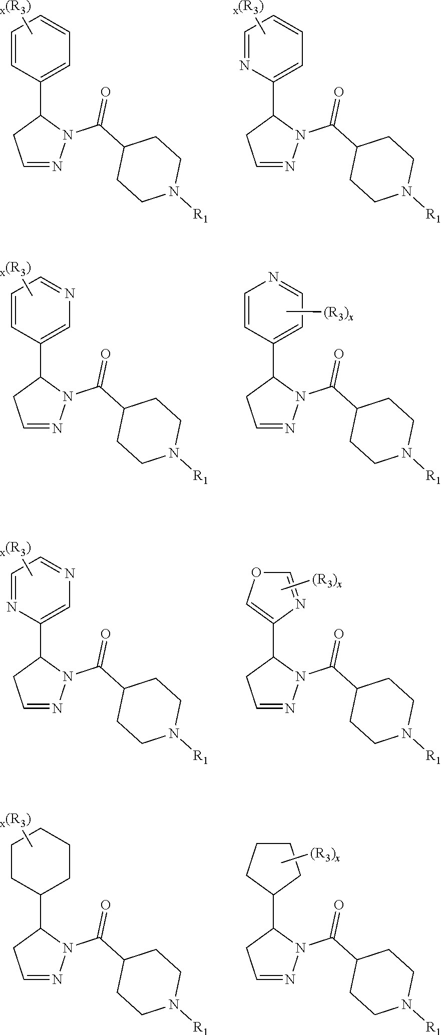

Additionally, in the formula shown above, the ELM group is selected from the group consisting of a cereblon ligase-binding moiety (CLM), a VHL ligase-binding moiety (VLM), and a MDM2 ligase-binding moiety (MLM).

Optionally, the ELM is a CLM selected from the group consisting of:

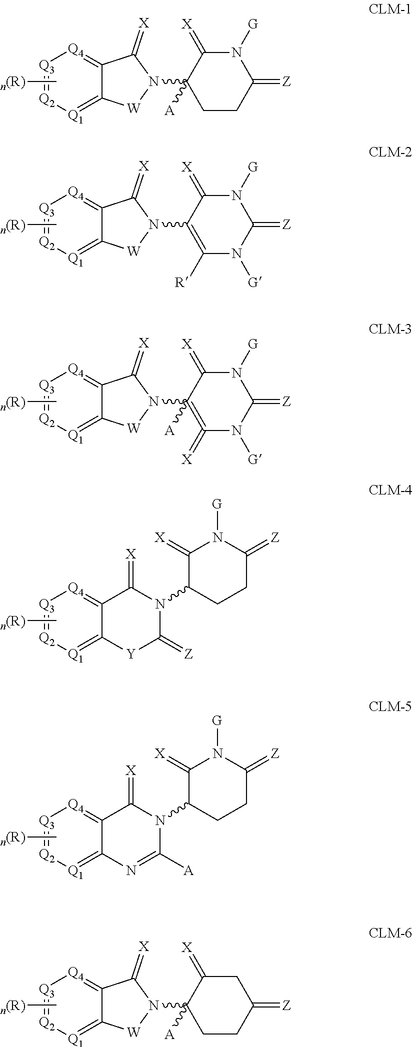

In CLM-1, CLM-2, CLM-3, CLM-4, CLM-5, and/or CLM-6, W (if present) is independently selected from the group consisting of CH₂, CHR, C═O, SO₂, NH, and N-alkyl.

Also in CLM-1, CLM-2, CLM-3, CLM-5, and CLM-6, X is independently selected from the group consisting of 0, S and 1-12.

Additionally in CLM-4, Y is independently selected from the group consisting of CH₂, —C═CR′, NH, N-alkyl, N-aryl, N-heteroaryl, N-cycloalkyl, N-heterocyclyl, O, and S.

Further in CLM-1, CLM-2, CLM-3, CLM-4, CLM-5, and CLM-6, Z is independently selected from the group consisting of O, S, and H₂.

Also in CLM-1, CLM-2, CLM-3, CLM-4, CLM-5, and/or CLM-6, G and G′ are independently selected from the group consisting of alkyl (linear, branched, optionally substituted with R′), OH, R′OCOOR, ROCONRR″, CH₂-heterocyclyl optionally substituted with R′, and benzyl optionally substituted with R′.

Additionally in CLM-1, CLM-2, CLM-3, CLM-4, CLM-5, and/or CLM-6, Q₁, Q₂, Q₃ and Q₄ represent a carbon C substituted with a group independently selected from R′, N, and N—O.

Further in CLM-1, CLM-2, CLM-3, CLM-4, CLM-5, and/or CLM-6, A is independently selected from the group consisting of H, alkyl, cycloalkyl, Cl, and F.

In CLM-1, CLM-2, CLM-3, CLM-4, CLM-5, and/or CLM-6, R is selected from the group consisting of halogen, —CF₃, —CN, —CONR′R″, —OR′, —NR′R″, —SR′, —SO₂R′, SO₂NR′R″, —CR′R″—, —CR′NR′R″—, -aryl, -hetaryl, -alkyl (linear, branched, optionally substituted), -cycloalkyl, -heterocyclyl, —P(O)(OR′)R″, —OP(O)(OR′)R″, —OP(O)R′R″, —NR′SO₂NR′R″, —NR′CONR′R″—, —CONR′COR″, —NR′C(═N—CN)NR′R″, —C(═N—CN)NR′R″, —NRC(═N—CN)R″, —NR′C(═C—NO₂)NR′R″, SO₂NR′COR″, —CO₂R′, —C(C═N—OR′)R″, —CR′═CR′R″, —CCR′, —S(C═O)(C═N—R′)R″, —SF₅ and —OCF₃.

Also in CLM-1, CLM-2, CLM-3, CLM-4, CLM-5, and/or CLM-6, R′ and R″ are independently selected from the group consisting of a bond, H, N, N—O, alkyl (linear, branched), cycloalkyl, aryl, heteroaryl, heterocyclic, —C(═O)R, or heterocyclyl, each of which is optionally substituted.

In CLM-1, CLM-2, CLM-3, CLM-4, CLM-5, and/or CLM-6, n represents an integer from 1 to 4. In some cases, Rn comprises 1-4 independent functional groups or atoms, and optionally, one of which is modified to be covalently joined to a chemical linker group (L).

Further in CLM-1, CLM-3, CLM-4, CLM-5, and/or CLM-6, [symbol] represents a bond that may be stereospecific ((R) or (S)) or non-stereospecific.

In some cases, the CLM group has the following formula:

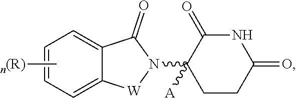

In this formula, W is independently selected from the group CH₂, C═O, NH, and N-alkyl; A is independently selected from a H, methyl, or optionally substituted linear or branched alkyl; each R is independently selected from a H, O, OH, N, NH, NH₂, methyl, optionally substituted linear or branched alkyl, optionally substituted C₁₋₆ alkoxy, optionally substituted heterocyclyl, optionally substituted -alkyl-amyl, optionally substituted aryl, optionally substituted heteroaryl aryl, amine, amide, or carboxy; n represents an integer from 1 to 4; [symbol] and, represents a bond that may be stereospecific ((R) or (S)) or non-stereospecific.

In some cases, the CLM is selected from the group consisting of:

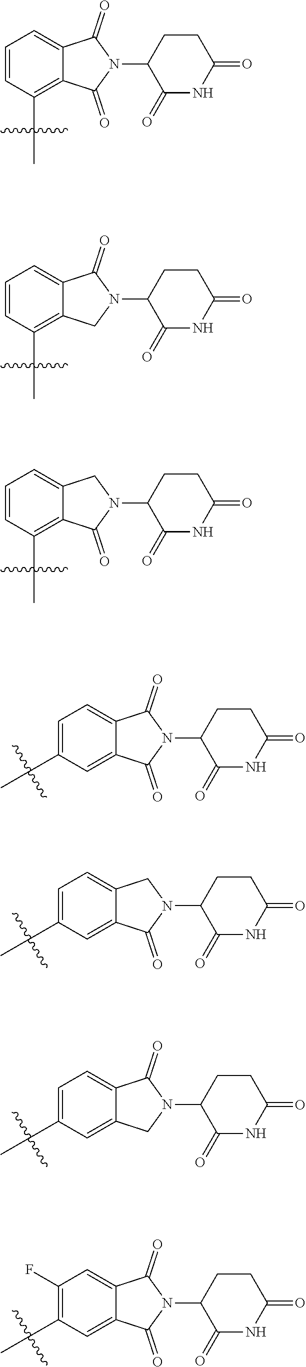

In some cases, the CLM group has the following formula:

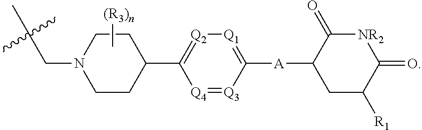

In this formula, Q₁, Q₂, Q₃ and Q₄ represent a carbon C substituted with a group independently selected from R′, N, and N—O; A is NH or O; each R′ is a bond, H, N, N—O, alkyl (linear, branched), cycloalkyl, aryl, heteroaryl, heterocyclic, —C(═O)R, or heterocyclyl, each of which is optionally substituted; R₁ and R₂ are independently hydrogen or linear or branched alkyl; each R₃ is independently selected from a H, OH, NH₂, methyl, optionally substituted linear or branched alkyl, optionally substituted C₁₋₆ alkoxy, optionally substituted heterocyclyl, optionally substituted -alkyl-aryl, optionally substituted aryl, optionally substituted heteroaryl aryl, amine, amide, or carboxy; n represents an integer from 1 to 4; and [symbol] represents a bond that may be stereospecific ((R) or (S)) or non-stereospecific. Optionally, the CLM group has the following structure:

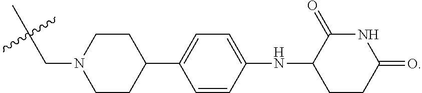 [SMILES: *CN1CCC(c2ccc(NC3CCC(=O)NC3=O)cc2)CC1]

In some cases, the CLM group has the following formula:

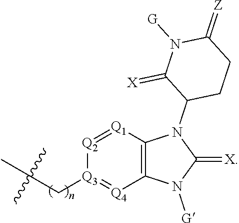

In this formula, each X is independently selected from the group consisting of O, S and H₂; Z is independently selected from the group consisting of O, S, and H₂; G and G′ are independently selected from the group consisting of H, alkyl (linear, branched, optionally substituted with R′), OH, R′OCOOR, ROCONRR″, CH₂-heterocyclyl optionally substituted with R′, and benzyl optionally substituted with R′; Q₁, Q₂, Q₃ and Q₄ represent a carbon C substituted with a group independently selected from R′, N, and N—O; R is selected from the group consisting of halogen, —CF₃, —CN, —CONR′R″, —OR′, —NR′R″, —SR′, —SO₂R′, —SO₂NR′R″, —CR′R″—, —CR′NR′R″—, -aryl, -hetaryl, -alkyl (linear, branched, optionally substituted), -cycloalkyl, -heterocyclyl, —P(O)(OR′)R″, —OP(O)(OR′)R″, —OP(O)R′R″, —NR′SO₂NR′R″, —NR′CONR′R″—, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —CO₂R′, —C(C═N—OR′)R″, —CR′═CR′R″, —CCR′, —S(C═O)(C═N—R′)R″, —SF₅ and —OCF₃; R′ and R″ are independently selected from the group consisting of a bond, H, N, N—O, alkyl (linear, branched), cycloalkyl, aryl, heteroaryl, heterocyclic, —C(S)R, or heterocyclyl, each of which is optionally substituted: n represents an integer from 1 to 4; and [symbol] represents a bond that may be stereospecific ((R) or (S)) or non-stereospecific. Optionally, the CLM group has the following structure:

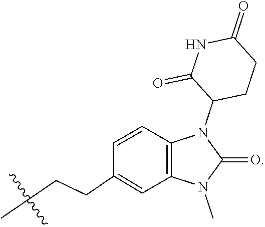 [SMILES: *CCc1ccc2c(c1)n(C)c(=O)n2C1CCC(=O)NC1=O]

In some cases, the CLM group has the following formula:

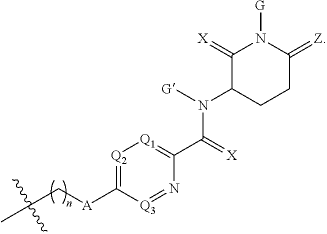

In this formula, each X is independently selected from the group consisting of O, S and H₂; Z is independently selected from the group consisting of 0, S, and H₂; G and G′ are independently selected from the group consisting of H, alkyl (linear, branched, optionally substituted with R), OH, R′OCOOR, ROCONRR″, CH₂-heterocyclyl optionally substituted with R′, and benzyl optionally substituted with R′; Q₁, Q₂, and Q₃ represent a carbon C substituted with a group independently selected from R′, N, and N—O; R is selected from the group consisting of halogen, —CF₃, —CN; CONR′R″, —OR′, —NR′R″, —SR′, —SO₂R′, —SO₂NR′R″, —CR′R″—CR′NR′R″—, -aryl, -hetaryl, -alkyl (linear, branched, optionally substituted), -cycloalkyl, -heterocyclyl, —P(O)(OR′)R″, —OP(O)(OR′)R″, —OP(O)R′R″, —NR′SO₂NR′R″, —NR′CONR′R″—, —CONR′COR″, —NR′C(═N—CN)NR′R″, —C(═N—CN)NR′R″, —NR′C(═N—CN)R″, —NR′C(═C—NO2)NR′R″, —SO₂NR′COR″, —CO₂R′, —C(C═N—OR′)R″, —CR′═CR′R″, —CCR′, —S(C═O)(C═N—R′)R″, —SF₅ and —OCF₃; R′ and R″ are independently selected from the group consisting of a bond, H, N. N—O, alkyl (linear, branched), cycloalkyl, aryl, heteroaryl, heterocyclic, —C(S)R, or heterocyclyl, each of which is optionally substituted; A is NH or O; n represents an integer from 1 to 4; and [symbol] represents a bond that may be stereospecific ((R) or (S)) or non-stereospecific. Optionally, the CLM group has the following structure:

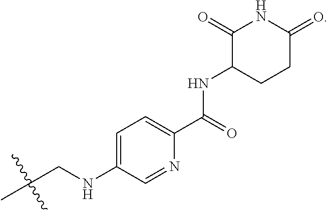 [SMILES: *CNc1ccc(C(=O)NC2CCC(=O)NC2=O)nc1]

In some cases, the CLM group has the following formula:

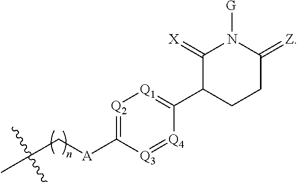

In this formula, X is selected from the group consisting of O, S and H₂; Z is independently selected from the group consisting of O, S, and H₂; G is selected from the group consisting of H, alkyl (linear, branched, optionally substituted with R′), OH, R′OCOOR, ROCONRR″, CH₂-heterocyclyl optionally substituted with R. and benzyl optionally substituted with R′; Q₁, Q₂, Q₃, and Q₄ represent a carbon C substituted with a group independently selected from R′, N, and N—O; R is selected from the group consisting of halogen, —CF₃, —CN, —CONR′R″, —OR′, —NR′R″, —SR′, —SO₂R′, —SO₂NR′R″, —CR′R″—, —CR′NR′R″—, -aryl, -hetaryl, -alkyl (linear, branched, optionally substituted), -cycloalkyl, -heterocyclyl, —P(O)(OR′)R″, —OP(O)(OR′)R″, —OP(O)R′R″, —NR′SO₂NR′R″, —NR′CONR′R″—, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —CO₂R′, —C(C═N—OR′)R″, —CCR′, —S(C═O)(C═N—R′)R″, —SF₅ and —OCF₃; R′ and R″ are independently selected from the group consisting of a bond, H, N, N—O, alkyl (linear, branched), cycloalkyl, aryl, heteroaryl, heterocyclic, —C(S)R, or heterocyclyl, each of which is optionally substituted; A is NH or O; n represents an integer from 1 to 4; and [symbol] represents a bond that may be stereospecific ((R) or (S)) or non-stereospecific. Optionally, the CLM group has the following structure:

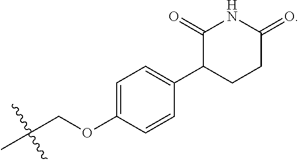 [SMILES: *COc1ccc(C2CCC(=O)NC2=O)cc1]

Optionally, the ELM is a VLM. In some cases, the VLM can have the following formula VLM-1:

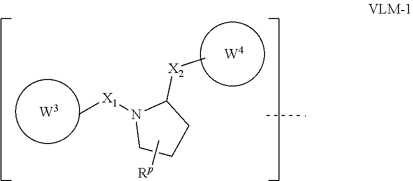

In VLM-1 and the other VLM structures shown herein, the dashed line indicates the attachment a chemical linker moiety coupling at least one TPM.

Also in VLM-1, X¹ and X² are independently selected from the group of a bond, O, NR^{Y3}, CR^{Y3}R^{Y4}, C═O, C═S, SO, and SO₂.

Additionally in VLM-1, R^{Y3} and R^{Y4} are each independently selected from the group of hydrogen, linear or branched C₁₋₆ alkyl, optionally substituted by one or more halo, optionally substituted C₁₋₆ alkoxyl (e.g., optionally substituted by 0-3 R^{p} groups, wherein R^{p} is one to three groups, each independently selected from the group hydrogen, halogen, —OH, C₁₋₃ alkyl, C═O).

Further in VLM-1, W³ is selected from the group of an optionally substituted T, an optionally substituted -TN(R^{1a}R^{1b})X³, optionally substituted -T-N(R^{1a}R^{1b}), optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR^{X}-T-Aryl, an optionally substituted —NR′-T-Heteroaryl or an optionally substituted —NR′-T-Heterocycle.

Also in VLM-1, X³ is C═O, R^{1a}, R^{1b},

In VLM-1, R¹, R^{1a}, and R^{1b} are each independently selected from the group consisting of hydrogen, linear or branched C₁₋₆ alkyl group optionally substituted by one or more halogen or —OH groups, R^{Y3}CO, R^{Y3}C═S, R^{Y3}SO, R^{Y3}SO₂, N(R^{Y3}R^{Y4}) C═O, N(R^{Y3}R^{Y4})C═S, N(R^{Y3}R^{Y4})SO, and N(R^{Y3}R^{Y4})SO₂.

Additionally in VLM-1, T is selected from the group of an optionally substituted alkyl, —(CH₂)ₙ— group, wherein each one of the methylene groups is optionally substituted with one or two substituents selected from the group consisting of halogen, methyl, optionally substituted alkoxy, a linear or branched C1-C6 alkyl group optionally substituted by 1 or more halogen, C(O)NR^{x}R^{1a}, or NR^{x}R^{1a} or R¹ and R^{1a} are joined to form an optionally substituted heterocycle, or —OH groups or an amino acid side chain optionally substituted.

Also in VLM-1, n is 0 to 6, e.g., 0, 1, 2, or 3, preferably 0 or 1.

Further in VLM-1, W⁴ is an optionally substituted —NR¹-T-Aryl wherein the aryl group may be optionally substituted with an optionally substituted 5-6 membered heteroaryl or aryl, an optionally substituted —NR1-T-Heteroaryl group or an optionally substituted —NR¹-T-Heterocycle, where NR¹ is covalently bonded to X² and R¹ is H or CH₃, preferably H.

In any of the embodiments described herein, T is selected from the group consisting of an optionally substituted alkyl, —(CH₂)ₙ— group, wherein each one of the methylene groups is optionally substituted with one or two substituents selected from the group of halogen, methyl, optionally substituted alkoxy, a linear or branched C₁₋₆ alkyl group optionally substituted by 1 or more halogen, C(O)NR^{x}R^{1a}, or NR^{x}R^{1a} or R¹ and R^{1a} are joined to form an optionally substituted heterocycle, or —OH groups or an amino acid side chain optionally substituted; and n is 0 to 6, often 0, 1, 2, or 3, preferably 0 or 1.

In certain embodiments, W⁴ of Formula VLM-1 is selected from the group consisting of:

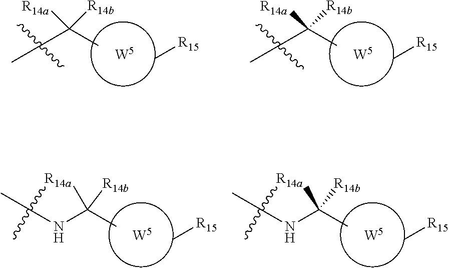

wherein R₁₄ₐ, R_{14b}, are independently selected from the group of H, haloalkyl, or optionally substituted alkyl; W⁵ is optionally substituted (e.g., W⁵ is an optionally substituted phenyl, an optionally substituted napthyl, or an optionally substituted 5-10 membered heteroaryl). In any of the embodiments, W⁵ is selected from the group consisting of a phenyl or a 5-10 membered heteroaryl, R₁₅ of Formula VLM-1 is selected from the group of H, halogen, CN, OH, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl.

In additional embodiments, W⁴ substituents for use in the present disclosure also include specifically (and without limitation to the specific compound disclosed) the W⁴ substituents which are found in the identified compounds disclosed herein. Each of these W⁴ substituents may be used in conjunction with any number of W³ substituents which are also disclosed herein.

In certain additional embodiments, VLM-1 is optionally substituted by 0-3 R^{p} groups in the pyrrolidine moiety. Each R^{p} is independently H, halo, —OH, C₁₋₃ alkyl, or C═O. In any of the embodiments described herein, the W³ and/or W⁴ groups of Formula VLM-1 can independently be covalently coupled to a linker which is attached one or more TPM groups.

In certain embodiments, ELM is VLM and is represented by the structure VLM-2:

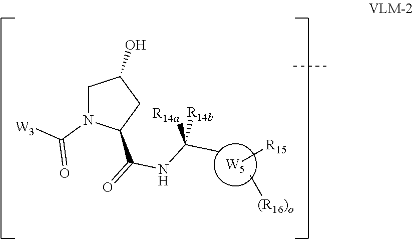

In VLM-2, the dashed line indicates the attachment a chemical linker moiety coupling at least one TPM.

Optionally, the W³ group of Formula VLM-2 is selected from the group of an optionally substituted aryl, optionally substituted heteroaryl, or

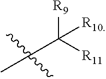

In Formula VLM-2, R₉ and R₁₀, when present, are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl.

Also in Formula VLM-2, R₁₁, when present, is selected from the group of an optionally substituted heterocyclic, optionally substituted alkoxy, optionally substituted heteroaryl, optionally substituted aryl,

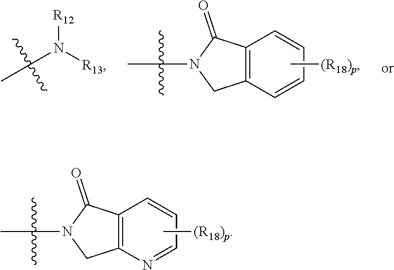

R₁₂ of Formula VLM-2 is selected from the group of H or optionally substituted alkyl and R₁₃ of Formula VLM-2 is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; Additionally in VLM-2, R₁₄ₐ and R_{14b} are each independently selected from the group of H, haloalkyl, or optionally substituted alkyl.

Further, W₅ of Formula VLM-2 is selected from the group of an optionally substituted phenyl or an optionally substituted 5-10 membered heteroaryl, R₁₅ of Formula ULM-b is selected from the group of H, halogen, CN, OH, NO2, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl.

Also in VLM-2, each R₁₆ is independently selected from the group of H, CN, halogen, optionally substituted alkyl, optionally substituted haloalkyl, hydroxy, or optionally substituted haloalkoxy.

Additionally in VLM-2, o is 0, 1, 2, 3, or 4.

Further in VLM-2, R₁₈ is independently selected from the group of H, halogen, optionally substituted alkoxy, cyano, optionally substituted alkyl, haloalkyl, haloalkoxy or a linker.

Also in VLM-2, p is 0, 1, 2, 3, or 4, and wherein the dashed line indicates the site of attachment of a chemical linker moiety coupling at least one TPM.

In certain embodiments, R₁₅ of Formula VLM-2 is

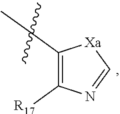

wherein R₁₇ is H, halogen, optionally substituted C₃₋₆ cycloalkyl, optionally substituted C₁₋₆ alkyl, optionally substituted C₁₋₆ alkenyl, and C₁₋₆haloalkyl; and Xa is S or O.

In certain embodiments, R₁₇ of Formula VLM-2 is selected from the group methyl, ethyl, isopropyl, and cyclopropyl.

In certain additional embodiments, R₁₅ of Formula VLM-2 is selected from the group consisting of:

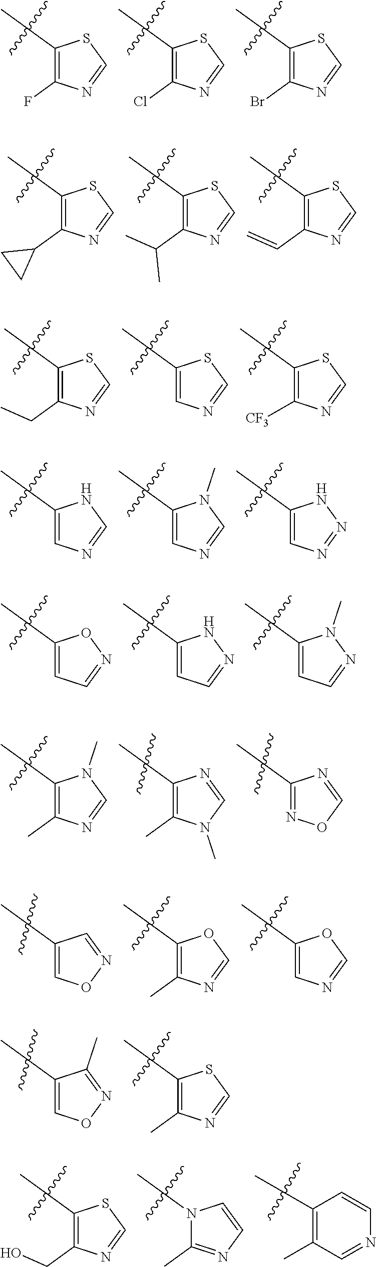

In certain additional embodiments, R₁₁ of Formula VLM-2 is selected from the group consisting of:

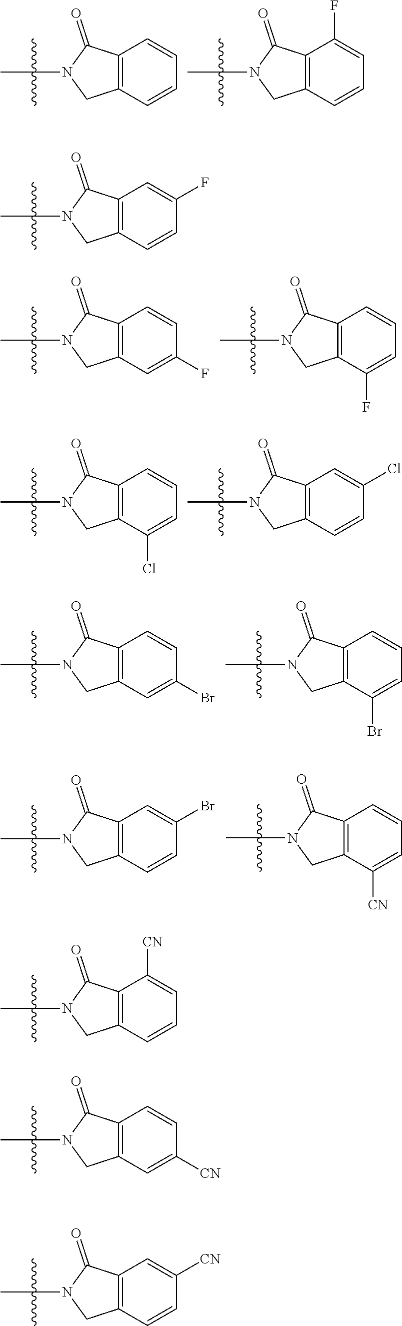

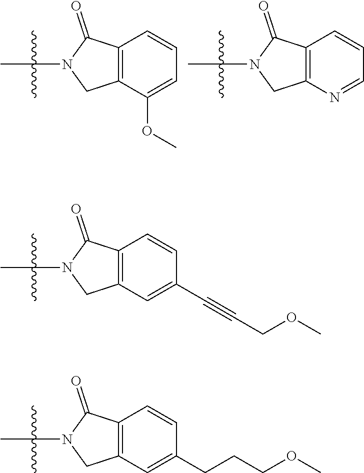 [SMILES: *N1Cc2c(OC)cccc2C1=O ; *N1Cc2ncccc2C1=O ; *N1Cc2cc(C#CCOC)ccc2C1=O ; *N1Cc2cc(CCCOC)ccc2C1=O]

Optionally, the VLM has a chemical structure selected from the group of:

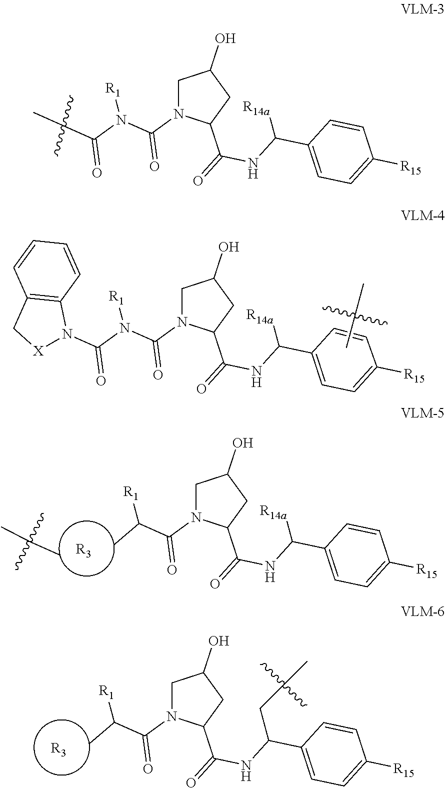

In Formulas VLM-3, VLM-4, VLM-5, and VLM-6, R₁ is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl.

Also in Formulas VLM-3, VLM-4, VLM-5, and VLM-6, R₁₄ₐ is H, haloalkyl, optionally substituted alkyl, methyl, fluoromethyl, hydroxymethyl, ethyl, isopropyl, or cyclopropyl.

Additionally in Formulas VLM-3, VLM-4, VLM-5, and VLM-6, R₁₅ is selected from the group consisting of H, halogen, CN, OH, NO₂, optionally substituted heteroaryl, optionally substituted aryl, optionally substituted alkyl, optionally substituted haloalkyl, optionally substituted haloalkoxy, optionally substituted cycloalkyl, or optionally substituted cycloheteroalkyl (each optionally substituted).

Further in Formulas VLM-3, VLM-4, VLM-5, and VLM-6, X is C, CH₂, or C═O.

Also in Formulas VLM-3, VLM-4, VLM-5, and VLM-6, R₃ is absent or an optionally substituted 5 or 6 membered heteroaryl.

Additionally in Formulas VLM-3, VLM-4, VLM-5, and VLM-6, the dashed line indicates the site of attachment of a chemical linker moiety coupling at least one TPM.

In certain embodiments, VLM is represented by the structure VLM-7 shown below:

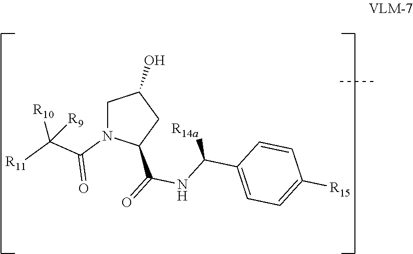

In Formula VLM-7, R₁₄ₐ is H, haloalkyl, optionally substituted alkyl, methyl, fluoromethyl, hydroxymethyl, ethyl, isopropyl, or cyclopropyl.

Also in Formula VLM-7, R₉ is H.

Additionally in Formula VLM-7, R₁₀ is H, ethyl, isopropyl, tert-butyl, sec-butyl, cyclopropyl, cyclobutyl, cyclopentyl, or cyclohexyl.

Further in Formula VLM-7, R₁₁ is

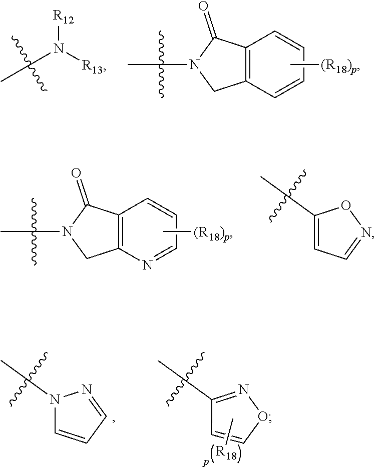

or optionally substituted heteroaryl.

Also in Formula VLM-7, p is 0, 1, 2, 3, or 4.

Additionally in Formula VLM-7, each R₁₈ is independently halo, optionally substituted alkoxy, cyano, optionally substituted alkyl, haloalkyl, haloalkoxy or a linker.

Further in Formula VLM-7, R₁₂ is H or C═O.

Also in Formula VLM-7, R₁₃ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl.

Additionally in Formula VLM-7, R₁₅ is selected from the group consisting of H, halogen, Cl, CN, OH, NO₂, optionally substituted heteroaryl, optionally substituted aryl:

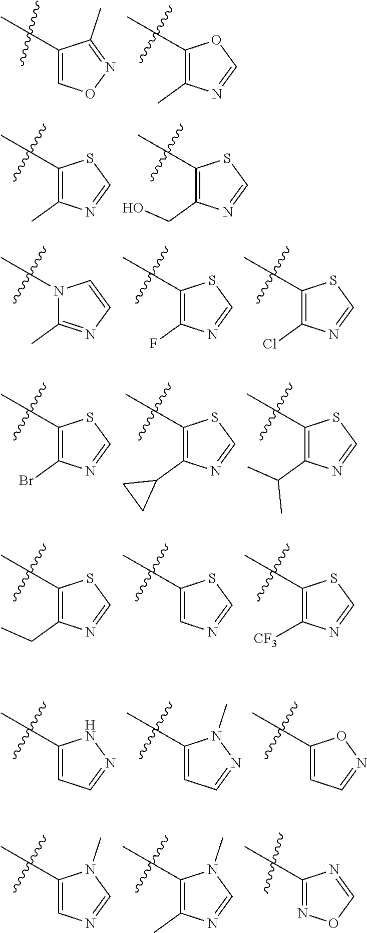

In VLM-7, the dashed line indicates the site of attachment of a chemical linker moiety coupling at least one TPM.

In certain embodiments, the VLM is selected from the following structures:

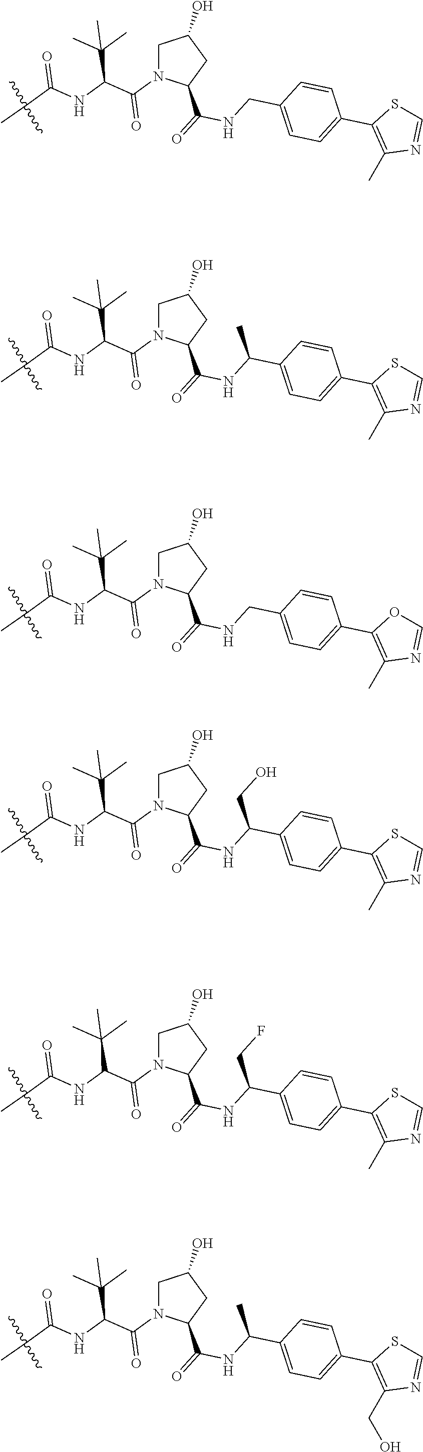

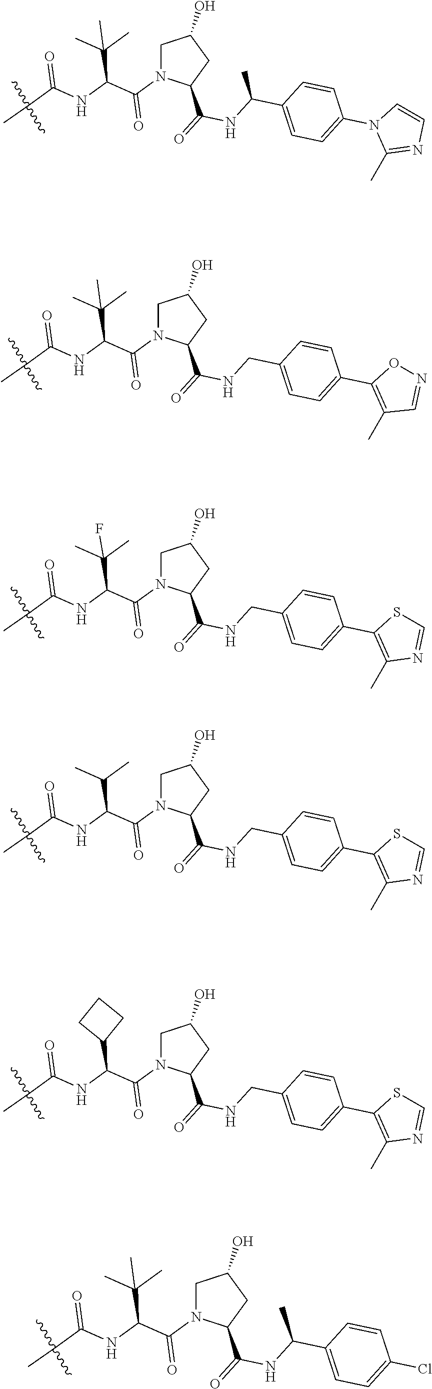 [SMILES: *C(=O)N[C@H](C(=O)N1C[C@H](O)C[C@H]1C(=O)N[C@@H](C)c1ccc(-n2ccnc2C)cc1)C(C)(C)C ; *C(=O)N[C@H](C(=O)N1C[C@H](O)C[C@H]1C(=O)NCc1ccc(-c2oncc2C)cc1)C(C)(C)C ; *C(=O)N[C@H](C(=O)N1C[C@H](O)C[C@H]1C(=O)NCc1ccc(-c2scnc2C)cc1)C(C)(C)F ; *C(=O)N[C@H](C(=O)N1C[C@H](O)C[C@H]1C(=O)NCc1ccc(-c2scnc2C)cc1)C(C)C ; *C(=O)N[C@H](C(=O)N1C[C@H](O)C[C@H]1C(=O)NCc1ccc(-c2scnc2C)cc1)C1CCC1 ; *C(=O)N[C@H](C(=O)N1C[C@H](O)C[C@H]1C(=O)N[C@@H](C)c1ccc(Cl)cc1)C(C)(C)C]

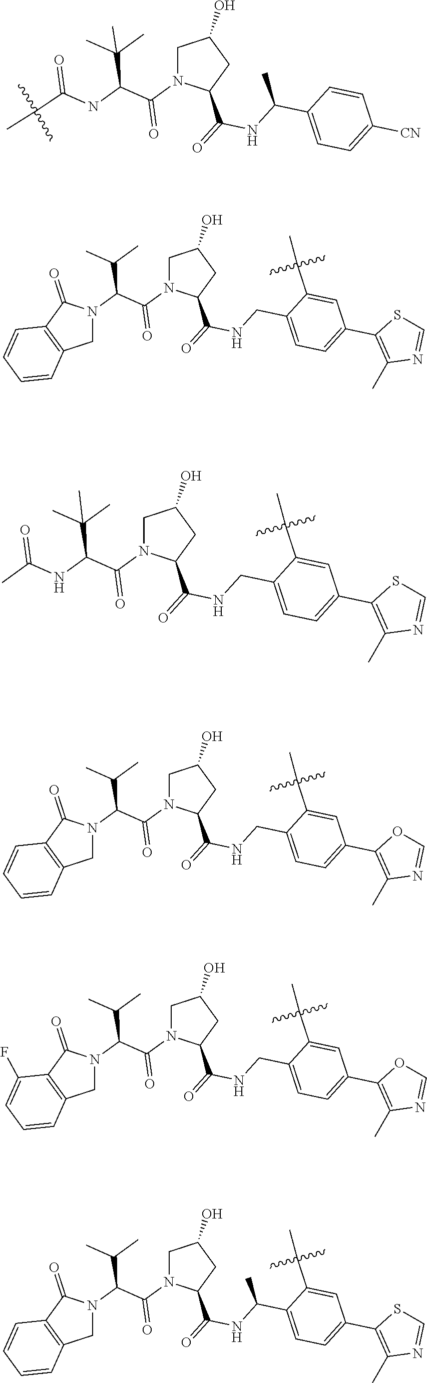 [SMILES: *C(=O)N[C@H](C(=O)N1C[C@H](O)C[C@H]1C(=O)N[C@@H](C)c1ccc(C#N)cc1)C(C)(C)C ; *c1cc(-c2scnc2C)ccc1CNC(=O)[C@@H]1C[C@@H](O)CN1C(=O)[C@H](C(C)C)N1Cc2ccccc2C1=O ; *c1cc(-c2scnc2C)ccc1CNC(=O)[C@@H]1C[C@@H](O)CN1C(=O)[C@@H](NC(C)=O)C(C)(C)C ; *c1cc(-c2ocnc2C)ccc1CNC(=O)[C@@H]1C[C@@H](O)CN1C(=O)[C@H](C(C)C)N1Cc2ccccc2C1=O ; *c1cc(-c2ocnc2C)ccc1CNC(=O)[C@@H]1C[C@@H](O)CN1C(=O)[C@H](C(C)C)N1Cc2cccc(F)c2C1=O ; *c1cc(-c2scnc2C)ccc1[C@H](C)NC(=O)[C@@H]1C[C@@H](O)CN1C(=O)[C@H](C(C)C)N1Cc2ccccc2C1=O]

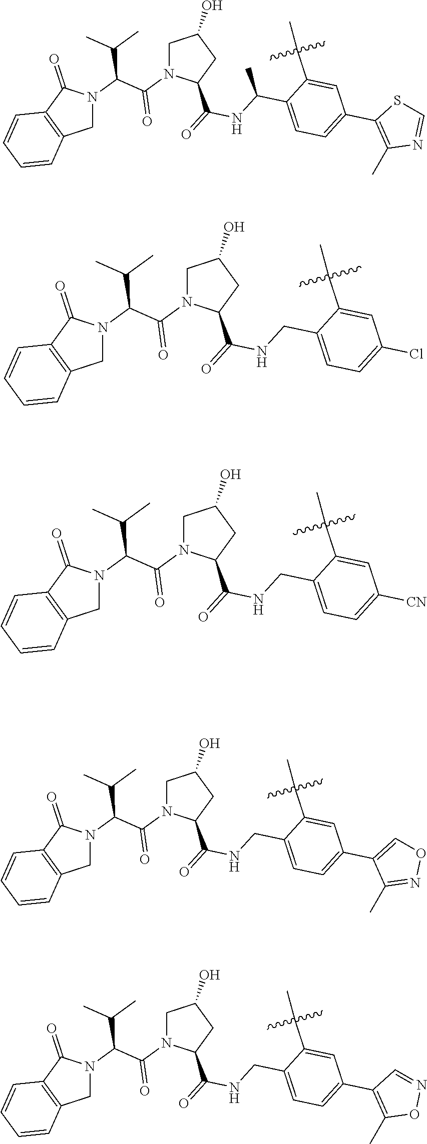 [SMILES: *c1cc(-c2scnc2C)ccc1[C@H](C)NC(=O)[C@@H]1C[C@@H](O)CN1C(=O)[C@H](C(C)C)N1Cc2ccccc2C1=O ; *c1cc(Cl)ccc1CNC(=O)[C@@H]1C[C@@H](O)CN1C(=O)[C@H](C(C)C)N1Cc2ccccc2C1=O ; *c1cc(C#N)ccc1CNC(=O)[C@@H]1C[C@@H](O)CN1C(=O)[C@H](C(C)C)N1Cc2ccccc2C1=O ; *c1cc(-c2conc2C)ccc1CNC(=O)[C@@H]1C[C@@H](O)CN1C(=O)[C@H](C(C)C)N1Cc2ccccc2C1=O ; *c1cc(-c2cnoc2C)ccc1CNC(=O)[C@@H]1C[C@@H](O)CN1C(=O)[C@H](C(C)C)N1Cc2ccccc2C1=O]

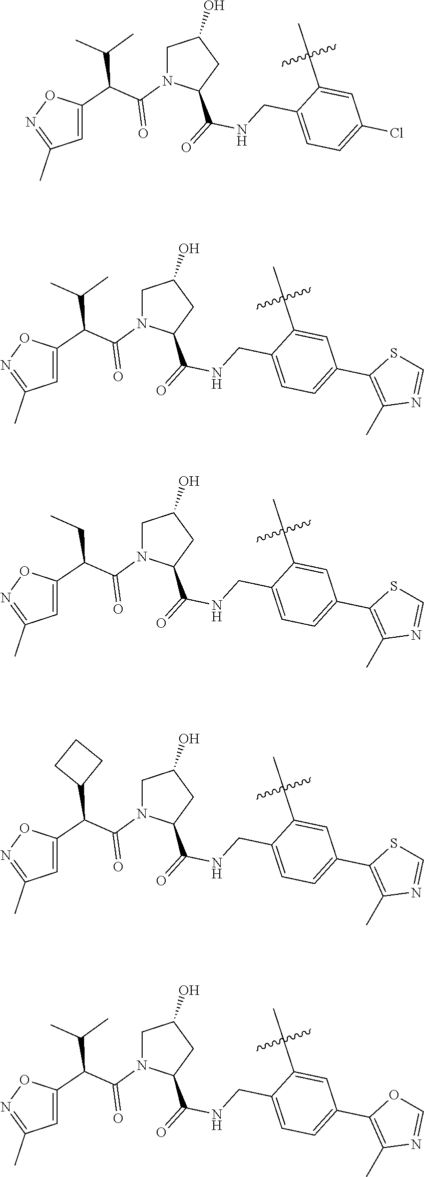 [SMILES: *c1cc(Cl)ccc1CNC(=O)[C@@H]1C[C@@H](O)CN1C(=O)[C@@H](c1cc(C)no1)C(C)C ; *c1cc(-c2scnc2C)ccc1CNC(=O)[C@@H]1C[C@@H](O)CN1C(=O)[C@@H](c1cc(C)no1)C(C)C ; *c1cc(-c2scnc2C)ccc1CNC(=O)[C@@H]1C[C@@H](O)CN1C(=O)[C@H](CC)c1cc(C)no1 ; *c1cc(-c2scnc2C)ccc1CNC(=O)[C@@H]1C[C@@H](O)CN1C(=O)[C@@H](c1cc(C)no1)C1CCC1 ; *c1cc(-c2ocnc2C)ccc1CNC(=O)[C@@H]1C[C@@H](O)CN1C(=O)[C@@H](c1cc(C)no1)C(C)C]

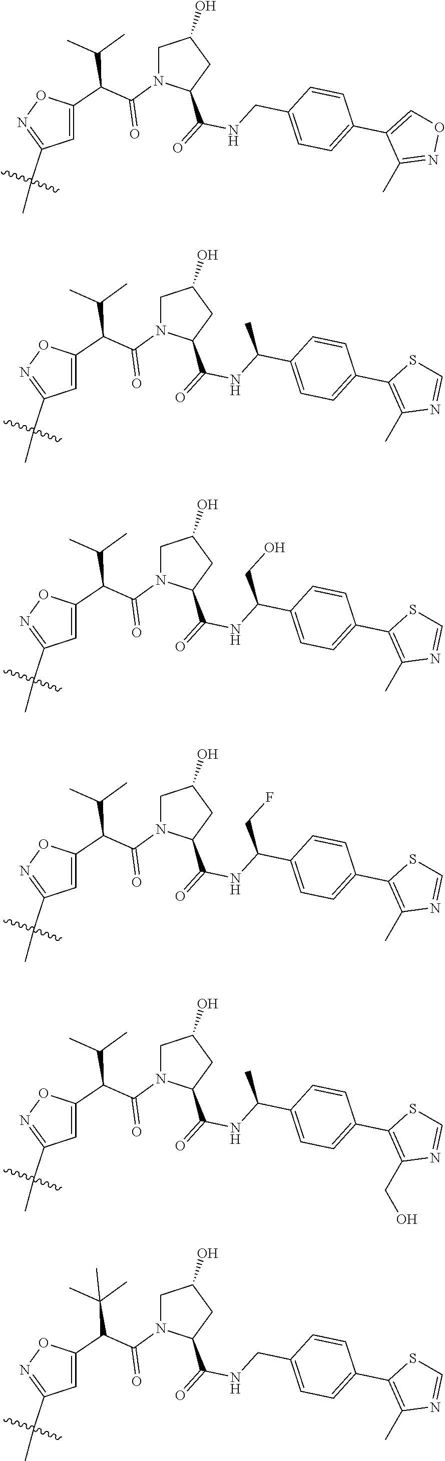

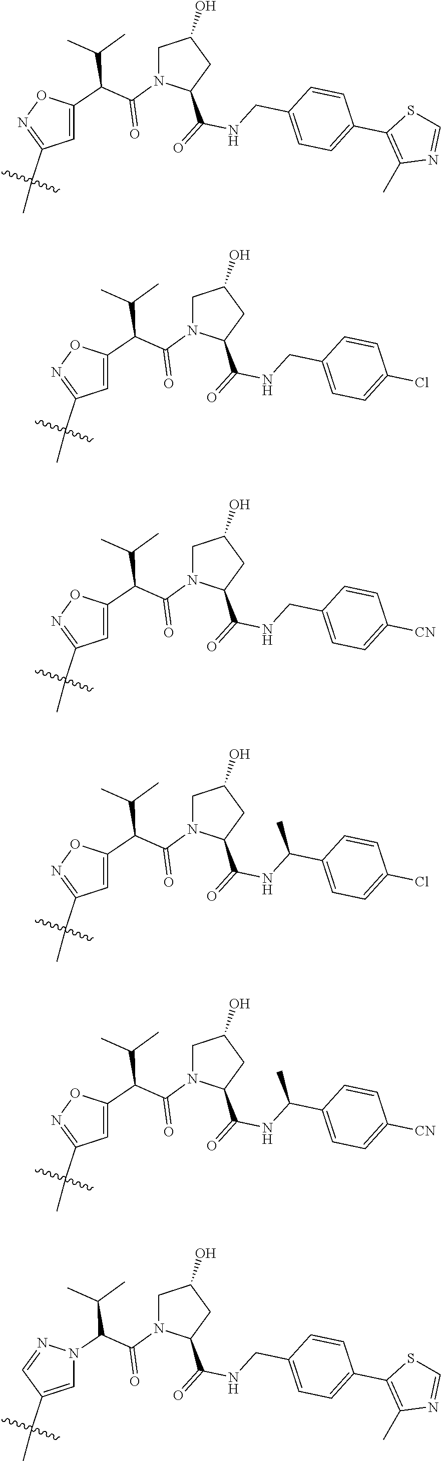

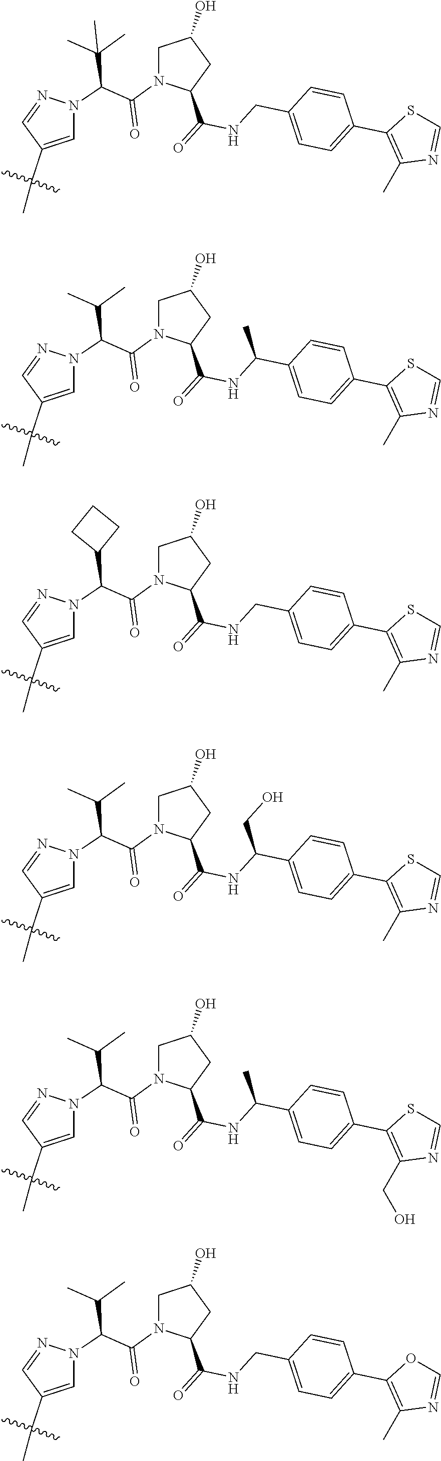 [SMILES: *c1cnn([C@H](C(=O)N2C[C@H](O)C[C@H]2C(=O)NCc2ccc(-c3scnc3C)cc2)C(C)(C)C)c1 ; *c1cnn([C@H](C(=O)N2C[C@H](O)C[C@H]2C(=O)N[C@@H](C)c2ccc(-c3scnc3C)cc2)C(C)C)c1 ; *c1cnn([C@H](C(=O)N2C[C@H](O)C[C@H]2C(=O)NCc2ccc(-c3scnc3C)cc2)C2CCC2)c1 ; *c1cnn([C@H](C(=O)N2C[C@H](O)C[C@H]2C(=O)N[C@@H](CO)c2ccc(-c3scnc3C)cc2)C(C)C)c1 ; *c1cnn([C@H](C(=O)N2C[C@H](O)C[C@H]2C(=O)N[C@@H](C)c2ccc(-c3scnc3CO)cc2)C(C)C)c1 ; *c1cnn([C@H](C(=O)N2C[C@H](O)C[C@H]2C(=O)NCc2ccc(-c3ocnc3C)cc2)C(C)C)c1]

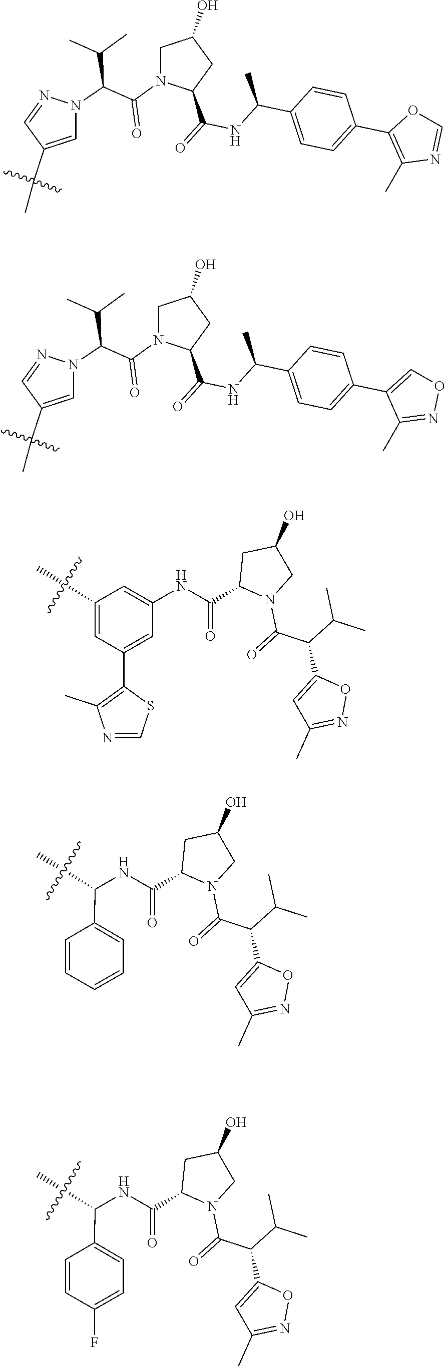

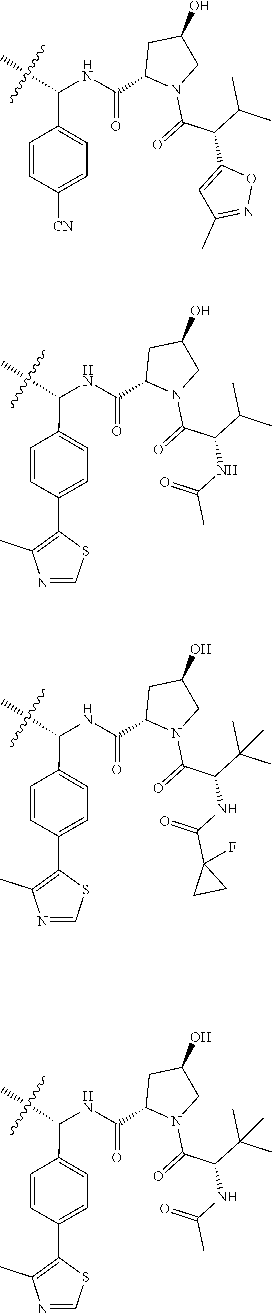

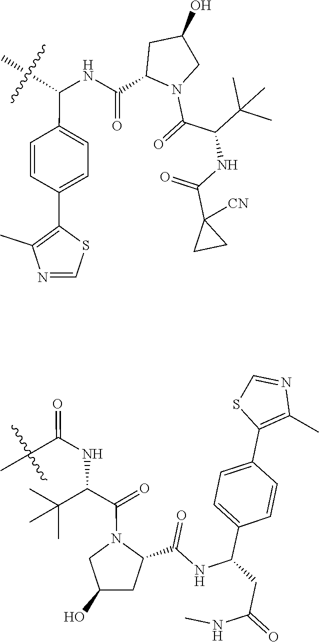

In certain cases, the ELM is a MLM. Optionally, the ELM comprises part of structural features as in RG7112, RG7388, SAR405838, AMG-232, AM-7209, DS-5272, MK-8242, and NVP-CGM-097, and analogs or derivatives thereof.

In some cases, the MLM is a derivative of substituted imidazoline represented as Formula (MLM-1), or thiazoloimidazoline represented as Formula (MLM-2), or spiro indolinone represented as Formula (MLM-3), or pyrolidine represented as Formula (MLM-4), or piperidinone/morpholinone represented as Formula (MLM-5), or isoquinolinone represented as Formula (MLM-6), or pyrrolopyrimidine/imidazolo pyridine represented as Formula (MLM-7), or pyrrolo pyrrolidinone/imidazolopyrrolidinone represented as Formula (MLM-8).

Optionally, the ELM is a MLM selected from the group consisting of:

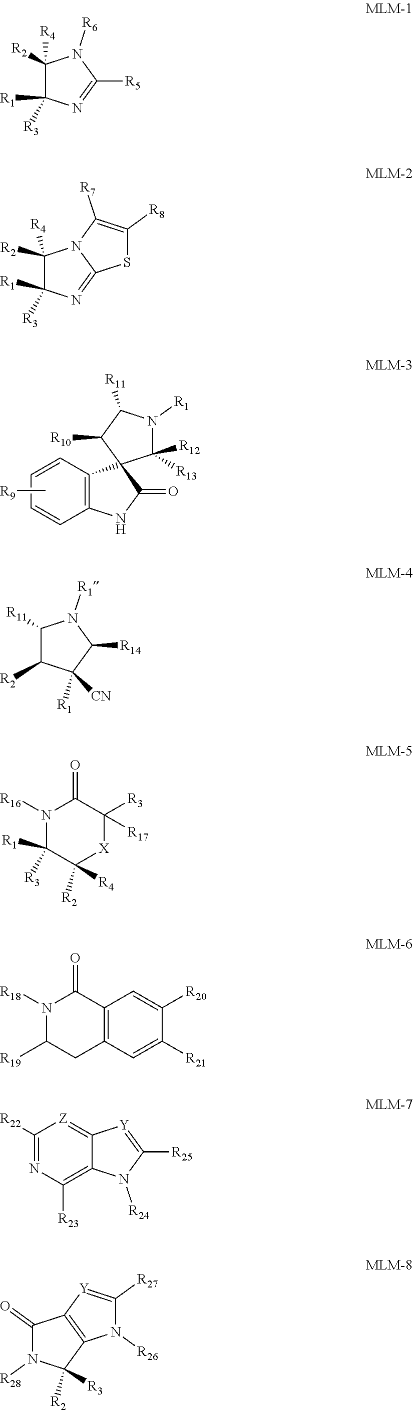

In MLM-1, R₁ and R₂ are each independently selected from the group consisting of an aryl or heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of: halogen, —CN, C₁₋₆ alkyl group, C₃₋₆ cycloalkyl, —OH, unsubstituted or fluorine substituted C₁₋₆ alkoxy, C₁₋₆ sulfoxide, C₁₋₆ sulfone, C₂₋₆ ketone, C₂₋₆ amides, and di-C₂₋₆ alkyl amine.

Also in MLM-1, R₃ and R₄ are independently selected from the group consisting of H and C₁₋₆ alkyl(e.g., methyl).

Additionally in MLM-1, R₅ is selected from the group consisting of an aryl or heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of: halogen, —CN, C₁₋₆alkyl, C₃₋₆ cycloalkyl, —OH, unsubstituted or fluorine substituted C₁₋₆ alkoxy, C₁₋₆ sulfoxide, C₁₋₆ sulfone, C₂₋₆, ketone, C₂₋₆ amides, di-C₂₋₆ alkyl amine, morpholinyl, C₃₋₆ alkyl ester, and C₃₋₆ alkyl cyanide.

Further in MLM-1, R₆ is H or —C(═O)R^{b}, wherein R^{b} is selected from the group consisting of alkyl, cycloalkyl, mono-, di- or tri-substituted aryl or heteroaryl, 4-morpholinyl, 1-(3-oxopiperazinyl), 1-piperidinyl, 4-N—R^{c}-morpholinyl, 4-R^{c}-1-piperidinyl, and 3-R^{c}-1-piperidinyl, wherein R^{c} is selected from the group consisting of alkyl, fluorine substituted alkyl, cyano alkyl, hydroxyl-substituted alkyl, cycloalkyl, alkoxyalkyl, amide alkyl, alkyl sulfone, alkyl sulfoxide, alkyl amide, aryl, heteroaryl, mono-, bis- and tri-substituted aryl or heteroaryl, CH₂CH₂R^{d}, and CH₂CH₂CH₂R^{d}, wherein R^{d} is selected from the group consisting of alkoxy, alkyl sulfone, alkyl sulfoxide, N-substituted carboxamide, —NHC(O)-alkyl, —NH—SO₂-alkyl, aryl, substituted aryl, heteroaryl, and substituted heteroaryl.

In MLM-2, R₁, R₂, R₃, and R₄ are defined as above for MLM-1.

Also in MLM-2, R₇ is selected from the group consisting of H, C₁₋₆ alkyl, cyclic alkyl, fluorine-substituted alkyl, cyan-substituted alkyl, 5- or 6-membered heteroaryl or aryl, substituted 5- or 6-membered heteroaryl or aryl.

Additionally in MLM-2, R₈ is selected from the group consisting of —R^{e}—C(O)R^{f}, —R^{e}-alkoxy, —R^{e}-aryl, —R^{e}-heteroaryl, and —R^{e}—C(O)—R^{f}—C(O)—R^{g}, wherein R^{e} is an C₁₋₆ alkylene or a bond and R^{f} and R^{g} are each independently substituted pyrrolidine, substituted piperidine, or substituted piperazine.

In MLM-3, R₉ is selected from the group consisting of a mono-, bis-, or tri-substituent on the fused bicyclic aromatic ring in MLM-3, wherein the substitutents are each independently selected from the group consisting of halogen, alkene, alkyne, alkyl, unsubstituted or substituted with Cl or F. In other words, in MLM-3, n can be 1, 2, or 3 and R₉ can be a halogen, substituted or unsubstituted alkene, substituted or unsubstituted alkyne, or substituted or unsubstituted alkyl, wherein the alkene, alkyne, or alkyl can be substituted with Cl or F.

Also in MLM-3, R₁₀ is selected from the group consisting of an aryl or heteroaryl group. Optionally, the heteroatoms of the heteroaryl group can be sulfur or nitrogen. In some cases, the aryl or heteroaryl group can be monocyclic or bicyclic. The aryl or heteroaryl groups can optionally be unsubstituted or substituted with one to three substituents, including a halogen (e.g., F or Cl), —CN, alkene, alkyne, C₁₋₆ alkyl, C₃₋₆ cycloalkyl, —OH, unsubstituted or fluorine substituted C₁₋₆ alkoxy, C₁₋₆ sulfoxide, C₁₋₆ sulfone, or C₂₋₆ ketone.

Additionally in MLM-3, R₁₁ is —C(O)—N(R^{h})(Rⁱ), wherein R^{h} and Rⁱ are selected from groups consisting of the following: H; optionally substituted linear or branched C₁₋₆ alkyl; alkoxy substituted alkyl; mono- and di-hydroxy substituted alkyl, sulfone substituted alkyl; optionally substituted aryl; optionally substituted heteroaryl; mono-, bis- or tri-substituted aryl or heteroaryl: phenyl-4-carboxylic acid: substituted phenyl-4-carboxylic acid, alkyl carboxylic acid; optionally substituted heteroaryl carboxylic acid: alkyl carboxylic acid; fluorine substituted alkyl carboxylic acid; optionally substituted cycloalkyl, 3-hydroxycyclobutane, 4-hydroxycyclohehexane, aryl substituted cycloalkyl; heteroaryl substituted cycloalkyl: or R^{h} and Rᵢ taken together can form a ring.

Further in MLM-3, R₁₂ and R₁₃ are each independently selected from H, C₁₋₆ alkyl, C₂₋₆ alkenyl, C₂₋₆alkynyl, C₄₋₆cycloalkyl, substituted cycloalkyl, cycloalkenyl, substituted cycloalkenyl, 5- and 6-membered aryl, and heteroaryl. Optionally, R₁₂ and R₁₃ can be connected to form a 5- and 6-membered ring with or without a substitution on the ring.

Also in MLM-3, R₁″ is selected from the group consisting of H, alkyl, aryl substituted alkyl, alkoxy substituted alkyl, cycloalkyl, aryl-substituted cycloalkyl, and alkoxy substituted cycloalkyl.

In MLM-4, R₁, R₂, R₁₁, and R₁″ are as defined above for MLM-1 and MLM-3.

Also in MLM-4, R₁₄ is selected from the group consisting of alkyl, substituted alkyl, alkenyl, substituted alkenyl, aryl, substituted aryl, heteroaryl, substituted heteroaryl, heterocycle, substituted heterocycle, cycloalkyl, substituted cycloalkyl, cycloalkenyl, and substituted cycloalkenyl.

In MLM-5, X is selected from the group consisting of carbon, oxygen, sulfur, sulfoxide, sulfone, and N—R^{a}, wherein R^{a} is independently H or C₁₋₆ alkyl.

Also in MLM-5, R₁, R₂, R₃, and R₄ are as defined above for MLM-1.

Additionally in MLM-5, R₁₆ is selected from the group consisting of C₁₋₆ alkyl, C₁₋₆ cycloalkyl, C₂₋₆ alkenyl, C₁₋₆ alkyl and C₃₋₆ cycloalkyl with one or multiple hydrogens replaced by fluorine, alkyl, or cycloalkyl. Optionally, for the cycloalkyl substituent, one CH₂ can be replaced by SO, —S, or —SO₂. Optionally, the alkyl or cycloalkyl groups present in R16 can have a terminal CH₃ replaced by SO₂N(alkyl)(alkyl), —CON(alkyl)(alkyl), —N(alkyl)SO₂(alkyl), —CO₂(alkyl), —O(alkyl), C₁₋₆ alkyl or alkyl-cycloalkyl with one or more hydrogens replaced by a hydroxyl group, a 3 to 7 membered cycloalkyl or heterocycloalkyl, optionally containing a —CO— group, or a 5 to 6 membered aryl or heteroaryl group, which heterocycloalkyl or heteroaryl group can contain from one to three heteroatoms independently selected from O, N or S. and the cycloalkyl, heterocycloalkyl, aryl or heteroaryl group can be unsubstituted or substituted with from one to three substituents independently selected from halogen, C₁₋₆ alkyl, hydroxylated C₁₋₆ alkyl, C₁₋₆ alkyl containing thioether, ether, sulfone, sulfoxide, fluorine substituted ether, or cyano group.

Further in MLM-5, R₁₇ is selected from the group consisting of (CH₂)ₙC(O)NR^{k}R^{l}, wherein R^{k} and R^{l} are each independently selected from H, C₁₋₆ alkyl, hydroxylated C₁₋₆ alkyl, C₁₋₆ alkoxy alkyl, C₁₋₆ alkyl with one or multiple hydrogens replaced by fluorine, C₁₋₆ alkyl with one carbon replaced by SO, SO₂, C₁₋₆ alkoxyalkyl with one or multiple hydrogens replaced by fluorine, C₁₋₆ alkyl with hydrogen replaced by a cyano group, 5 and 6 membered aryl or heteroaryl, alkyl aryl with an alkyl group containing from 1-6 carbons (C₁₋₆ alkyl), and alkyl heteroaryl with a C₁₋₆ alkyl, wherein the aryl or heteroaryl group can be further substituted.

In MLM-6, R₁₈ is selected from the group consisting of substituted aryl, heteroaryl, alkyl, and cycloalkyl. The substitution is optionally —N(C₁₋₄ alkyl)(cycloalkyl), —N(C₁₋₄ alkyl)alkyl-cycloalkyl, or —N(C₁₋₄ alkyl)[(alkyl)-(heterocycle-substituted)-cycloalkyl].

Also in MLM-6, R₁₉ is selected from the group consisting of aryl, heteroaryl, and bicyclic heteroaryl, and these aryl or heteroaryl groups can be substituted with halogen, C₁₋₆ alkyl, C₁₋₆cycloalkyl, CF₃, F, CN, alkyne, alkyl sulfone, Optionally, the aryl, heteroaryl, and bicyclic heteroaryl groups can be mono-substituted, bi-substituted, or tri-substituted.

Additionally in MLM-6, R₂₀ and R₂₁ are each independently selected from C₁₋₆ alkyl, C₁₋₆ cycloalkyl, C₁₋₆ alkoxy, hydroxylated C₁₋₆ alkoxy, and fluorine substituted C₁₋₆ alkoxy, wherein R₂₀ and R₂₁ can further be connected to form a 5, 6 and 7-membered cyclic or heterocyclic ring, which can further be substituted.

In MLM-7, Y and Z are each independently C or N.

Also in MLM-7, R₂₂ is selected from the group consisting of H, C₁₋₆ alkyl, C₁₋₆ cycloalkyl, carboxylic acid, carboxylic acid ester, amide, reverse amide, sulfonamide, reverse sulfonamide, N-acyl urea, and nitrogen-containing 5-membered heterocycle. Optionally, the 5-membered heterocycle can be further substituted with C₁-6 alkyl, alkoxy, fluorine-substituted alkyl, CN, and alkylsulfone.

Additionally in MLM-7, R₂₃ is selected from aryl, heteroaryl, —O-aryl, —O-heteroaryl, O-alkyl, —O-alkyl-cycloalkyl, —NH-alkyl, —NH-alkyl-cycloalkyl, —N(H)-aryl, —N(H)-heteroaryl, —N(alkyl)-aryl, or —N(alkyl)-heteroaryl. The aryl or heteroaryl groups can be substituted with halogen, C₁₋₆ alkyl, hydroxylated C₁₋₆ alkyl, cycloalkyl, fluorine-substituted C₁₋₆ alkyl, CN, alkoxy, alkyl sulfone, amide, and/or sulfonamide.

Further in MLM-7, R₂₄ is selected from the group consisting of —CH₂-C₁₋₆ alkyl, —CH₂-cycloalkyl, —CH₂-aryl, and —CH₂-heteroaryl. Optionally, the alkyl, cycloalkyl, aryl and heteroaryl groups can be substituted with halogen, alkoxy, hydroxylated alkyl, cyano-substituted alkyl, cycloalkyl, and/or substituted cycloalkyl.

Also in MLM-7, R₂₅ is selected from the group consisting of C₁₋₆ alkyl, C₁₋₆ alkyl-cycloalkyl, alkoxy-substituted alkyl, hydroxylated alkyl, aryl, heteroaryl, substituted aryl or heteroaryl, 5, 6, and 7-membered nitrogen-containing saturated heterocycles, 5,6-fused and 6,6-fused nitrogen-containing saturated heterocycles. Optionally, the saturated heterocycles can be substituted with C₁₋₆ alkyl, fluorine-substituted C₁₋₆ alkyl, alkoxy, aryl and heteroaryl group.

In MLM-8, R², R³, and Y are as defined above for MLM-7.

Also in MLM-8, R₂₆ is selected from the group consisting of C₁₋₆ alkyl and C₃₋₆ cycloalkyl. Optionally, the alkyl or cycloalkyl groups can be substituted with —OH, alkoxy, fluorine-substituted alkoxy, fluorine-substituted alkyl, —NH₂, —NH-alkyl, NH—C(O)alkyl, —NH—SO₂— alkyl, and/or —SO₂-alkyl.

Additionally in MLM-8, R₂₇ is selected from the group consisting of aryl, heteroaryl, and bicyclic heteroaryl. Optionally, the aryl or heteroaryl groups can be substituted with C₁₋₆ alkyl, alkoxy, NH₂, NH-alkyl, halogen, or —CN, and the substitution can be independently a mono-substitution, a bi-substitution, or a tri-substitution.

Further in MLM-8, R₂₈ is selected from the group consisting of aryl, 5 and 6-membered heteroaryl, bicyclic heteroaryl, cycloalkyl, and saturated heterocycle, such as piperidine, piperidinone, tetrahydropyran, or N-acyl-piperidine. Optionally, the cycloalkyl, saturated heterocycle, aryl, or heteroaryl can be further substituted with —OH, alkoxy, mono-, bis- or tri-substitution including halogen, —CN, alkyl sulfone, and fluorine substituted alkyl groups.

In some cases, the MLMs include those shown herein as well as hybrid molecules that arise from the combination of one or more of the features shown in the molecules. Optionally, exemplary MDM2 binding moieties can include those described in the following, which are incorporated herein by reference for their descriptions of groups corresponding to MLM groups:

US Published Patent Application 2010/0317661: WO 2012/155066; Gessier et al., J. Med. Chem., 58(16): 6348-58 (2015); Liu et al, Science, 303(5659): 844-8 (2004); and Graves et al., J. Med. Chem. 56(14): 5979-83 (2013).

Also, in the formula shown above, L is a bond or a chemical linker group. In some cases, L is any group corresponding to an L group recited in U.S. Published Patent Application 2020/0085793, which is incorporated herein by reference for their descriptions of groups corresponding to L.

Optionally, L is selected from the group consisting of substituted or unsubstituted alkylene, substituted or unsubstituted alkenylene, substituted or unsubstituted alkenylene, substituted or unsubstituted carbocyclylene, substituted or unsubstituted heterocyclylene, substituted or unsubstituted arylene, substituted or unsubstituted heteroarylene, substituted or unsubstituted heteroalkylene, a bond, —O—, —NH(R^{A})—, —S—, —CO—, —COO—, —CONR^{A}—, —NR^{A}CO, —NR^{A}COR^{A}—, and —COR^{A}—. Each RA is independently selected from C₁-6 alkyl, wherein one or more carbon is optionally replaced with halo. CO, CONH, O, S, SO, SO₂, N, NHCO, and/or heterocycle.

Optionally, L is represented by the formula -(A^{L})_{q}-, wherein A^{L} is a chemical moiety and q is greater than or equal to 0. In some cases, each A^{L} is independently selected from the group consisting of a bond, CR^{L1}R^{L2}, O, S, SO, SO₂, NR^{L3}, SO₂NR^{L3}, SONR^{L3}, CONR^{L3}, NR^{L3}CONR^{W}, NR^{L3}SO₂NR^{W}, CO, CR^{L1}═CR^{L2}, C═C, SiR^{L1}R^{L2}, P(O)R^{L1}, P(O)OR^{L1}, NR^{L3}C(═NCN)NR^{W}, NR^{L3}C(═NCN), NR^{L3}C(═NO₂)NR^{L4}, C₃₋₁₁ cycloalkyl optionally substituted with 0-6 R^{L1} and/or R^{L2} groups, C₅₋₁₃ spirocycloalkyl optionally substituted with 0-9 R^{L1} and/or R^{L2} groups, C3-diheterocyclyl optionally substituted with 0-6 R^{L1} and/or R^{L2} groups, C₅₋₁₃ spiroheterocycloalkyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R¹⁵ groups: and R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁ heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═H(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NHSO₂NH(C₁₋₈alkyl), NHSO₂N(C₁₋₈alkyl)₂, NHSO₂NH₂.

In some cases, q is greater than or equal to 1 (e.g., 1 to 100, 1 to 75, 1 to 50, 1 to 25, 1 to 15, 1 to 10). In some cases, q is 0, 1, 2, 3, 4, 5, 6, 7, 8, 9, or 10.

Each A^{L} is independently selected from the group consisting of: —NR(CH₂)ₙ-(lower alkyl, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(hetero cycloalkyl)-, —NR(CH₂CH₂O)ₙ-(lower alkyl)-O—CH₂—, —NR(CH₂CH₂O)ₙ-(hetero cycloalkyl)-O—CH₂—, —NR(CH₂CH₂O)ₙ-aryl-O—CH₂—, NR(CH₂CH₂O)ₙ-(hetero aryl)-O—CH₂—, —NR(CH₂CH₂O)ₙ-(cycloalkyl)-O-(hetero aryl)-O—CH₂—, —NR(CH₂CH₂O)ₙ-(cycloalkyl)-O-Aryl-O—CH₂—, —NR(CH₂CH₂O)ₙ-(lower alkyl)-NH-aryl-O—CH₂—, —NR(CH₂CH₂O)ₙ-(lower alkyl)-O-aryl-CH₂, —NR(CH₂CH₂O)ₙ-cycloalkyl-O-aryl-, —NR(CH₂CH₂O)ₙ-cycloalkyl-O-(heteroaryl)1-, —NR(CH₂CH₂)ₙ-(cycloalkyl)-O-(heterocycle)-CH₂, —NR(CH₂CH₂)ₙ-(heterocycle)-(heterocycle)-CH₂, or —N(R₁R₂)-(heterocycle)CH₂. Then group of the linker can be 0 to 10; the R of the linker can be H, lower alkyl; and R₁ and R₂ of the linker can form a ring with the connecting N.

Optionally, A^{L} is selected from the group consisting of:

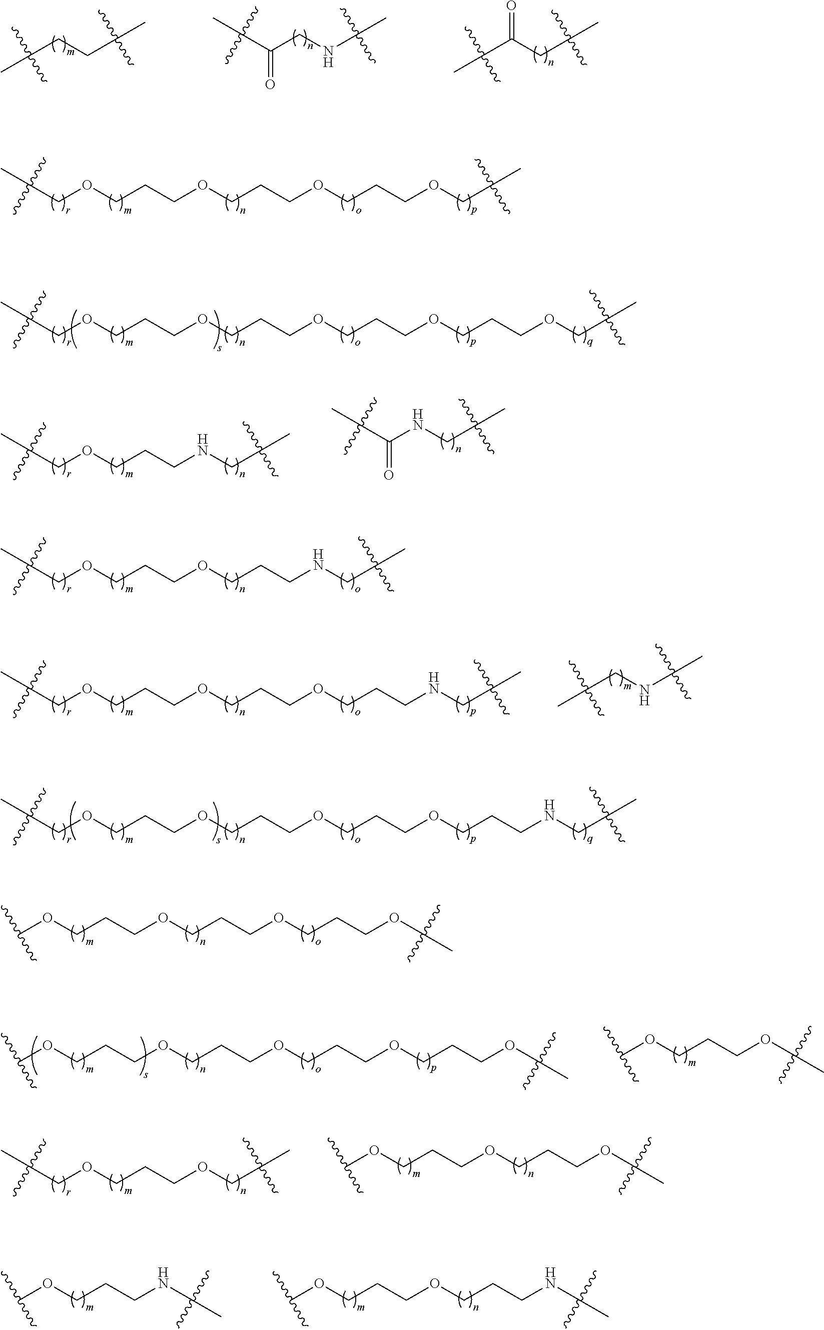

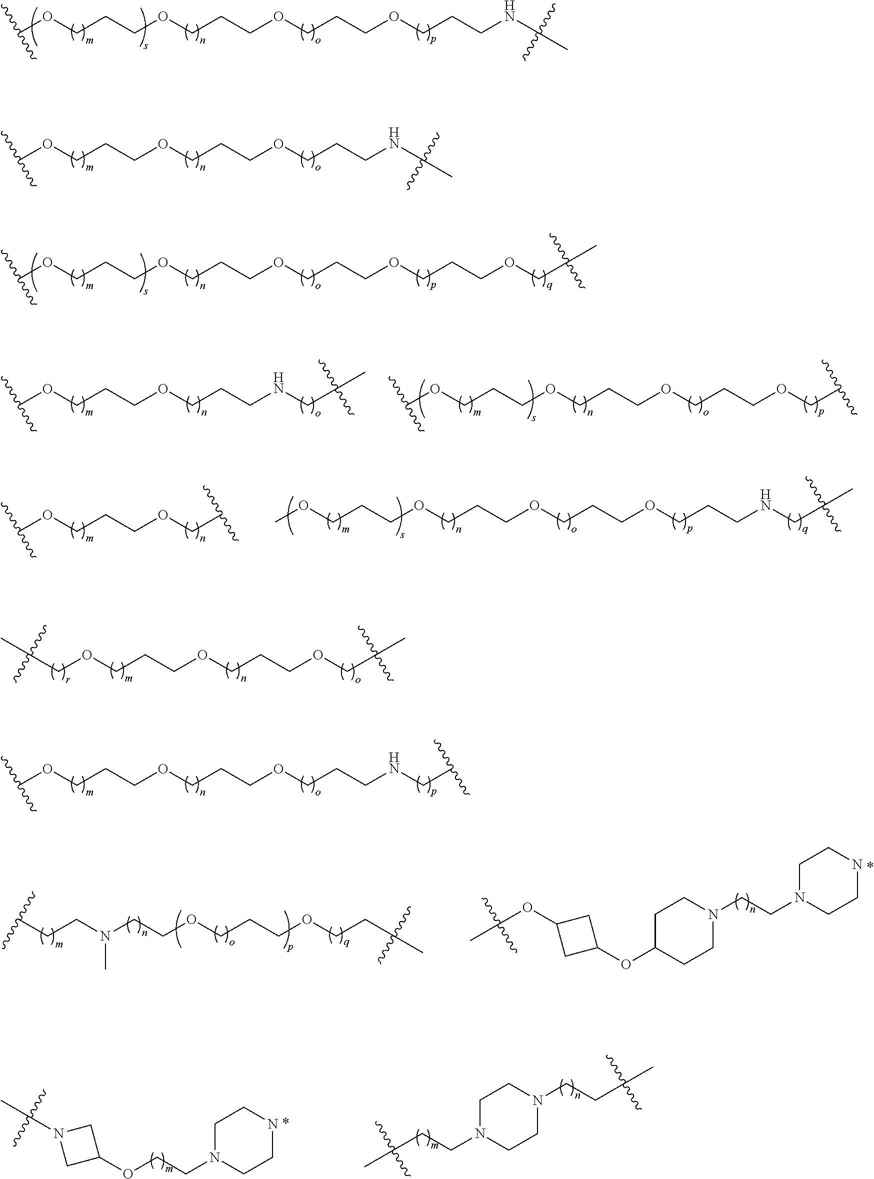

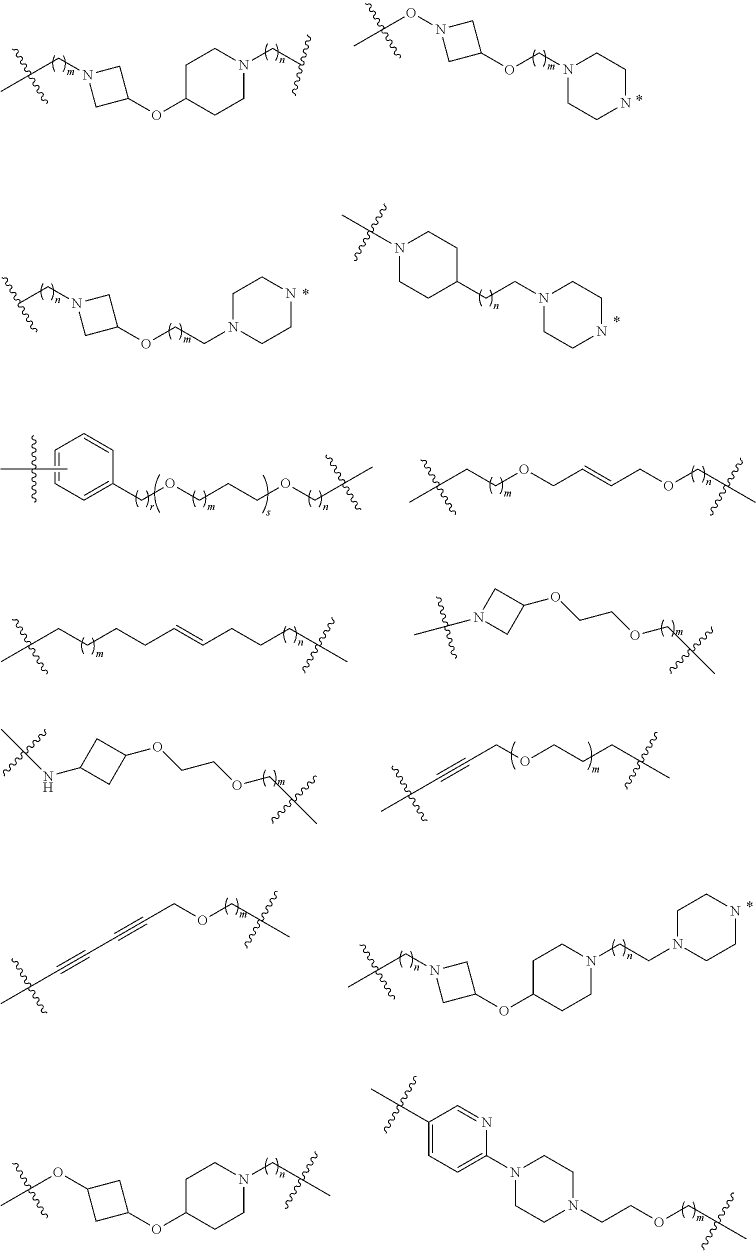

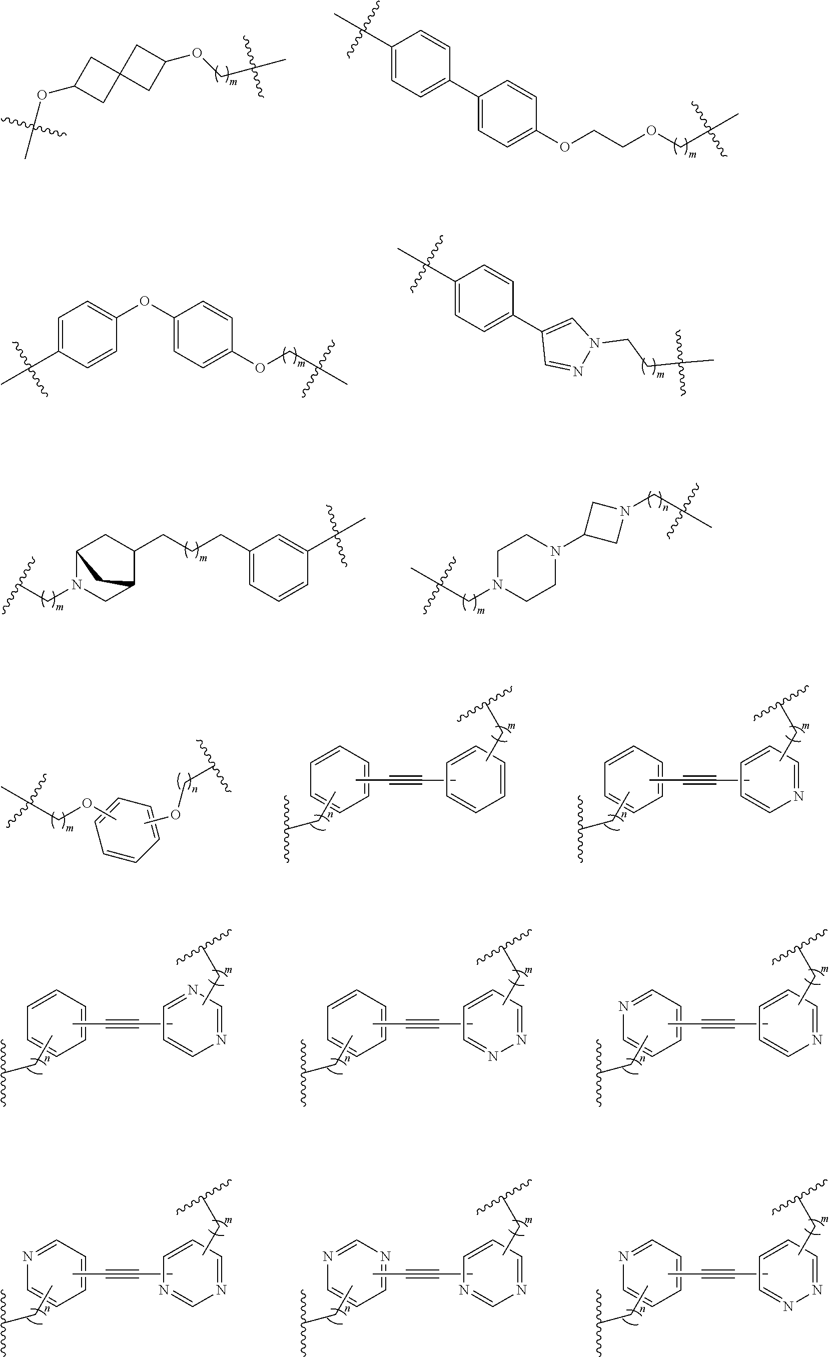

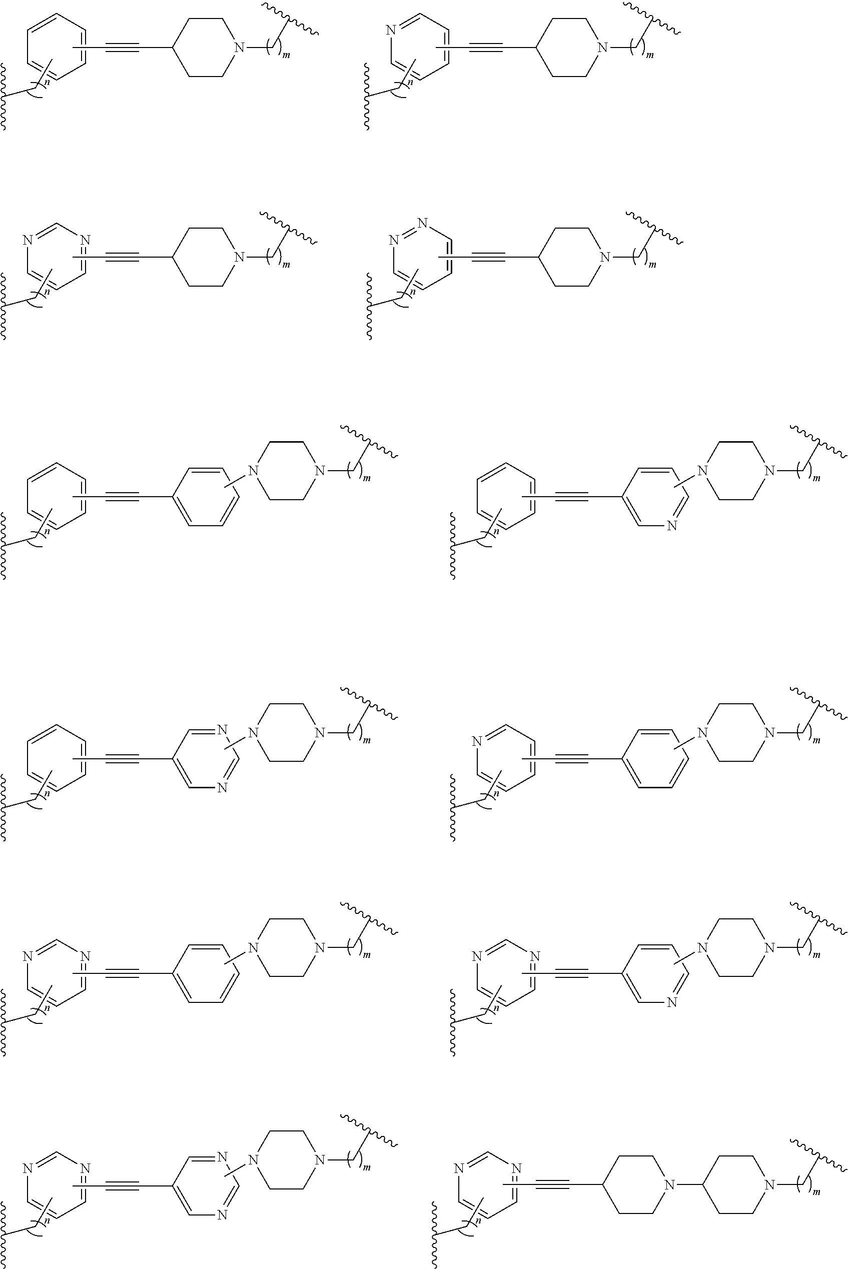

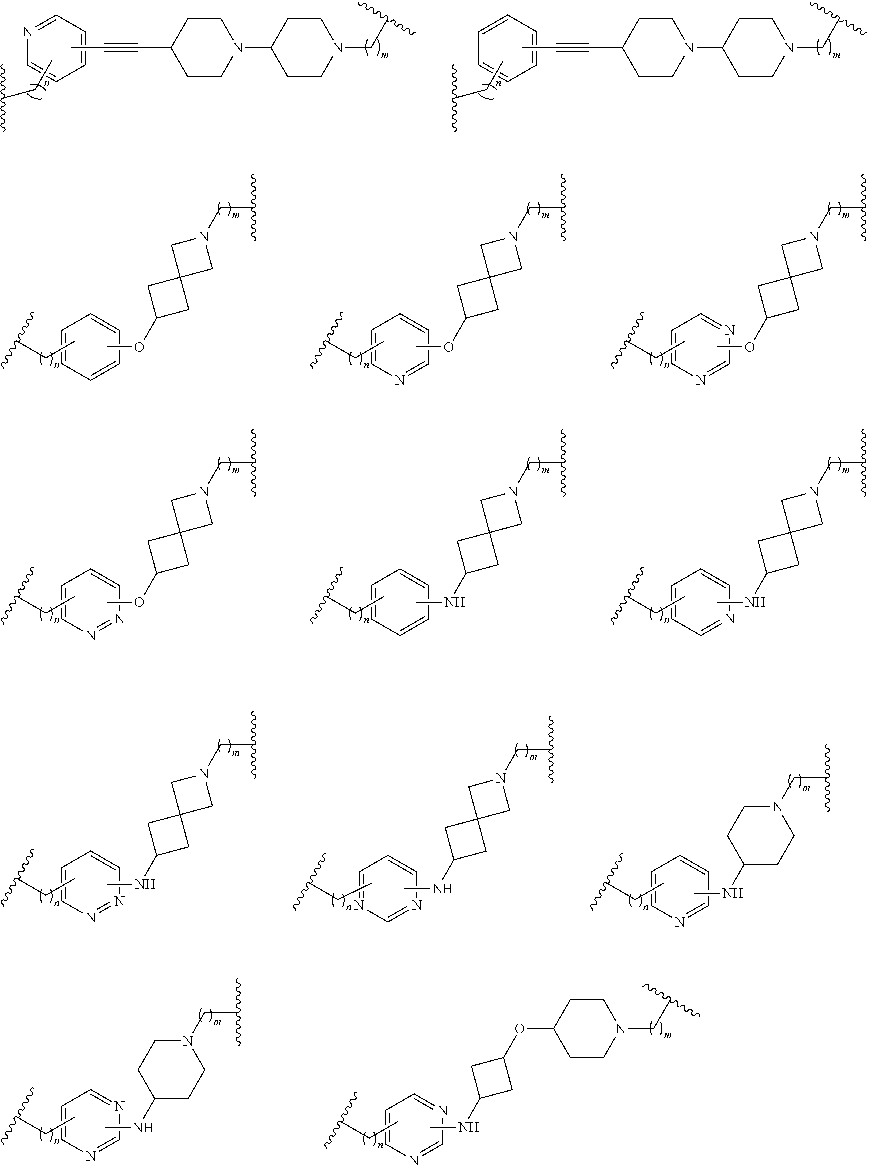

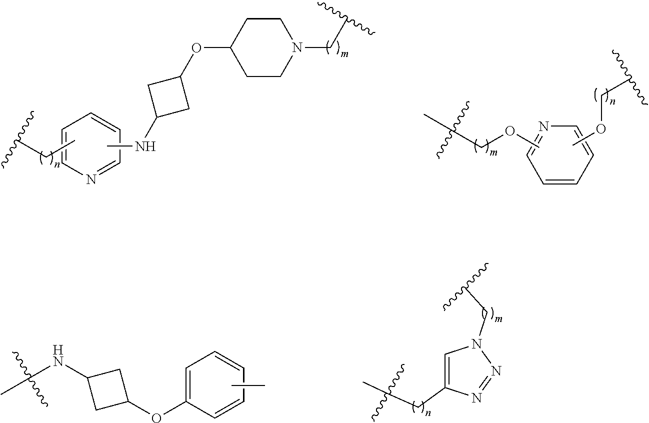

wherein each of m, n, o, p, q, r, and s are independently selected from 0-10, and N* of the heterocycloalkyl is shared with the TPM or the ELM or is linked to the TPM or the ELM via a bond.

An exemplary class of RIPK1 kinase degraders according to the structure shown above is provided below as Formula I:

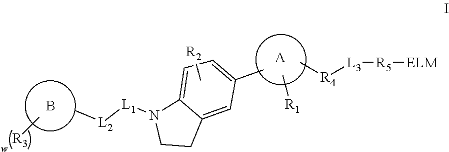

and pharmaceutically acceptable salts, co-crystals, tautomers, stereoisomers, solvates, hydrates, polymorphs, isotopically enriched derivatives, and prodrugs thereof.

In Formula I, ELM is an E3 ubiquitin ligase binding moiety.

Also in Formula I, Ring A and Ring B are each independently selected from a 5-10 membered aryl ring or a heterocycle ring.

Additionally in Formula I, L₁ is —S(O)—, —S(O)₂—, or —C(O)—.

Further in Formula I, L₂ is a bond, —N(R₁)—, —CH(R₁)—, or —R₁—.

Also in Formula I, L₃ is —(CH₂)ₙ— or —(OCH₂CH₂)ₙ—.

Additionally in Formula I, each of R₁, R₂, R₃ are independently hydrogen, halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, R₅, tetrazole, aryl, aryl substituted with from one to three substituents independently selected from halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, R₄, heterocycle, heterocycle substituted with from one to three substituents independently selected from halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, and R₄.

Further in Formula I, R₄ and R₅ are independently a bond or C₁₋₁₂ alkyl, wherein one or more carbons are optionally replaced with halo, CO, CONH, O, S, SO, SO₂, N, NHCO, heterocycle.

Also in Formula I, n is independently an integer from 0 to 20 (e.g., 0 to 18 or 0 to 15). For example, n can be 0, 2, 3, 4, 5, 6, 7, 8, 9, 10, 11, 12, 13, 14, or 15.

Additionally in Formula I, w is independently an integer from 0 to 4. For example, w can be 0, 1, 2, 3, or 4.

In some embodiments of Formula I, Ring A is selected from the following bicycloheteroaryls:

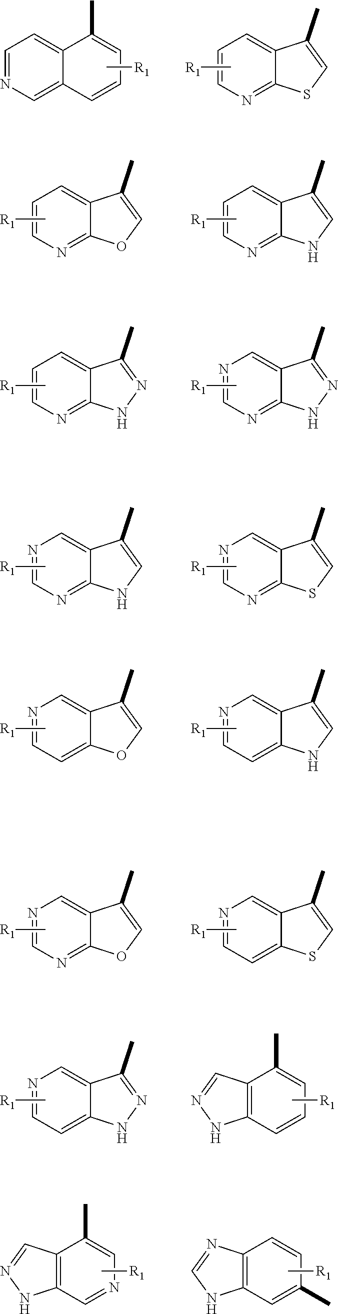

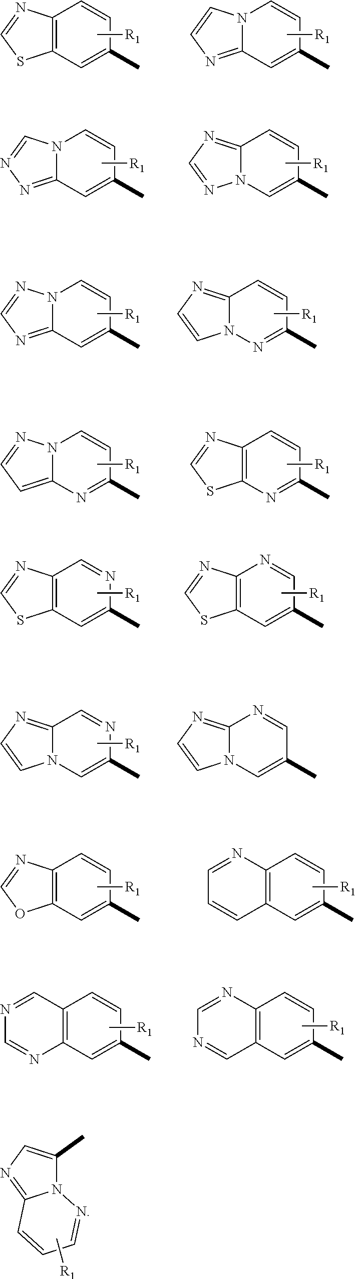

Ring B is independently phenyl or 6 membered heterocycle ring; L₁ is —C(O)—; L₂ is —N(R₁)— or —CH(R₁)—; L₃ is —(CH₂)ₙ— or —(OCH₂CH₂)ₙ—; R₁ is selected from hydrogen, halogen, —OH, —OR₄, —NH₂, —NR₄R₅, —NR₄COR₅, —CN, —CONH₂, —CONR₄R₅, —CF₃, —OCF₃, tetrazole, aryl, heterocycle; R₂, R₃ are independently hydrogen, halogen, —OH, —OR₄, —NH₂, —NR₄R₅, —NR₄COR₅, —CN, —CONR₄R₅, —CF₃, —OCF₃; R₄ and R₅ are independently a bond or C₁₋₁₂ alkyl, wherein one or more carbon is optionally replaced with halo, CO, CONH, O, SO₂, N, NHCO; n is independently an integer from 0 to 10; and w is independently an integer from 0 to 3.

In some embodiments of Formula I, Ring A is selected from the following bicycloheteroaryls:

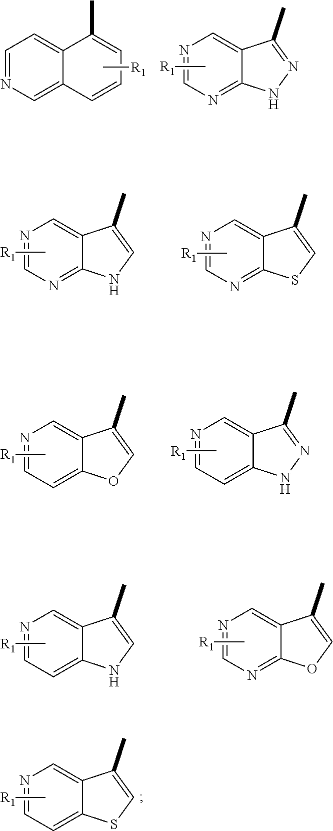

Ring B is independently phenyl, pyridine, pyrimidine, pyridazine, pyrazine; L₁ is —C(O)—; L₂ is —N(R₁)— or —CH(R₁)—; L₃ is —(CH₂)ₙ— or —(OCH₂CH₂)ₙ—; R₁ is selected from hydrogen, halogen —OR₄, —NH₂, —NR₄R₅, —NR₄COR₅, —CONR₄R₅, —CF₃, —OCF₃; R₂ and R₃ are independently hydrogen, halogen, —OH, —OMe, —NH₂, —CN, —CONH₂, —CF₃, —OCF₃; R₄ and R₅ are independently a bond or C₁-6 alkyl, wherein one or more carbon is optionally replaced with halo, CO, CONH, O, SO₂, N, NHCO; n is independently an integer from 0 to 10; and w is independently an integer from 0 to 3.

An exemplary class of RIPK1 kinase degraders according to the structure shown above is provided below as Formula II:

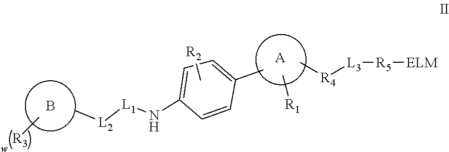

and pharmaceutically acceptable salts, co-crystals, tautomers, stereoisomers, solvates, hydrates, polymorphs, isotopically enriched derivatives, or prodrugs thereof.

In Formula II, ELM is an E3 ubiquitin ligase binding moiety.

Also in Formula II, Ring A and Ring B is independently selected from 5-10 membered aryl ring or heterocycle ring.

Additionally in Formula II, L₁ is —S(O)—, —S(O)₂— or —C(O)—.

Further in Formula II, L₂ is a bond, —N(R₁)—, —CH(R₁)—, or —R₁.

Also in Formula II, L₃ is —(CH₂)ₙ— or —(OCH₂CH₂)ₙ—.

Additionally in Formula II, each of R₁, R₂, R₃ are independently hydrogen, halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, R₄, tetrazole, aryl, aryl substituted with from one to three substituents independently selected from halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, R₄, heterocycle, heterocycle substituted with from one to three substituents independently selected from halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, and R₄.

Further in Formula II, R₄ and R₅ are independently a bond or C₁₋₁₂ alkyl, wherein one or more carbon is optionally replaced with halo, CO, CONH, O, S, SO, SO₂, N, NHCO, or heterocycle.

Also in Formula II, n is independently an integer from 0 to 15. For example, n can be 0, 1, 2, 3, 4, 5, 6, 7, 8, 9, 10, 11, 12, 13, 14, or 15.

Additionally in Formula II, w is independently an integer from 0 to 4. In some cases, w can be 0, 1, 2, 3, or 4.

In some embodiments of Formula II, Ring A is selected from the following bicycloheteroaryls:

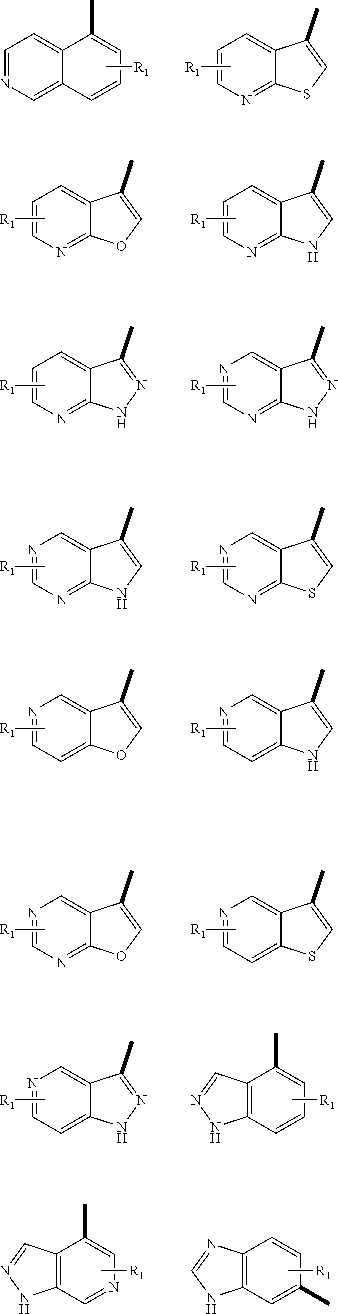

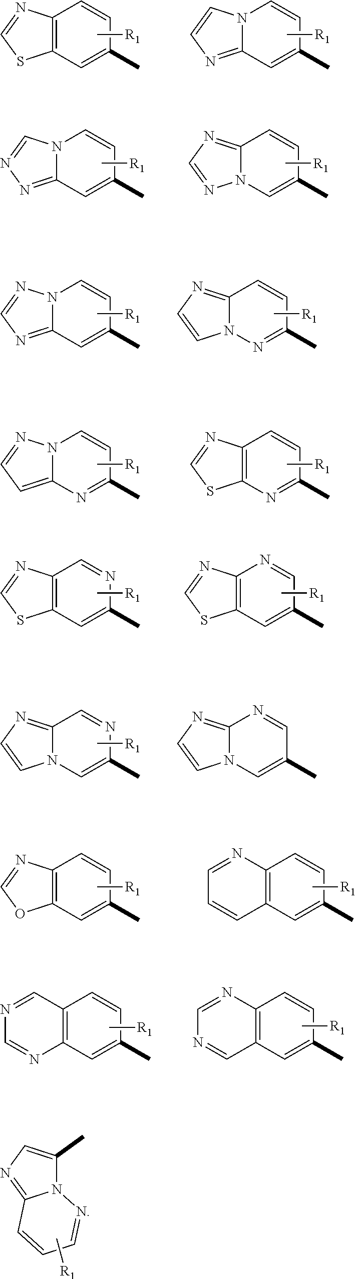

Ring B is independently phenyl or 6 membered heterocycle ring; L₁ is —C(O)—; L₂ is —N(R₁)— or —CH(R₁)—; L₁ is —(CH₂)ₙ— or —(OCH₂CH₂)ₙ—; R₁ is selected from hydrogen, halogen, —OH, —OR₄, —NH₂, —NR₄R₅, —NR₄COR₅, —CN, —CONH₂, —CONR₄R₅, —CF₃, —OCF₃, tetrazole, aryl, and heterocycle; R₂ and R; are each independently hydrogen, halogen, —OH, —OR₄, —NH₂, —NR₄R₅, —NR₄COR₅, —CN, —CONR₄R₅, —CF₃, or —OCF₃; R₄ and R₅ are independently a bond or C₁₋₁₂ alkyl, wherein one or more carbon is optionally replaced with halo, CO, CONH, O, SO₂, N, or NHCO; n is independently an integer from 0 to 10; and w is independently an integer from 0 to 3.

In some embodiments. Ring A is selected from the following bicycloheteroaryls:

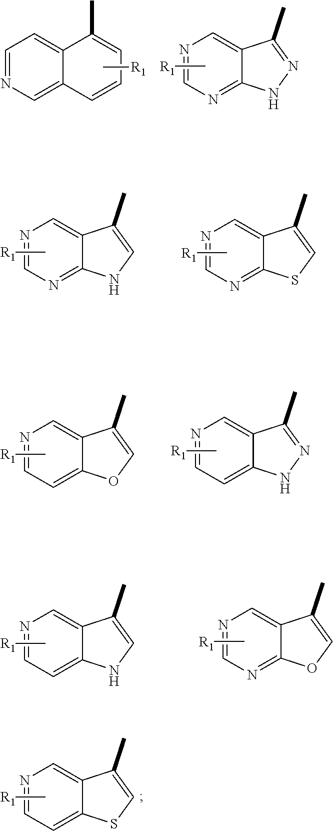

Ring B is independently phenyl, pyridine, pyrimidine, pyridazine, or pyrazine; L₁ is —C(O)—; L₂ is —N(R₁)— or —CH(R₁)—; L₃ is —(CH₂)ₙ— or —(OCH₂CH₂)ₙ—; R₁ is selected from hydrogen, halogen —OR₄, —NH₂, —NR₄R₅, —NR₄COR₅, —CONR₄R₅, —CF₃, and —OCF₃; R₂ and R₃ are independently hydrogen, halogen, —OH, —OMe, —NH₂, —CN, —CONH₂, —CF₃, or —OCF₃; R₄ and R₅ are independently a bond or C₁₋₁₂ alkyl, wherein one or more carbon is optionally replaced with halo, CO, CONH, O, SO₂, N, or NHCO; n is independently an integer from 0 to 18; and w is independently an integer from 0 to 3.

An exemplary class of RIPK1 kinase degraders according to the structure shown above is provided below as Formula III:

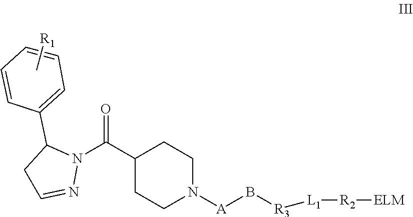

and pharmaceutically acceptable salts, co-crystals, tautomers, stereoisomers, solvates, hydrates, polymorphs, isotopically enriched derivatives, or prodrugs thereof.

In Formula III, ELM is an E3 ubiquitin ligase binding moiety.

Also in Formula III, A is —CO—, one or two R₁ substituted 5-10 membered aryl ring or one or two R₁ substituted heteroaryl ring.

Additionally in Formula III, B is a bond, one or two R₁ substituted 5-10 membered aryl ring, or one or two R₁ substituted heteroaryl ring.

Also in Formula III, R₁ is independently hydrogen, halogen, —OH, —OR₂, —NH₃, —NR₂R₃, —NR₂COR₃, —CN, —COOH, —COOR₃, —CONH₂, —CONR₂R₃, —CF₃, —OCF₃, —SO₃H, —SO₃R₃, or R₃;

Additionally in Formula III, R₂ and R₃ are each independently a bond or C₁₋₆ alkyl, wherein one or more carbon is optionally replaced with halo, CO, CONH, O, S, SO, SO₂, N, or NHCO.

Further in Formula III, L₁ is —(CH₂)ₙ— or —(OCH₂CH₂)ₙ—.

Also in Formula III, n is independently an integer from 0 to 20.

In some embodiments of Formula III, A is —CO—, 5-10 membered aryl ring or heteroaryl ring; B is a bond or phenyl; R₁ is independently hydrogen, halogen, —OH, —OMe, NH₃, —CN, —CONH₂, —CONR₂R₃, —CF₃, —OCF₃; R₂ and R₃ are independently a bond or C₁-6 alkyl, wherein one or more carbon is optionally replaced with halo, CO, CONH, O, S, SO, SO₂, N, NHCO; L₁ is —(CH₂)ₙ— or —(OCH₂CH₂)ₙ—; and n is independently an integer from 0 to 10.

Examples of the compounds according to Formula I, Formula II, and Formula III as described herein include the following compounds:

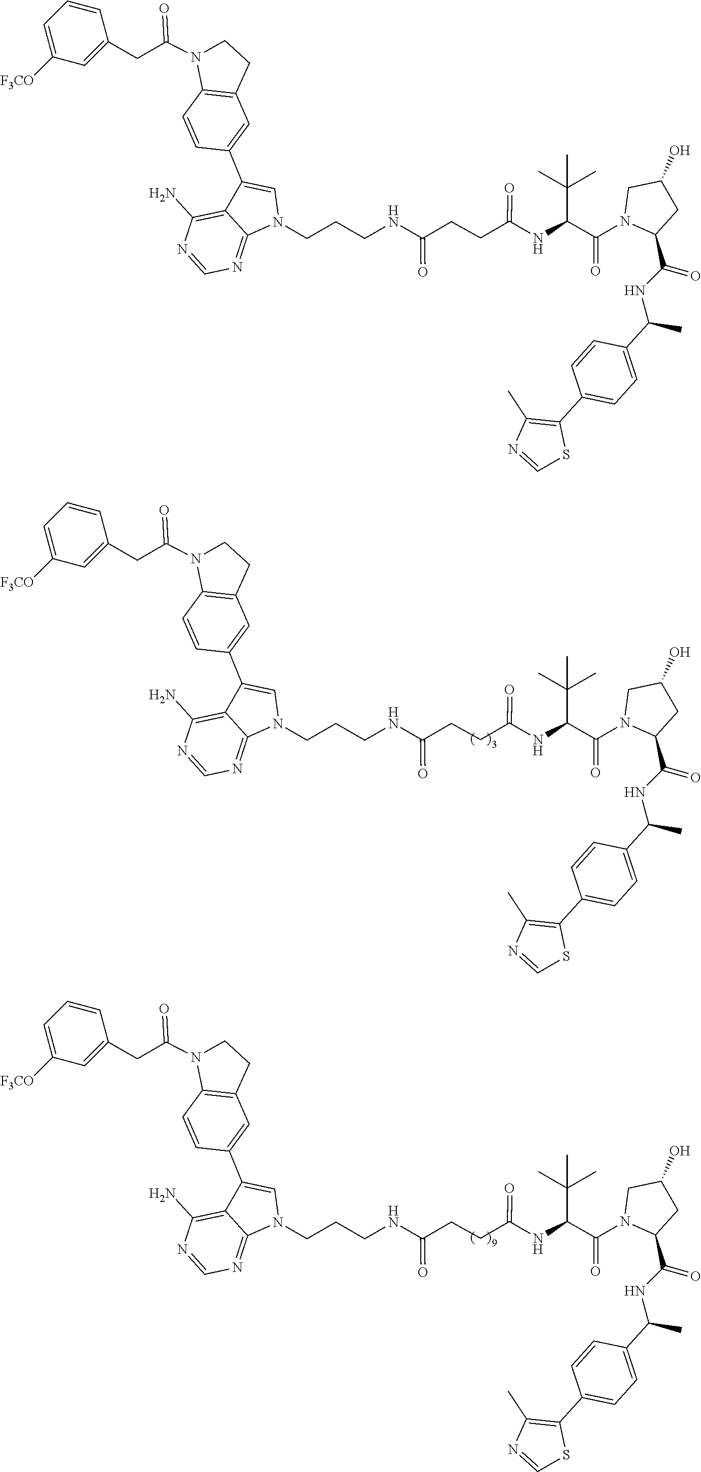

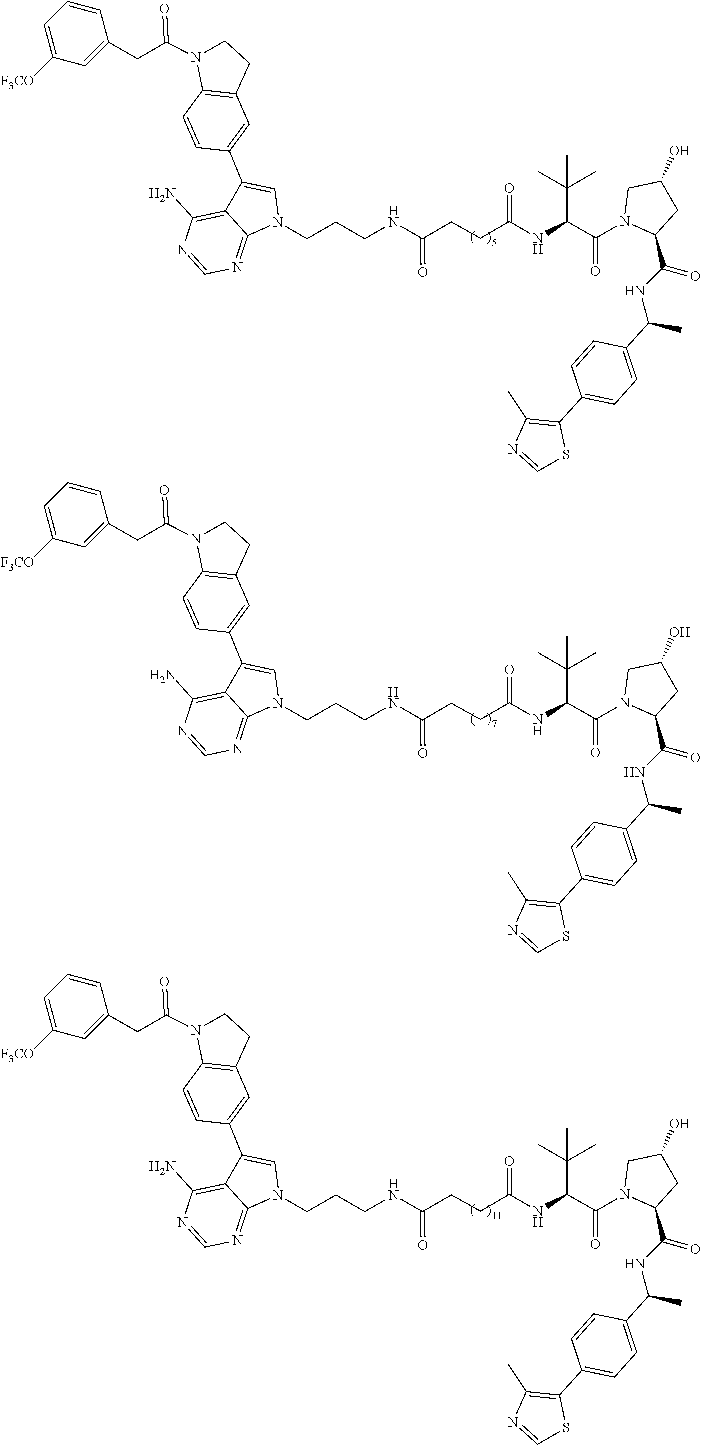

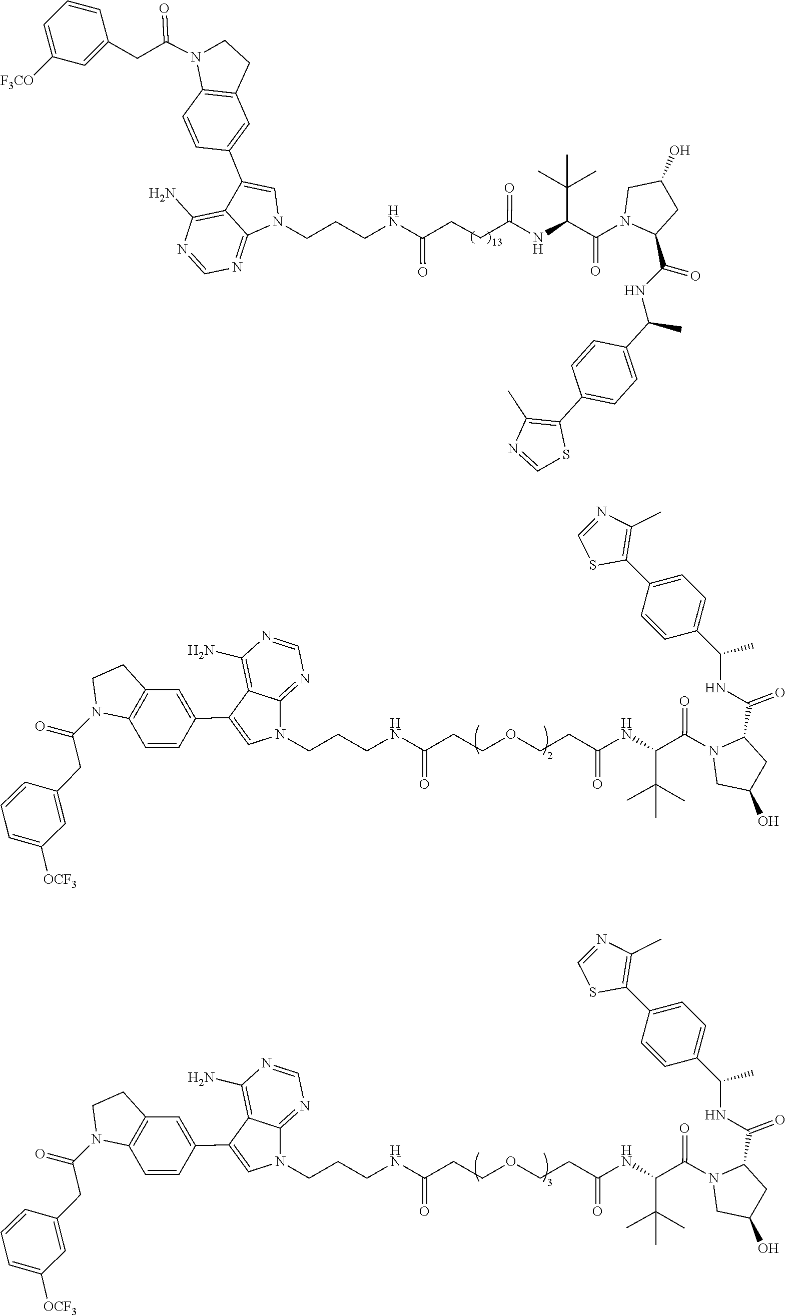

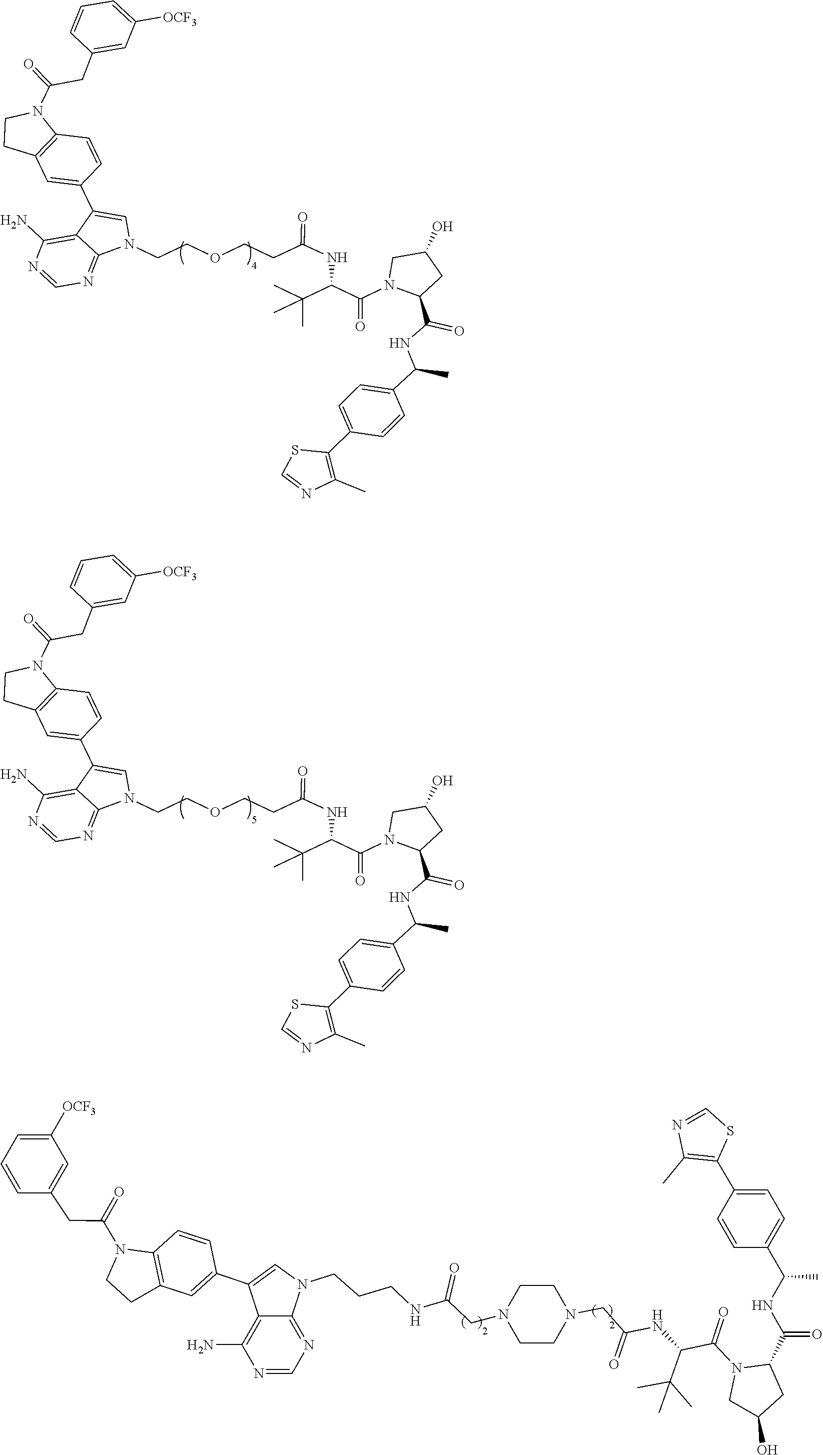

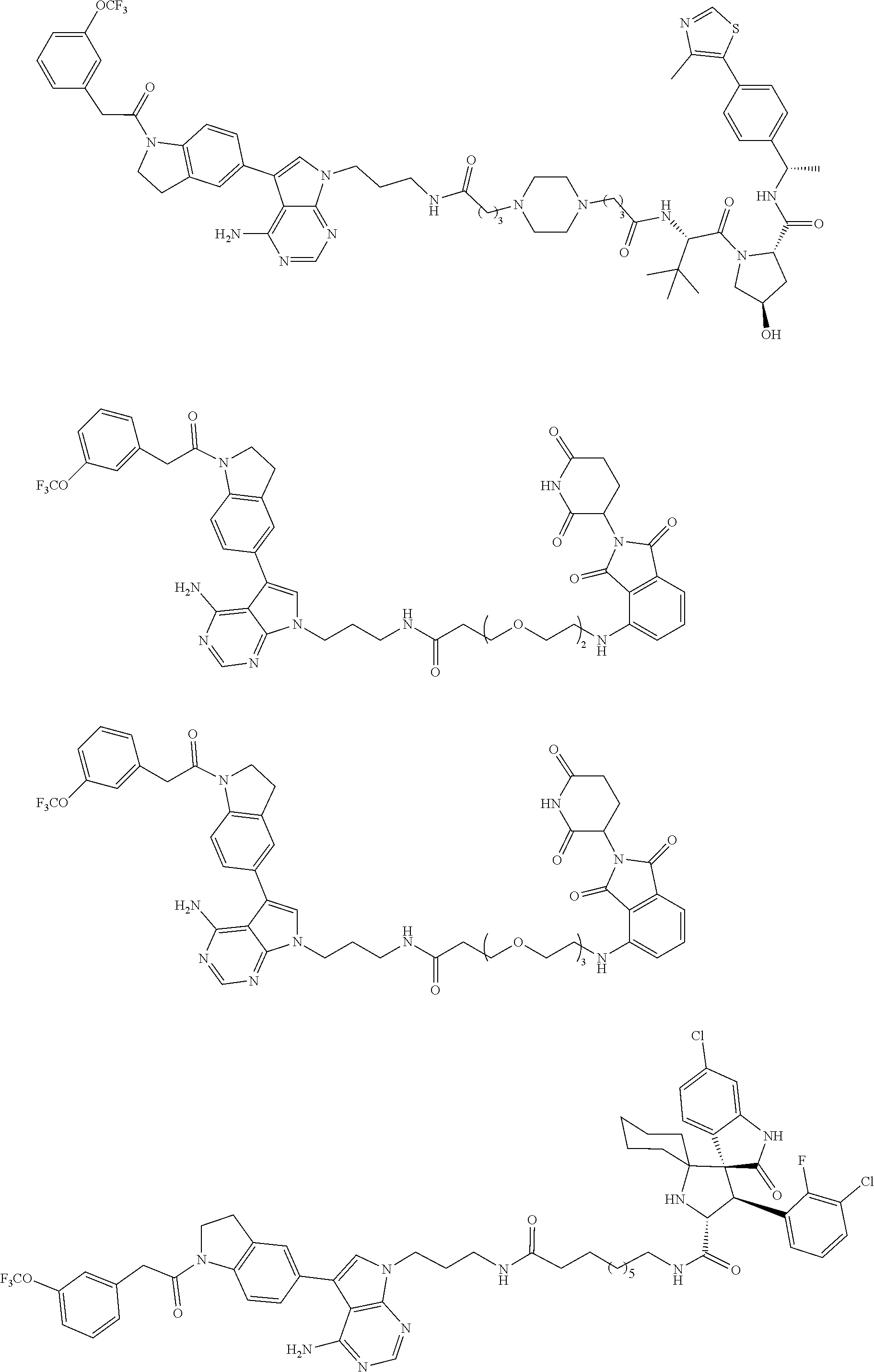

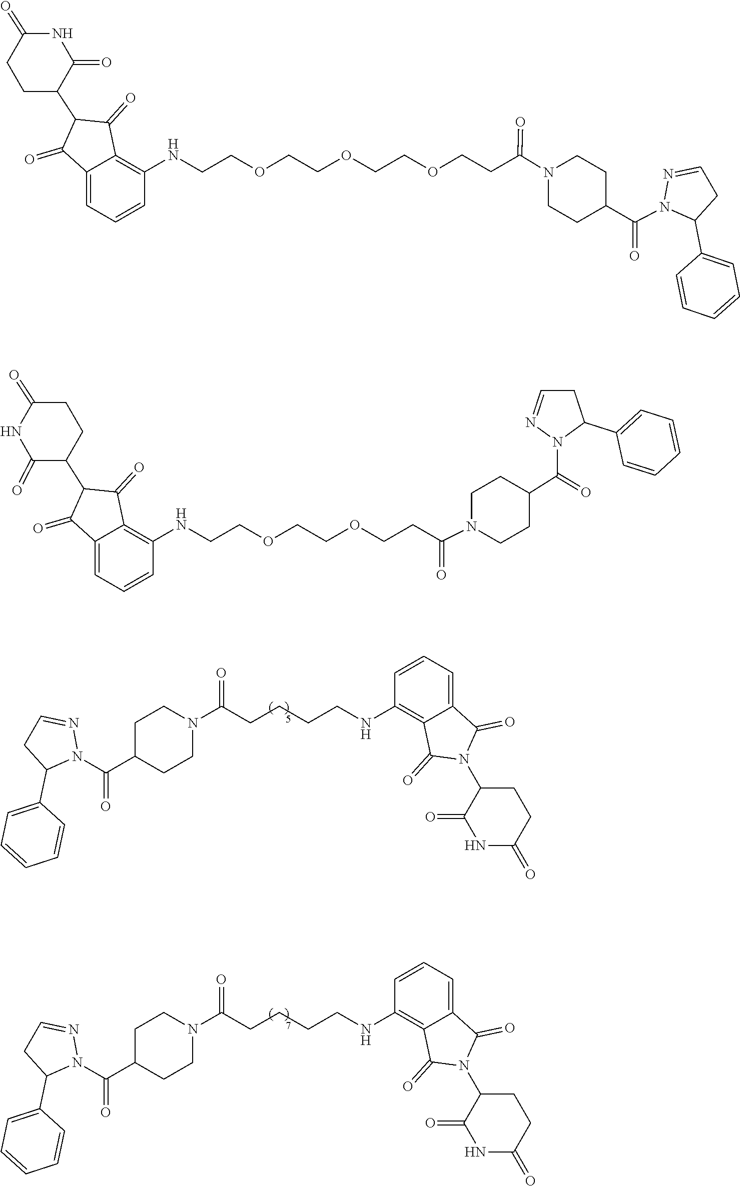

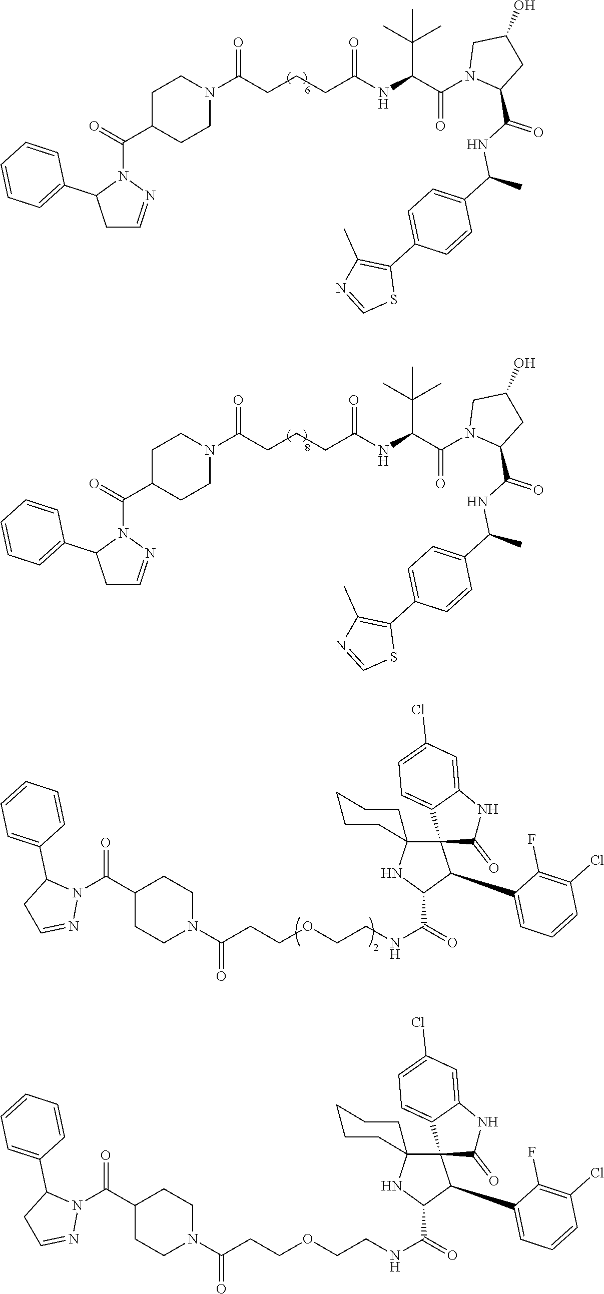

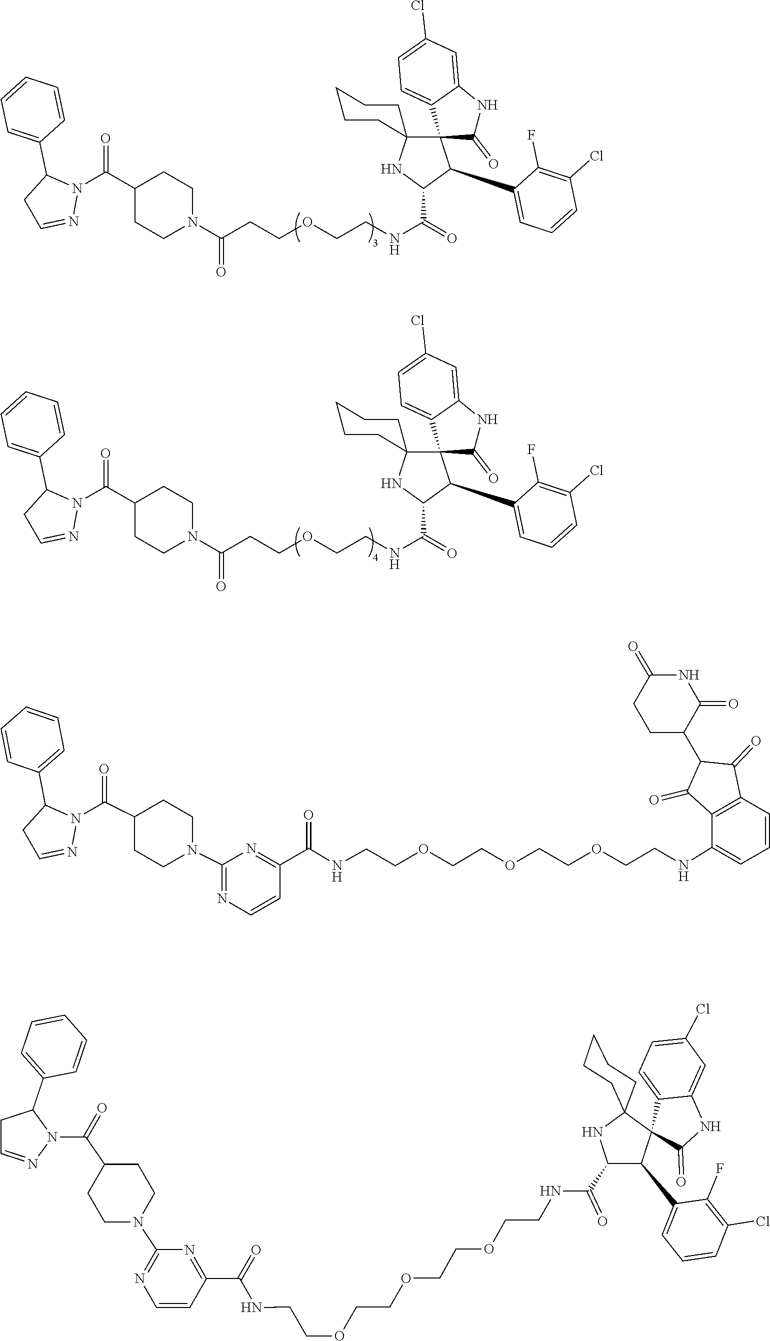

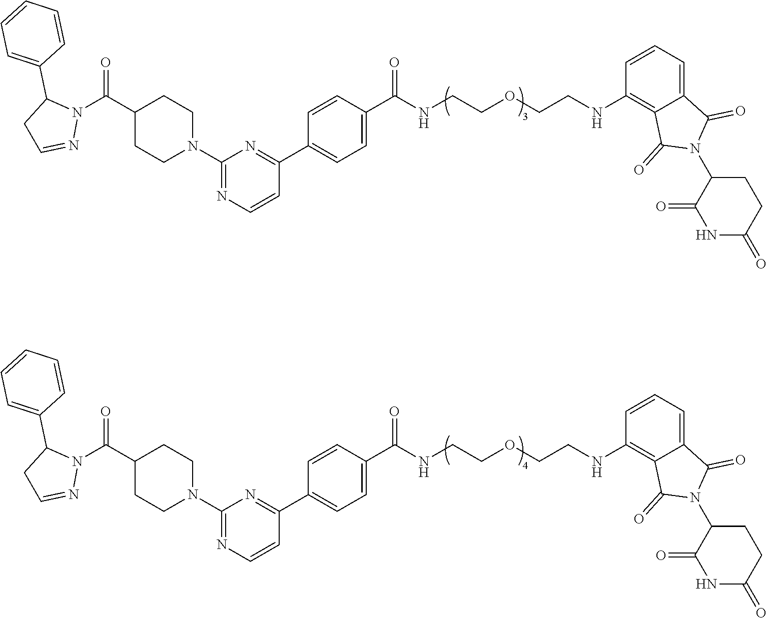

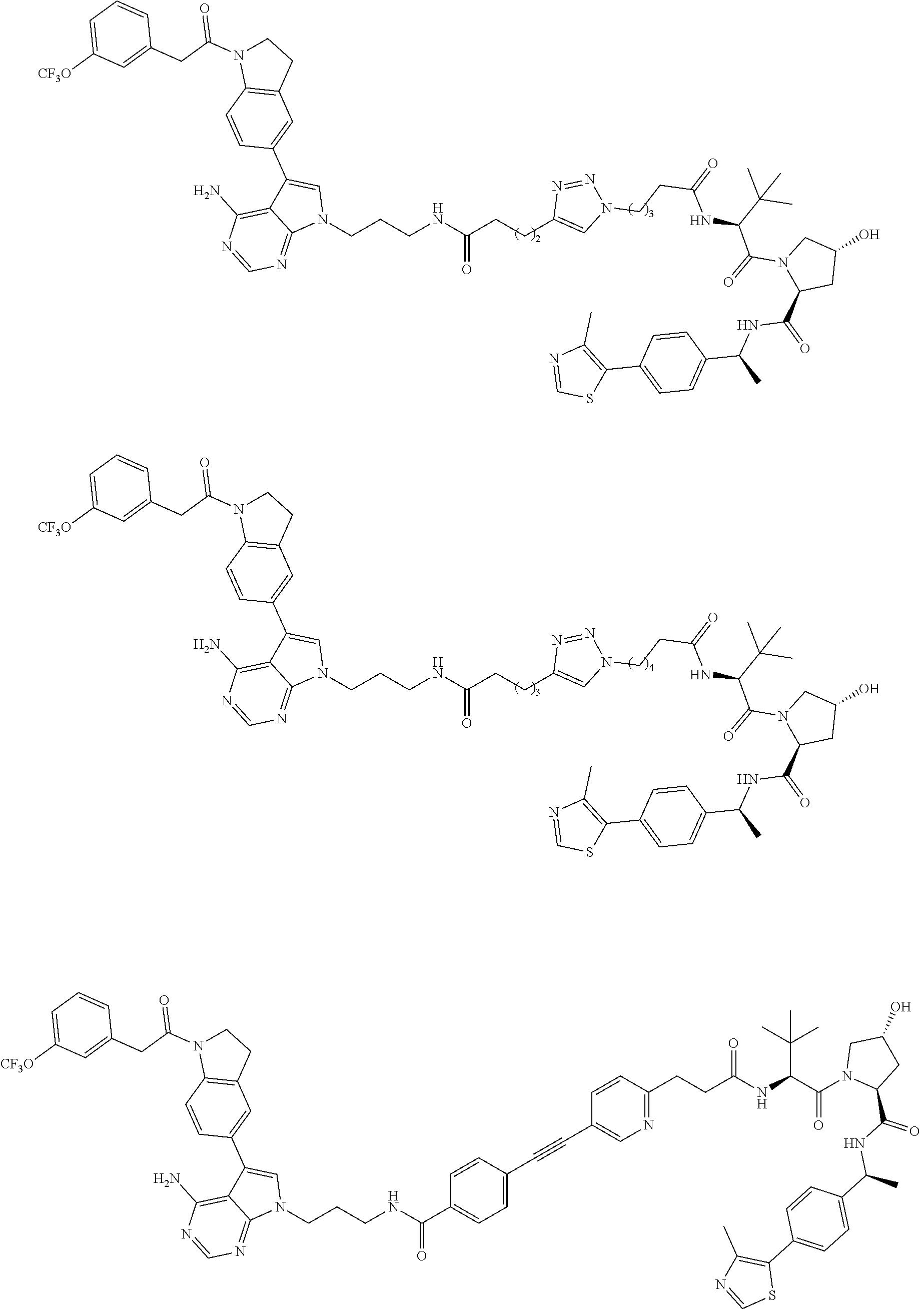

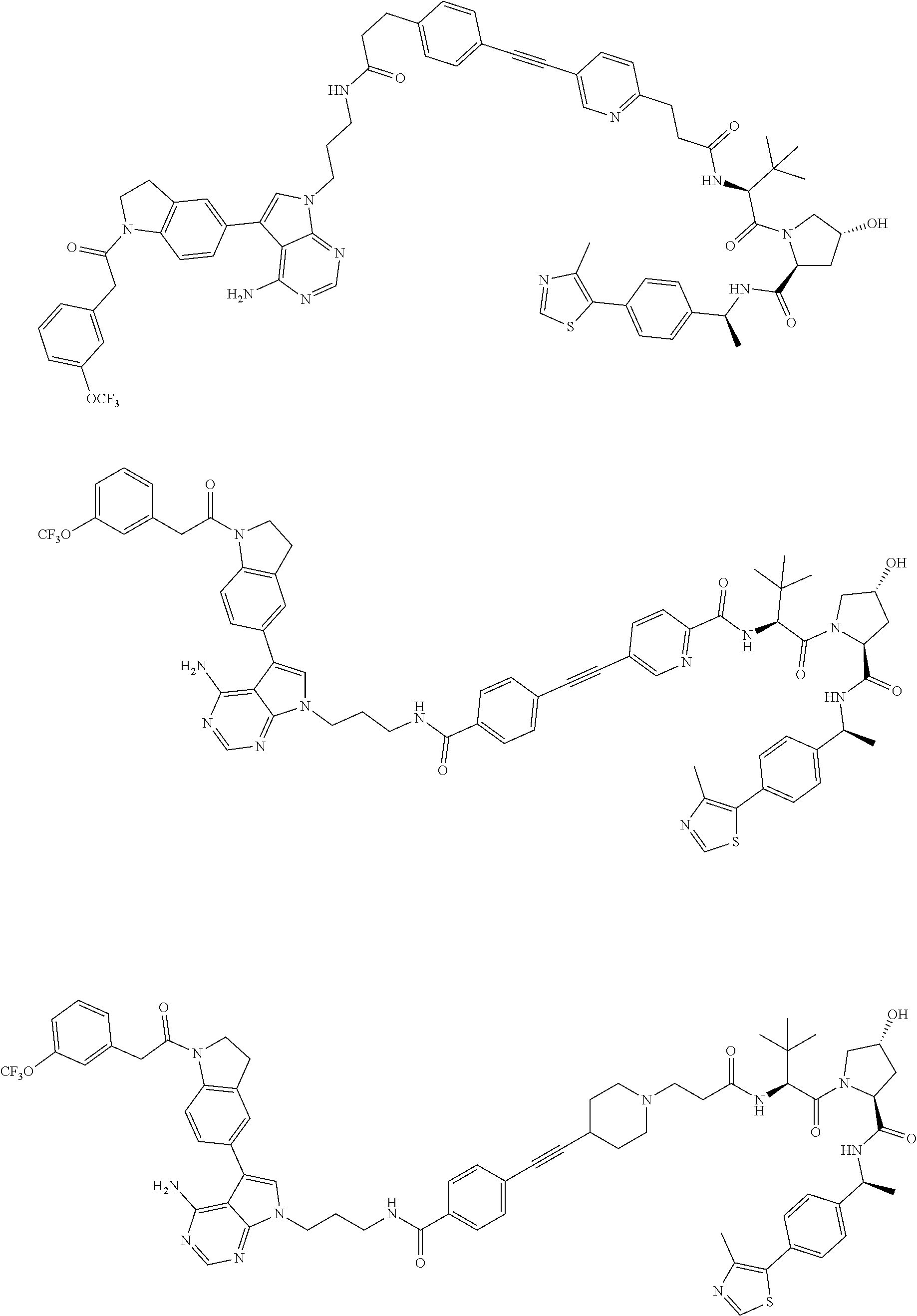

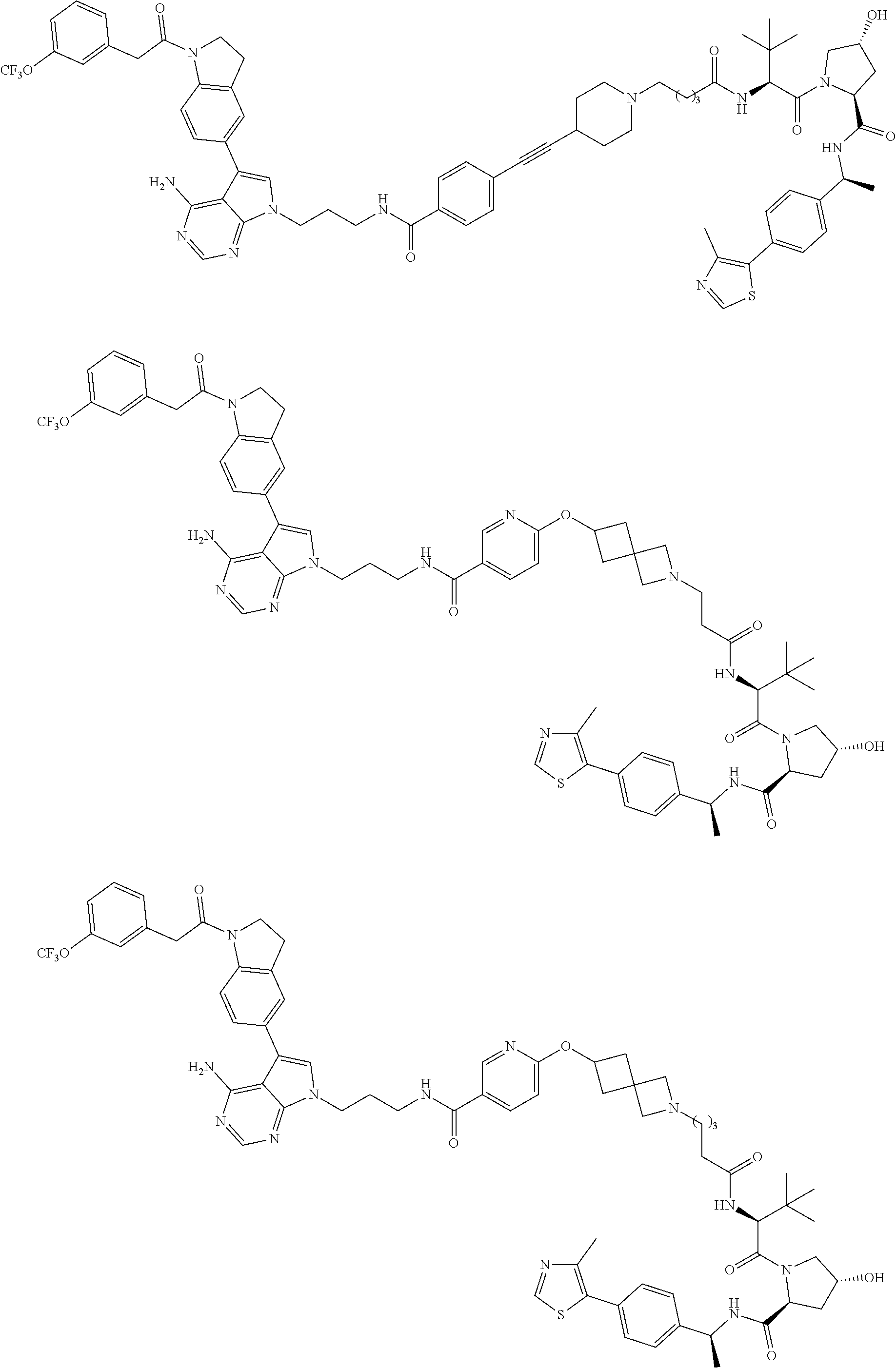

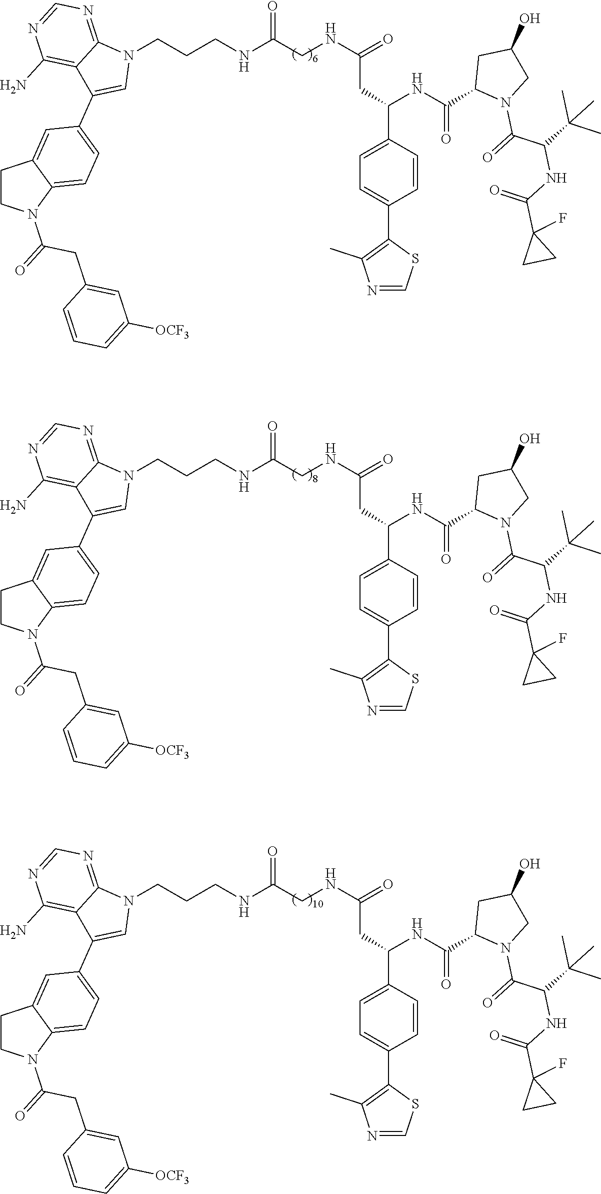

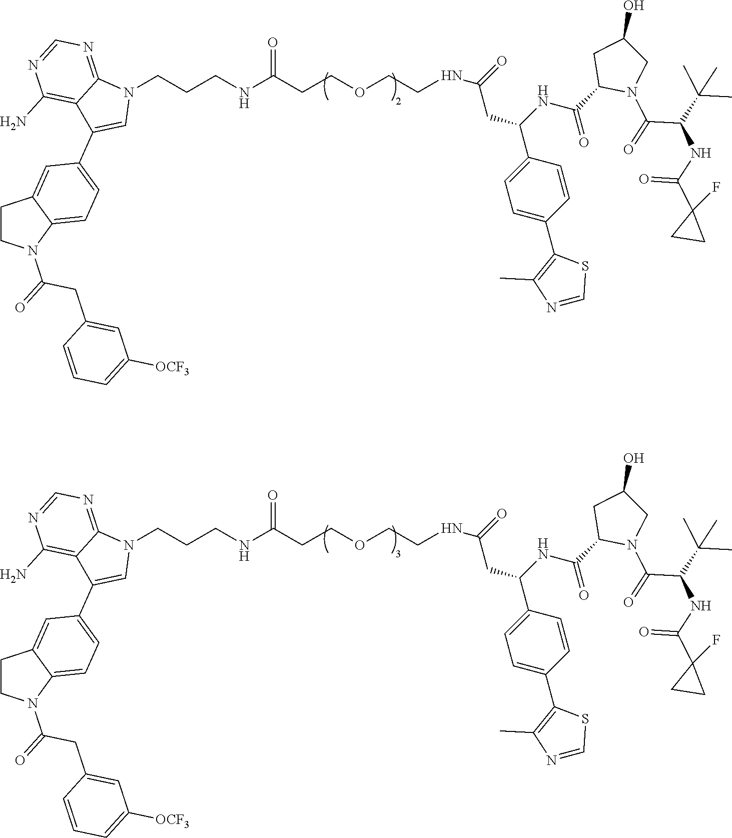

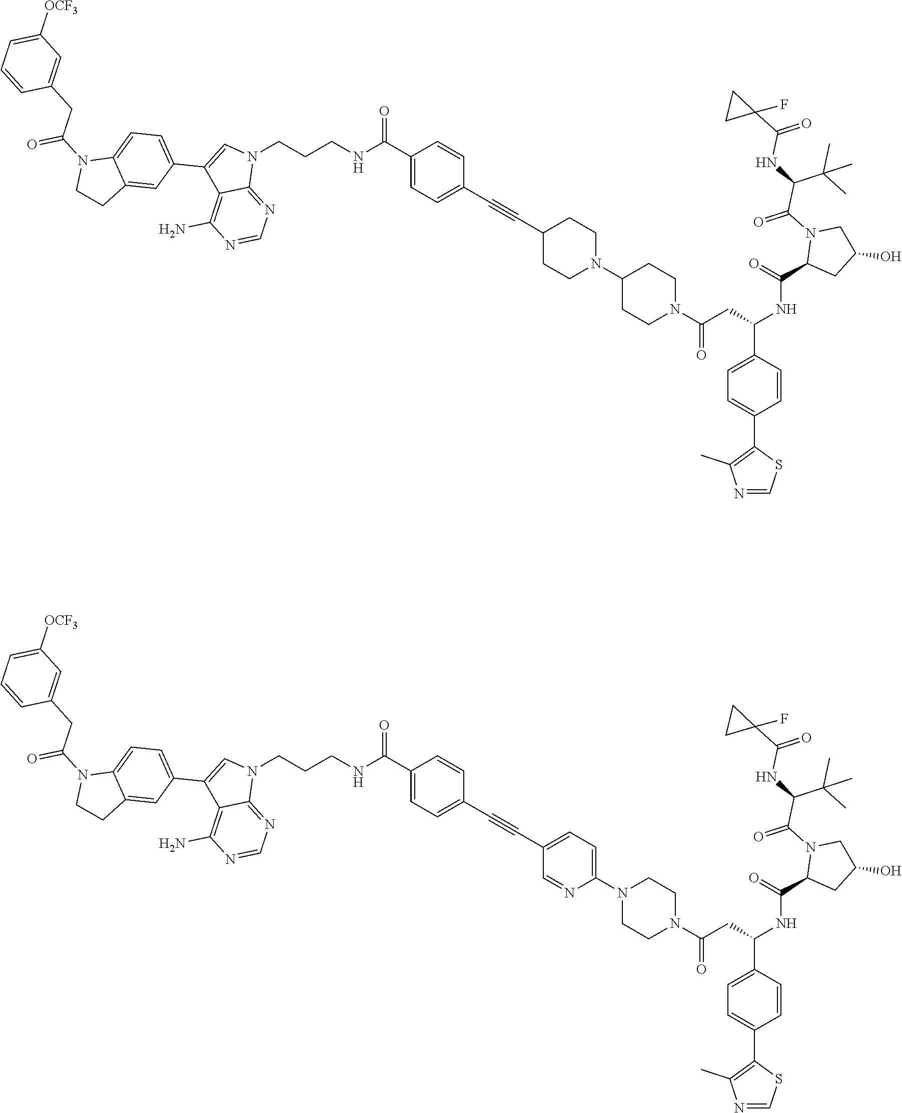 [SMILES: Cc1ncsc1-c1ccc([C@H](CC(=O)N2CCC(N3CCC(C#Cc4ccc(C(=O)NCCCn5cc(-c6ccc7c(c6)CCN7C(=O)Cc6cccc(OC(F)(F)F)c6)c6c(N)ncnc65)cc4)CC3)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)N2CCN(c3ccc(C#Cc4ccc(C(=O)NCCCn5cc(-c6ccc7c(c6)CCN7C(=O)Cc6cccc(OC(F)(F)F)c6)c6c(N)ncnc65)cc4)cn3)CC2)NC(=O)[C@@H]2C[C@@H](O)CN2C(=O)[C@@H](NC(=O)C2(F)CC2)C(C)(C)C)cc1]

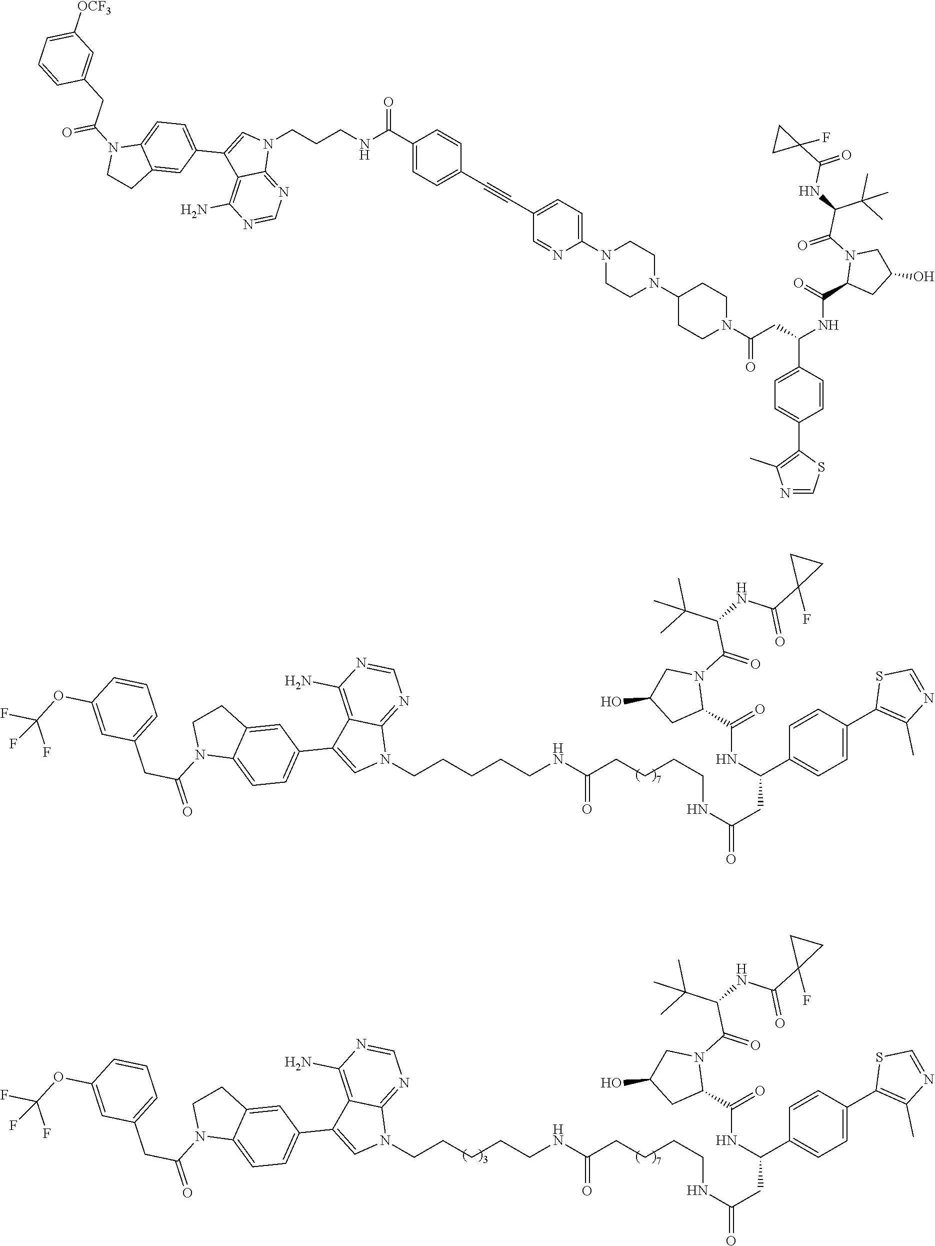

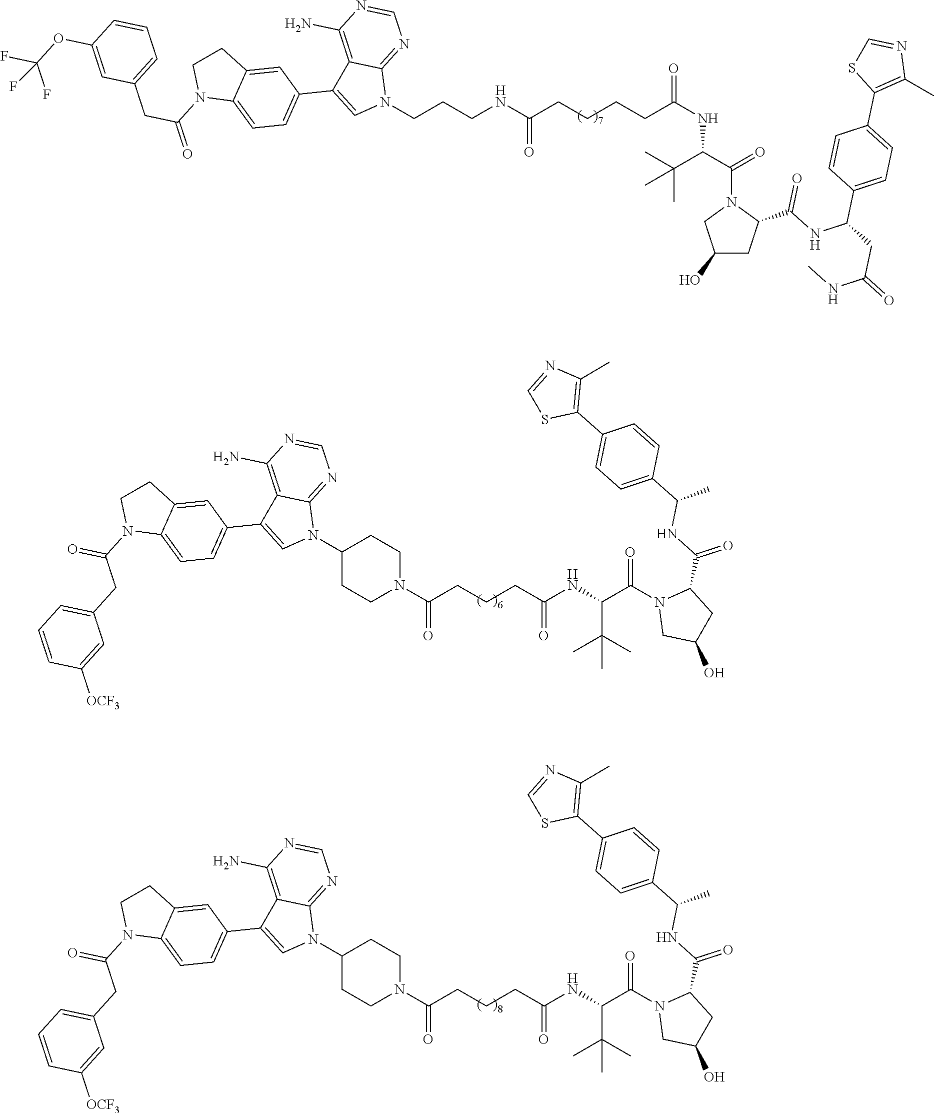

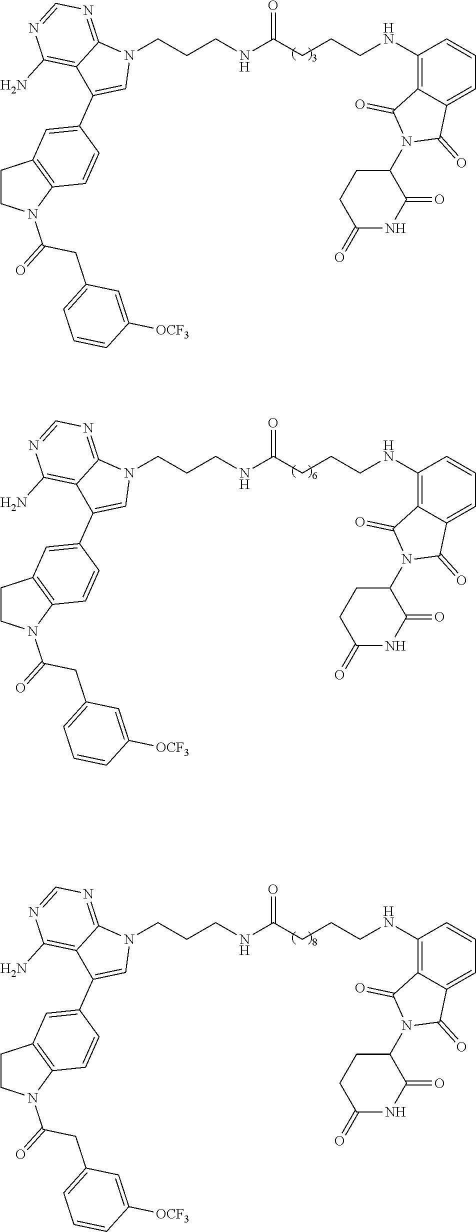

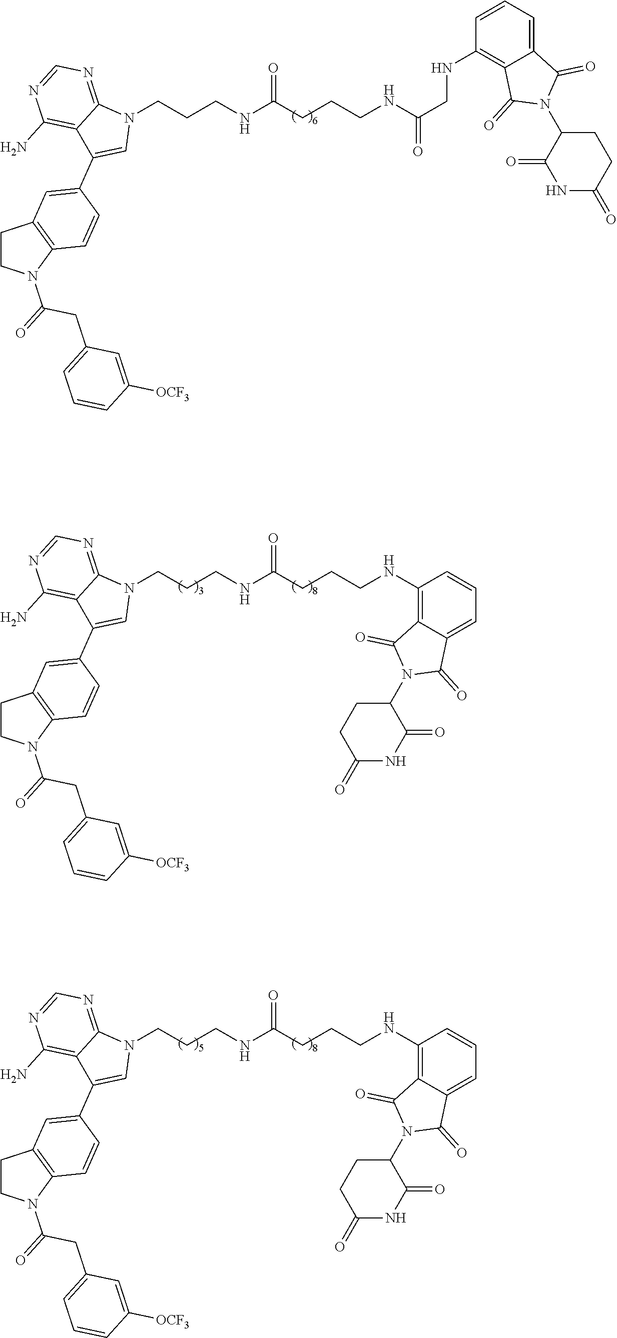

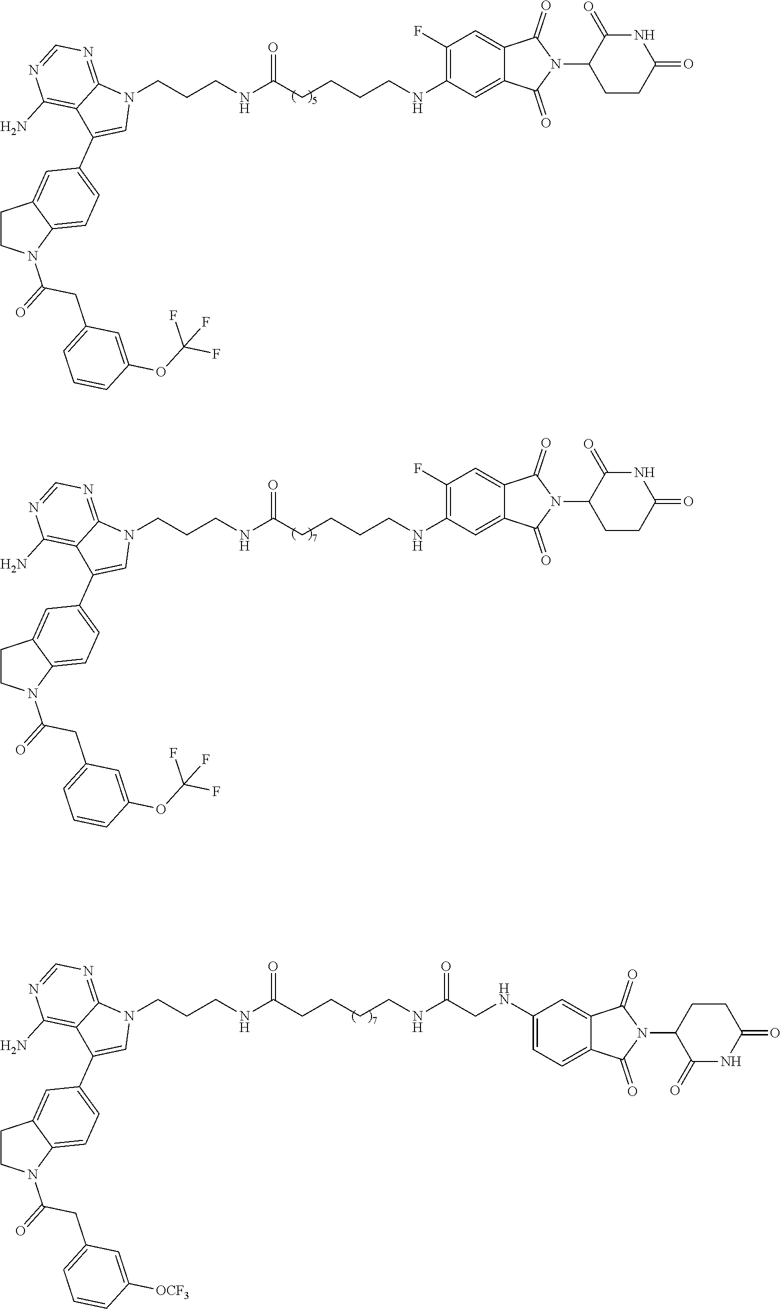

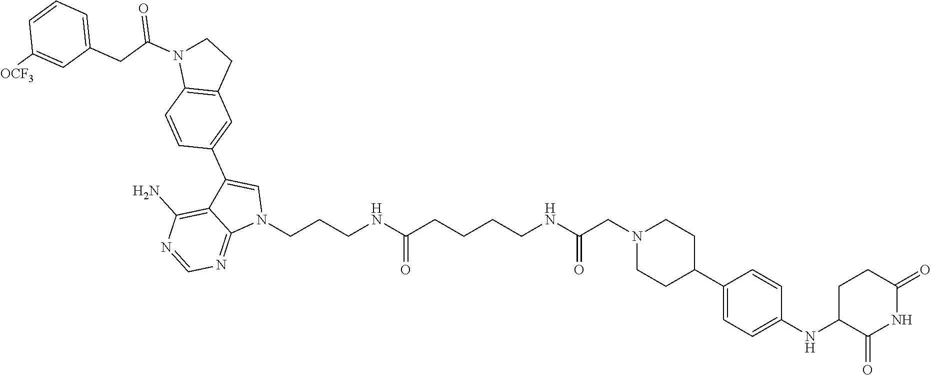 [SMILES: Nc1ncnc2c1c(-c1ccc3c(c1)CCN3C(=O)Cc1cccc(OC(F)(F)F)c1)cn2CCCNC(=O)CCCCNC(=O)CN1CCC(c2ccc(NC3CCC(=O)NC3=O)cc2)CC1]

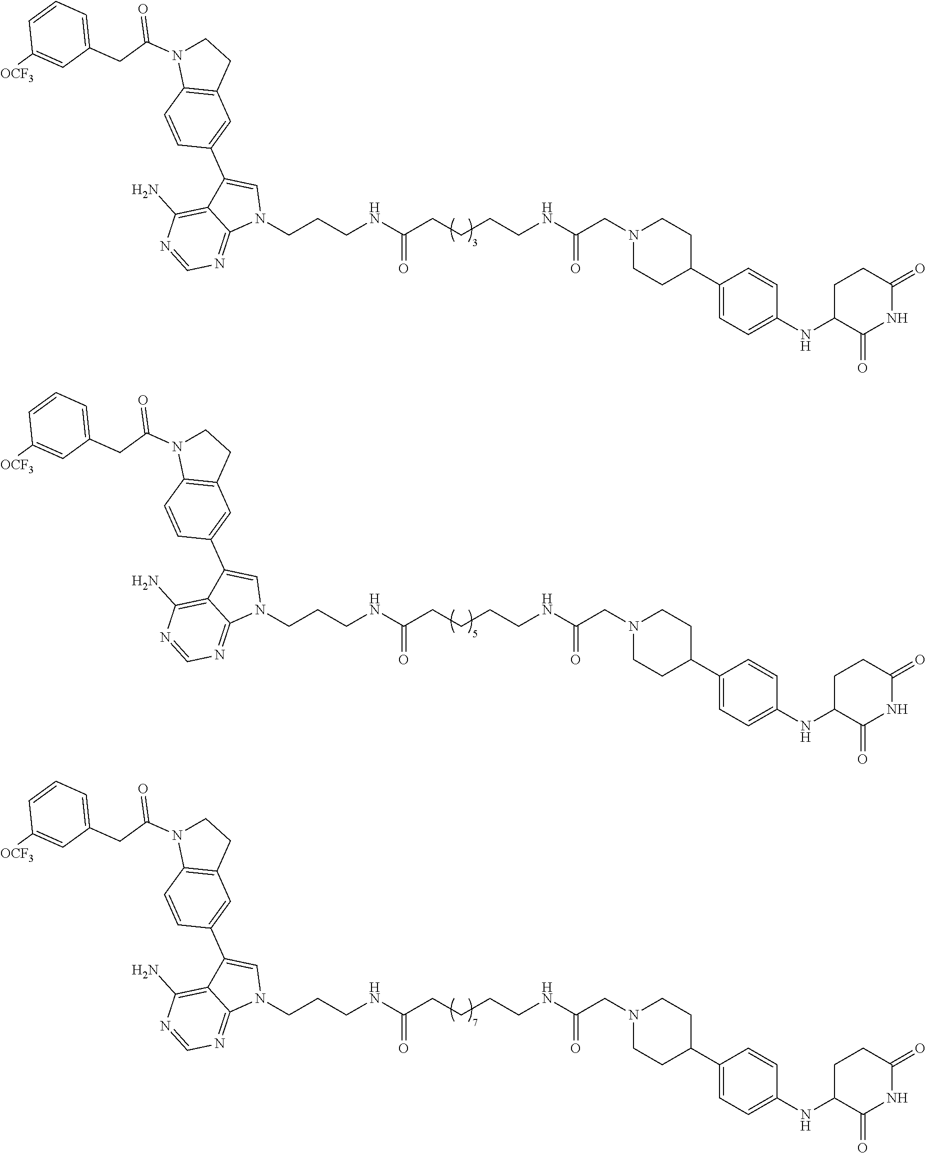

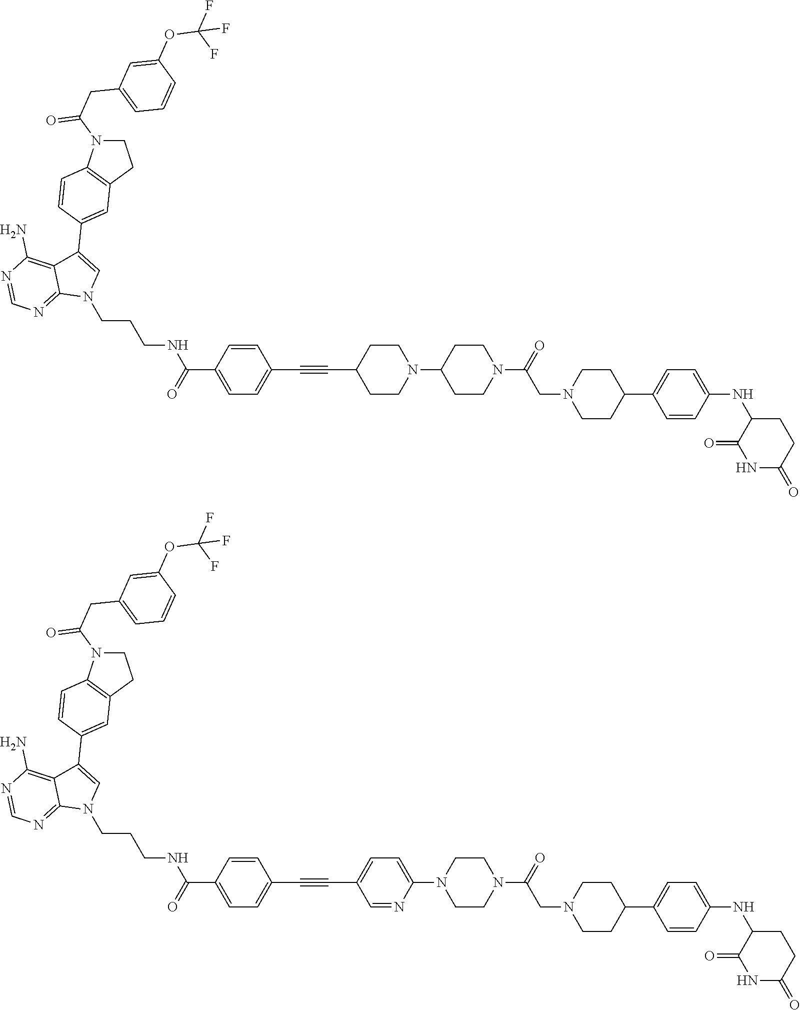 [SMILES: Nc1ncnc2c1c(-c1ccc3c(c1)CCN3C(=O)Cc1cccc(OC(F)(F)F)c1)cn2CCCNC(=O)c1ccc(C#CC2CCN(C3CCN(C(=O)CN4CCC(c5ccc(NC6CCC(=O)NC6=O)cc5)CC4)CC3)CC2)cc1 ; Nc1ncnc2c1c(-c1ccc3c(c1)CCN3C(=O)Cc1cccc(OC(F)(F)F)c1)cn2CCCNC(=O)c1ccc(C#Cc2ccc(N3CCN(C(=O)CN4CCC(c5ccc(NC6CCC(=O)NC6=O)cc5)CC4)CC3)nc2)cc1]

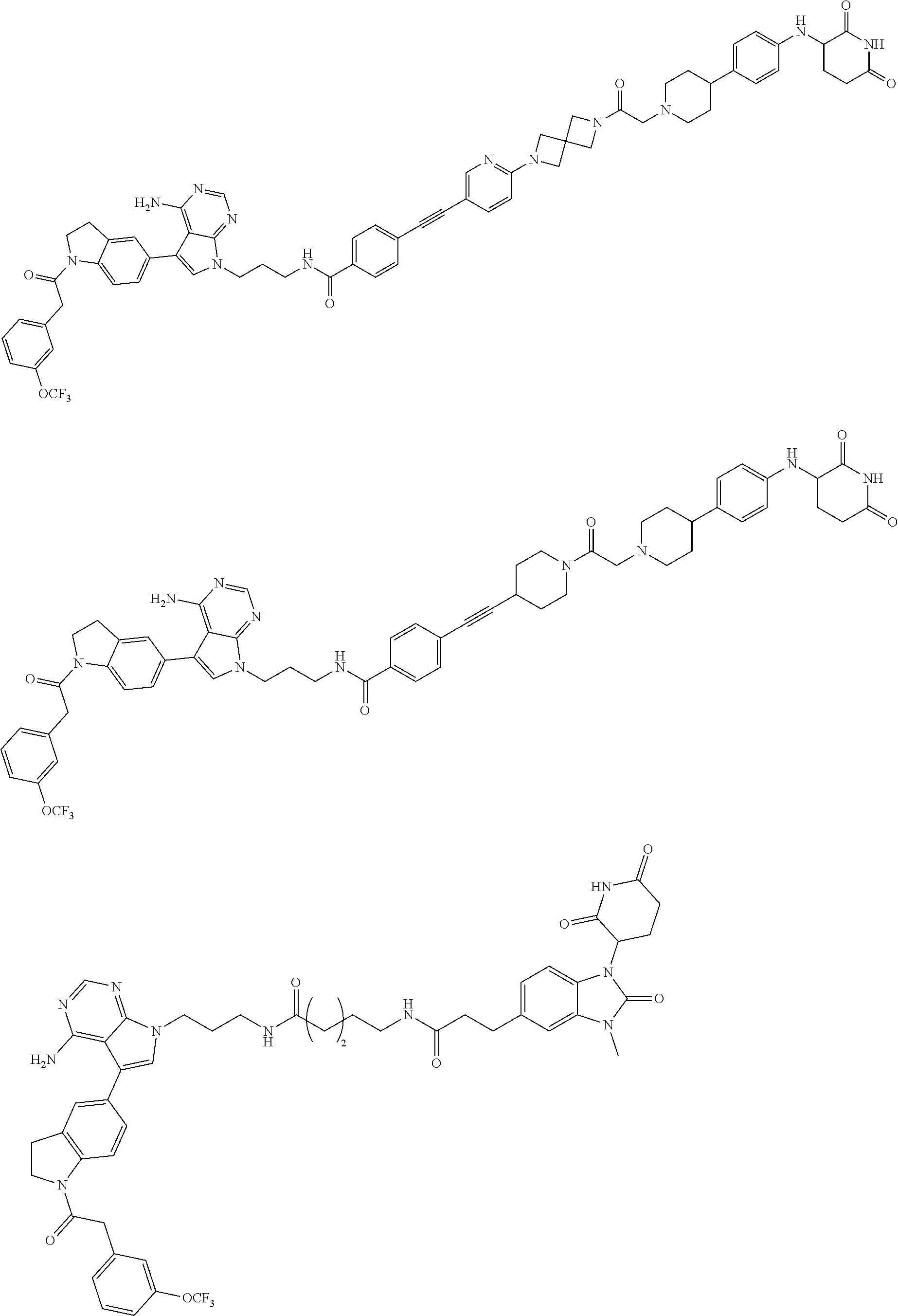

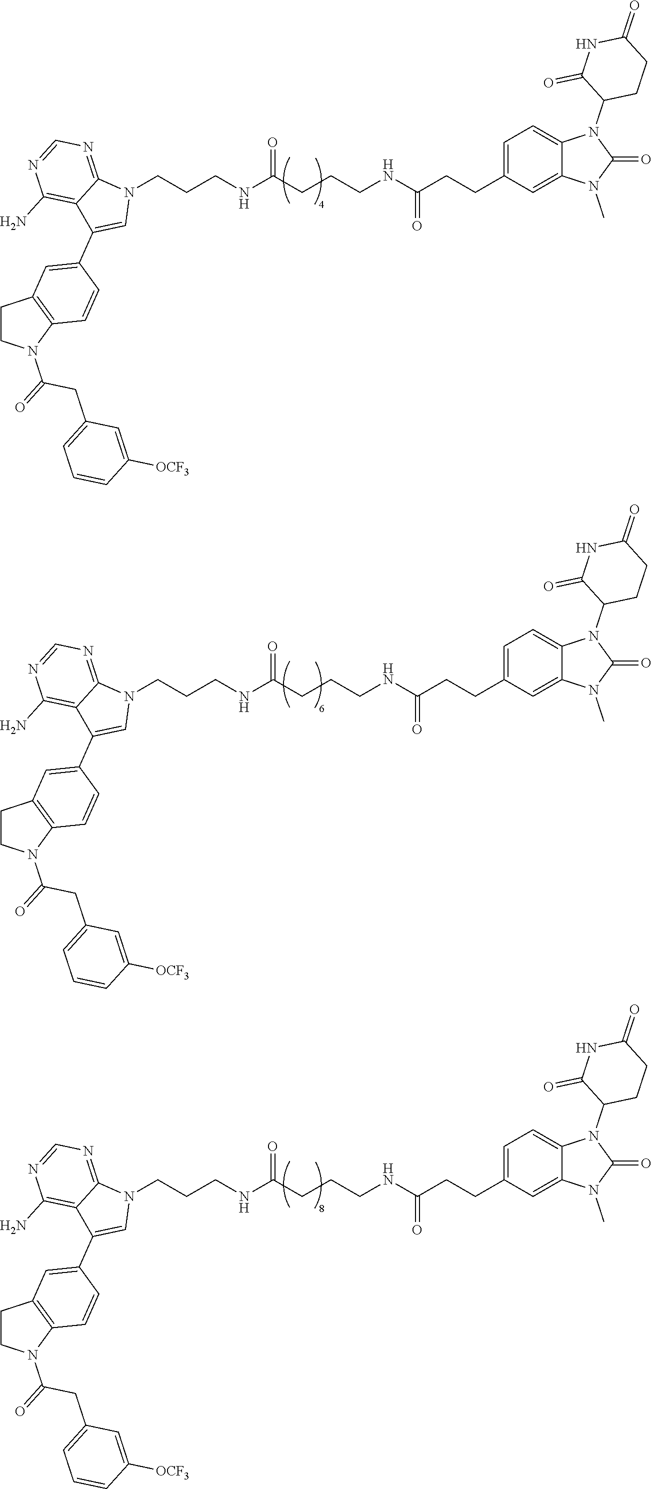

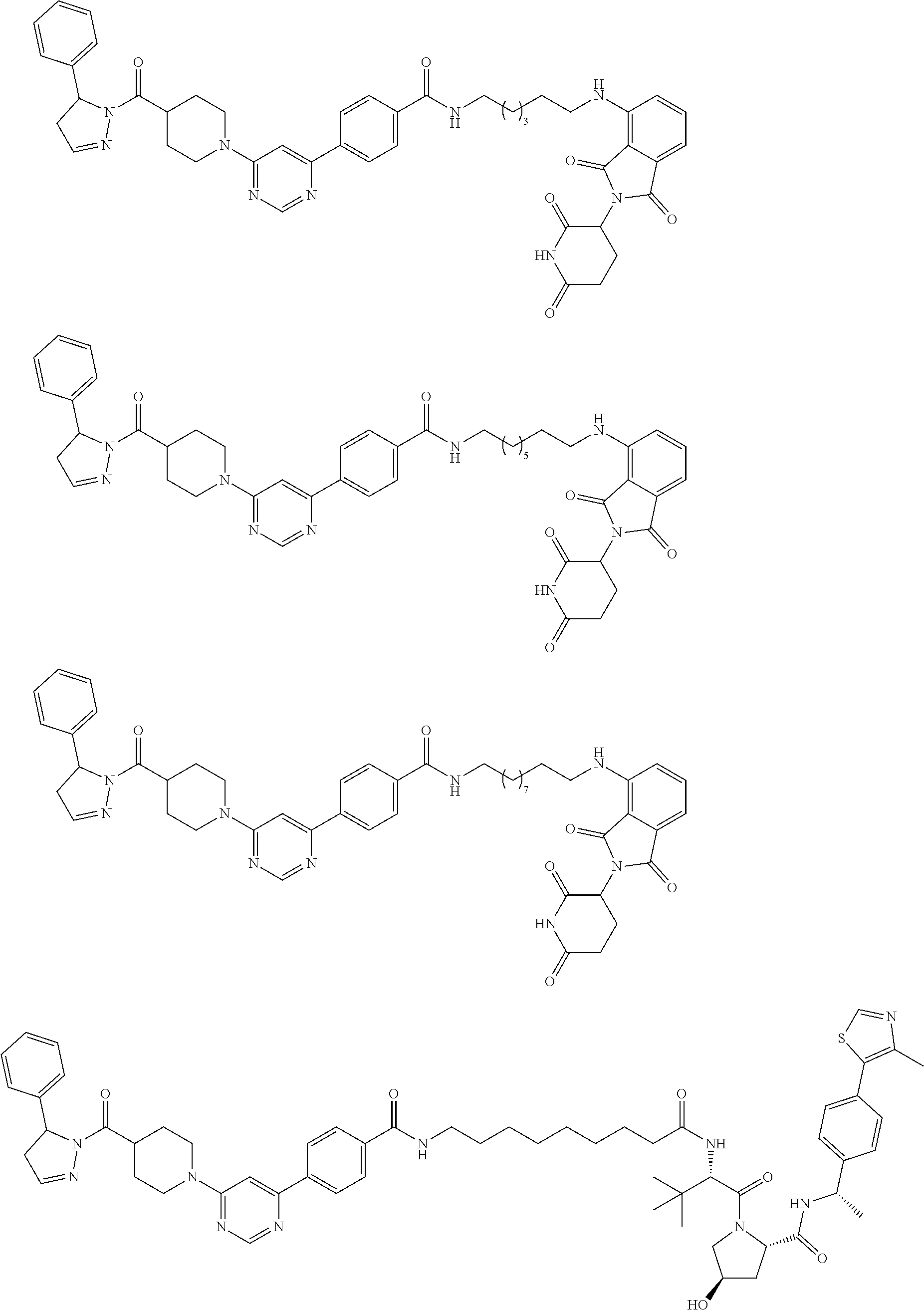

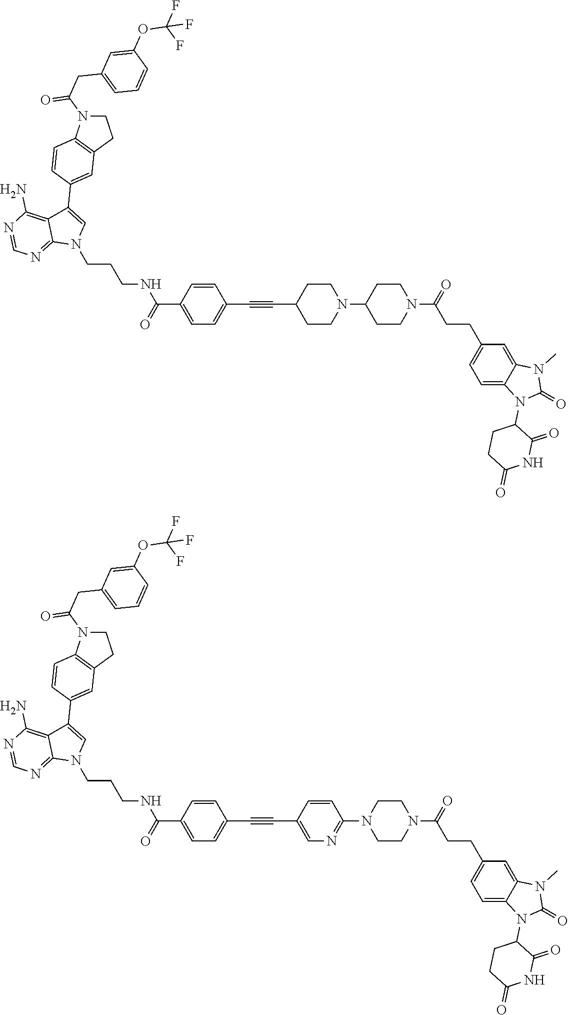 [SMILES: Cn1c(=O)n(C2CCC(=O)NC2=O)c2ccc(CCC(=O)N3CCC(N4CCC(C#Cc5ccc(C(=O)NCCCn6cc(-c7ccc8c(c7)CCN8C(=O)Cc7cccc(OC(F)(F)F)c7)c7c(N)ncnc76)cc5)CC4)CC3)cc21 ; Cn1c(=O)n(C2CCC(=O)NC2=O)c2ccc(CCC(=O)N3CCN(c4ccc(C#Cc5ccc(C(=O)NCCCn6cc(-c7ccc8c(c7)CCN8C(=O)Cc7cccc(OC(F)(F)F)c7)c7c(N)ncnc76)cc5)cn4)CC3)cc21]

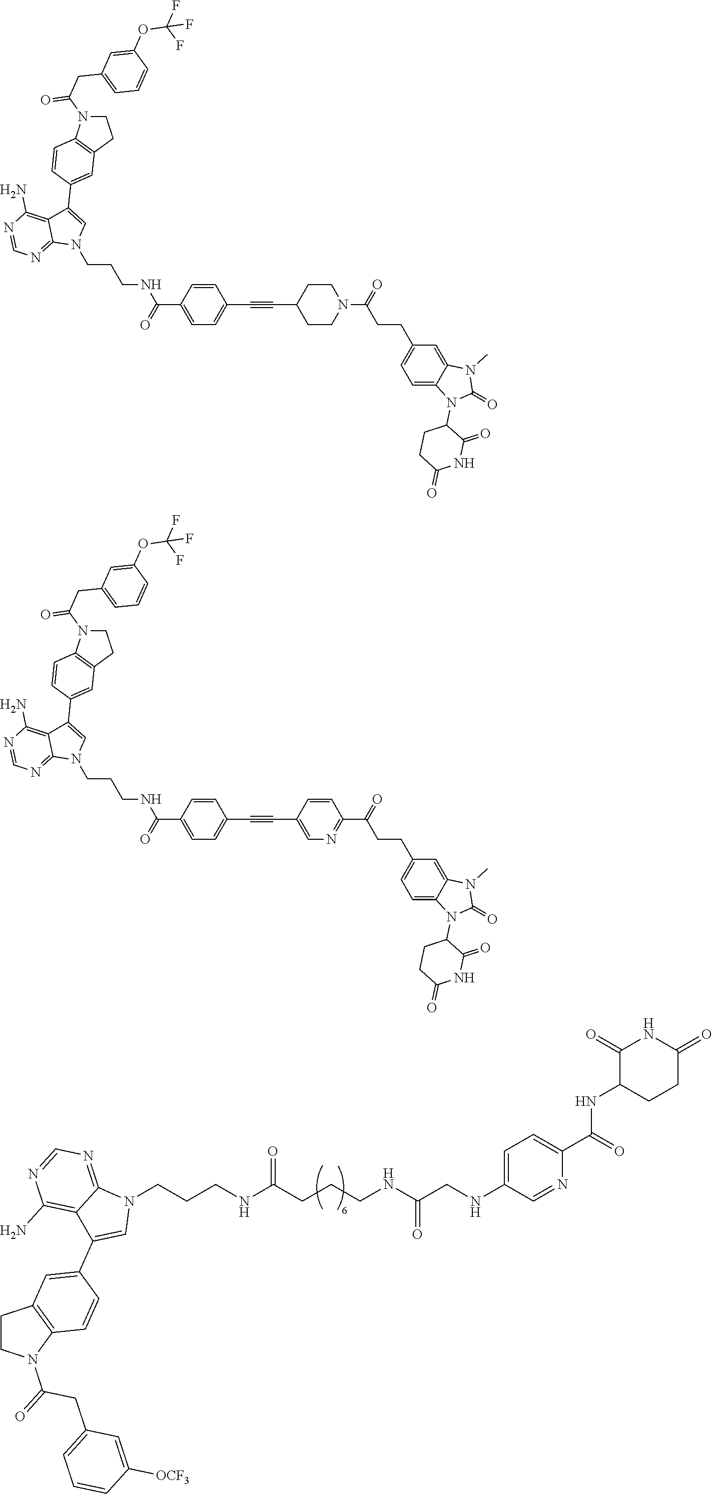

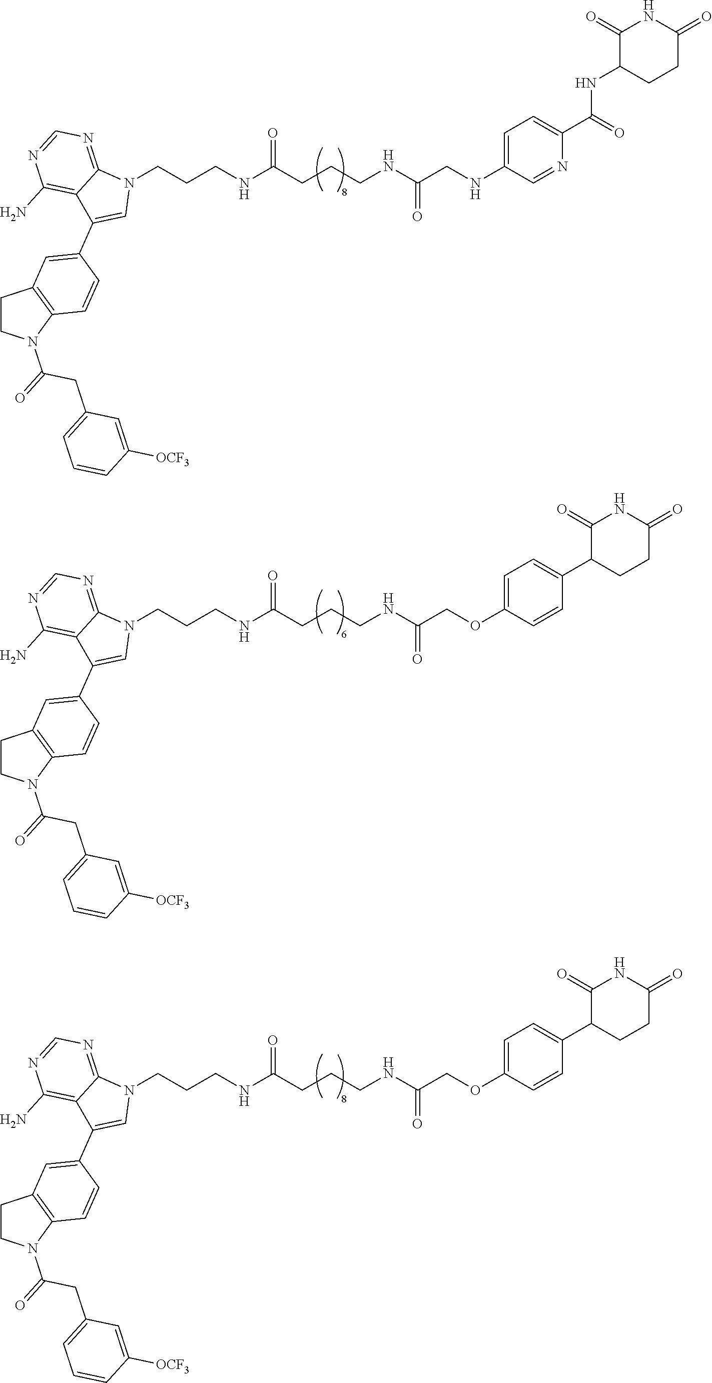

or a pharmaceutically acceptable salt, co-crystal, tautomer, stereoisomer, solvate, hydrate, polymorph, isotopically enriched derivative, or prodrug thereof.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and all-ynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkenyl, C₅-C₆ cycloalkyl, C₅-C₆ cycloalkenyl, and C₅-C₆ cycloalkenyl.

The terms heterocycloalkyl, heterocycloalkenyl, and heterocycloalkenyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkenyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bound through a single, terminal ether linkage. The term aryloxy as used herein is an aryl group bound through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bound through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, aryloxy, amino, alkyl, alkenyl, alkynyl, aryl, heteroalkyl, heteroalkenyl, heteroalkanyl, heteroaryl, cycloalkyl, or heterocycloalkyl molecules used herein can be substituted or unsubstituted. As used herein, the term substituted includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

II. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Formula I, Formula II, and Formula III include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Exemplary methods for synthesizing the compounds as described herein are provided in Example 1 below.

III. Pharmaceutical Formulations

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS (BASF: Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, inhalants, and skin patches. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the compounds described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinal), skill in the art and includes exemplary dosage amounts for a mammal of from about 0.0001 to about 2(X) mg/kg of body weight of active compound per day, which may be administered in a single dose or in the form of individual divided doses, such as from 1 to 4 times per day. Alternatively, the dosage amount can be from about 0.01 to about 150 mg/kg of body weight of active compound per day, about 0.1 to 100 mg/kg of body weight of active compound per day, about 0.5 to about 75 mg/kg of body weight of active compound per day, about 0.5 to about 50 mg/kg of body weight of active compound per day, about 0.01 to about 50 mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg. Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

IV. Methods of Use

Provided herein are methods to treat, prevent, or ameliorate a RIPK1 kinase-related disease in a subject. The methods include administering to a subject an effective amount of one or more of the compounds or compositions described herein, or a pharmaceutically acceptable salt or prodrug thereof. Effective amount, when used to describe an amount of compound in a method, refers to the amount of a compound that achieves the desired pharmacological effect or other biological effect. The effective amount can be, for example, the concentrations of compounds at which RIPK1 kinase is degraded in vitro, as provided herein. Also contemplated is a method that includes administering to the subject an amount of one or more compounds described herein such that an in vivo concentration at a target cell in the subject corresponding to the concentration administered in vitro is achieved.

The compounds and compositions described herein or pharmaceutically acceptable salts thereof are useful for treating RIPK1 kinase-related diseases in humans, including, without limitation, pediatric and geriatric populations, and in animals, e.g., veterinary applications.

In some embodiments, the RIPK1 kinase-related disease is cancer. Optionally, the cancer is a poor prognosis cancer. The term poor prognosis, as used herein, refers to a prospect of recovery from a disease, infection, or medical condition that is associated with a diminished likelihood of a positive outcome. In relation to a disease such as cancer, a poor prognosis may be associated with a reduced patient survival rate, reduced patient survival time, higher likelihood of metastatic progression of said cancer cells, and/or higher likelihood of chemoresistance of said cancer cells. Optionally, a poor prognosis cancer can be a cancer associated with a patient survival rate of 50% or less. Optionally, a poor prognosis cancer can be a cancer associated with a patient survival time of five years or less after diagnosis. In some embodiments, the cancer is an invasive cancer.

Optionally, the cancer is bladder cancer, brain cancer, breast cancer (e.g., triple negative breast cancer), bronchus cancer, colorectal cancer (e.g., colon cancer, rectal cancer), cervical cancer, chondrosarcoma, endometrial cancer, gastrointestinal cancer, gastric cancer, genitourinary cancer, glioblastoma, head and neck cancer, hepatic cancer, hepatocellular carcinoma, leukemia, liver cancer, lung cancer, lymphoma, melanoma of the skin, ovarian cancer, pancreatic cancer, prostate cancer, renal cancer, skin cancer, testicular cancer, thyroid cancer, or uterine cancer. Optionally, the cancer is a cancer that affects one or more of the following sites: oral cavity and pharynx (e.g., tongue, mouth, pharynx, or other oral cavity); digestive system (e.g., esophagus, stomach, small intestine, colon, rectum, anus, anal canal, anorectum, liver and intrahepatic bile duct, gallbladder and other biliary, pancreas, or other digestive organs); respiratory system (e.g., larynx, lung and bronchus, or other respiratory organs); bones and joints; soft tissue (e.g., heart); skin (e.g., melanoma of the skin or other nonepithelial skin); breast; genital system (e.g., uterine cervix, uterine corpus, ovary, vulva, vagina and other female genital areas, prostate, testis, penis and other male genital areas); urinary system (e.g., urinary bladder, kidney and renal pelvis, and ureter and other urinary organs); eye and orbit; brain and other nervous system endocrine system (e.g., thyroid and other endocrine); lymphoma (e.g., Hodgkin lymphoma and non-Hodgkin lymphoma); myeloma: or leukemia (e.g., acute lymphocytic leukemia, chronic lymphocytic leukemia, acute myeloid leukemia, chronic myeloid leukemia, or other leukemia). Optionally, the cancer is a drug resistant cancer, such as an ibrutinib-resistant cancer.

Optionally, the RIPK1 kinase-related disease is a neurodegenerative disorder. Optionally, the neurodegenerative disorder is Parkinson's disease. Optionally, the neurodegenerative disorder is Alexander disease, Alper's disease, Alzheimer disease, amyotrophic lateral sclerosis, ataxia telangiectasia, Batten disease (also known as Spielmeyer-Vogt-Sjogren-Batten disease), Canavan disease, Cockayne syndrome, corticobasal degeneration, Creutzfeldt-Jakob disease, Huntington's disease, Kennedy's disease, Krabbe disease. Lewy body dementia, Machado-Joseph disease, spinocerebellar ataxia type 3, multiple sclerosis, multiple system atrophy, Pelizaeus-Merzbacher disease, Pick's disease, primary lateral sclerosis, Refsum's disease, Sandhoff disease, Schilder's disease, Spielmeyer-Vogt-Sjogren-Batten disease (also known as Batten disease), spinocerebellar ataxia (multiple types with varying characteristics), spinal muscular atrophy, Steele-Richardson-Olszewski disease, Tay-Sachs, transmissible spongiform encephalopathies (TSE), or tabes dorsalis.

Optionally, the RIPK1 kinase-related disease is an inflammatory disease. Generally, inflammatory disorders include, but are not limited to, respiratory or pulmonary disorders (including asthma. COPD, chronic bronchitis and cystic fibrosis); cardiovascular related disorders (including atherosclerosis, post-angioplasty, restenosis, coronary artery diseases and angina); inflammatory diseases of the joints (including rheumatoid and osteoarthritis); skin disorders (including dermatitis, eczematous dermatitis and psoriasis); post transplantation late and chronic solid organ rejection; multiple sclerosis; autoimmune conditions (including systemic lupus erythematosus, dermatomyositis, polymyositis, Sjogren's syndrome, polymyalgia rheumatica, temporal arteritis, Behcet's disease, Guillain Barre, Wegener's granulomatosus, polyarteritis nodosa); inflammatory neuropathies (including inflammatory polyneuropathies); vasculitis (including Churg-Strauss syndrome, Takayasu's arteritis); inflammatory disorders of adipose tissue; and proliferative disorders (including Kaposi's sarcoma and other proliferative disorders of smooth muscle cells).

Optionally, the RIPK1 kinase-related disease is ischemia, a gastrointestinal disorder, a viral infection (e.g., human immunodeficiency virus (HIV), including HIV type 1 (HIV-1) and HIV type 2 (HIV-2)), a bacterial infection, a central nervous system disorder, a spinal cord injury, or peripheral neuropathy.

The compounds and compositions described herein or pharmaceutically acceptable salts and prodrugs thereof can also be useful in treating any genetic disease related to RIPK1, including in individuals homozygous or heterozygous for RIPK1 mutations or deletions. In some cases, the compounds described herein can be used to protect tissues from inflammatory bowel diseases (e.g., ulcerative colitis and Crohn's disease), psoriasis, retinal-detachment-induced photoreceptor necrosis, retinitis pigmentosa, cerulein-induced acute pancreatitis and sepsis/systemic inflammatory response syndrome (SIRS) and alleviating ischemic brain injury, retinal ischemia/reperfusion injury, Huntington's disease, renal ischemia reperfusion injury, cisplatin induced kidney injury, traumatic brain injury, hematological and solid organ malignancies, bacterial infections and viral infections (e.g., tuberculosis and influenza) and lysosomal storage diseases.

The receptor interacting protein kinase 1 inhibitors of the present disclosure are therefore useful for treating diseases and conditions mediated by receptor interacting protein kinase 1, including but not limited to inflammatory diseases or disorders, necrotic cell diseases, neurodegenerative diseases, central nerve system (CNS) diseases, ocular diseases, infections and malignancies. In certain embodiments, the receptor interacting protein kinase 1 inhibitors described herein can inhibit inflammation, protect tissue or cell from damage or undesired cell death (e.g., necrosis or apoptosis), ameliorate symptoms and improve immune response in a patient suffering from any of the prescribed diseases or conditions.

Moreover, the compounds may be suitable for treatment of immune-mediated disease, such as but not limited to, allergic diseases, autoimmune diseases and prevention of transplant rejection.

The compounds described herein may be used for the treatment of diseases/disorders caused or otherwise associated with cellular necrosis. In particular, the disclosure provides methods for preventing or treating a disorder associated with cellular necrosis in a mammal, comprising the step of administering to said mammal a therapeutically effective amount of a compound or composition described herein. The term“necrotic cell disease” refers to diseases associated with or caused by cellular necrosis, for example trauma, ischemia, stroke, cardiac infarction, infection, Gaucher's disease, Krabbe disease, sepsis, Parkinson's disease, Alzheimer's disease, amyotrophic lateral sclerosis, Huntington's disease, HIV-associated dementia, retinal degenerative disease, glaucoma, age-related macular degeneration, rheumatoid arthritis, psoriasis, psoriatic arthritis or inflammatory bowel disease.

The necrotic cell diseases can be acute diseases such as trauma, ischemia, stroke, cardiac infarction, anthrax lethal toxin induced septic shock, sepsis, cell death induced by LPS and HIV induced T-cell death leading to immunodeficiency. In certain embodiments the disorder is an ischemic disease of organs including but not limited to brain, heart, kidney and liver. The necrotic cell diseases also include chronic neurodegenerative diseases, such as Parkinson's disease, Huntington's disease, amyotrophic lateral sclerosis, Alzheimer's disease, infectious enteropathies, dementia such as HIV associated dementia

In some embodiments, the disorder is an ocular disorder such as retinal degenerative disease, glaucoma or age-related macular degeneration. In some different embodiments, the disorder is a central nervous system (CNS) disorder.

The RIPK1 kinase inhibitors described herein may be used to treat inflammatory diseases and disorders. Inflammatory diseases and disorders typically exhibit high levels of inflammation in the connective tissues or degeneration of these tissues. Non-limiting examples of inflammatory diseases and disorders include Alzheimer's disease, ankylosing spondylitis, arthritis including osteoarthritis, rheumatoid arthritis (RA), psoriasis, asthma, atherosclerosis, Crohn's disease, colitis, dermatitis, diverticulitis, fibromyalgia, hepatitis, irritable bowel syndrome (IBS), inflammatory bowel disease (IBD), systemic lupus erythematous (SLE), nephritis, Parkinson's disease and ulcerative colitis. In certain embodiments, the compounds and compositions of the present disclosure are useful for treating an autoimmune disorder, such as rheumatoid arthritis, psoriasis, psoriatic arthritis, encephalitis, allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), insulin-dependent diabetes mellitus, multiple sclerosis, pernicious anemia, sarcoidosis, scleroderma, and systemic lupus erythematosus. In an embodiment, the receptor interacting protein kinase 1 inhibitors described herein are useful for treating autoimmune encephalitis.

In certain embodiments, the compounds and compositions are useful for treating rheumatoid arthritis (RA). In certain embodiments, the compounds and compositions are useful for treating ulcerative colitis. In certain embodiments, the compounds and compositions are useful for treating psoriasis.

In certain embodiments, the disorder is an inflammatory disease of the intestines such as Crohn's disease or ulcerative colitis (both generally known together as inflammatory bowel disease). In certain embodiments, the mammal is a primate, canine or feline subject. In certain embodiments, the mammal is a human subject. While not wishing to be bound by theory, it is believed that inhibition of receptor interacting protein kinase 1 by the presently disclosed compounds is responsible, at least in part, for their anti-inflammatory activity. Accordingly, embodiments of the disclosure also include methods for inhibiting receptor interacting protein kinase 1, either in vitro or in a subject in need thereof, the method comprises contacting a receptor interacting protein kinase 1 with a compound disclosed herein. In some of these embodiments, inhibiting receptor interacting protein kinase 1 is effective to block (partially or fully) the release of inflammatory mediators such as TNF and/or IL6.

The RIPK1 kinase inhibitors described herein can also be used to treat ocular conditions, for example to reduce or prevent the loss of photoreceptor and/or retinal pigment epithelial cell viability. In one aspect, the disclosure provides a method of preserving the visual function of an eye of a subject with an ocular condition, wherein a symptom of the ocular condition is the loss of photoreceptor cell viability in the retina of the eye with the condition. The method comprises administering to the eye of the subject an effective amount of a compound or composition described herein, thereby preserving the viability of the photoreceptor cells disposed within the retina of the eye. After administration, the visual function (e.g., visual acuity) of the eye may be preserved or improved relative to the visual function of the eye prior to administration.

The ocular condition may be a condition selected from the group consisting of age-related macular degeneration (AMD), retinosis pigmentosa (RP), macular edema, diabetic retinopathy, central areolar choroidal dystrophy, BEST disease, adult vitelliform disease, pattern dystrophy, myopic degeneration, central serous retinopathy, Stargardt's disease, Cone-Rod dystrophy, North Carolina dystrophy, infectious retinitis, inflammatory retinitis, uveitis, toxic retinitis and light-induced toxicity. AMD may be the neovascular or the dry form of AMD. Retinal detachment may be a rhegmatogenous, a serous or a tractional retinal detachment.

In another aspect, the disclosure provides a method of preserving the viability of retinal pigment epithelial (RPE) cells within the retina of a subject with an ocular condition, wherein a symptom of the ocular condition is the loss of retinal pigment epithelial cells in the retina of the eye with the condition. The method comprises administering to the eye of the subject an effective amount of a compound or composition described herein, thereby preserving the viability of the retinal pigment epithelial cells. The ocular condition may be selected from the group consisting of AMD, BEST disease, myopic degeneration, Stargardt's disease, uveitis, adult foveomacular dystrophy, fundus falvimaculatus, multiple evanescent white dot syndrome, serpiginous choroidopathy, acute multifocal posterior placoid epitheliopathy (AMPPE) and other uveitis disorders.

The ocular condition may be a condition selected from the group consisting of age-related macular degeneration (AMD), retinosis pigmentosa (RP), macular edema, diabetic retinopathy, central areolar choroidal dystrophy, BEST disease, adult vitelliform disease, pattern dystrophy, myopic degeneration, central serous retinopathy, Stargardt's disease, Cone-Rod dystrophy. North Carolina dystrophy, infectious retinitis, inflammatory retinitis, uveitis, toxic retinitis and light-induced toxicity. Therefore, in certain embodiments, the method comprises administering to the eye an effective amount of a compound or composition described herein, thereby preserving the viability of the photoreceptor cells disposed within the retina of the subject with a condition.

In another aspect, the disclosure provides a method of preserving the viability of photoreceptor cells disposed within a retina of a mammalian eye following retinal detachment. The method comprises administering a compound or composition described herein to the eye in which a region of the retina has been detached in amounts sufficient to preserve the viability of photoreceptor cells disposed within the region of the detached retina.

In certain embodiments, the retinal detachment may be a rhegmatogenous retinal detachment, tractional retinal detachment or serous retinal detachment. In certain embodiments, the retinal detachment may occur as a result of a retinal tear, retinoblastoma, melanoma or other cancers, diabetic retinopathy, uveitis, choroidal neovascularization, retinal ischemia, pathologic myopia or trauma.

In another aspect, the disclosure provides a method of preserving visual function of an eye of a subject with an ocular condition selected from the group consisting of AMD, RP, macular edema, central areolar choroidal dystrophy, retinal detachment, diabetic retinopathy, BEST disease, adult vitelliform disease, pattern dystrophy, myopic degeneration, central serous retinopathy, Stargardt's disease, Cone-Rod dystrophy. North Carolina dystrophy, infectious retinitis, inflammatory retinitis, uveitis, toxic retinitis and light-induced toxicity, wherein a symptom of the ocular condition is the loss of photoreceptor cells viability in the retina of the eye, wherein the method comprises treating the subject with a compound or composition described herein to the subject.

In another aspect, the disclosure provides a method of preserving the visual function of an eye of a subject with an ocular condition, wherein a symptom of the ocular condition is the loss of photoreceptor cell viability and/or RPE viability in the retina of the eye wherein the method comprises treating the subject with a compound or composition described herein to the subject.

In certain embodiments is provided a method of preserving the visual function of an eye of a subject with ocular conditions, wherein a symptom of the ocular condition is the loss of retinal ganglion cell viability in the retina of the eye with the conditions. The method comprises administering to the eye of the subject an effective amount of a compound or composition, thereby preserving the viability of the retinal ganglion cells disposed within the retina of the eye. After administration of the compound or composition, the visual function of the eye may be preserved or improved relative to the visual function of the eye prior to administration. Further, after the administration, the preserved retinal ganglion cell is capable of supporting axonal regeneration.

In each of the foregoing methods, the ocular condition, wherein a symptom of the condition is the loss of retinal ganglion cell viability in the retina of the eye, includes but is not limited to glaucoma, optic nerve injury, optic neuritis, optic neuropathies, diabetic retinopathy, central retinal artery occlusion and central retinal vein occlusion. It is contemplated that the forgoing methods may be used for the treatment of optic neuropathies such as ischemic optic neuropathy (e.g., arteritic or non-arteritic anterior ischemic neuropathy and posterior ischemic optic neuropathy), compressive optic neuropathy, infiltrative optic neuropathy, traumatic optic neuropathy, mitochondrial optic neuropathy (e.g., Leber's optic neuropathy), nutritional optic neuropathy, toxic optic neuropathy and hereditary optic neuropathy (e.g., Leber's optic neuropathy, Dominant Optic Atrophy, Behr's syndrome).

Also disclosed is a method of preserving the visual function of an eye of a subject with an ocular condition selected from the group consisting of glaucoma, optic nerve injury, optic neuropathies, diabetic retinopathy, central retinal artery occlusion and central retinal vein occlusion. The method comprises administering to the eye of the subject an effective amount of a compound or composition described herein, thereby preserving the viability of the retinal ganglion cells disposed within the retina of the eye and the visual function of the eye.

In another aspect, disclosed herein is a method of preserving the viability of retinal ganglion cells disposed within a retina of a mammalian eye affected by, for example, glaucoma, optic nerve injury, optic neuritis, optic neuropathies, diabetic retinopathy, central retinal artery occlusion and central retinal vein occlusion. The method comprises administering a compound or composition described herein to the eye in which a region of the retina has been affected in amounts sufficient to preserve the viability of retinal ganglion cells disposed within the region of the affected retina. The preserved retinal ganglion cell is capable of supporting axonal regeneration.

Also disclosed is a method for promoting axon regeneration in an eye of a subject with an ocular condition, wherein a symptom of the ocular condition is the loss of retinal ganglion cell viability in the retina of the eye with the condition. The method comprises administering to the eye of the subject an effective amount of a compound or composition described herein, thereby promoting axon regeneration of the retinal ganglion cell within the retina of the eye.

In each of the foregoing embodiments, it is understood that the methods and compositions described herein can be used to preserve the viability and/or promote axon regeneration of retinal ganglion cells during treatment of the underlying conditions including, but not limited to, glaucoma, optic nerve injury, optic neuritis, optic neuropathies, diabetic retinopathy, central retinal artery occlusion and central retinal vein occlusion.

The receptor interacting protein kinase 1 inhibitors described herein may also be used to treat neurodegenerative diseases. Neurodegenerative diseases can affect many of the body's activities, such as balance, movement, talking, breathing and heart function. Neurodegenerative diseases can be genetic or caused by medical conditions such as alcoholism, tumors, strokes, toxins, chemicals and viruses. Non-limiting examples of neurodegenerative diseases and CNS diseases include Niemann-Pick disease, type C1 (NPC1), Alzheimer's disease, amyotrophic lateral sclerosis (ALS), Friedreich's ataxia, Huntington's disease, Lewy body disease, Parkinson's disease and spinal muscular atrophy.

In an embodiment, the receptor interacting protein kinase 1 inhibitors described herein may be used to treat NPC1 via inhibiting necroptosis that causes neuronal loss. In certain embodiments, the compounds and compositions of the present disclosure are useful for treating Alzheimer's disease. In certain embodiments, the compounds and compositions of the present disclosure are useful for treating Parkinson's disease. In certain embodiments, the compounds and compositions of the present disclosure are useful for treating amyotrophic lateral sclerosis (ALS).

More generally, the receptor interacting protein kinase 1 inhibitors described herein can be used to preserve neuron viability and promote axon growth and nerve functions within the central nervous system (CNS). Accordingly, the compounds may be used to reduce or even reverse the loss of cognitive, motor and sensory functions associated with a CNS disease or disorder, by preserving neuron viability and/or promoting axon regeneration and/or nerve functions.

The receptor interacting protein kinase 1 inhibitors described herein can be used in a method for promoting axon regeneration in a CNS neuron, such as a CNS sensory neuron, a motor neuron, a cortical neuron, a cerebellar neuron, a hippocampal neuron and a midbrain neuron. The receptor interacting protein kinase 1 inhibitors described herein can be used in a method for promoting nerve function or preserving the viability following injury to a CNS neuron. In certain embodiments, these compounds can be used to promote regeneration of an axon in a CNS neuron that is degenerated in the CNS disease or disorder. The receptor interacting protein kinase 1 inhibitors may be administered by any conventional means, such as locally to the neuron or applied ex vivo before re-implantation.

Accordingly, in one aspect, the disclosure provides a method of treating a CNS disorder in a subject in need thereof, wherein a symptom of the CNS disorder is axon degeneration or injury within a CNS neuron. The method comprises administering to the subject an effective amount of a compound or composition disclosed herein thereby to promote regeneration of an axon in a CNS neuron affected by the CNS disorder. Following administration, neural functions may be measured, for example, as an indication of axon regeneration. It is also contemplated that, following administration of the compound or composition, the neuron function of the CNS neuron is preserved or improved relative to the neuron function prior to administration.

The CNS disorder includes, but is not limited to, brain injury, spinal cord injury, dementia, stroke, Alzheimer's disease, amyotrophic lateral sclerosis (ALS/Lou Gehrig's Disease), Parkinson's disease, Huntington's disease, multiple sclerosis, diabetic neuropathy, polyglutamine (polyQ) diseases, stroke, Fahr disease, Menke's disease, Wilson's disease, cerebral ischemia and a prion disorder. In exemplary embodiments, the CNS disorder is brain injury or spinal cord injury.

Also provided herein are methods for promoting neuron survival and axon regeneration in the CNS. CNS disorders characterized by impaired or failing axon growth or axon degeneration may arise from CNS neuron injury (e.g., trauma, surgery, nerve compression, nerve contusion, nerve transection, neurotoxicity or other physical injury to the brain or spinal cord) or neurodegenerative CNS disease, wherein a symptom of the disorder is axon degeneration (e.g., Alzheimer's disease, amyotrophic lateral sclerosis (ALS/Lou Gehrig's Disease), Parkinson's disease, multiple sclerosis, diabetic neuropathy, polyglutamine (polyQ) diseases, stroke, Fahr disease, Menke's disease, Wilson's disease, cerebral ischemia, prion disorder (e.g., Creutzfeldt-Jakob disease). In certain embodiments, the CNS disorder is brain injury (e.g., traumatic brain injury) or spinal cord injury (e.g., chronic, acute or traumatic spinal cord injury). In certain embodiments, the CNS disorder affects a subject's basic vital life functions such as breathing, heart beat and blood pressure, e.g., an injury to or aneurysm in the brain stem.

In certain embodiments, the CNS disorder affects a subject's cognitive ability, such as, brain injury to the cerebral cortex or a neurodegenerative CNS disorder, such as, Alzheimer's disease, frontotemporal dementia, dementia with Lewy bodies, corticobasal degeneration, progressive supranuclear palsy and prion disorders. In certain embodiments, the CNS disorder affects a subject's movement and/or strength, such as injury to the brain or spinal cord or a neurodegenerative CNS disorder such as Parkinson's disease, frontotemporal dementia, dementia with Lewy bodies, corticobasal degeneration, progress supranuclear palsy, Huntington's disease, multiple system atrophy, amyotrophic lateral sclerosis and hereditary spastic paresis.

In certain embodiments, the CNS disorder affects a subject's coordination, such as brain injury to the cerebellum or a neurodegenerative CNS disorder such as spinocerebellar atrophies, Friedreich's ataxia and prion disorders.

In each of the foregoing methods, the CNS disorder includes, but is not limited to, brain injury, spinal cord injury, Alzheimer's disease, amyotrophic lateral sclerosis (ALS/Lou Gehrig's Disease). Parkinson's disease, multiple sclerosis, diabetic neuropathy, polyglutamine (polyQ) diseases, stroke, Fahr disease, Menke's disease, Wilson's disease, cerebral ischemia, a prion disorder (e.g., Creutzfeldt-Jakob disease), dementia (e.g., frontotemporal dementia, dementia with Lewy bodies), corticobasal degeneration, progressive supranuclear palsy, multiple system atrophy, hereditary spastic paraparesis and spinocerebellar atrophies.

The ability of the compounds described herein to inhibit inflammation and cell death makes them suitable for ameliorating tissue injuries or damages. The tissue injuries or damages may be a result of any of the diseases or conditions described above. For example, the compounds may be used for amelioration of brain tissue injury or damage following ischemic brain injury or traumatic brain injury or for amelioration of heart tissue injury or damage following myocardial infarction or for amelioration of brain tissue injury or damage associated with Huntington's disease, Alzheimer's disease or Parkinson's disease or for amelioration of liver tissue injury or damage associated with non-alcohol steatohepatitis, alcohol steatohepatitis, autoimmune hepatitis autoimmune hepatobiliary diseases or primary sclerosing cholangitis or for the amelioration of liver tissue injury or damage associated with overdose of acetaminophen or for amelioration of kidney tissue injury or damage following renal transplant or the administration of nephrotoxic drugs or substances.

Non-limiting examples of brain injury or damage include stroke (e.g., hemorrhagic and non-hemorrhagic), traumatic brain injury (TBI), cerebral hemorrhage, subarachnoid hemorrhage, intracranial hemorrhage secondary to cerebral arterial malformation, cerebral infarction, perinatal brain injury, non-traumatic brain injury, Alzheimer's disease, Parkinson's disease, Huntington's disease, multiple sclerosis, amyotrophic lateral sclerosis, brain hemorrhage, brain infections, brain tumor, subclinical brain injury, spinal cord injury, anoxic-ischemic brain injury, focal cerebral ischemia, global cerebral ischemia, and hypoxic hypoxia.

In an embodiment, the compounds and compositions of the present disclosure may be used to treat peritoneal tissue injury. Non-limiting examples of peritoneal tissue injury include peritoneal deterioration, peritoneal sclerosis, and peritoneal cancer. For example, the receptor interacting protein kinase 1 inhibitors described herein may be used to treat peritoneal damage caused by peritoneal dialysis fluid (PDF) and PD-related side effects.

In an embodiment, the compounds and compositions of the present disclosure may be used to treat liver injury and diseases. Non-limiting examples of liver injury or damage include not only degeneration or necrosis of liver parenchyma cells which results from injury caused by a certain factor, but also undesirable phenomena caused by biological reactions to the injury, such as mobilization, infiltration, activation of Kupffer cells, leukocytes and the like, fibrosis of the liver tissue, etc., which reactions occur alone or in combination. In an embodiment, the receptor interacting protein kinase 1 inhibitors described herein may be used to treat steatohepatitis and hepatocellular carcinoma via inhibiting receptor interacting protein kinase 1 activity-dependent apoptosis of hepatocytes and hepatocarcinogenesis.

In an embodiment, the compounds and compositions of the present disclosure may be used to treat kidney injury and diseases. Non-limiting examples of kidney diseases include chronic kidney disease (CKD) (e.g., glomerular diseases, tubulointerstitial diseases, obstruction, polycystic kidney disease), acute kidney injury (AKI), diabetic nephropathy, glomerulonephritis, focal glomerulosclerosis, immune complex nephropathy or lupus nephritis. Kidney disease may be caused by drug-induced renal injury or kidney graft rejection. Kidney disease may be characterized as nephrotic syndrome or renal insufficiency. In an embodiment, the receptor interacting protein kinase 1 inhibitors described herein may be used to treat kidney diseases (e.g., AKI) via inhibiting cell death pathway in kidney diseases. In an embodiment, the receptor interacting protein kinase 1 inhibitors described herein may be used to treat patient with kidney stones and to prevent crystal-induced cytotoxicity and acute kidney injury via inhibiting receptor interacting protein kinase 3-MLKL-mediated necroptosis.

In an embodiment, the compounds and compositions of the present disclosure are useful for treating malignancies/cancers such as carcinoma, sarcoma, melanoma, lymphoma or leukemia. Non-limiting examples of malignancies suitably treated by the receptor interacting protein kinase 1 inhibitors described herein include lung cancer (e.g. non-small cell lung cancer, small-cell lung cancer), hepatocellular cancer, melanoma, pancreatic cancer, urological cancer, bladder cancer, colorectal cancer, colon cancer, breast cancer, prostate cancer, renal cancer, thyroid cancer, gall bladder cancer, peritoneal cancer, ovarian cancer, cervical cancer, gastric cancer, endometrial cancer, esophageal cancer, head and neck cancer, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, Kaposi's sarcomas, myxoid carcinoma, round cell carcinoma, squamous cell carcinomas, esophageal squamous cell carcinomas, oral carcinomas, vulval cancer, cancers of the adrenal cortex, ACTH producing tumors, lymphoma, and leukemia.

In an embodiment, the compounds and compositions of the present disclosure are useful for treating infectious diseases resulting from the presence of pathogenic agents, including pathogenic viruses, pathogenic bacteria, fungi, protozoa, multicellular parasites and aberrant proteins known as prions. Non-limiting examples of infectious diseases suitably treated by the receptor interacting protein kinase 1 inhibitors described herein include virus infectious diseases and bacterial infectious diseases. The virus infectious disease is not particularly limited and includes, for example, infectious diseases with respiratory infectious viruses (e.g., infectious diseases due to respiratory infectious viruses such as influenza virus, rhino virus, corona virus, parainfluenza virus, RS virus, adeno virus, reo virus and the like), herpes zoster caused by herpes virus, diarrhea caused by rotavirus, viral hepatitis, AIDS and the like. The bacterial infectious disease is not particularly limited and includes, for example, infectious diseases caused by Bacillus cereus, Vibrio parahaemolyticus, Enterohemorrhagic Escherichia coli, Staphylococcus aureus, MRSA, Salmonella, Botulinus, Candida and the like.

In an embodiment, the compounds and compositions of the present disclosure are useful for treating bone diseases that may result from a bone remodeling disorder whereby the balance between bone formation and bone resorption is shifted. Non-limiting examples of bone remodeling disorders include osteoporosis, Paget's disease, osteoarthritis, rheumatoid arthritis, achondroplasia, osteochodrytis, hyperparathyroidism, osteogenesis imperfecta, congenital hypophosphatasia, fribromatous lesions, fibrous displasia, multiple myeloma, abnormal bone turnover, osteolytic bone disease and periodontal disease. Additional examples of bone diseases suitably treated by the receptor interacting protein kinase 1 inhibitors described herein include bone fracture, bone trauma, or a bone deficit condition associated with post-traumatic bone surgery, post-prosthetic joint surgery, post-plastic bone surgery, post-dental surgery, bone chemotherapy treatment or bone radiotherapy treatment. Additional examples of diseases affecting bone or bone joints suitably treated by the receptor interacting protein kinase 1 inhibitors described herein include metastatic bone cancer, rheumatic diseases such as rheumatoid arthritis, osteoarthritis and other inflammatory arthropathies. In an embodiment, the receptor interacting protein kinase 1 inhibitors described herein may be used to treat postmenopausal osteoporosis via inhibiting osteocyte necroptosis and trabecular deterioration.

In an embodiment, the compounds and compositions of the present disclosure are useful for treating cardiovascular diseases that may be relate to the cardiovascular disorders of fragile plaque disorder, occlusive disorder and stenosis. Non-limiting cardiovascular diseases include coronary artery disorders and peripheral arterial disorders, including, among others, atherosclerosis, arterial occlusion, aneurysm formation, thrombosis, post-traumatic aneurysm formation, restenosis, and post-operative graft occlusion. It is believed that atherosclerosis results from maladaptive inflammation driven primarily by macrophages. Thus, the compounds and compositions of the present disclosure may be used to treat atherosclerosis via inhibiting macrophage necroptosis.

In an embodiment, the compounds and compositions of the present disclosure are useful for treating transplant patients. Non-limiting examples of transplant patient suitably treated by the receptor interacting protein kinase 1 inhibitors described herein include patients with solid and non-solid organ and tissue transplantations and transplants, such as liver, heart, kidney, and heterologous and autologous bone marrow transplantations/transplants. Typically, immunosuppressive therapy is used to avoid graft rejection in recipients of solid organ transplants. Recipients of bone marrow transplants are usually subjected to extensive irradiation and chemotherapy prior to transplantation. It is believed that receptor interacting protein kinase 1 and NF-κB signaling in dying cells determines cross-priming of CD8+ T cells. Thus, the receptor interacting protein kinase 1 inhibitors described herein may be used to treat transplant patient and avoid graft rejection by modulating cross-priming of CD8+ T cells.

Additional examples of diseases and disorders suitably treated by the receptor interacting protein kinase 1 inhibitors described herein include Gaucher disease, organ failure, pancreatitis, atopic dermatitis, spondyloarthritis, gout, systemic onset juvenile idiopathic arthritis (SoJIA), systemic lupus erythematosus (SLE), Sjogren's syndrome, systemic scleroderma, anti-phospholipid syndrome (APS), vasculitis, primary sclerosing cholangitis (PSC), acetaminophen toxicity, kidney damage/injury (nephritis, renal transplant, surgery, administration of nephrotoxic drugs e.g. cisplatin, acute kidney injury (AKI)), Celiac disease, autoimmune idiopathic thrombocytopenic purpura (autoimmune ITP), cerebrovascular accident (CVA, stroke), myocardial infarction (MI), allergic diseases (including asthma), diabetes, Wegener's granulomatosis, pulmonary sarcoidosis, Behcet's disease, interleukin-1 converting enzyme (ICE/caspase-1) associated fever syndrome, chronic obstructive pulmonary disease (COPD), tumor necrosis factor receptor-associated periodic syndrome (TRAPS), peridontitis, NEMO-deficiency syndrome (F-kappa-B essential modulator gene (also known as IKK gamma or IKKG) deficiency syndrome), HOIL-1 deficiency ((also known as RBCK1) heme-oxidized IRP2 ubiquitin ligase-1 deficiency), linear ubiquitin chain assembly complex (LUBAC) deficiency syndrome, hematological and solid organ malignancies, bacterial infections and viral infections (e.g., tuberculosis and influenza) and lysosomal storage diseases.

Non-limiting examples of lysosomal storage diseases include Gaucher disease, GM2 Gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency. Niemann-Pick disease, neuronal ceroid lipofuscinoses, Pompe disease, pycnodysostosis, Sandhoff disease, Schindler disease, sialic acid storage disease, Tay-Sachs and Wolman disease. In certain embodiments, provided are compounds and compositions for use in medicine. In certain embodiments, the compounds and compositions are for use in the treatment of a receptor interacting protein kinase 1-mediated disease or disorder.

The methods of treating or preventing a RIPK1 kinase-related disease (e.g., cancer) in a subject can further comprise administering to the subject one or more additional agents. The one or more additional agents and the compounds described herein or pharmaceutically acceptable salts or prodrugs thereof can be administered in any order, including concomitant, simultaneous, or sequential administration. Sequential administration can be administration in a temporally spaced order of up to several days apart. The methods can also include more than a single administration of the one or more additional agents and/or the compounds described herein or pharmaceutically acceptable salts or prodrugs thereof. The administration of the one or more additional agents and the compounds described herein or pharmaceutically acceptable salts or prodrugs thereof can be by the same or different routes and concurrently or sequentially.

Additional therapeutic agents include, but are not limited to, chemotherapeutic agents, anti-depressants, anxiolytics, antibodies, antivirals, steroidal and non-steroidal anti-inflammatories, conventional immunotherapeutic agents, cytokines, chemokines, and/or growth factors. The additional therapeutic agents can be biomolecules.

A chemotherapeutic agent is a compound or composition effective in inhibiting or arresting the growth of an abnormally growing cell. Thus, such an agent may be used therapeutically to treat cancer as well as other diseases marked by abnormal cell growth. Illustrative examples of chemotherapeutic compounds include, but are not limited to, bexarotene, gefitinib, erlotinib, gemcitabine, paclitaxel, docetaxel, topotecan, irinotecan, temozolomide, carmustine, vinorelbine, capecitabine, leucovorin, oxaliplatin, bevacizumab, cetuximab, panitumumab, bortezomib, oblimersen, hexamethylmelamine, ifosfamide, CPT-11, deflunomide, cycloheximide, dicarbazine, asparaginase, mitotant, vinblastine sulfate, carboplatin, colchicine, etoposide, melphalan, 6-mercaptopurine, teniposide, vinbriastine, antibiotic derivatives (e.g. anthracyclines such as doxorubicin, liposomal doxorubicin, and diethylstilbestrol doxorubicin, bleomycin, daunorubicin, and dactinomycin); antiestrogens (e.g., tamoxifen); antimetabolites (e.g., fluorouracil (FU), 5-FU, methotrexate, floxuridine, interferon alpha-2B, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cisplatin, vincristine and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chlorambucil, mechlorethamine (nitrogen mustard) and thiotepa); and steroids (e.g., bethamethasone sodium phosphate).

Therapeutic agents further include, but are not limited to, levadopa, a dopamine agonist, an anticholinergic agent, a monoamine oxidase inhibitor, a COMT inhibitor, amantadine, rivastigmine, an NMDA antagonist, a cholinesterase inhibitor, riluzole, an anti-psychotic agent, an antidepressant, and tetrabenazine.

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

Optionally, a compound or therapeutic agent as described herein may be administered in combination with a radiation therapy, an immunotherapy, a gene therapy, or a surgery.

The methods and compounds as described herein are useful for both prophylactic and therapeutic treatment. For prophylactic use, a therapeutically effective amount of the compounds and compositions or pharmaceutically acceptable salts thereof as described herein are administered to a subject prior to onset (e.g., before obvious signs of a RIPK1 kinase-related disease), during early onset (e.g., upon initial signs and symptoms of a RIPK1 kinase-related disease), or after the development of a RIPK1 kinase-related disease. Prophylactic administration can occur for several days to years prior to the manifestation of symptoms of a RIPK1 kinase-related disease. Therapeutic treatment involves administering to a subject a therapeutically effective amount of the compounds and compositions or pharmaceutically acceptable salts thereof as described herein after a RIPK1 kinase-related disease is diagnosed.

The compounds described herein are also useful in modulating RIPK1 kinase in a cell. Optionally, the compounds and compositions described herein are useful for inducing RIPK1 kinase degradation in a cell. The methods for inducing RIPK1 kinase degradation in a cell includes contacting a cell with an effective amount of one or more of the compounds as described herein. Optionally, the contacting is performed in vivo. Optionally, the contacting is performed in vitro.

The methods herein for prophylactic and therapeutic treatment optionally comprise selecting a subject with or at risk of developing a RIPK1 kinase-related disease. A skilled artisan can make such a determination using, for example, a variety of prognostic and diagnostic methods, including, for example, a personal or family history of the disease or condition, clinical tests (e.g., imaging, biopsy, genetic tests), and the like. Optionally, the methods herein can be used for preventing relapse of cancer in a subject in remission (e.g., a subject that previously had cancer).

V. Kits

Also provided herein are kits for treating or preventing a RIPK1 kinase-related disease (e.g., cancer, a neurodegenerative disorder, and/or an inflammatory disease) in a subject. A kit can include any of the compounds or compositions described herein. For example, a kit can include one or more compounds of Formula I, Formula H, and/or Formula III. A kit can further include one or more additional agents, such as one or more anti-inflammatory agents and/or chemotherapeutic agents. A kit can include an oral formulation of any of the compounds or compositions described herein. A kit can include an intravenous formulation of any of the compounds or compositions described herein. A kit can additionally include directions for use of the kit (e.g., instructions for treating a subject), a container, a means for administering the compounds or compositions (e.g., a syringe), and/or a carrier.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs (e.g., size of the tumor or rate of tumor growth) of the disease in a subject as compared to a control. As used herein, control refers to the untreated condition (e.g., the tumor cells not treated with the compounds and compositions described herein). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs: and goats. Non-mammals include, for example, fish and birds.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Compound Synthesis

Compounds of Formula I to III can be prepared using the synthetic schemes and procedures described in detail below.

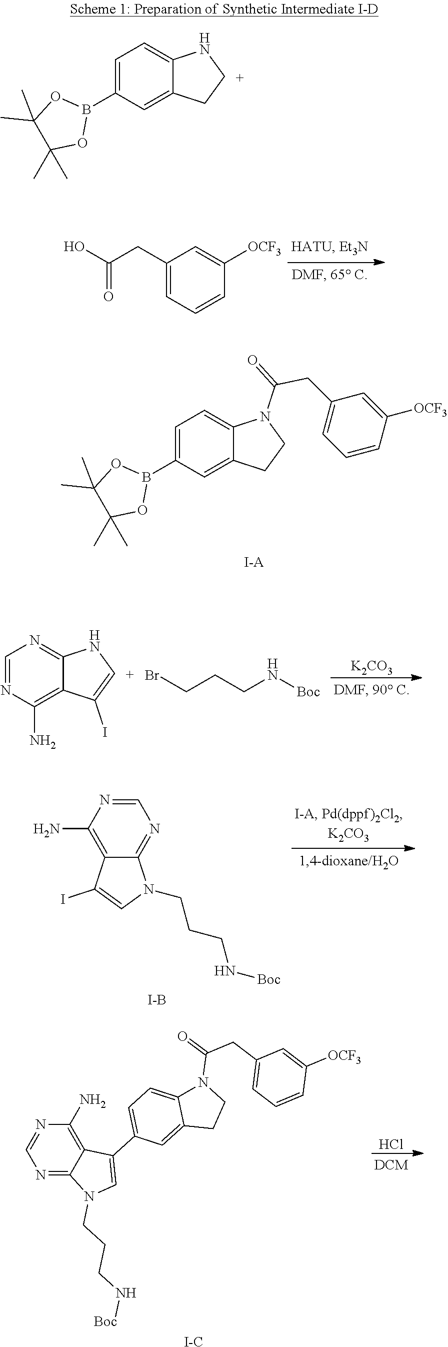

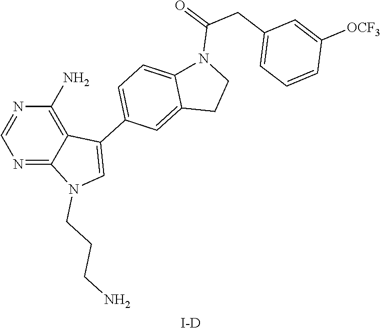 [SMILES: NCCCn1cc(-c2ccc3c(c2)CCN3C(=O)Cc2cccc(OC(F)(F)F)c2)c2c(N)ncnc21 ; "I-D"]

1-(5-(4,4,5,5-tetramethyl-1,3,2-dioxaborolan-2-yl)indolin-1-yl)-2-(3-(trifluoromethoxy)phenyl)ethan-1-one (I-A): To a solution of 5-(4,4,5,5-tetramethyl-1,3,2-dioxaborolan-2-yl)indoline (1.5 g, 6.12 mmol) in DMF (50 mL) was added 2-(3-(trifluoromethoxy)phenyl)acetic acid (2.81 g, 6.7 mmol) and Et₃N (1.77 mL, 12.24 mmol). The mixture was stirred at 65° C. for 3 hours before adding water (100 mL). The resulting mixture was extracted with ethyl acetate (2×100 mL), the combined organic phases were dried over anhydrous Na. SO₄, and concentrated under reduced pressure to give the title compound (2.18 g, 80% yield). MS (ESI) m/z: [M+H]⁺, 448.2.

tert-butyl (3-(4-amino-5-iodo-7H-pyrrolo[2,3-d]pyrimidin-7-yl)propyl)carbamate (I-B): To a solution of 5-iodo-7H-pyrrolo[2,3-d]pyrimidin-4-amine (1.0 g, 3.8 mmol) and tert-butyl (3-bromopropyl)carbamate (1.1 g, 4.6 mmol) in DMF (40 mL) was added K₂CO₃ (0.63 g, 4.6 mmol). The mixture was stirred at 90° C. for 5 hours before adding water (100 mL). The resulting mixture was extracted with CH₂Cl₂ (2×100 mL), the combined organic phases were dried over anhydrous Na₂SO₄ and concentrated under reduced pressure to give the title compound (1.15 g, 72% yield). MS (ESI) m/z: [M+H]⁺, 418.1.

tert-butyl (3-(4-amino-5-(1-(2-(3-(trifluoromethoxy)phenyl)acetyl)indolin-5-yl)-7H-pyrrolo[2,3-d]pyrimidin-7-yl)propyl)carbamate (I-C): A solution of I-A (1.0 g, 2.2 mmol), I-B (766 mg, 1.83 mmol), K₂CO₃ (505 mg, 3.66 mmol) and Pd(dppf)₂Cl₂·CH₂Cl₂ (75 mg, 0.09 mmol) in 1,4-dioxane (50 mL) and water (10 mL) was degassed and fulfilled with N₂ for three times. Then the mixture was stirred at 100° C. for 12 hours before it was quenched with water. The resulting mixture was extracted with CH₂Cl₂ (3×100 mL), the combined organic phases were dried over anhydrous Na₂SO₄ and concentrated under reduced pressure. The residue was purified by flash column chromatography to give the title compound (500 mg, 45% yield). MS (ESI) m % z: [M+H]⁺, 611.2.

1-(5-(4amino-7-(3-aminopropyl)-7H-pyrrolo[2,3-d]pyrimidin-5-yl)indolin-1-yl)-2-(3-(trifluoromethoxy)phenyl)ethan-1-one (I-D): To a solution of I-C (500 mg, 0.82 mmol) in CH₂Cl₂ (30 mL) was slowly added 2 N HCl in ether (2 mL) in an ice bath. Then the solution was stirred at room temperature overnight. The mixture was filtered and the solid was dried under vacuum to give the title compound (330 mg, 79% yield). MS (ESI) m/z: [M+H]⁺, 511.2.

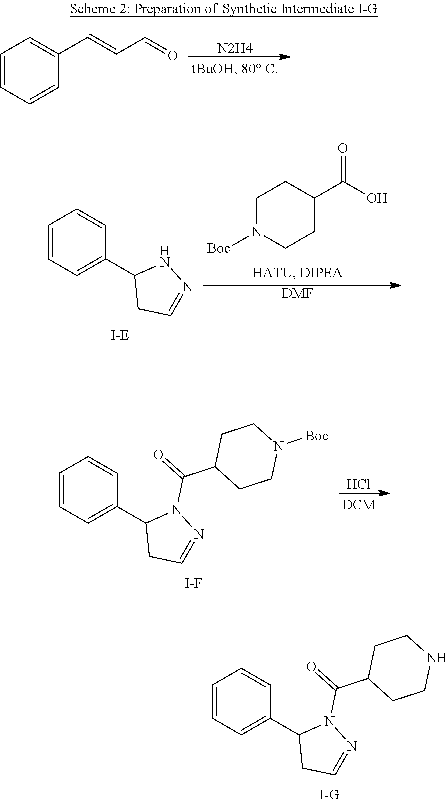

5-phenyl-4,5-dihydro-1H-pyrazole (I-E): Hydrazine (0.684 mL, 19 mmol) was heated to reflux. A solution of cinnamaldehyde (1.0 g, 7.6 mmol) in tert-butanol (20 mL) was added dropwise, and the mixture was refluxed for 6 hours. The reaction mixture was concentrated under reduced pressure. The crude material was then diluted with DCM (2×100 mL) and washed with water. The combined organic layers were washed with water and then dried over Na₂SO₄ and concentrated under reduced pressure to provide the title compound (0.94 g, 85% yield) of a yellow oil. The product was carried onto the next reaction without further purification. MS (ESI) m/z: [M+H]⁺, 147.2.

tert-butyl 4-(5-phenyl-4,5-dihydro-1H-pyrazole-1-carbonyl)piperidine-1-carboxylate (I-F): To a solution of 1-(tert-butoxycarbonyl)piperidine-4-carboxylic acid (I-E) (1.04 g, 4.51 mmol) in DMF (30 mL) was added DIPEA (2.15 mL, 12.31 mmol) and HATU (1.87 g, 4.93 mmol) followed by 5-phenyl-4,5-dihydro1H-pyrazole (I-E) (600 mg, 4.1 mmol). The reaction mixture was stirred overnight at room temperature before adding water (200 mL). The reaction mixture was extracted with CH₂Cl₂ (3×100 mL), washed with water (3×100 mL), dried over anhydrous Na₂SO₄, filtered and concentrated in vacuo. The product was purified by flash column chromatography to give the title compound (450 mg, 30% yield). MS (ESI) m % z: [M−H]⁻, 356.2.

(5-phenyl-4,5-dihydro-1H-pyrazol-1-yl)(piperidin-4-yl)methanone (I-G): To a solution of tert-butyl 4-(5-phenyl-4,5-dihydro-1H-pyrazole-1-carbonyl)piperidine-1-carboxylate (I-F) (500 mg, 1.4 mmol) in DCM (20 mL) cooled to 0° C. was added 2N HCl in ether (2.8 mL, 5.6 mmol). The reaction mixture was stirred at room temperature for 8 hours. The reaction was concentrated and then taken up in DMF and purified by reverse phase preparative HPLC to provide the product as a white solid (270 mg, 75% yield) MS (ESI) m/z: [M+H]⁺, 258.2. 1H NMR (400 MHz, DMSO) δ 8.98 (s, 1H), 8.53-8.10 (m, 1H), 7.29 (t, J=7.2 Hz, 2H), 7.25-7.18 (m, 2H), 7.07 (d, 1=7.9 Hz, 2H), 5.27 (dd, J=11.9, 4.5 Hz, 1H), 3.46 (dd, J=19.0, 11.9 Hz, 2H), 3.22 (d, J=11.8 Hz, 2H), 2.93 (dd, J=11.5, 8.0 Hz, 2H), 2.65 (dd, J=18.9, 4.5 Hz, 1H), 1.94 (d, J=12.5 Hz, 1H), 1.82-1.58 (m, 3H).

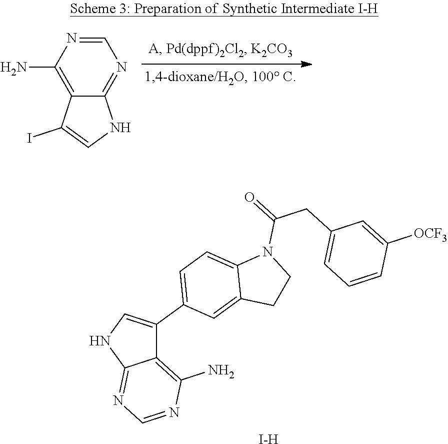 [SMILES: Nc1ncnc2[nH]cc(I)c12 ; Nc1ncnc2[nH]cc(-c3ccc4c(c3)CCN4C(=O)Cc3cccc(OC(F)(F)F)c3)c12 ; "I-H"]

1-(5-(4-amino-7H-pyrrolo[2,3-d]pyrimidin-5-yl)indolin-1-yl)-2-(3-(trifluoromethoxy)phenyl)ethan-1-one (I-H): A solution of 5-iodo-7H-pyrrolo[2,3-d]pyrimidin-4-amine (500 mg, 1.9 mmol), I-A (1 g, 2.28 mmol), K₂CO₃ (525 mg, 3.8 mmol) and Pd(dppf)₂Cl₂·CH₂Cl₂ (155 mg, 0.19 mmol) in 1,4-dioxane (50 mL) and water (10 mL) was degassed and fulfilled with N₂ for three times. Then the mixture was stirred at 100° C. for 12 hours before it was quenched with water. The resulting mixture was extracted with CH₂Cl₂ (3×100 mL), the combined organic phases were dried over anhydrous Na₂SO₄ and concentrated under reduced pressure. The residue was purified by flash column chromatography to give the title compound (350 mg, 20% yield). MS (ESI) m % z: [M+H]⁺, 454.2.

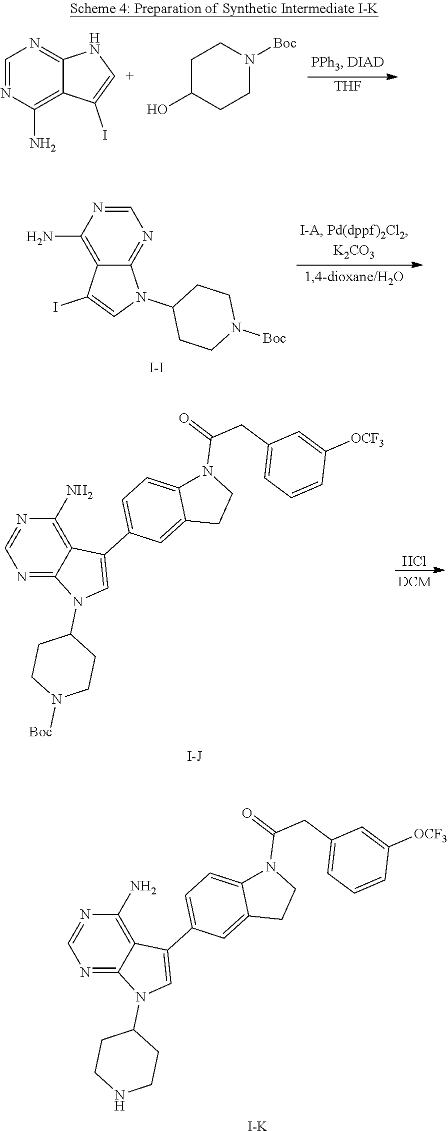

tert-butyl 4-(4-amino-5-iodo-7H-pyrrolo[2,3-d]pyrimidin-7-yl)piperidine-1-carboxylate (I-I): To a solution of 5-iodo-7H-pyrolo[2,3-d]pyrimidin-4-amine (1.0 g, 3.8 mmol), PPh₃ (1.5 g, 5.7 mmol) and N-Boc-4-hydroxypiperidine (1.5 g, 5.7 mmol) in THE (40 mL) was degassed and fulfilled with N2 for three times. Then the mixture was added DIAD (1.13 mL, 5.7 mmol) dropwise at 0° C. The mixture was stirred at room temperature overnight before adding water (100 mL). The resulting mixture was extracted with CH₂Cl₂ (2×100 mL), the combined organic phases were dried over anhydrous Na₂SO₄ and concentrated under reduced pressure to give the title compound (1.14 g, 68% yield). MS (ESI) m/z: [M+H]⁺, 444.1.

tert-butyl 4-(4-amino-5-(1-(2-(3-(trifluoromethoxy)phenyl)acetyl)indolin-5-yl)-7H-pyrrolo[2,3-d]pyrimidin-7-yl)piperidine-1-carboxylate (I-J): A solution of I-A (8(X) mg, 1.8 mmol), I-I (872 mg, 1.97 mmol), K₂CO₃ (500 mg, 3.58 mmol) and Pd(dppf)₂Cl₂·CH₂Cl₂ (146 mg, 0.18 mmol) in 1,4-dioxane (50 mL) and water (10 mL) was degassed and fulfilled with N₂ for three times. Then the mixture was stirred at 90° C. for 12 hours before it was quenched with water. The resulting mixture was extracted with CH₂Cl₂ (3×100 mL), the combined organic phases were dried over anhydrous Na₂SO₄ and concentrated under reduced pressure. The residue was purified by flash column chromatography to give the title compound (680 mg, 60% yield). MS (ESI) m/z: [M+H]⁺, 637.3.

1-(5-(4-amino-7-(piperidin-4-yl)-7H-pyrrolo[2,3-d]pyrimidin-5-yl)indolin-1-yl)-2-(3-(trifluoromethoxy)phenyl)ethan-1-one (I-IQ: To a solution of I-J (680 mg, 1.08 mmol) in CH₂Cl₂ (30 mL) was slowly added 2 N HCl in ether (3 mL) in an ice bath. Then the solution was stirred at room temperature overnight. The mixture was filtered and the solid was dried under vacuum to give the title compound (538 mg, 93% yield). MS (ESI) m/z: [M+H]⁺, 537.2.

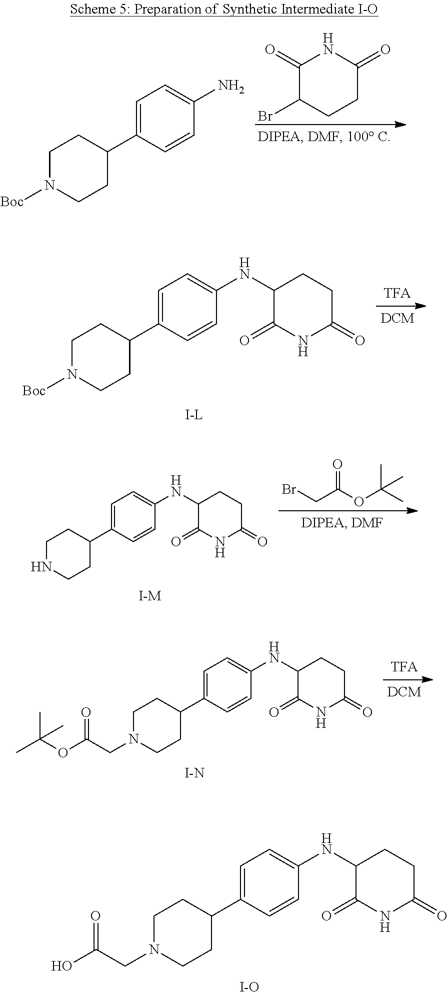

tert-butyl 4(4((2,6-dioxopiperidin-3-yl)amino)phenyl)piperidine-1-carboxylate (I-L): To a solution of tert-butyl 4-(4-aminophenyl)piperidine-1-carboxylate (1.0 g, 3.62 mmol) in DMF (30 mL) was added DIPEA (1.04 mL, 7.24 mmol) and 3-bromopiperidine-2,6-dione (1.39 g, 7.24 mmol). The mixture was stirred at 100° C. for 24 hours before it was quenched with water. The resulting mixture was extracted with ethyl acetate (3×100 mL), the combined organic phases were dried over anhydrous Na₂SO₄ and concentrated under reduced pressure. The residue was purified by flash column chromatography to give the title compound (1.05 g, 75% yield). MS (ESI) m z: [M+H]⁺, 388.2.

3-((4-(piperidin-4-yl)phenyl)amino)piperidine-2,6-dione (I-M): To a solution of I-L (1.05 g, 2.7 mmol) in CH₂Cl₂ (30 mL) was slowly added TFA (2 mL) in an ice bath. Then the solution was stirred at room temperature overnight. The mixture was concentrated under vacuum to give the title compound (711 mg, 91% yield). MS (ESI) m/z: [M+H]⁺, 288.2.

tert-butyl 2-(4-(4((2,6-dioxopiperidin-3-yl)amino)phenyl)piperidin-1-yl)acetate (I-N): To a solution of 1-M (711 mg, 2.5 mmol) in DMF (30 mL) was added DIPEA (3.58 mL, 25 mmol) and tert-butyl 2-bromoacetate (0.44 mL, 3.0 mmol). The mixture was stirred at room temperature overnight before it was quenched with water. The resulting mixture was extracted with DCM (2×100 mL), the combined organic phases were dried over anhydrous Na₂SO₄ and concentrated under reduced pressure. The residue was purified by flash column chromatography to give the title compound (522 mg, 52% yield). MS (ESI) m/z: [M+H]⁺, 402.2.

2-(4-(4-((2,6-dioxopiperidin-3-yl)amino)phenyl)piperidin-1-yl)acetic acid (I-O): To a solution of I-N(522 mg, 1.3 mmol) in CH₂Cl₂ (20 mL) was slowly added TFA (1 mL) in an ice bath. Then the solution was stirred at room temperature for 5 hours. The mixture was concentrated under vacuum to give the title compound (390 mg, 87% yield). MS (ESI) [M+H]⁺, 346.2.

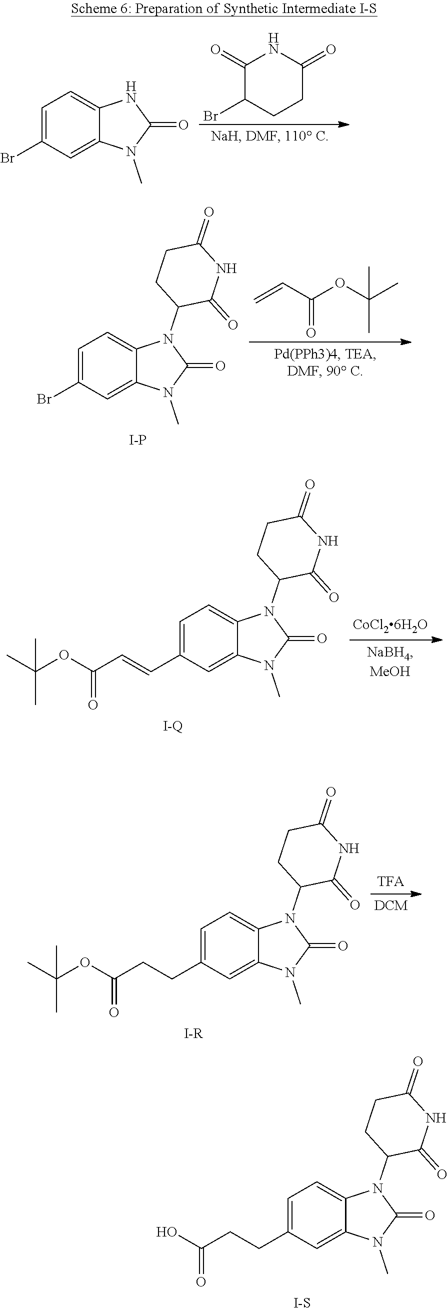

3-(5-bromo-3-methyl-2-ozo-2,3-dihydro-1H-benzo[d]imidazol-1-yl)piperidine-2,6-dione (I-P): To a solution of 6-bromo-1-methyl-1,3-dihydro-2H-benzo[d]imidazol-2-one (2.0 g, 8.8 mmol) in DMF (50 mL) was added NaH (530 mg, 13.2 mmol). Then mixture was stirred at 0° C. for 30 mins, then 3-bromopiperidine-2,6-dione (3.36 g, 17.6 mmol). The mixture was stirred at 110° C. for 24 hours before it was quenched with water. The resulting mixture was extracted with ethyl acetate (3×100 mL), the combined organic phases were dried over anhydrous Na₂SO₄ and concentrated under reduced pressure. The residue was purified by flash column chromatography to give the title compound (680 mg, 23% yield). MS (ESI) m/z: [M+H]⁺, 338.1, 340.1.

tert-butyl (E)-3-(1-(2,6-dioxopiperidin-3-yl)-3-methyl-2-oxo-2,3-dihydro-1H-benzo[d]imidazol-5-yl)acrylate (1-Q): A solution of I-P (680 mg, 2.0 mmol), tert-butyl acrylate (0.585 mL, 4.0 mmol), triethylamine (0.865 mL, 6.0 mmol) and Pd(PPh₃)₄ (231 mg, 0.2 mmol) in DMF (30 mL) was degassed and fulfilled with N₂ for three times. Then the mixture was stirred at 90° C. for 12 hours before it was quenched with water. The resulting mixture was extracted with ethyl acetate (2×100 mL), the combined organic phases were dried over anhydrous Na₂SO₄ and concentrated under reduced pressure. The residue was purified by flash column chromatography to give the title compound (480 mg, 62% yield). MS (ESI) m/z: [M+H]⁺, 386.1.

tert-butyl 3-(1-(2,6-dioxopiperidin-3-yl)-3-methyl-2-oxo-2,3-dihydro-1H-benzo[d]imidazol-5-yl)propanoate (I-R): To a solution of I-Q (480 mg, 1.24 mmol) in methanol (20 mL) was added Cobalt chloride hexahydrate (60 mg, 0.25 mmol) and NaBH₄ (230 mg, 6 mmol). The mixture was stirred at room temperature overnight before it was quenched with water. The resulting mixture was extracted with ethyl acetate (2×50 mL), the combined organic phases were dried over anhydrous Na₂SO₄ and concentrated under reduced pressure. The residue was purified by flash column chromatography to give the title compound (327 mg, 68% yield). MS (ESI) m/z: [M+H]⁺, 388.2.

3-(1-(2,6-dioxopiperidin-3-yl)-3-methyl-2-oxo-2,3-dihydro-1H-benzo[d]imidazol-5-yl)propanoic acid (I-S): To a solution of 1-R (327 mg, 0.84 mmol) in CH₂Cl₂ (20 mL) was slowly added TFA (1 mL) in an ice bath. Then the solution was stirred at room temperature for 8 hours. The mixture was concentrated under vacuum to give the title compound (245 mg, 89% yield). MS (ESI) m % z: [M+H]⁺, 332.1.

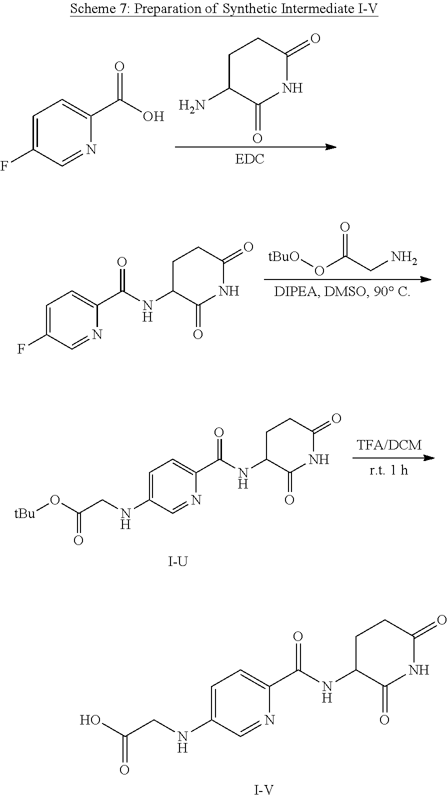

N-(2,6-dioxopiperidin-3-yl)-5-fluoropicolinamide (I-T): To a solution of 5-fluoropicolinic acid (500 mg, 3.54 mmol) in DMF (30 mL) was added HATU (1.7 g, 4.6 mmol), DIPEA (1.5 mL, 10.6 mmol). The mixture was stirred at room temperature for 10 mins followed adding 3-aminopiperidine-2,6-dione (543 mg, 4.25 mmol). The mixture was stirred at room temperature for 3 hours. The resulting mixture was extracted with ethyl acetate (2×30 mL), the combined organic phases were dried over anhydrous Na₂SO₄ and concentrated under reduced pressure. The residue was purified by flash column chromatography to give the title compound (293 mg, 33% yield). MS (ES) m/z: [M+H]⁺, 252.1.

tert-butyl (5-((2,6-dioxopiperidin-3-yl)carbamoyl)pyridin-2-yl)glycinate (I-U): I-T (293 mg, 1.16 mmol), tert-butyl glycinate (230 mg, 1.75 mmol) was dissolved in DMSO (20 mL). The solution was added DIPEA (0.5 mL, 3.48 mmol) and stirred at 90° C. for 12 hours before it was quenched with water. The resulting mixture was extracted with ethyl acetate (2×50 mL), the combined organic phases were dried over anhydrous Na₂SO₄ and concentrated under reduced pressure. The residue was purified by flash column chromatography to give the title compound (302 mg, 72% yield). MS (ESI) m/z: [M+H]⁺, 363.2.

(5-((2,6-dioxopiperidin-3-yl)carbamoyl)pyridin-2-yl)glycine (I-V): To a solution of I-U (302 mg, 0.84 mmol) in CH₂Cl₂ (20 mL) was slowly added TFA (1 mL) in an ice bath. Then the solution was stirred at room temperature for 8 hours. The mixture was concentrated under vacuum to give the title compound (185 mg, 72% yield). MS (ESI) m/z: [M+H]⁺, 307.1.

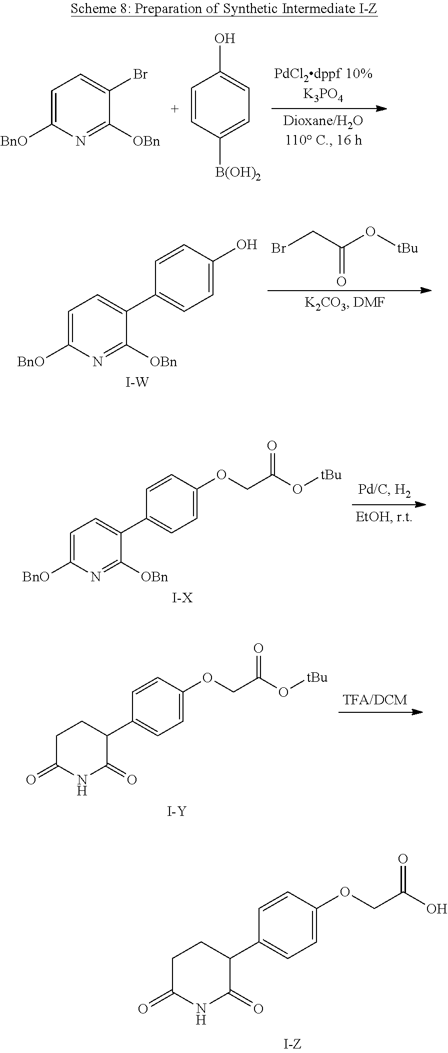

Intermediate I-Z was prepared according to the procedure described in Min et al., Phenyl-Glutarimides: Alternative Cereblon Binders for the Design of PROTACs. Anger. Chem. Int. Ed. (2021). doi: 10.1002/anie.202108848”.

### Preparation of Exemplary Compounds

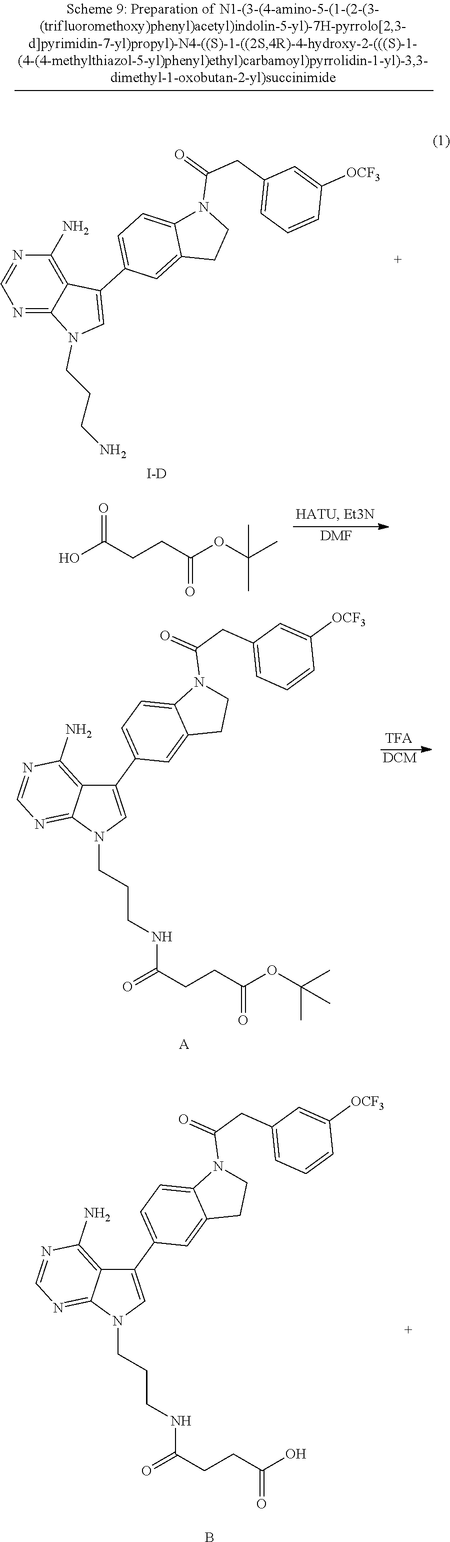

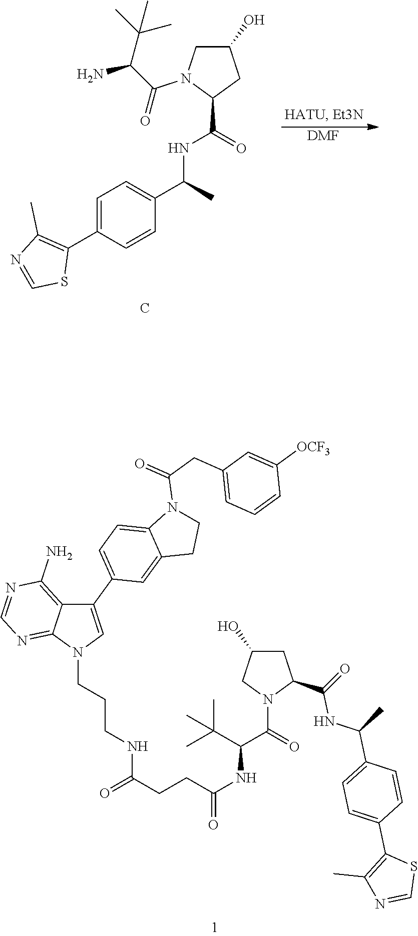

tert-butyl 4-((3-(4-amino-5-(1-(2-(3-(trifluoromethoxy)phenyl)acetyl)indolin-5-yl)-7H-pyrrolo[2,3-d]pyrimidin-7-yl)propyl)amino)-4-oxobutanoate (A): To a solution of I-D (50 mg, 0.1 mmol), 4-(tert-butoxy)-4-oxobutanoic acid (20 mg, 0.12 mmol) in DMF (5 mL) was added HATU (57 mg, 0.15 mmol) and Et₃N (43 uL, 0.3 mmol). The mixture was stirred at room temperature overnight. The reaction was concentrated and then was purified by flash column chromatography to give the title compound (43 mg, 67% yield). MS (ESI) m % z: [M+H]⁺, 667.2.

4((3-(4-amino-5-(1-(2-(3-(trifluoromethoxy)phenyl)acetyl)indolin-5-yl)-7H-pyrrolo[2,3-d]pyrimidin-7-yl)propyl)amino)-4-oxobutanoic acid (B): To a solution of A (43 mg, 0.06 mmol) in CH₂Cl₂ (2 mL) and TFA (1 mL) was stirred at 25° C. for 12 hours before it was concentrated under reduced pressure to give compound B (40 mg, quant yield). MS (ESI) m/z: [M+H]⁺, 611.2.

N1-(3-(4-amino-5-(1-(2-(3-(trifluoromethoxy)phenyl)acetyl)indolin-5-yl)-7H-pyrrolo[2,3-d]pyrimidin-7-yl)propyl)-N4-((S)-1-((2S,4R)-4-hydroxy-2-(((S)-1-(4-(4 methylthiazol-5-yl)phenyl)ethyl)carbamoyl)pyrrolidin-1-yl)-3,3-dimethyl-1-oxobutan-2-yl)succinimide (1): To a solution of B (40 mg, 0.04 mmol) and amino C (22 mg, 0.05 mmol) in DMF (5 mL) was added HATU (28 mg, 0.07 mmol) and Et₃N (17 uL, 0.12 mmol). The mixture was stirred at room temperature overnight. The reaction was concentrated and then was purified by reverse phase preparative HPLC to provide compound 1 (27 mg, 41% yield) MS (ESI) m/z: [M+H]⁺, 1037.4.

Example compounds 2-7, 25-28, 31-39 and 51-52 were prepared in an analogous manner to compound 1, employing the corresponding carboxylic acid starting materials and I-D or I-K.

TABLE 1

| | | MS (ESI) m/z: |
|---|---|---|
| | Structure | [M + H]⁺ |
| 2 | 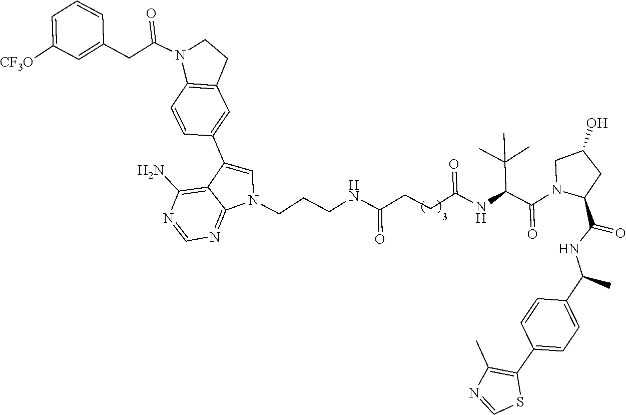 | 1065.5 |
| 3 | 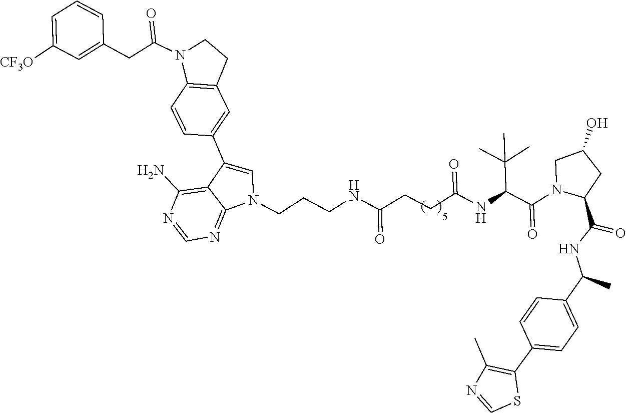 | 1093.5 |
| 4 | 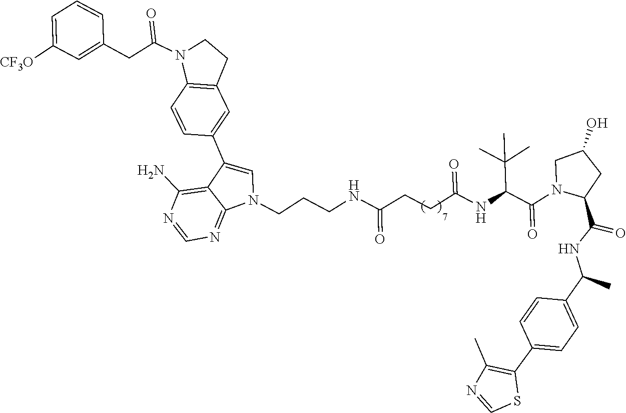 | 1121.5 |
| 5 | 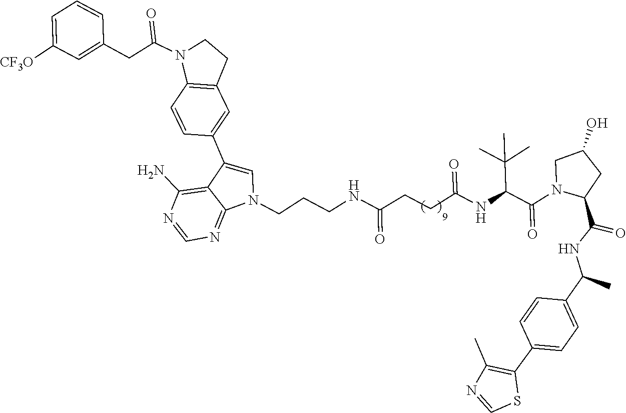 | 1149.5 |
| 6 | 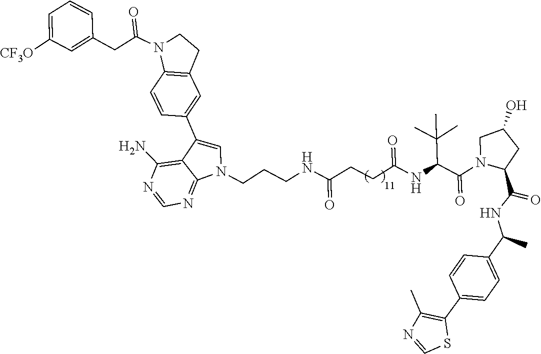 | 1177.6 |
| 7 | 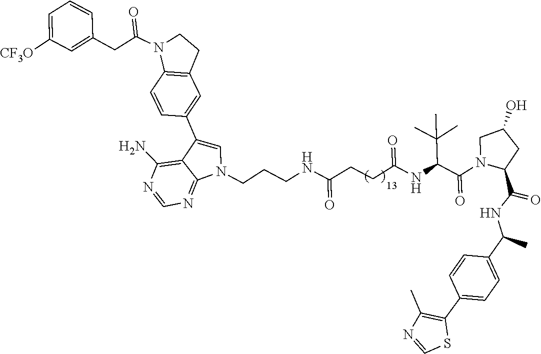 | 1205.6 |
| 25 | 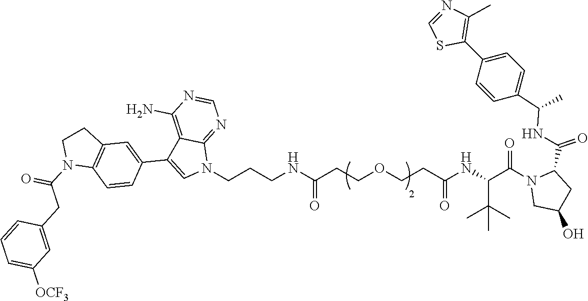 | 1125.5 |
| 26 | 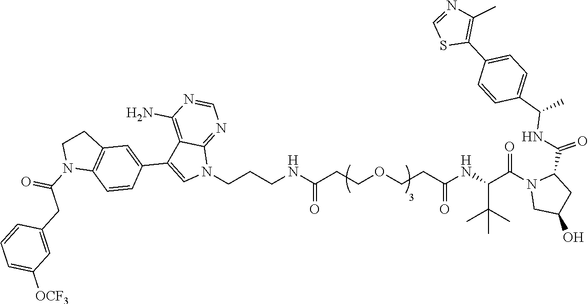 | 1169.5 |
| 27 | 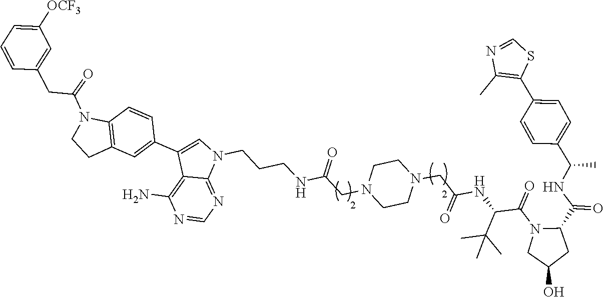 | 1149.5 |
| 28 | 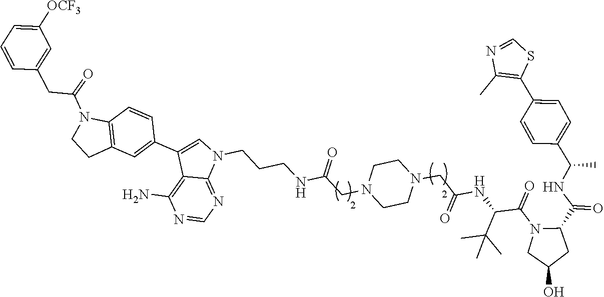 | 1177.6 |
| 31 | 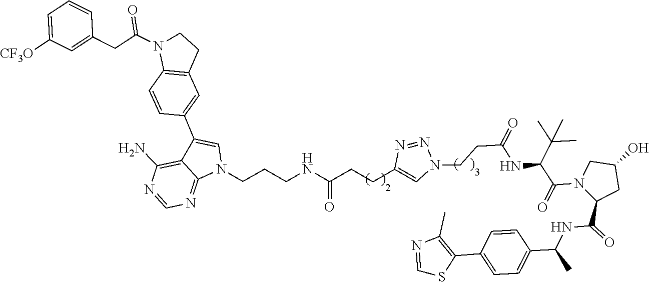 | 1174.6 |
| 32 | 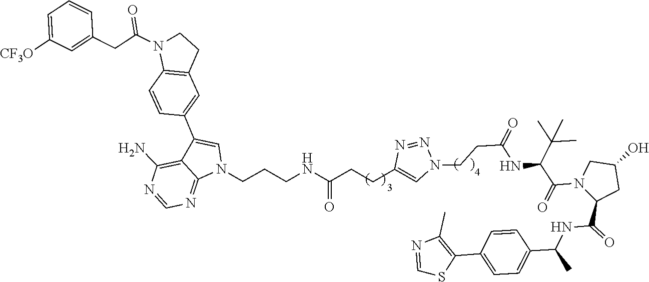 | 1202.6 |
| 33 | 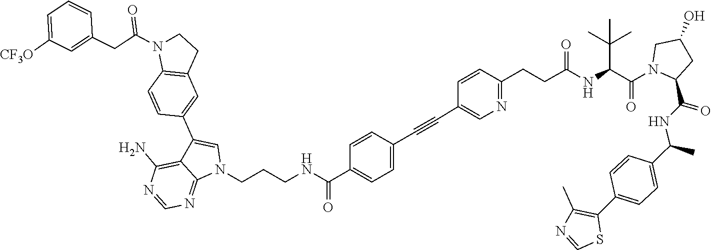 | 1214.5 |
| 34 | 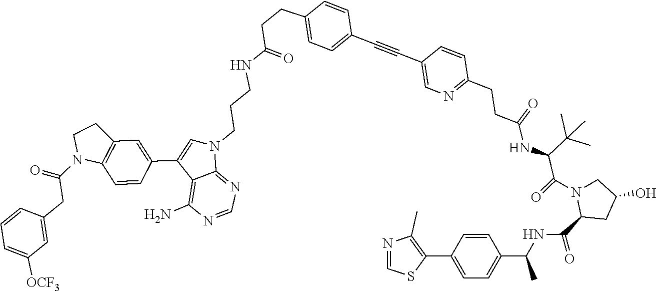 [SMILES: Cc1ncsc1-c1ccc([C@H](C)NC(=O)[C@@H]2C[C@@H](O)CN2C(=O)[C@@H](NC(=O)CCc2ccc(C#Cc3ccc(CCC(=O)NCCCn4cc(-c5ccc6c(c5)CCN6C(=O)Cc5cccc(OC(F)(F)F)c5)c5c(N)ncnc54)cc3)cn2)C(C)(C)C)cc1] | 1242.5 |
| 35 | 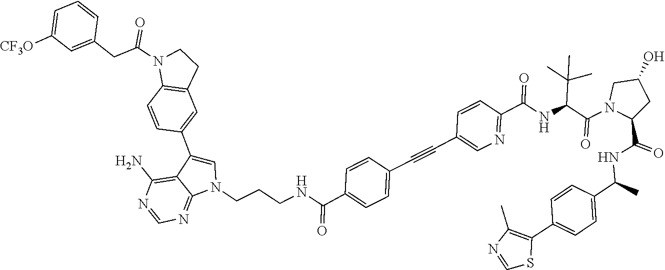 | 1186.5 |
| 36 | 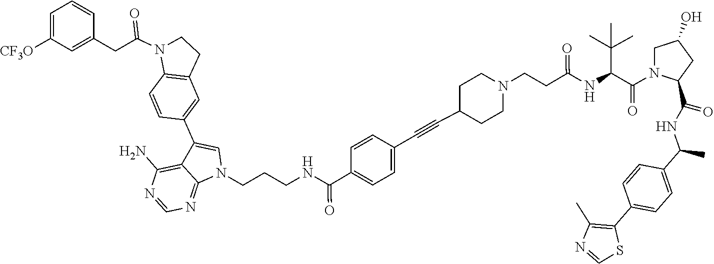 | 1220.5 |
| 37 | 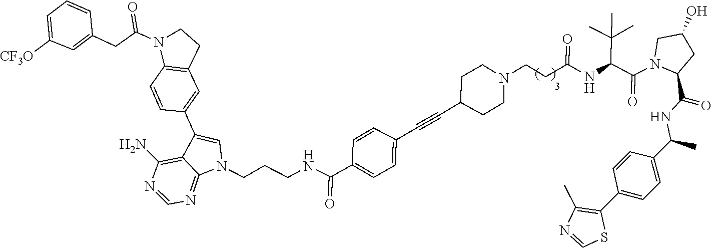 | 1248.6 |
| 38 | 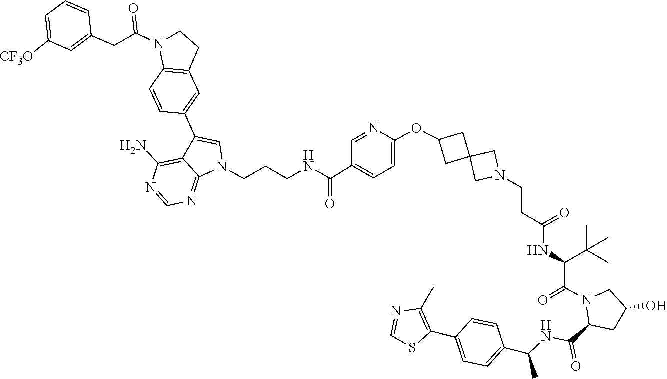 | 1225.5 |
| 39 | 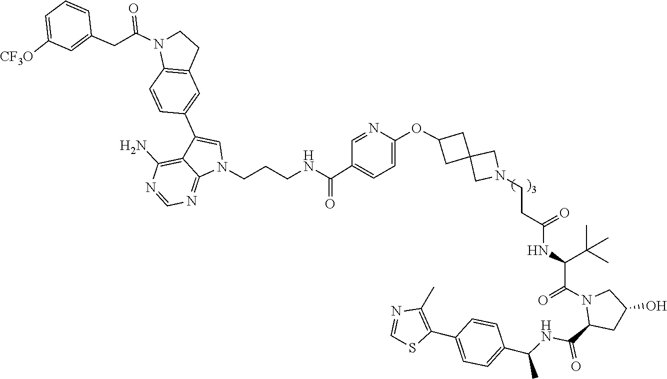 | 1253.6 |
| 51 | 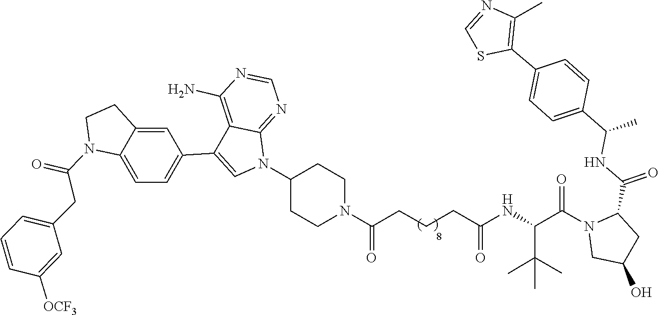 | 1163.6 |
| 52 | 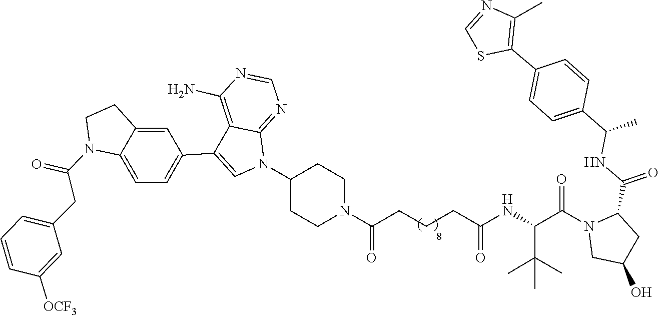 | 1191.6 |

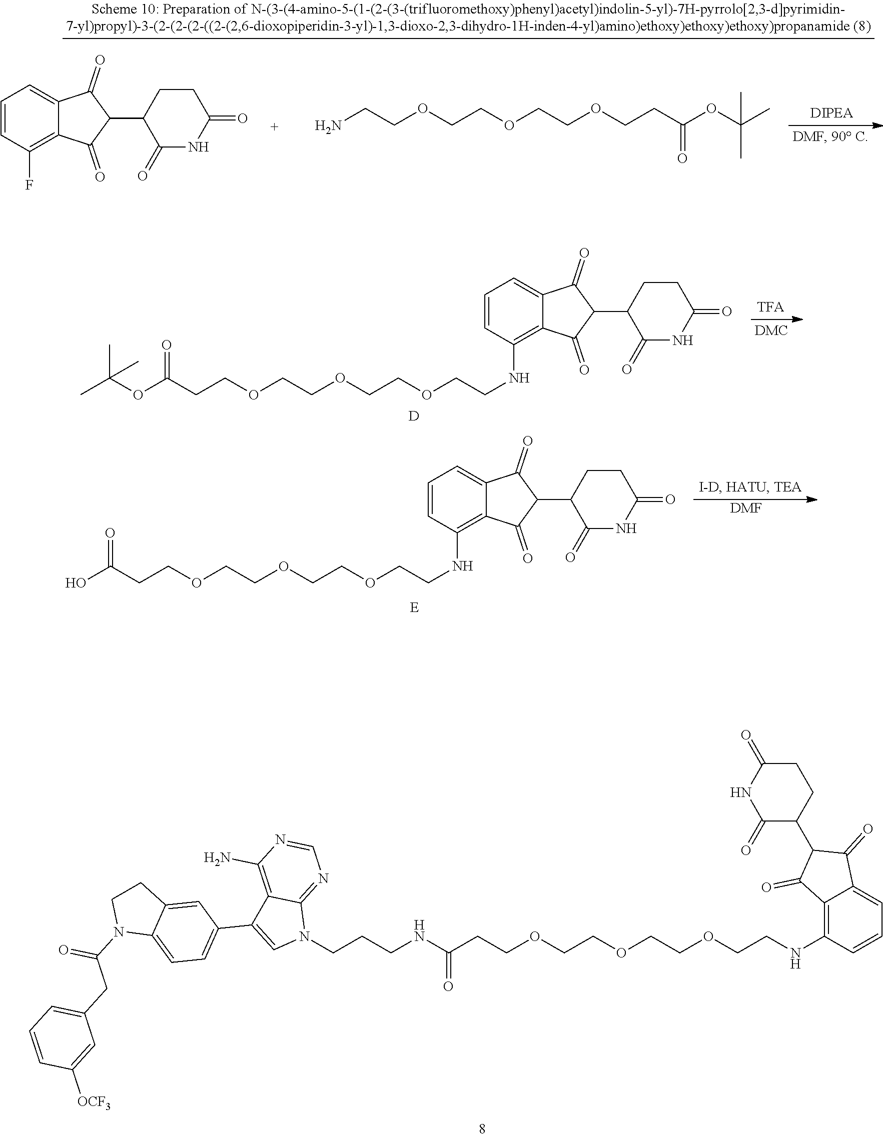

tert-butyl 3-(2-(2-(2-((2-(2,6-dioxopiperidin-3-yl)-1,3-dioxo-2,3-dihydro-1H-inden-4-yl)amino)ethoxy)ethoxy)ethoxy)propanoate (D): To a solution of 3-(4-fluoro-1,3-dioxo-2,3-dihydro-1H-inden-2-yl)piperidine-2,6-dione (50 mg, 0.18 mmol) in DMF (10 mL) was added tert-butyl 3-(2-(2-(2-aminoethoxy)ethoxy)ethoxy)propanoate (50 mg, 0.22 mmol) and DIPEA (51 uL, 0.36 mmol). The mixture was stirred at 90° C. for 2 hours. The reaction was concentrated and then was purified by flash column chromatography to give the title compound (74 mg, 84% yield) MS (ESI) m/z: [M+H]⁺, 533.2.

3-(2-(2-(2-((2-(2,6-dioxopiperidin-3-yl)-1,3-dioxo-2,3-dihydro-1H-inden-4-yl)amino)ethoxy)ethoxy)ethoxy)propanoic acid (E): To a solution of D (74 mg, 0.15 mmol) in CH₂Cl₂ (2 mL) and TFA (1 mL) was stirred at room temperature for 10 hours before it was concentrated under reduced pressure to give compound I (65 mg, quant yield). MS (ESI) m/z: [M+H]⁺, 477.1.

N-(3-(4-amino-5-(1-(2-(3-(trifluoromethoxy)phenyl)acetyl)indolin-5-yl)-7H-pyrrolo[2,3-d]pyrimidin-7-yl)propyl)-3-(2-(2-(2-((2-(2,6-dioxopiperidin-3-yl)-1,3-dioxo-2,3-dihydro-1H-inden-4-yl)amino)ethoxy)ethoxy)ethoxy)propanamide (8): To a solution of E (65 mg, 0.15 mmol), I-D (76 mg, 0.15 mmol) in DMF (10 mL) was added HATU (85 mg, 0.22 mmol) and Et₃N (65 uL, 0.45 mmol). The mixture was stirred at room temperature overnight. The reaction was concentrated and then was purified by reverse phase preparative HPLC to provide compound 8 (27 mg, 20% yield) MS (ESI) m/z: [M+H]⁺, 969.4.

Example compounds 9 and 53-61 was prepared in an analogous manner to compound 8, employing the corresponding amino starting materials and fluoride.

TABLE 2

| | Structure | MS (ESI) m/z: [M + H]⁺ |
|---|---|---|
| 9 | 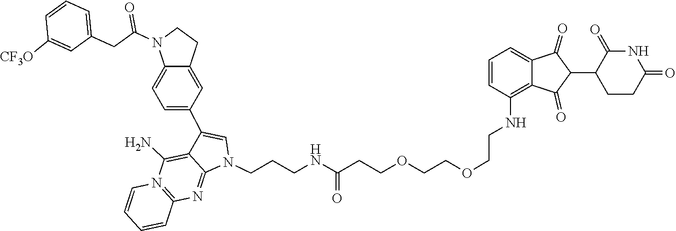 | 976.4 |
| 53 | 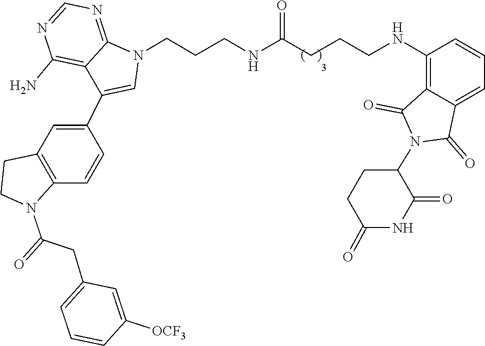 | 880.3 |
| 54 | 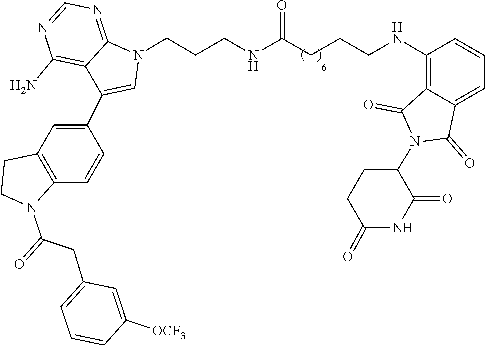 | 922.4 |
| 55 | 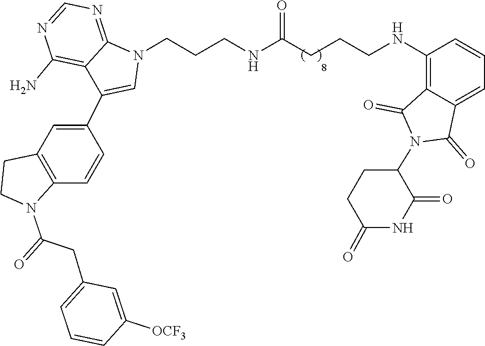 | 950.4 |
| 56 | 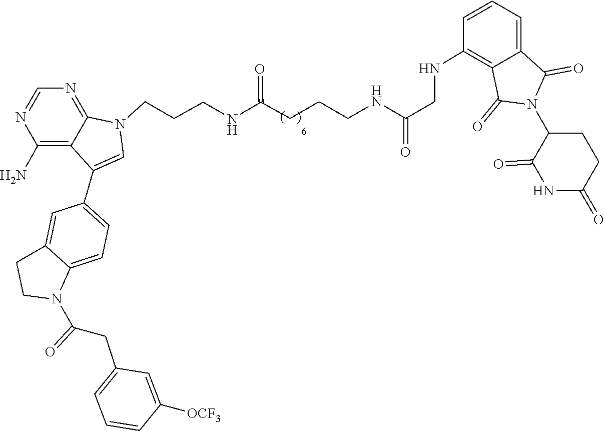 | 979.4 |
| 57 | 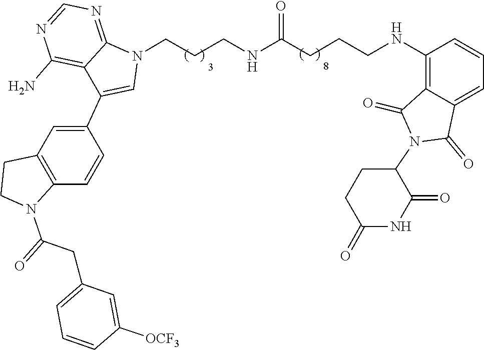 | 978.4 |
| 58 | 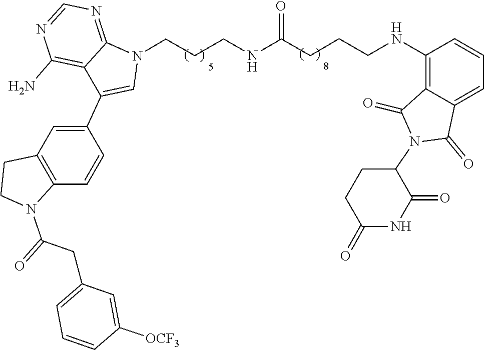 | 1006.5 |
| 59 | 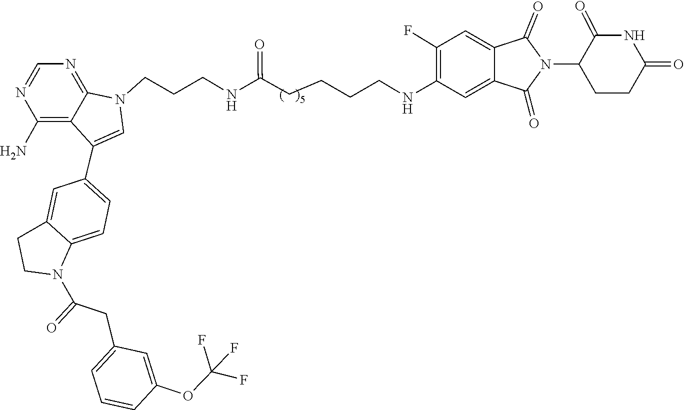 | 940.4 |
| 60 | 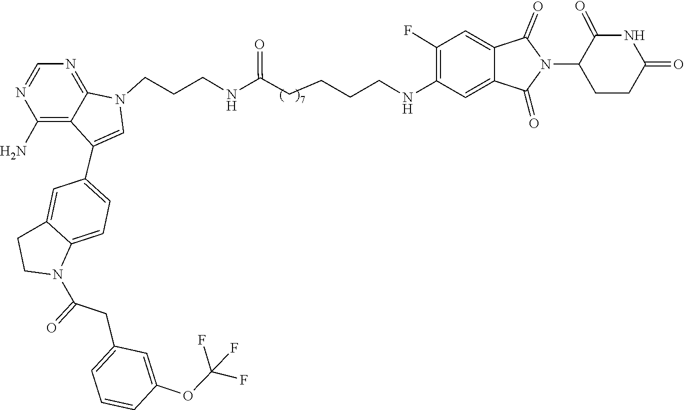 | 968.4 |
| 61 | 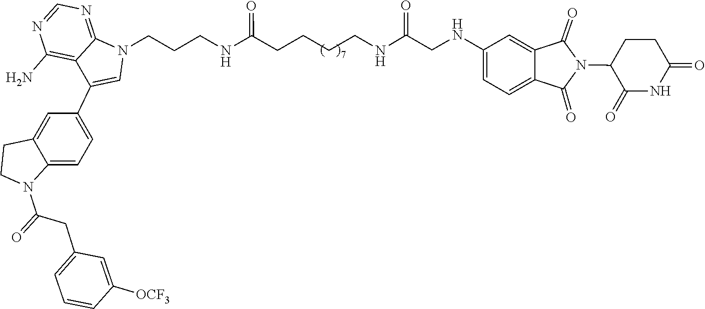 | 1007.5 |

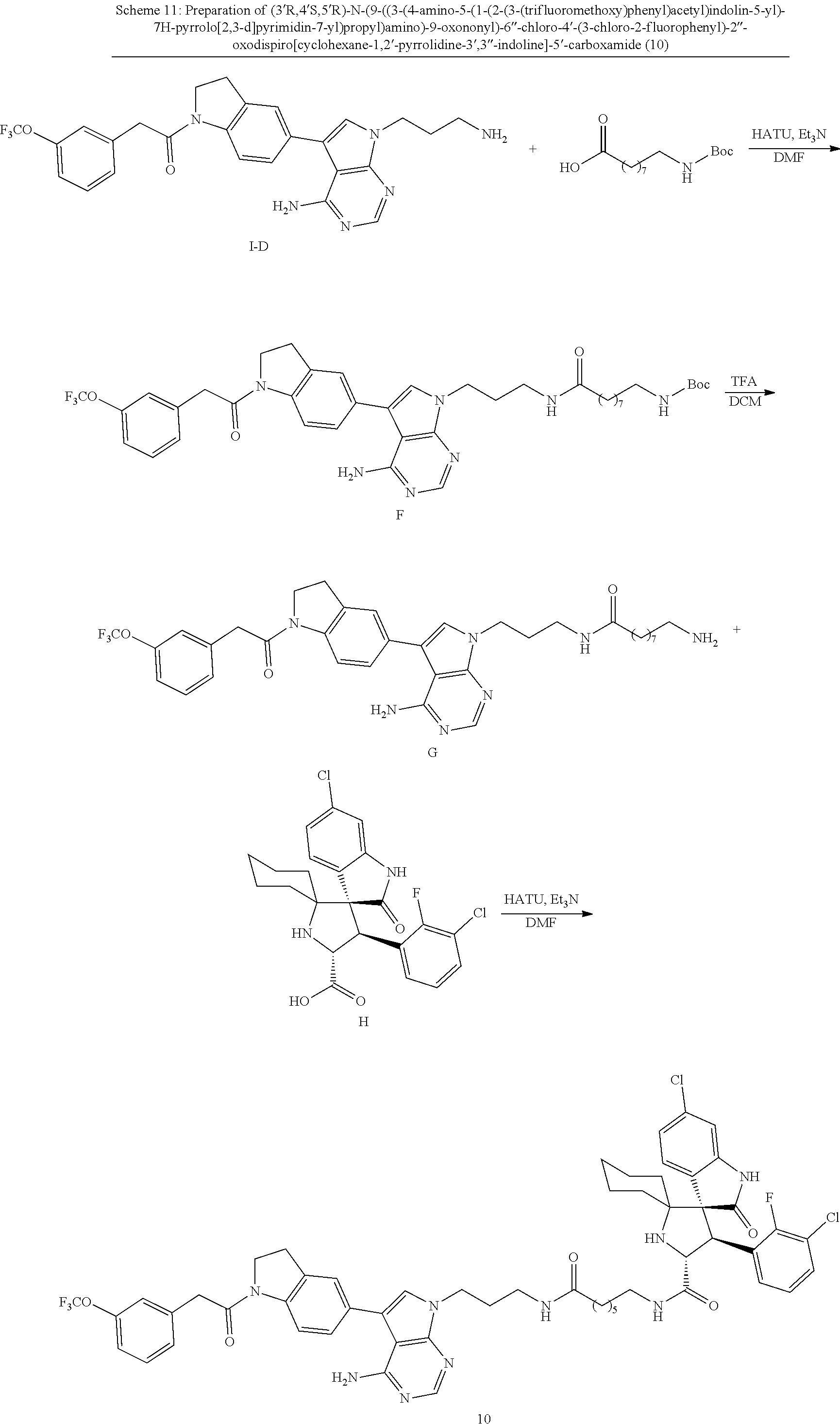

tert-butyl (9-((3-(4-amino-5-(1-(2-(3-(trifluoromethoxy)phenyl)acetyl)indolin-5-yl)-7H-pyrrolo[2,3-d]pyrimidin-7-yl)propyl)amino)-9-oxononyl)carbamate (F): To a solution of 1-D (50 mg, 0.1 mmol), 9-((tert-butoxycarbonyl)amino)nonanoic acid (33 mg, 0.12 mmol) in DMF (5 mL) was added HATU (57 mg, 0.15 mmol) and Et₃N (43 uL, 0.3 mmol). The mixture was stirred at room temperature overnight. The reaction was concentrated and then was purified by flash column chromatography to give the title compound (39 mg, 52% yield). MS (ESI) m/z: [M+H]⁺, 766.3.

tert-butyl (9-((3-(4-amino-5-(1-(2-(3-(trifluoromethoxy)phenyl)acetyl)indolin-5-yl)-7H-pyrrolo[2,3-d]pyrimidin-7-yl)propyl)amino)-9-oxononyl)carbamate (G): To a solution of F (39 mg, 0.05 mmol) in CH₂Cl₂ (2 mL) and TFA (1 mL) was stirred at room temperature for 1 h before it was concentrated under reduced pressure to give compound N (30 mg, 90% yield). MS (ESI) m/z: [M+H]⁺, 666.3.

(3′R,4'S,5′R)-N-(9-((3-(4-amino-5-(1-(2-(3-(trifluoromethoxy)phenyl)acetyl)indolin-5-yl)-7H-pyrrolo[2,3-d]pyrimidin-7-yl)propyl)amino)-9-oxononyl)-6″-chloro-4′-(3-chloro-2-fluorophenyl)-2″-oxodispiro[cyclohexane-1,2′-pyrrolidine-3′,3″-indoline]-5′-carboxamide (10): To a solution of G (30 mg, 0.045 mmol), H (20 mg, 0.045 mmol) in DMF (10 mL) was added HATU (25 mg, 0.068 mmol) and Et₃N (20 uL, 0.135 mmol). The mixture was stirred at room temperature overnight. The reaction was concentrated and then was purified by reverse phase preparative HPLC to provide compound 10 (17 mg, 35% yield) MS (ESI) m/z: [M+H]⁺, 1110.4.

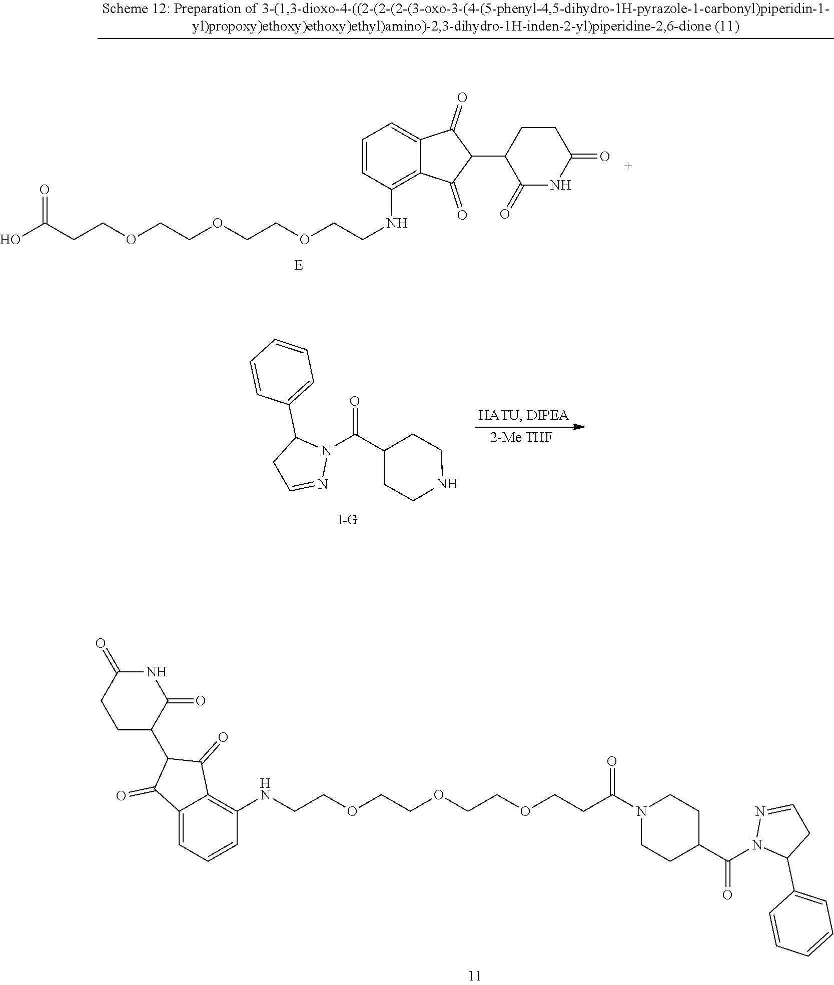 [SMILES: O=C(O)CCOCCOCCOCCNc1cccc2c1C(=O)C(C1CCC(=O)NC1=O)C2=O ; O=C(C1CCNCC1)N1N=CCC1c1ccccc1 ; O=C1CCC(C2C(=O)c3cccc(NCCOCCOCCOCCC(=O)N4CCC(C(=O)N5N=CCC5c5ccccc5)CC4)c3C2=O)C(=O)N1]

To a solution of E (15 mg, 0.035 mmol), I-G (9 mg, 0.035 mmol) in 2-methyltetrahydrofuran (5 mL) was added HATU (17 mg, 0.045 mmol) and DIPEA (15 uL, 0.1 mmol). The mixture was stirred at room temperature overnight. The reaction was concentrated and then was purified by reverse phase preparative HPLC to provide compound 11 (11 mg, 49% yield) MS (ESI) m % z: [M+H]⁺, 716.3.

Example compound 12 was prepared in an analogous manner to compound 11, employing the corresponding carboxylic acid starting materials L and G.

TABLE 3

| | | MS (ESI) m/z: |
|---|---|---|
| | Structure | [M + H]⁺ |
| 12 | 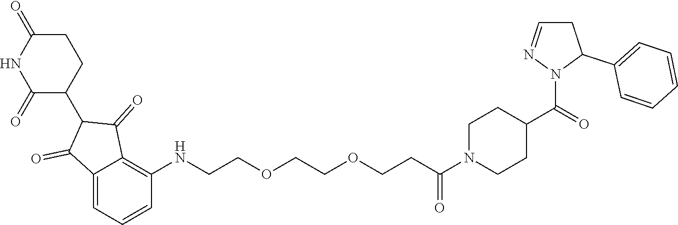 [SMILES: O=C1CCC(C2C(=O)c3cccc(NCCOCCOCCC(=O)N4CCC(C(=O)N5N=CCC5c5ccccc5)CC4)c3C2=O)C(=O)N1] | 672.3 |

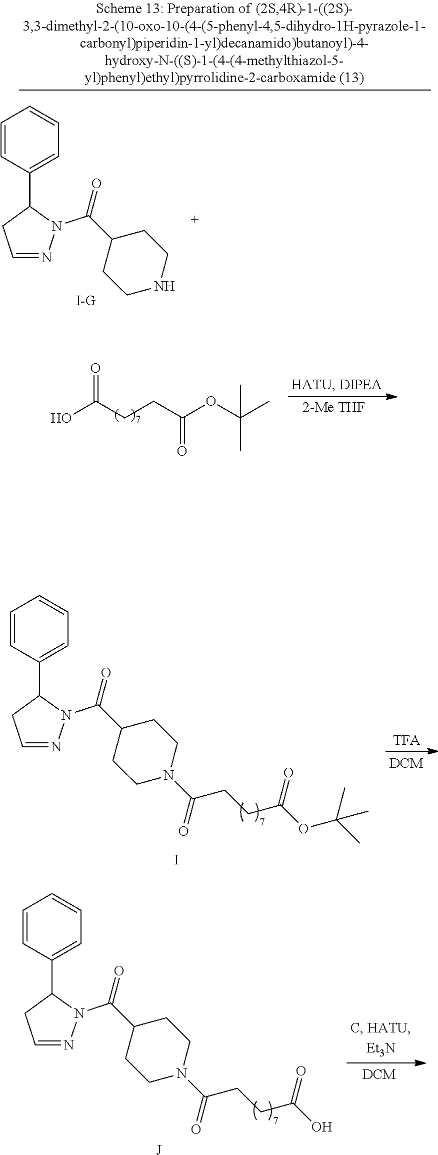

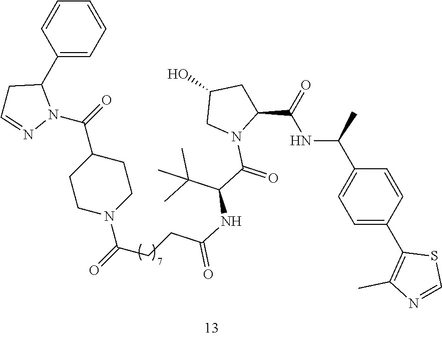

tert-butyl 10-oxo-10-(4-(5-phenyl-4,5-dihydro-1H-pyrazole-1-carbonyl)piperidin-1-yl)decanoate (1): To a solution of 10-(tert-butoxy)-10-oxodecanoic acid (10 mg, 0.042 mmol), I-G (9 mg, 0.035 mmol) in 2-methyltetrahydrofuran (5 mL) was added HATU (17 mg, 0.045 mmol) and DIPEA (15 uL, 0.1 mmol). The mixture was stirred at room temperature overnight. The reaction was concentrated and then was purified by flash column chromatography to give the title compound (11 mg, 67% yield). MS (ESI) m/z: [M+H]⁺, 868.5.

10-oxo-10-(4-(5-phenyl-4,5-dihydro-1H-pyrazole-1-carbonyl)piperidin-1-yl)decanoic acid (J): To a solution of 1 (11 mg, 0.022 mmol) in CH₂Cl₂ (2 mL) and TFA (1 mL) was stirred at room temperature for 5 h before it was concentrated under reduced pressure to give compound J (10 mg, quant yield). MS (ESI) m/z: [M+H]⁺, 442.2.

(2S,4R)-1-((2S)-3,3-dimethyl-2-(10-oxo-10-(4-(5-phenyl-4,5-dihydro-1H-pyrazole-1-carbonyl)piperid in-1-yl)decanamide)butanoyl)-4-hydroxy-N—((S)-1-(4-(4-methylthiazol-5-yl)phenyl)ethyl)pyrrolidine-2-carboxamide (13): To a solution of J (10 mg, 0.022 mmol), amino C (10 mg, 0.022 mmol) in DMF (5 mL) was added HATU (12 mg, 0.033 mmol) and Et₃N (10 uL, 0.066 mmol). The mixture was stirred at room temperature overnight. The reaction was concentrated and then was purified by reverse phase preparative HPLC to provide compound 13 (4.5 mg, 23% yield) MS (ESI) m/z: [M+H]⁺, 868.5.

Example compound 14 was prepared in an analogous manner to compound 13, employing the corresponding carboxylic acid starting materials and I-G.

TABLE 4

| | | MS (ESI) m/z: |
|---|---|---|
| | Structure | [M + H]⁺ |
| 14 | 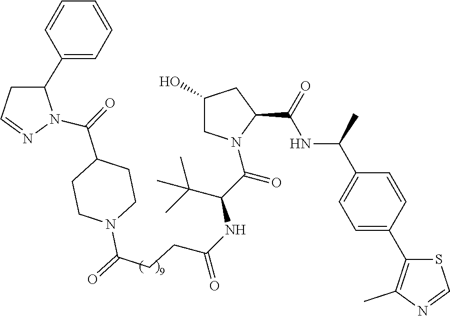 | 896.5 |

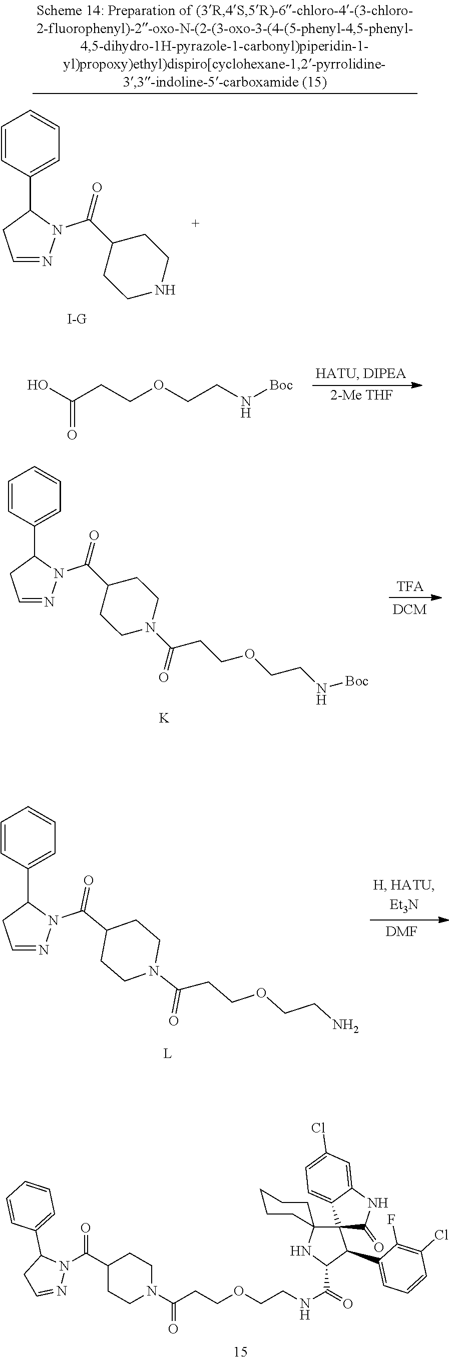

tert-butyl (2-(3-oxo-3-(4-(5-phenyl-4,5-dihydro-1H-pyrazole-1-carbonyl)piperidin-1-yl)propoxy)ethyl)carbamate (K): To a solution of 3-(2-((tert-butoxycarbonyl)amino)ethoxy)propanoic acid (54 mg, 0.23 mmol), I-G (50 mg, 0.19 mmol) in 2-methyltetrahydrofuran (5 mL) was added HATU (110 mg, 0.3 mmol) and DIPEA (86 uL, 0.6 mmol). The mixture was stirred at room temperature overnight. The reaction was concentrated and then was purified by flash column chromatography to give the title compound (68 mg, 75% yield). MS (ES) m/z: [M+H]⁺, 473.3.

3-(2-aminoethoxy)-1-(4-(5-phenyl-4,5-dihydro-1H-pyrazole-1-carbonyl)piperidin-1-yl)propan-1-one (L): To a solution of K (68 mg, 0.15 mmol) in CH₂Cl₂ (2.5 mL) and TFA (1 mL) was stirred at room temperature for 1 hour before it was concentrated under reduced pressure to give compound L (56 mg, quant yield). MS (ESI) m/z: [M+H]⁺, 373.3.

(3′R,4'S,5′R)-6″-chloro-4′-(3-chloro-2-fluorophenyl)-2″-oxo-N-(2-(3-oxo-3-(4-(5-phenyl-4,5-dihydro-1H-pyrazole-1-carbonyl)piperidin-1-yl)propoxy)ethyl)dispiro[cyclohexane-1,2′-pyrrolidine-3′,3″-indoline]-5′-carboxamide (15): To a solution of L (56 mg, 0.15 mmol), carboxyl acid H (83 mg, 0.18 mmol) in DMF (10 mL) was added HATU (85 mg, 0.22 mmol) and Et₃N (65 uL, 0.45 mmol). The mixture was stirred at room temperature overnight. The reaction was concentrated and then was purified by reverse phase preparative HPLC to provide compound 15 (31 mg, 25% yield) MS (ESI) m/z: [M+H]⁺, 817.3.

Example compounds 16-18 were prepared in an analogous manner to compound 15, employing the corresponding carboxylic acid starting materials and I-G.

TABLE 5

| | | MS (ESI) m/z: |
|---|---|---|
| | Structure | [M + H]⁺ |
| 16 | 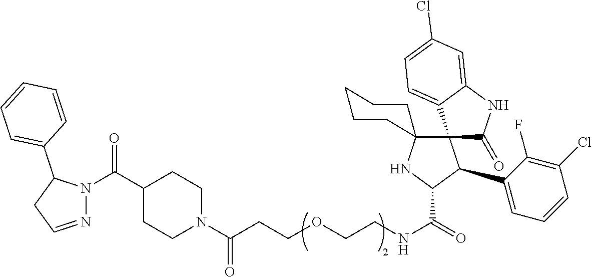 | 861.3 |
| 17 | 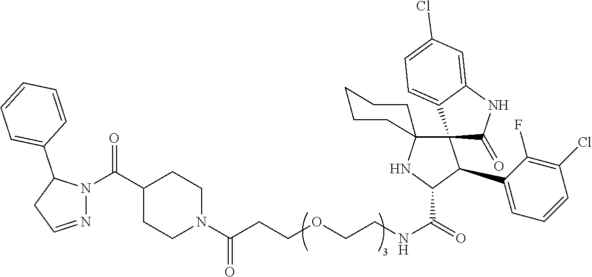 | 905.4 |
| 18 | 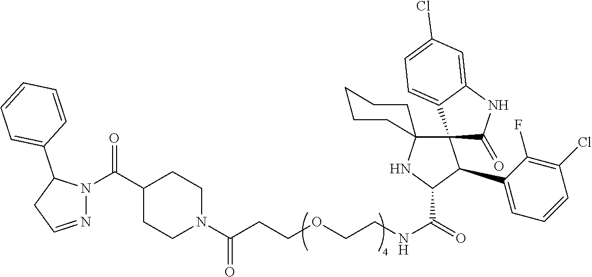 | 949.4 |

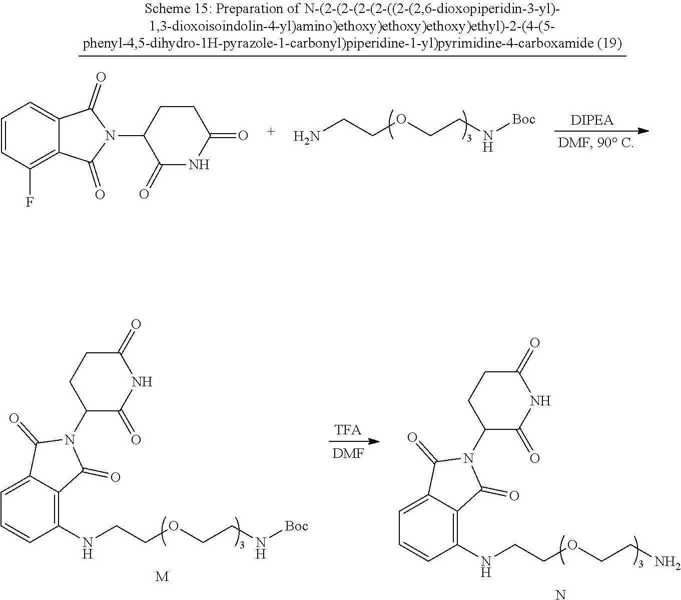

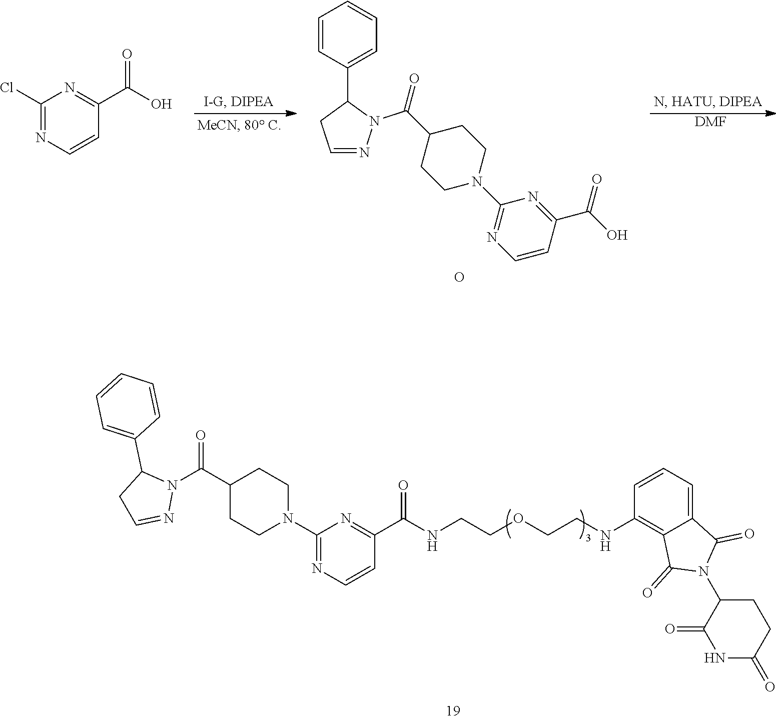

tert-butyl (2-(2-(2-(2-((2-(2,6-dioxopiperidin-3-yl)-1,3-dioxoisoindolin-4-yl)amino)ethoxy)ethoxy)ethoxy)ethyl)carbamate (M): To a solution of 3-(4-fluoro-1,3-dioxo-2,3-dihydro-1H-inden-2-yl)piperidine-2,6-dione (50 mg, 0.18 mmol) in DMF (10 mL) was added tert-butyl (2-(2-(2-(2-aminoethoxy)ethoxy)ethoxy)ethyl)carbamate (64 mg, 0.22 mmol) and DIPEA (51 uL, 0.36 mmol). The mixture was stirred at 90° C. for 2 hours. The reaction was concentrated and then was purified by flash column chromatography to give the title compound (74 mg, 80% yield) MS (ESI) m/z: [M+H]⁺, 549.2.

4-((2-(242-(2-aminoethoxy)ethoxy)ethoxy)ethyl)amino)-2-(2,6-dioxopiperidin-3-yl)isoindoline-1,3-dione (N): To a solution of M (74 mg, 0.15 mmol) in CH₂Cl₂ (2.5 mL) and TFA (1 mL) was stirred at room temperature for 1 hour before it was concentrated under reduced pressure to give compound N (61 mg, quant yield). MS (ESI) m/z: [M+H]⁺, 449.2.

2-(4-(5-phenyl-4,5-dihydro-1H-pyrazole-1-carbonyl)piperidin-1-yl)pyrimidine-4-carboxylic acid (O): To a solution of I-G (100 mg, 0.39 mmol), DIPEA (167 uL, 1.17 mmol) in acetonitrile (20 mL) was added 2-chloropyrimidine-4-carboxylic acid (74 mg, 0.49 mmol) and the mixture was stirred at reflex overnight. The reaction was concentrated and then was purified by flash column chromatography to give the title compound (1(X) mg, 68% yield) MS (ESI) m % z: [M+H]⁺, 380.1.

N-(2-(2-(2-(2-((2-(2,6-dioxopiperidin-3-yl)-1,3-dioxoisoindolin-4-yl)amino)ethoxy)ethoxy)ethoxy)ethyl)-2-(4-(5-phenyl-4,5-dihydro-1H-pyrazole-1-carbonyl)piperidin-1-yl)pyrimidine-4-carboxamide (19): To a solution of O (38 mg, 0.1 mmol), N (45 mg, 0.1 mmol) in DMF (10 mL) was added HATU (57 mg, 0.15 mmol) and Et₃N (43 uL, 0.3 mmol). The mixture was stirred at room temperature overnight. The reaction was concentrated and then was purified by reverse phase preparative HPLC to provide compound 19 (31 mg, 25% yield) MS (ESI) m % z: [M+H]⁺, 810.3.

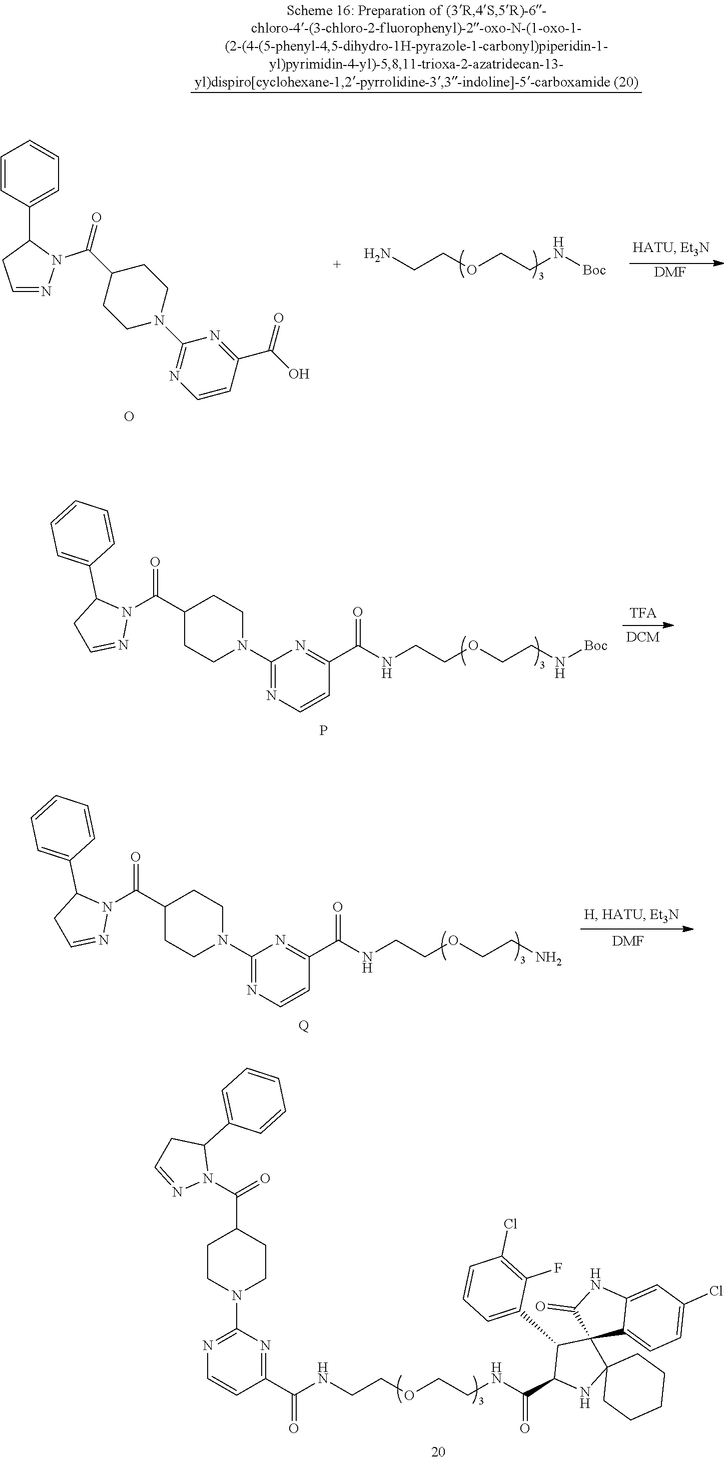

tert-butyl (1-oxo-1-(2-(4-(5-phenyl-4,5-dihydro-1H-pyrazole-1-carbonyl)piperidin-1-yl)pyrimidin-4-yl)-5,8,11-trioxa-2-azatridecan-13-yl)carbamate (P): To a solution of tert-butyl (2-(2-(2-(2-aminoethoxy)ethoxy)ethoxy)ethyl)carbamate (46 mg, 0.16 mmol), O (50 mg, 0.13 mmol) in DMF (5 mL) was added HATU (74 mg, 0.2 mmol) and DIPEA (58 uL, 0.4 mmol). The mixture was stirred at room temperature overnight. The reaction was concentrated and then was purified by flash column chromatography to give the title compound (32 mg, 38% yield). MS (ESI) m/z: [M+H]⁺, 654.3.

N-(2-(2-(2-(2-aminoethoxy)ethoxy)ethoxy)ethyl)-2-(4-(5-phenyl-4,5-dihydro-1H pyrazole-1-carbonyl)piperidin-1-yl)pyrimidine-4-carboxamide (Q): To a solution of P (32 mg, 0.049 mmol) in CH₂Cl₂ (2 mL) and TFA (1 mL) was stirred at room temperature for 3 h before it was concentrated under reduced pressure to give compound Q (27 mg, quant yield). MS (ESI) m/z: [M+H]⁺, 554.3.

(3′R,4'S,5′R)-6″-chloro-4′-(3-chloro-2-fluorophenyl)-2″-oxo-N-(1-oxo-1-(2-(4-(5-phenyl-4,5-dihydro-1H-pyrazole-1-carbonyl)piperidin-1-yl)pyrimidin-4-yl)-5,8,11-trioxa-2-azatridecan-13-yl)dispiro[cyclohexane-1,2′-pyrrolidine-3′,3″-indoline]-5′-carboxamide (20): To a solution of Q (27 mg, 0.049 mmol), H (23 mg, 0.049 mmol) in DMF (5 mL) was added HATU (38 mg, 0.073 mmol) and Et₃N (22 uL, 0.15 mmol). The mixture was stirred at room temperature overnight. The reaction was concentrated and then was purified by reverse phase preparative HPLC to provide compound 20 (9 mg, 20% yield) MS (ES) Piz: [M+H]⁺, 998.4.

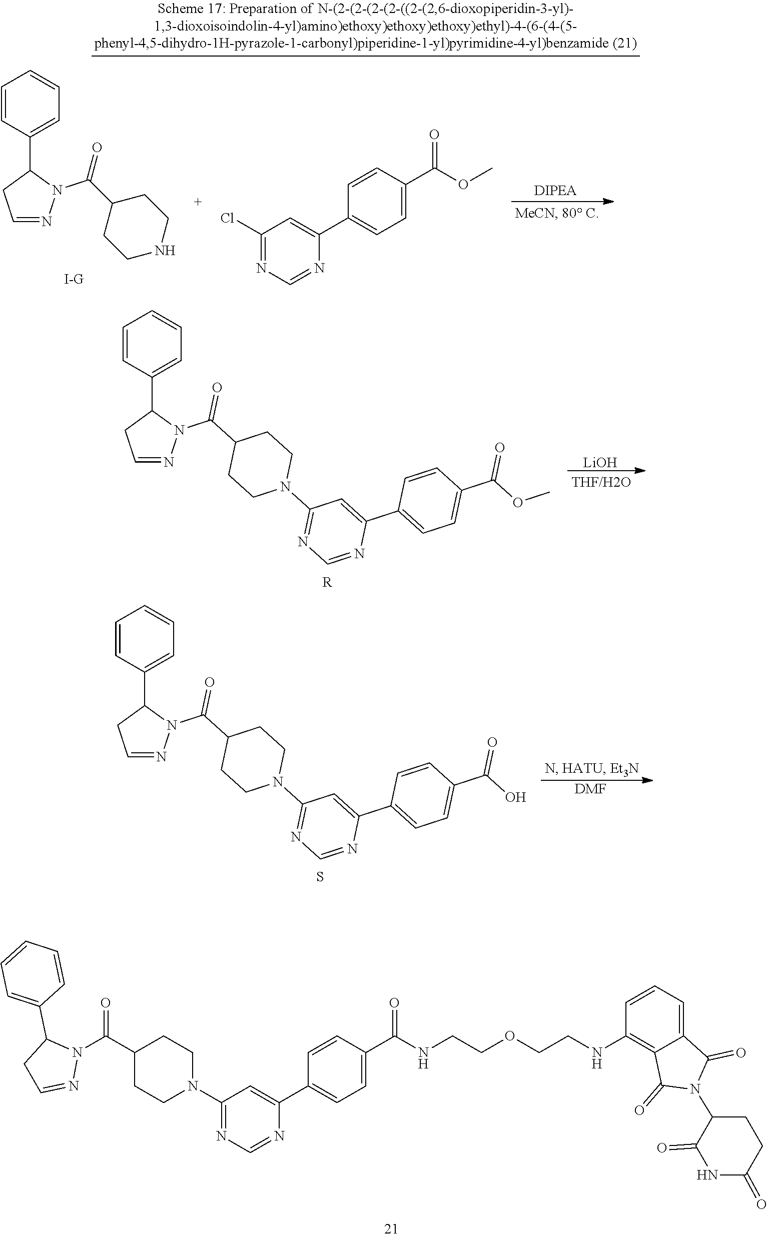

methyl 4-(6-(4-(5-phenyl-4,5-dihydro-1H-pyrazole-1-carbonyl)piperidin-1-yl)pyrimidin-4-yl)benzoate (R): To a solution of I-G (100 mg, 0.39 mmol), DIPEA (167 uL, 1.17 mmol) in acetonitrile (20 mL) was added methyl 4-(6-chloropyrimidin-4-yl)benzoate (116 mg, 0.47 mmol). The mixture was stirred at 80° C. overnight. The reaction was concentrated and then was purified by flash column chromatography to give the title compound (71 mg, 84% yield) MS (ESI) m/z: [M+H]⁺, 470.2.

4-(6-(4-(5-phenyl-4,5-dihydro-1H-pyrazole-1-carbonyl)piperidin-1-yl)pyrimidin-4-yl)benzoic acid (S): To a solution of R (71 mg, 0.32 mmol) in THE (10 mL) and water (2 mL) was added LiOH (15 mg, 0.64 mmol) and the solution was stirred at room temperature overnight. The mixture was concentrated and without further purification. MS (ESI) m/z: [M+H]⁺, 456.2.

N-(2-(2-(2-(2-((2-(2,6-dioxopiperidin-3-yl)-1,3-dioxoisoindolin-4-yl)amino)ethoxy)ethoxy)ethoxy)ethyl)-4-(6-(4-(5-phenyl-4,5-dihydro-1H-pyrazole-1-carbonyl)piperidin-1-yl)pyrimidin-4-yl)benzamide (21): a solution of S (30 mg, 0.065 mmol), N (29 mg, 0.065 mmol) in DMF (5 mL) was added HATU (38 mg, 0.098 mmol) and Et₃N (22 uL, 0.15 mmol). The mixture was stirred at room temperature overnight. The reaction was concentrated and then was purified by reverse phase preparative HPLC to provide compound 21 (15 mg, 27% yield) MS (ESI) m % z: [M+H]⁺, 886.4.

Example compounds 22 and 74-76 were prepared in an analogous manner to compound 21, employing the corresponding carboxylic acid starting materials and amine material.

TABLE 6

| | | MS (ESI) m/z: |
|---|---|---|
| | Structure | [M + H]⁺ |
| 22 | 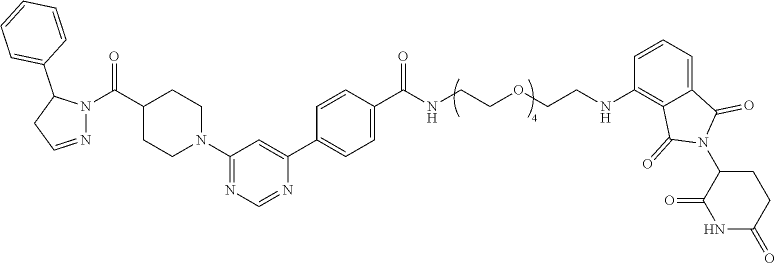 | 930.4 |
| 74 | 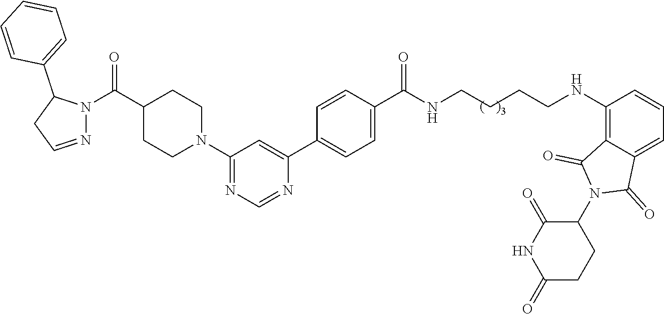 | 808.3 |
| 75 | 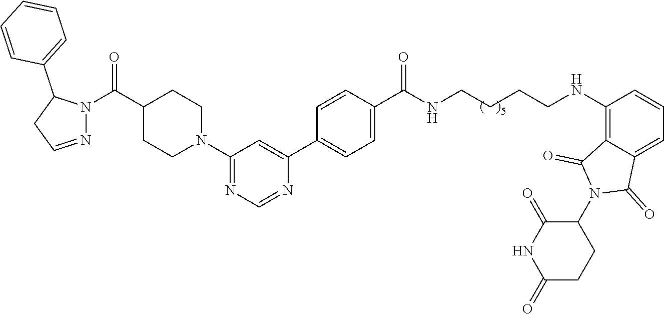 | 836.4 |
| 76 | 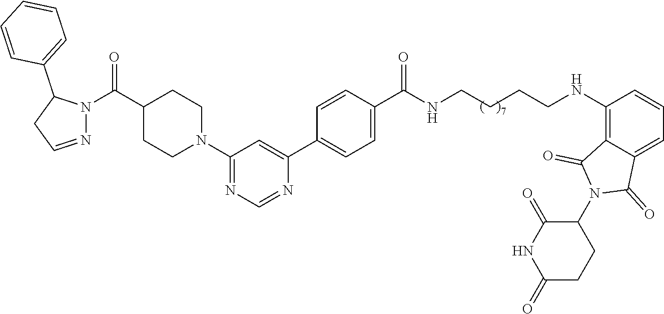 | 864.4 |

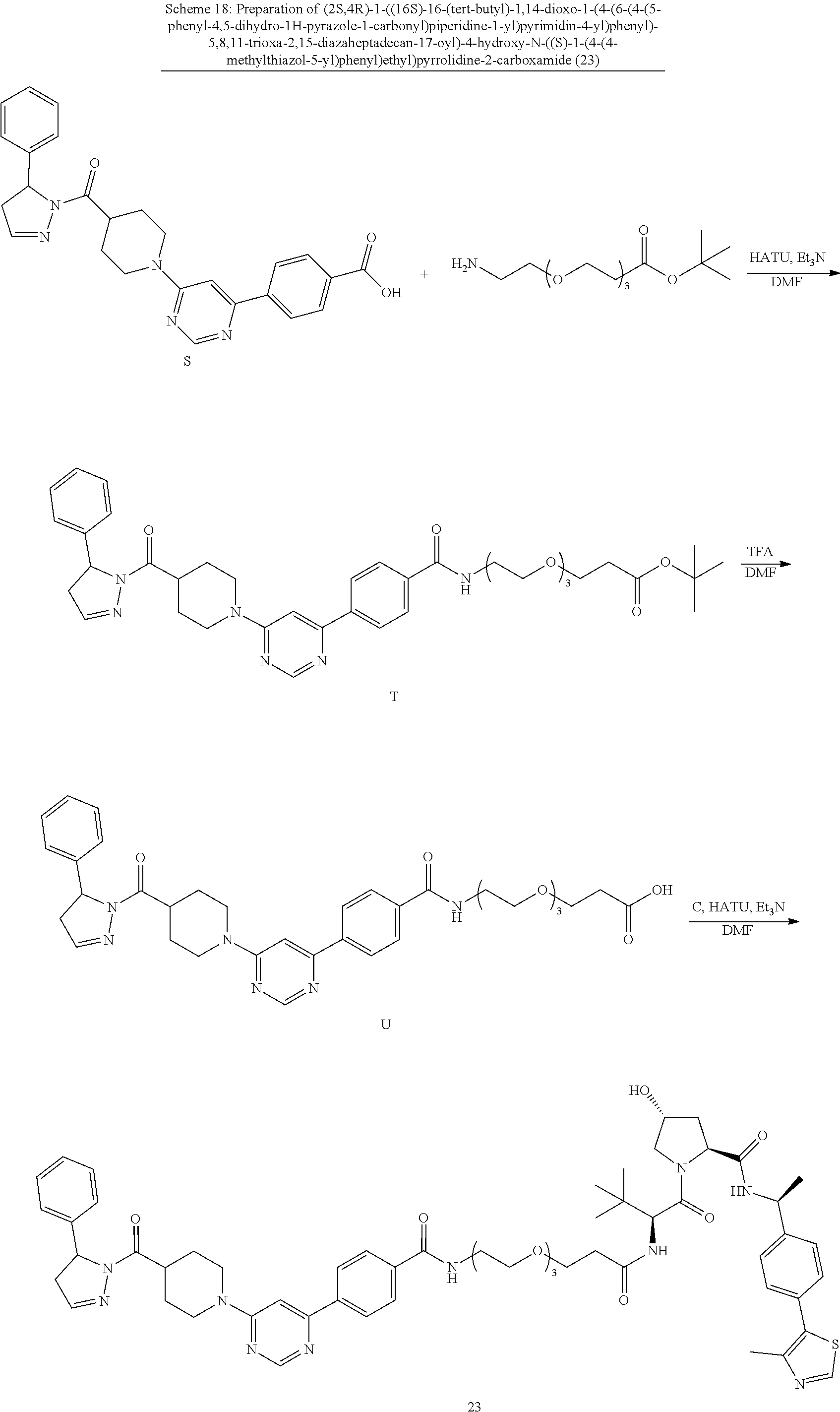

tert-butyl 1-oxo-1-(4(6-(4(5-phenyl-4,5-dihydro-1H-pyrazole-1-carbonyl)piperidin-1-yl)pyrimidin-4-yl)phenyl)-5,8,11-trioxa-2-azatetradecan-14-oate (T): a solution of tert-butyl 3-(2-(2-(2-aminoethoxy)ethoxy)ethoxy)propanoate (44 mg, 0.16 mmol), S (50 mg, 0.11 mmol) in DMF (8 mL) was added HATU (60 mg, 0.16 mmol) and Et₃N (58 uL, 0.4 mmol). The mixture was stirred at room temperature overnight. The reaction was concentrated and then was purified by flash column chromatography to give the title compound (55 mg, 71% yield). MS (ESI) m/z: [M+H]⁺, 715.4.

1-oxo-1-(4-(6-(4-(5-phenyl-4,5-dihydro-1H-pyrazole-1-carbonyl)piperidin-1-yl)pyrimidin-4-yl)phenyl)-5,8,11-trioxa-2-azatetradecan-14-oic acid (U): a solution of a (55 mg, 0.078 mmol) in CH₂Cl₂ (2 mL) and TFA (1 mL) was stirred at room temperature for 3 h before it was concentrated under reduced pressure to give compound U (50 mg, quant yield). MS (ES) m/z: [M+H]⁺, 659.3.

(2S,4R)-1-((16S)-16-(tert-butyl)-1,14-dioxo-1-(4-(6-(4-(5-phenyl-4,5-dihydro-1H-pyrazole-1-carbonyl)piperidin-1-yl)pyrimidin-4-yl)phenyl)-5,8,11-trioxa-2,15-diazaheptadecane-17-oyl)-4-hydroxy-N—((S)-1-(4-(4-methylthiazol-5-yl)phenyl)ethyl)pyrrolidine-2-carboxamide (23): a solution of U (20 mg, 0.03 mmol), amino C (13 mg, 0.03 mmol) in DMF (5 mL) was added HATU (17 mg, 0.045 mmol) and Et₃N (17 uL, 0.12 mmol). The mixture was stirred at room temperature overnight. The reaction was concentrated and then was purified by reverse phase preparative HPLC to provide compound 23 (10 mg, 30% yield) MS (ESI) m/z: [M+H]⁺, 1085.5.

Example compounds 24 and 77 were prepared in an analogous manner to compound 23, employing the corresponding amine starting materials and S.

TABLE 7

| | | MS |
|---|---|---|
| | | (ESI) |
| | | m/z: |
| | | [M + |
| | Structure | H]⁺ |
| 24 | 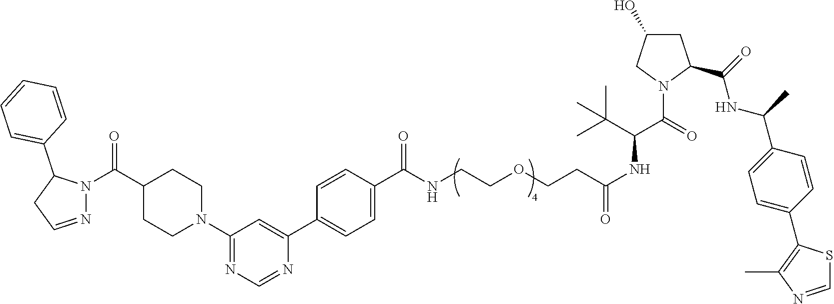 | 1129.6 |
| 77 | 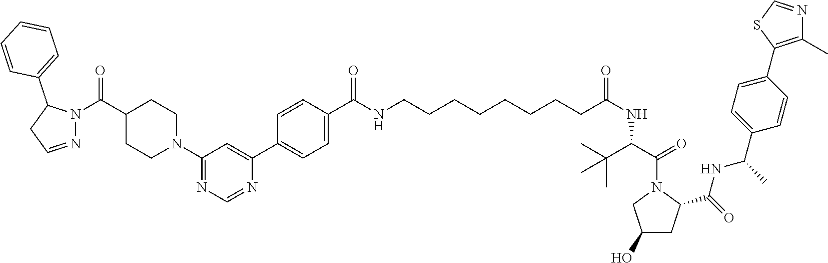 [SMILES: Cc1ncsc1-c1ccc([C@H](C)NC(=O)[C@@H]2C[C@@H](O)CN2C(=O)[C@@H](NC(=O)CCCCCCCCNC(=O)c2ccc(-c3cc(N4CCC(C(=O)N5N=CCC5c5ccccc5)CC4)ncn3)cc2)C(C)(C)C)cc1] | 1037.5 |

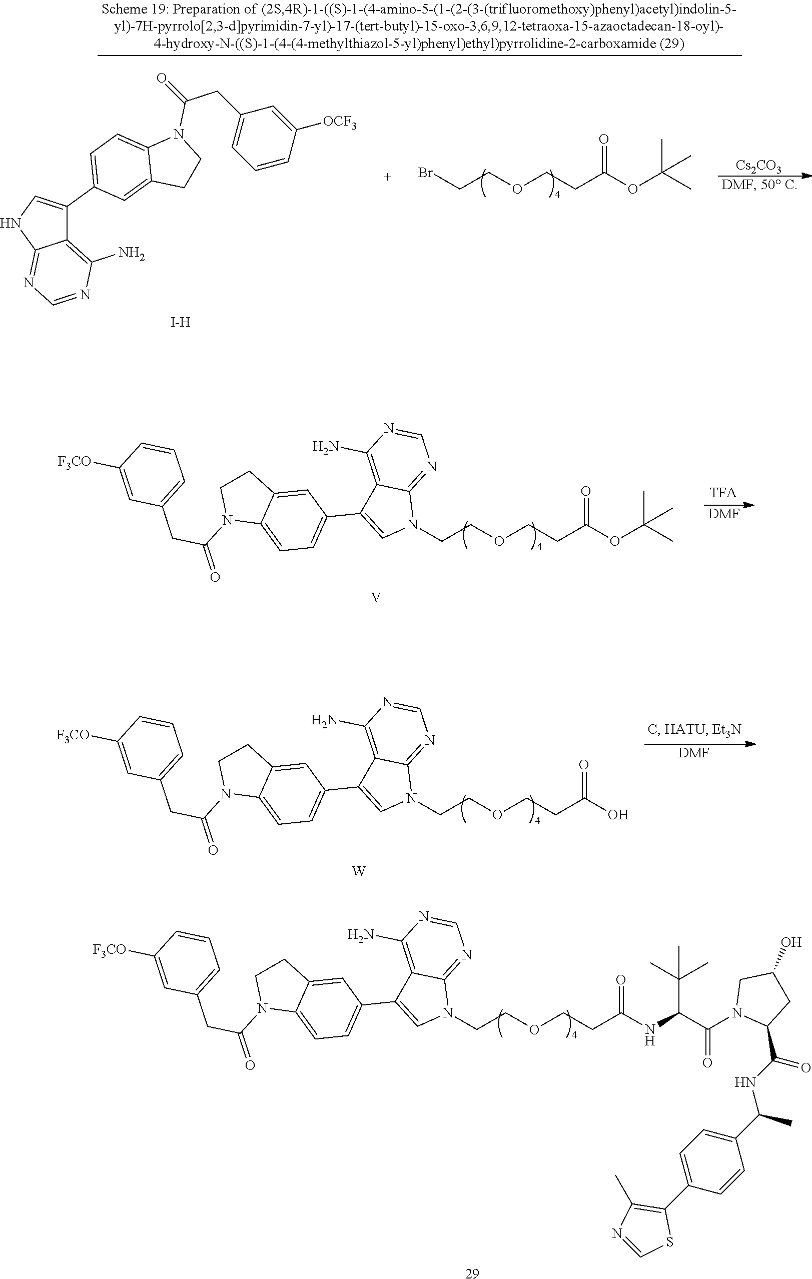

tert-butyl 1-(4-amino-5-(1-(2-(3-(trifluoromethoxy)phenyl)acetyl)indolin-5-yl)-7H-pyrrolo[2,3-d]pyrimidin-7-yl)-3,6,9,12-tetraoxapentadecan-15-oate (V): To a solution of I-H (45 mg, 0.1 mmol) and tert-butyl 1-bromo-3,6,9,12-tetraoxapentadecan-15-oate (46 mg, 0.12 mmol) in DMF (10 mL) was added Cs₂CO₃ (65 mg, 0.2 mmol). The mixture was stirred at 50° C. for 5 hours before adding water (20 mL). The resulting mixture was extracted with CH₂Cl₂ (2/50 mL), the combined organic phases were dried over anhydrous Na₂SO₄ and concentrated under reduced pressure to give the title compound (37 mg, 49% yield). MS (ESI) m/z: [M+H]⁺, 758.3.

1-(4-amino-5-(1-(2-(3-(trifluoromethoxy)phenyl)acetyl)indolin-5-yl)-7H-pyrrolo[2,3-d]pyrimidin-7-yl)-3,6,9,12-tetraoxapentadecan-15-oic acid (W): a solution of V (37 mg, 0.049 mmol) in CH₂Cl₂ (2 mL) and TFA (1 mL) was stirred at room temperature for 3 h before it was concentrated under reduced pressure to give compound d (30 mg, 88%). MS (ESI) m/z: [M+H]+, 702.3.

(2S,4R)-1-((S)-1-(4-amino-5-(1-(2-(3-(trifluoromethoxy)phenyl)acetyl)indolin-5-yl)-7H-pyrrolo[2,3-d]pyrimidin-7-yl)-17-(tert-butyl)-15-oxo-3,6,9,12-tetraoxa-16-azaoctadecan-18-oyl)-4-hydroxy-N—((S)-1-(4-(4-methylthiazol-5-yl)phenyl)ethyl)pyrrolidine-2-carboxamide (29): a solution of W (30 mg, 0.043 mmol), amino C (19 mg, 0.043 mmol) in DMF (5 mL) was added HATU (21 mg, 0.056 mmol) and Et₃N (18 uL, 0.13 mmol). The mixture was stirred at room temperature overnight. The reaction was concentrated and then was purified by reverse phase preparative HPLC to provide compound 29 (22 mg, 46% yield) MS (ESI) m/z: [M+H]⁺, 1128.5

Example compound 30 was prepared in an analogous manner to compound 29, employing the corresponding bromide starting materials and I-H.

TABLE 8

| | | MS (ESI) m/z: |
|---|---|---|
| | Structure | [M + H]⁺ |
| 30 | 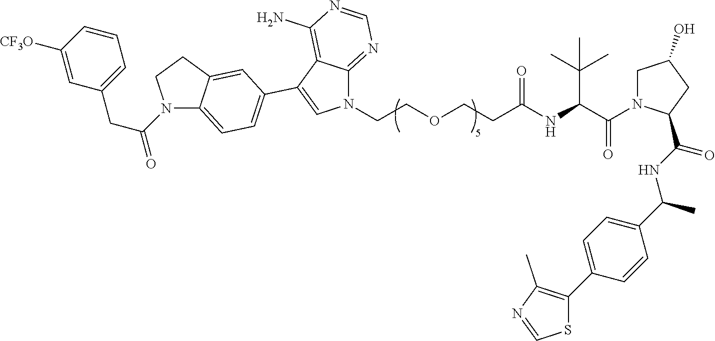 | 1172.5 |

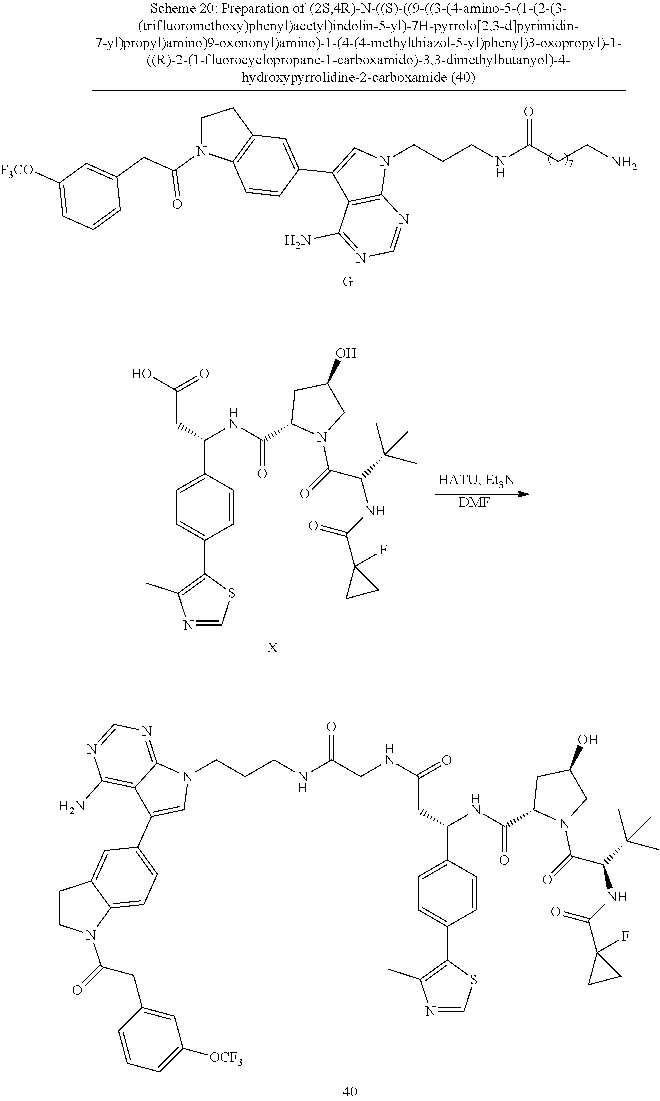

(2S,4R)—N—((S)-3-((9-((3-(4-amino-5-(1-(2-(3-(trifluoromethoxy)phenyl)acetyl)indolin-5-yl)-7H-pyrrolo[2,3-d]pyrimidin-7-yl)propyl)amino)-9-oxononyl)amino)-1-(4-(4-methylthiazol-5-yl)phenyl)-3-oxopropyl)-1-((R)-2-(1-fluorocyclopropane-1-carboxamido)-3,3-dimethylbutanoyl)-4-hydroxypyrrolidine-2-carboxamide (40): a solution of G (20 mg, 0.03 mmol), acid X (18 mg, 0.03 mmol) in DMF (5 mL) was added HATU (21 mg, 0.056 mmol) and Et₃N (18 uL, 0.13 mmol). The mixture was stirred at room temperature overnight. The reaction was concentrated and then was purified by reverse phase preparative HPLC to provide compound 40 (26 mg, 71% yield) MS (ESI) m/z: [M+H]⁺, 1221.5

Example compounds 41-50, 62-73, 78-85, and 88-92 were prepared in an analogous manner to compound 40, employing the corresponding amine starting materials and X, I-O, I-S, I-V, or I-Z.

TABLE 9

| | | MS |
|---|---|---|
| | | (ESI) |
| | | m/z: |
| | Structure | [M + H]⁺ |
| 41 | 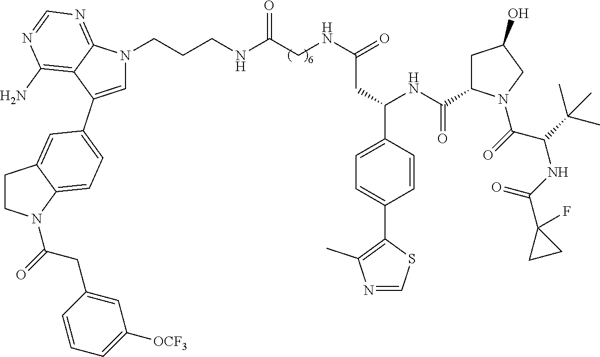 | 1194.5 |
| 42 | 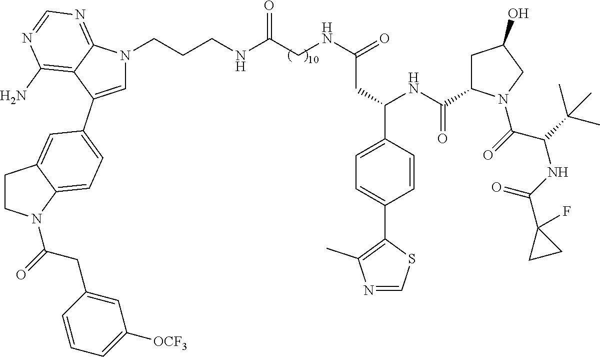 | 1250.6 |
| 43 | 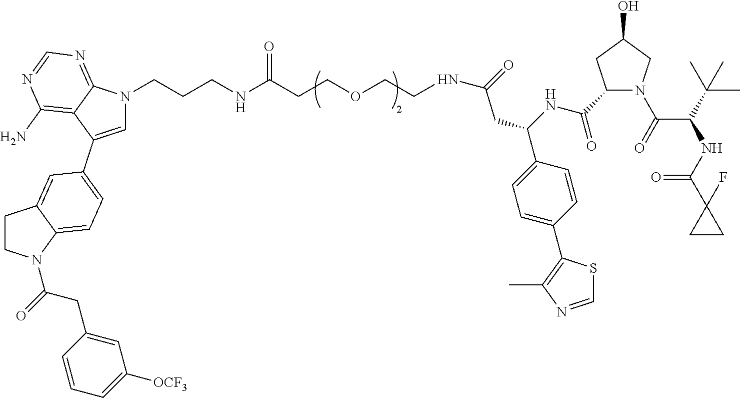 | 1226.5 |
| 44 | 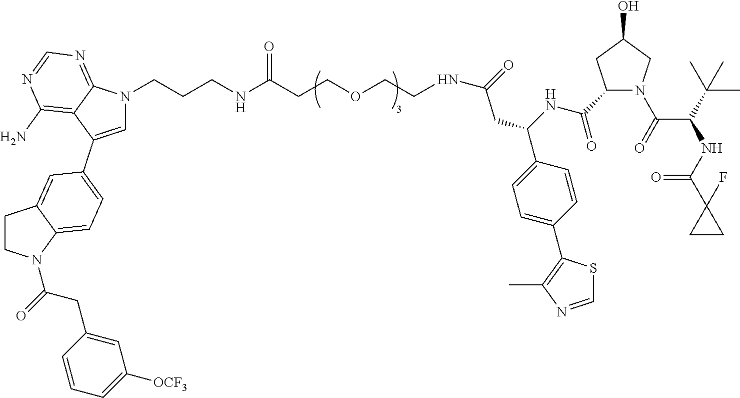 | 1270.5 |
| 45 | 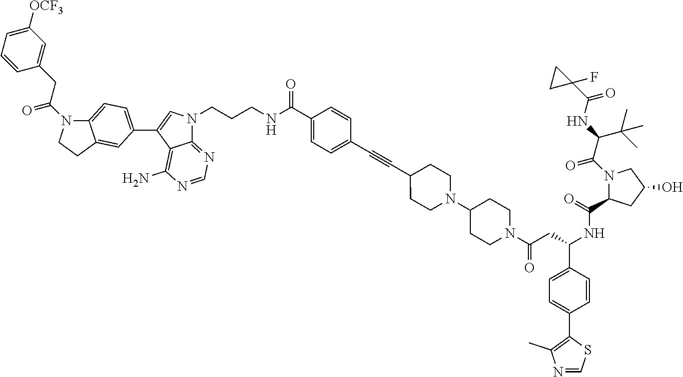 [SMILES: Cc1ncsc1-c1ccc([C@H](CC(=O)N2CCC(N3CCC(C#Cc4ccc(C(=O)NCCCn5cc(-c6ccc7c(c6)CCN7C(=O)Cc6cccc(OC(F)(F)F)c6)c6c(N)ncnc65)cc4)CC3)CC2)NC(=O)[C@@H]2C[C@@H](O)CN2C(=O)[C@@H](NC(=O)C2(F)CC2)C(C)(C)C)cc1] | 1361.6 |
| 46 | 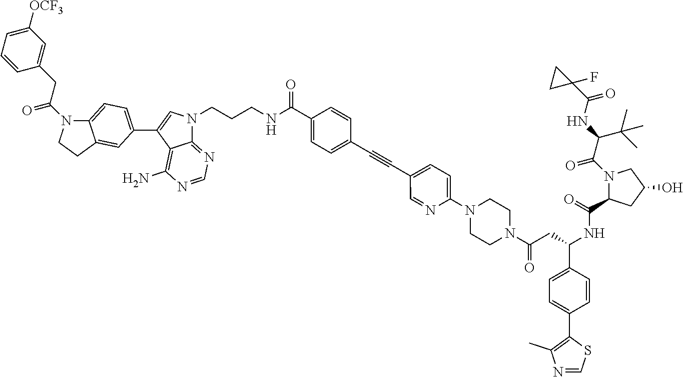 [SMILES: Cc1ncsc1-c1ccc([C@H](CC(=O)N2CCN(c3ccc(C#Cc4ccc(C(=O)NCCCn5cc(-c6ccc7c(c6)CCN7C(=O)Cc6cccc(OC(F)(F)F)c6)c6c(N)ncnc65)cc4)cn3)CC2)NC(=O)[C@@H]2C[C@@H](O)CN2C(=O)[C@@H](NC(=O)C2(F)CC2)C(C)(C)C)cc1] | 1356.5 |
| 47 | 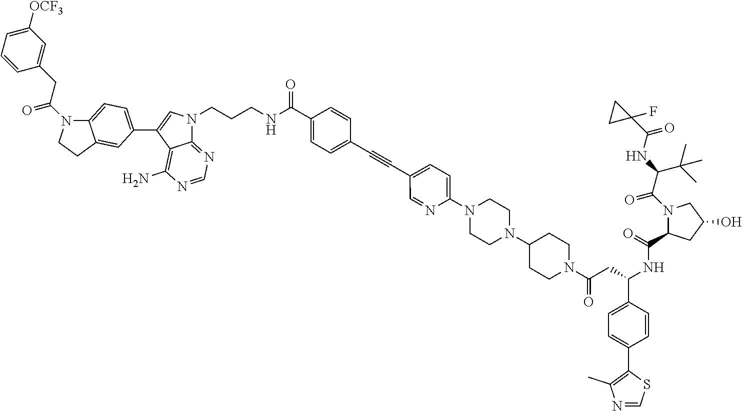 [SMILES: Cc1ncsc1-c1ccc([C@H](CC(=O)N2CCC(N3CCN(c4ccc(C#Cc5ccc(C(=O)NCCCn6cc(-c7ccc8c(c7)CCN8C(=O)Cc7cccc(OC(F)(F)F)c7)c7c(N)ncnc76)cc5)cn4)CC3)CC2)NC(=O)[C@@H]2C[C@@H](O)CN2C(=O)[C@@H](NC(=O)C2(F)CC2)C(C)(C)C)cc1] | 1439.6 |
| 48 | 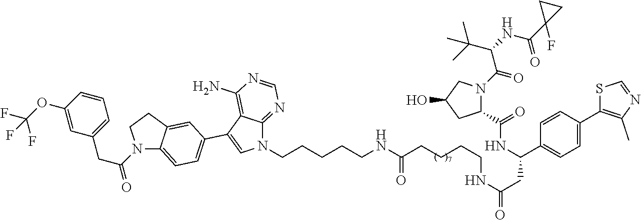 | 1278.6 |
| 49 | 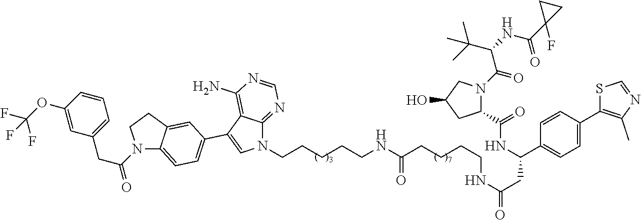 | 1306.6 |
| 50 | 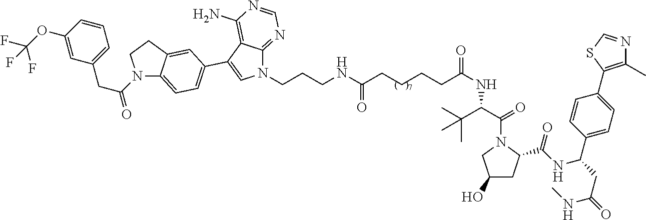 | 1206.6 |
| 62 | 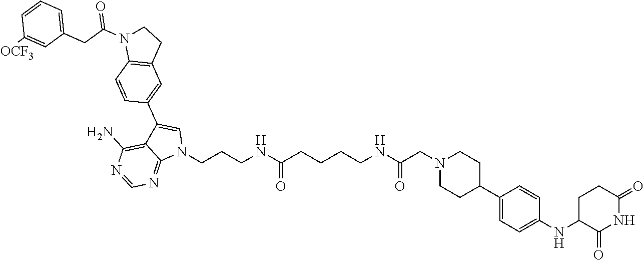 [SMILES: Nc1ncnc2c1c(-c1ccc3c(c1)CCN3C(=O)Cc1cccc(OC(F)(F)F)c1)cn2CCCNC(=O)CCCCNC(=O)CN1CCC(c2ccc(NC3CCC(=O)NC3=O)cc2)CC1] | 937.3 |
| 63 | 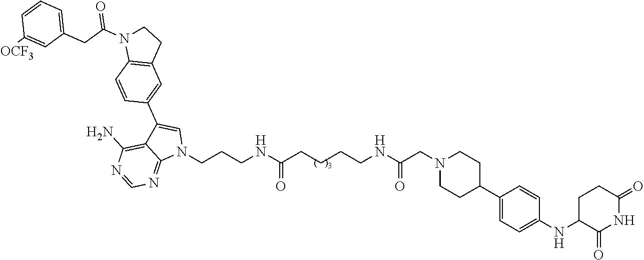 | 965.5 |
| 64 | 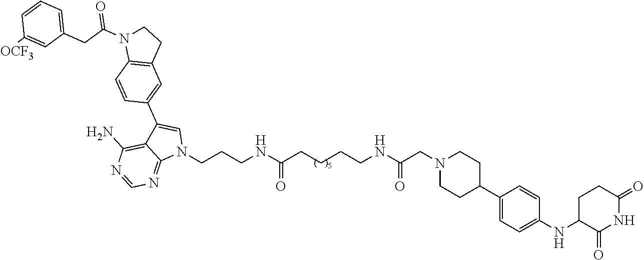 | 993.5 |
| 65 | 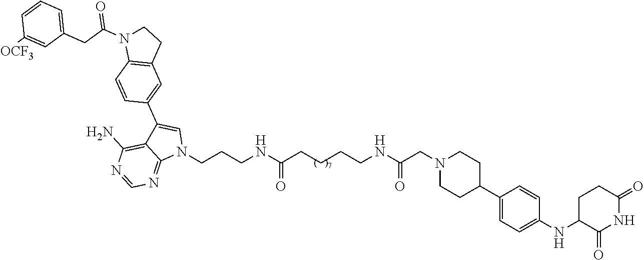 | 1021.6 |
| 66 | 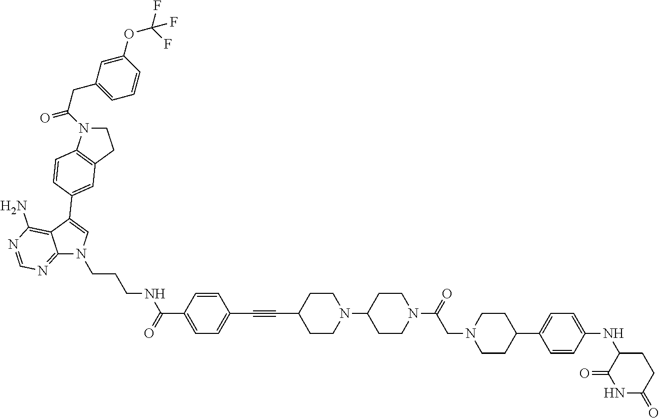 [SMILES: Nc1ncnc2c1c(-c1ccc3c(c1)CCN3C(=O)Cc1cccc(OC(F)(F)F)c1)cn2CCCNC(=O)c1ccc(C#CC2CCN(C3CCN(C(=O)CN4CCC(c5ccc(NC6CCC(=O)NC6=O)cc5)CC4)CC3)CC2)cc1] | 1132.5 |
| 67 | 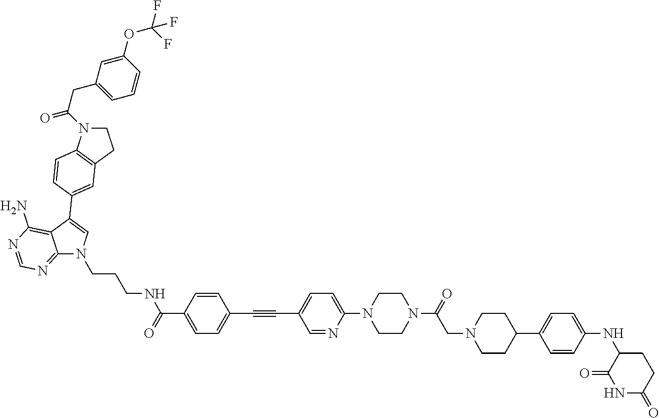 [SMILES: Nc1ncnc2c1c(-c1ccc3c(c1)CCN3C(=O)Cc1cccc(OC(F)(F)F)c1)cn2CCCNC(=O)c1ccc(C#Cc2ccc(N3CCN(C(=O)CN4CCC(c5ccc(NC6CCC(=O)NC6=O)cc5)CC4)CC3)nc2)cc1] | 1127.5 |
| 68 | 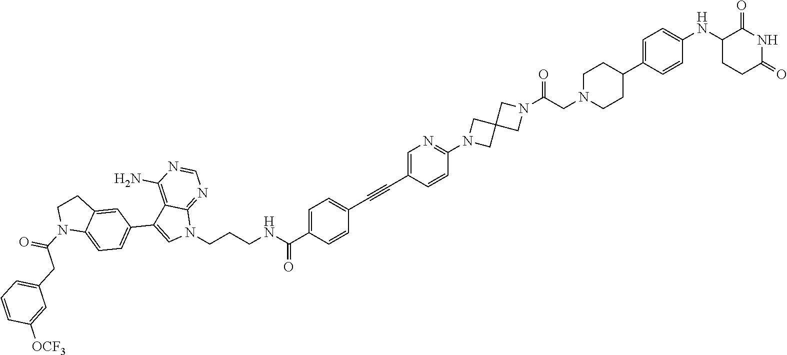 [SMILES: Nc1ncnc2c1c(-c1ccc3c(c1)CCN3C(=O)Cc1cccc(OC(F)(F)F)c1)cn2CCCNC(=O)c1ccc(C#Cc2ccc(N3CC4(CN(C(=O)CN5CCC(c6ccc(NC7CCC(=O)NC7=O)cc6)CC5)C4)C3)nc2)cc1] | 1139.5 |
| 69 | 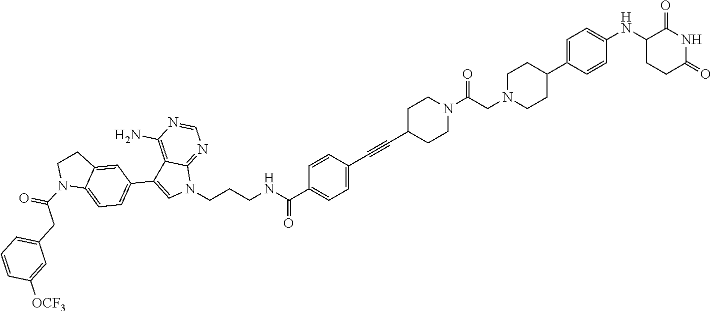 [SMILES: Nc1ncnc2c1c(-c1ccc3c(c1)CCN3C(=O)Cc1cccc(OC(F)(F)F)c1)cn2CCCNC(=O)c1ccc(C#CC2CCN(C(=O)CN3CCC(c4ccc(NC5CCC(=O)NC5=O)cc4)CC3)CC2)cc1] | 1049.5 |
| 70 | 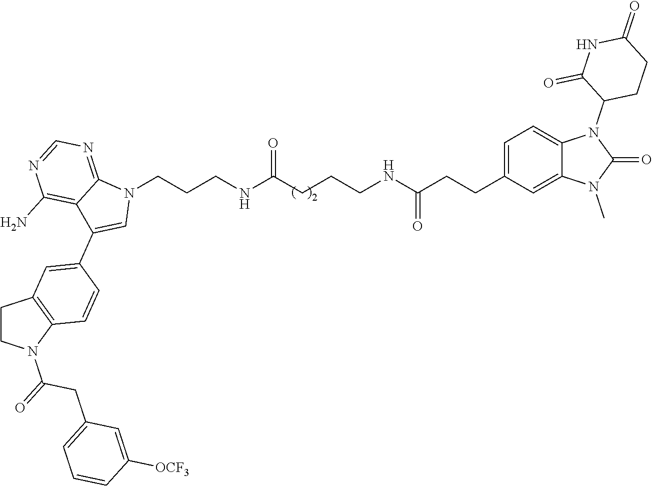 | 923.3 |
| 71 | 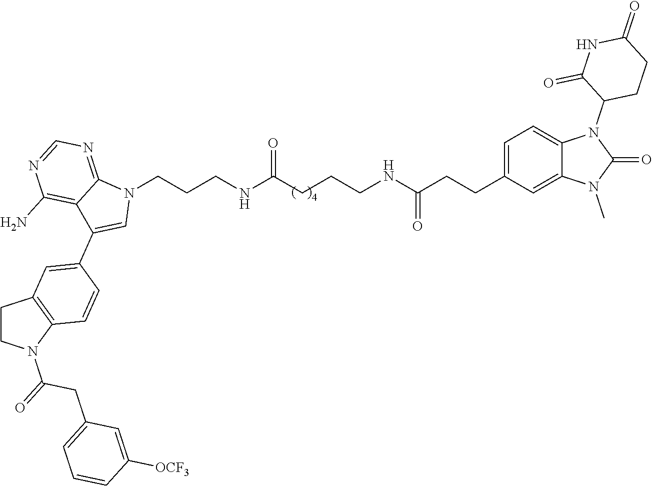 | 951.4 |
| 72 | 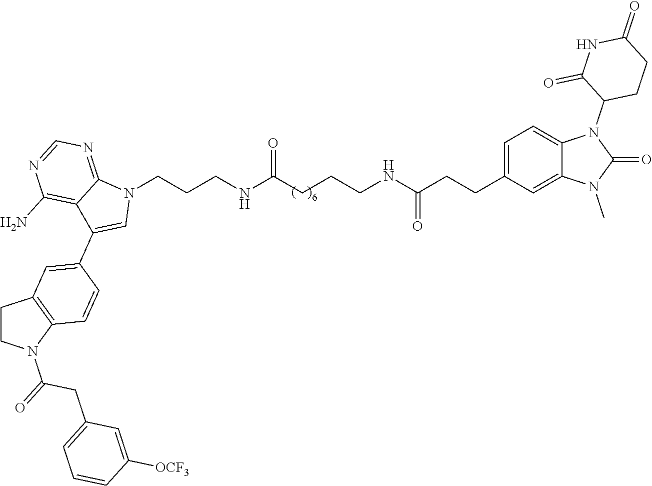 | 979.4 |
| 73 | 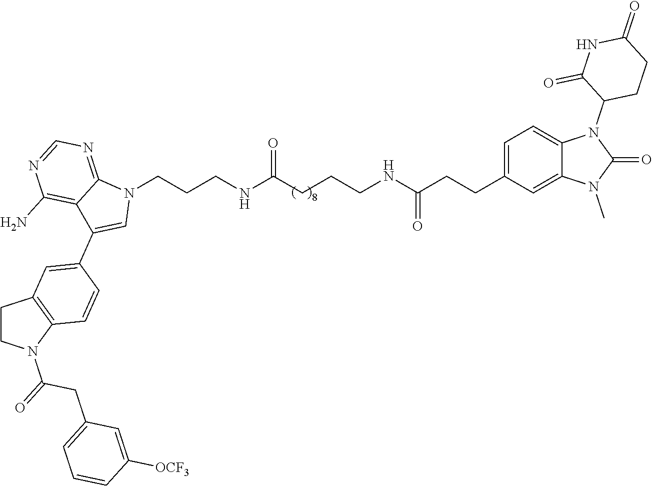 | 1007.5 |
| 78 | 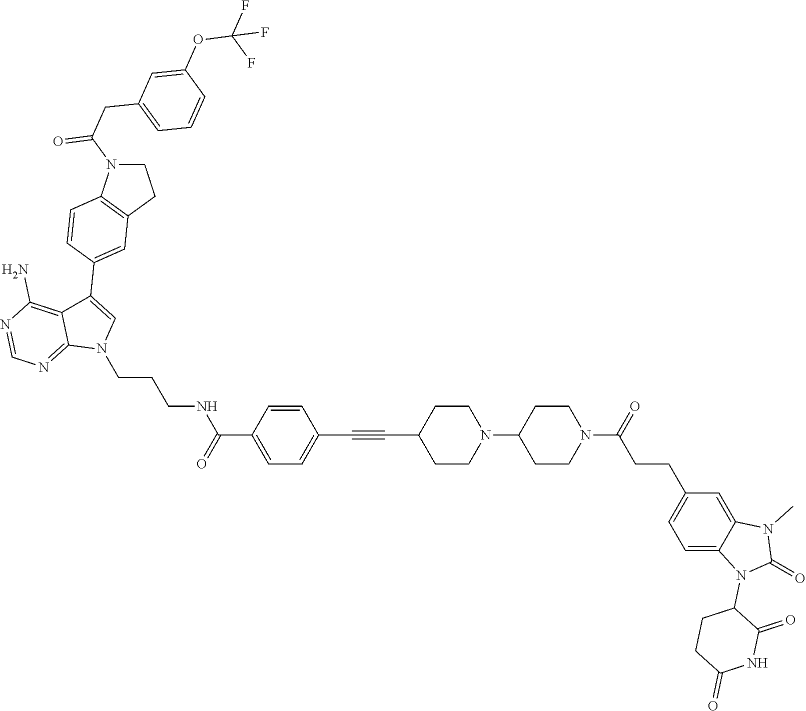 [SMILES: Cn1c(=O)n(C2CCC(=O)NC2=O)c2ccc(CCC(=O)N3CCC(N4CCC(C#Cc5ccc(C(=O)NCCCn6cc(-c7ccc8c(c7)CCN8C(=O)Cc7cccc(OC(F)(F)F)c7)c7c(N)ncnc76)cc5)CC4)CC3)cc21] | 1118.5 |
| 79 | 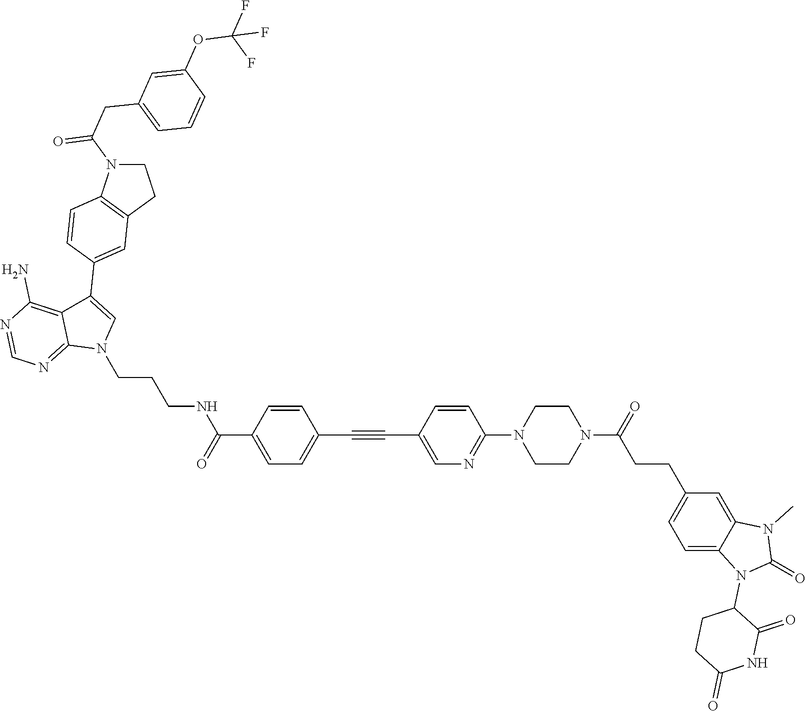 [SMILES: Cn1c(=O)n(C2CCC(=O)NC2=O)c2ccc(CCC(=O)N3CCN(c4ccc(C#Cc5ccc(C(=O)NCCCn6cc(-c7ccc8c(c7)CCN8C(=O)Cc7cccc(OC(F)(F)F)c7)c7c(N)ncnc76)cc5)cn4)CC3)cc21] | 1113.3 |
| 80 | 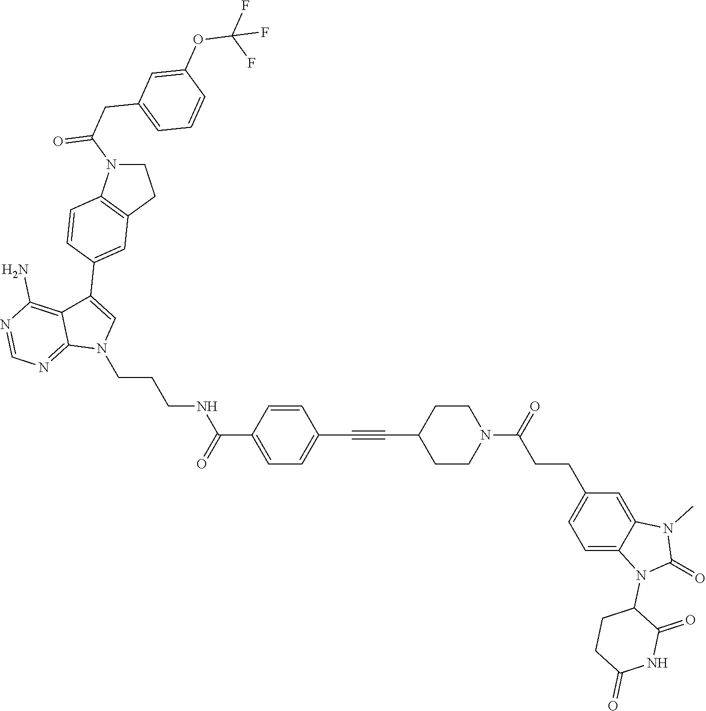 [SMILES: Cn1c(=O)n(C2CCC(=O)NC2=O)c2ccc(CCC(=O)N3CCC(C#Cc4ccc(C(=O)NCCCn5cc(-c6ccc7c(c6)CCN7C(=O)Cc6cccc(OC(F)(F)F)c6)c6c(N)ncnc65)cc4)CC3)cc21] | 1035.4 |
| 81 | 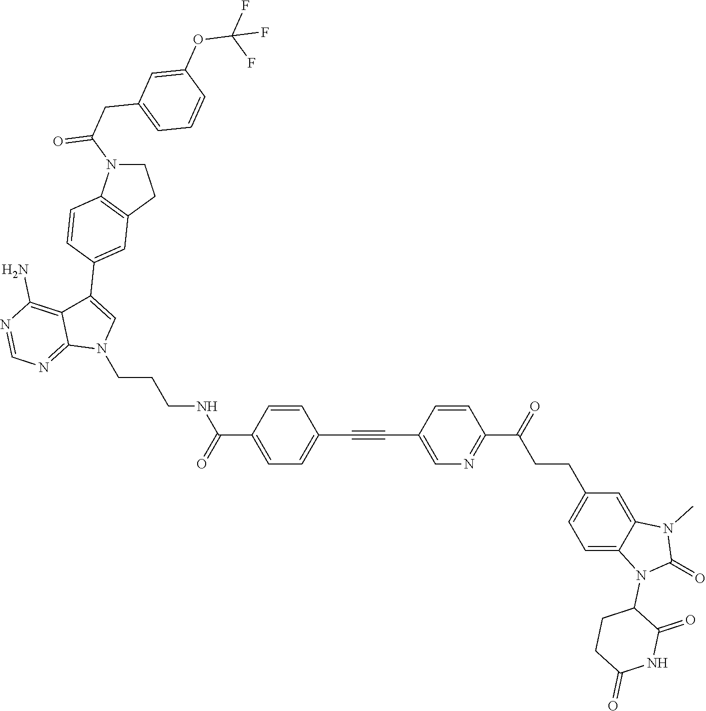 [SMILES: Cn1c(=O)n(C2CCC(=O)NC2=O)c2ccc(CCC(=O)c3ccc(C#Cc4ccc(C(=O)NCCCn5cc(-c6ccc7c(c6)CCN7C(=O)Cc6cccc(OC(F)(F)F)c6)c6c(N)ncnc65)cc4)cn3)cc21] | 1029.3 |
| 82 | 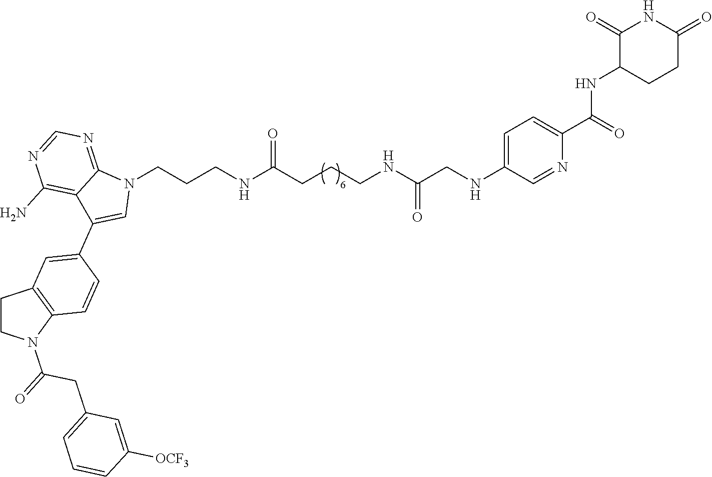 | 954.4 |
| 83 | 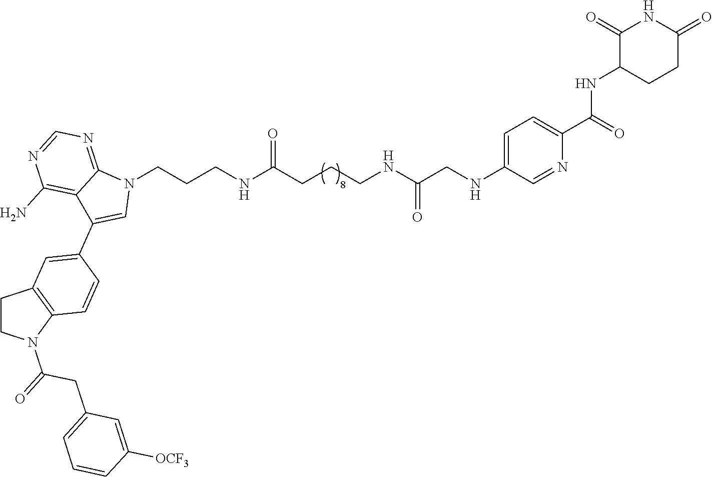 | 982.5 |
| 84 | 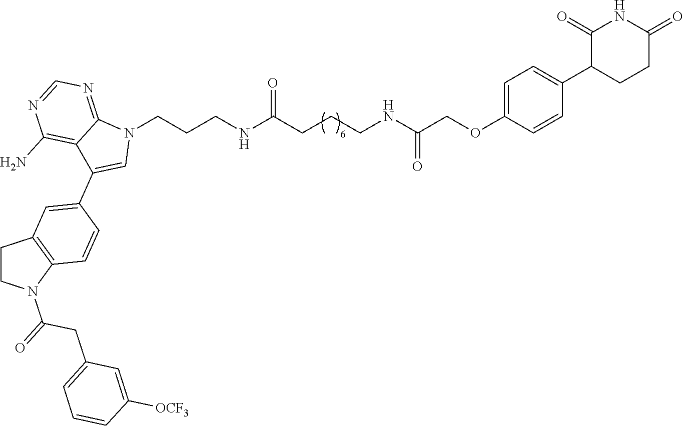 | 911.4 |
| 85 | 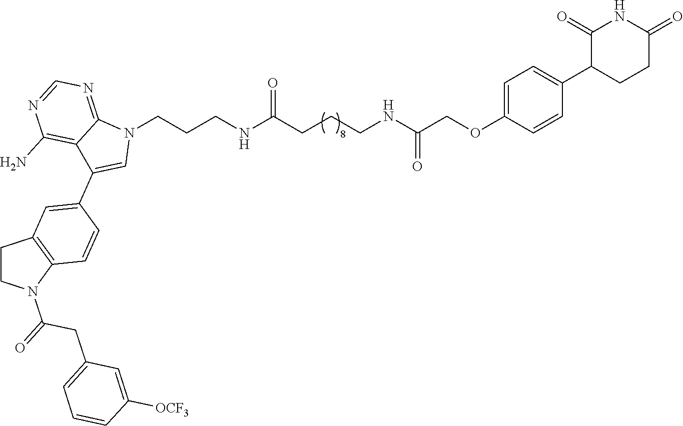 | 939.5 |
| 88 | 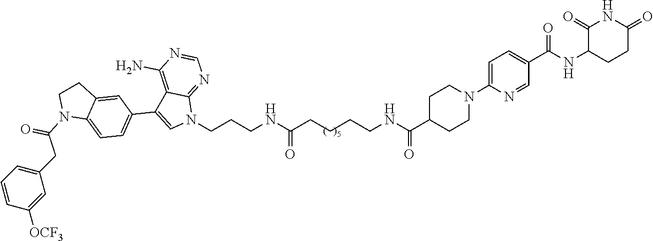 | 1008.5 |
| 89 | 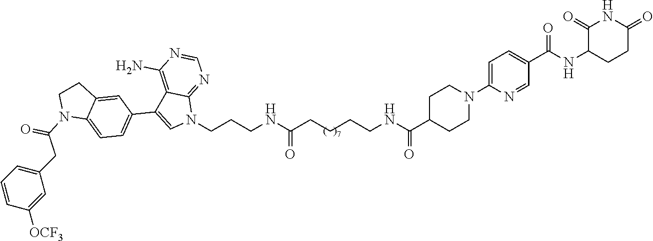 | 1036.5 |
| 90 | 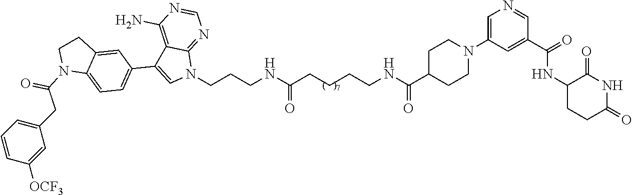 | 1036.5 |
| 91 | 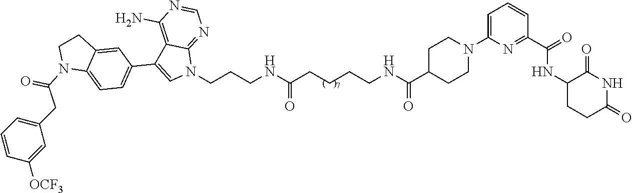 | 1036.5 |
| 92 | 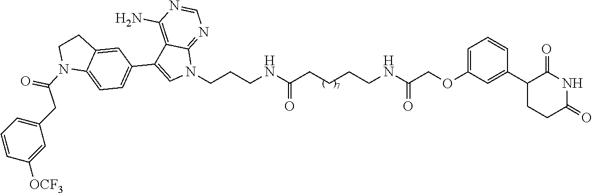 | 939.4 |

Additional compounds as described herein were synthesized and tested for activity in assays as described in Example 2. The additional compounds include Compounds 86 and 87, shown below in Table 10.

TABLE 10

| | Structure |
|---|---|
| 86 | 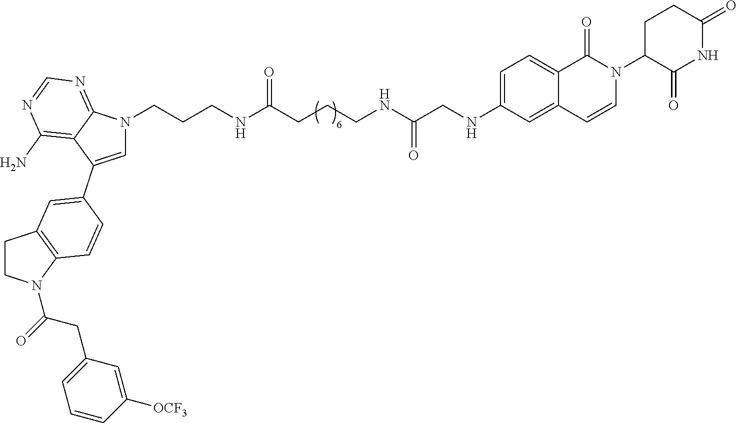 |
| 87 | 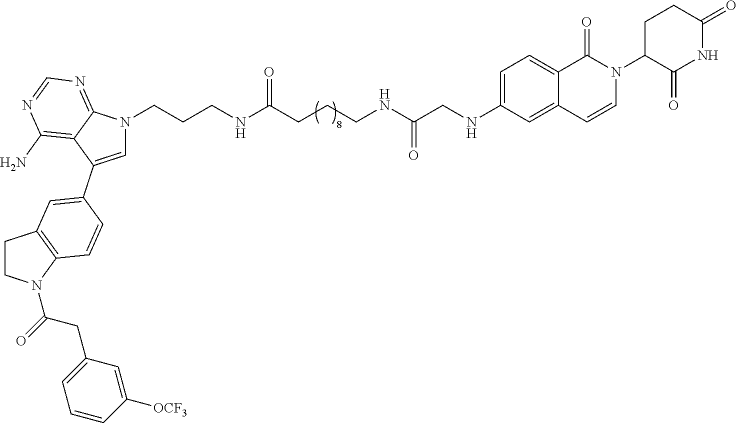 |

### Example 2: RIPK1 Degradation Assays

Western Blot assays were performed to demonstrate the ability of the exemplary compounds to degrade RIPK1 in human cells. Cells were seeded in 6-well cell culture plates overnight prior to treatment and incubated with the indicated doses of compounds for 24 h at 37° C. Then, cell pellets were collected and resuspended with RIPA lysis buffer, which was added with 1% proteasome inhibitor and phosphatase inhibitor Cocktail. Whole-cell protein lysates were sonicated on ice for 20 seconds and centrifuged at 12 000 rpm for 20 min at 4° C. The supernatants were determined using the BCA method. Proteins were separated by sodium dodecyl sulfate-polyacrylamide gel electrophoresis and transferred to polyvinylidene fluoride membranes. The membranes were incubated with blocking buffer for 1 h. Then, the membranes were incubated at 4° C. overnight with the primary antibodies. After washing, the membranes were incubated for 1 h at room temperature with the secondary antibody and washed again. Changes in protein expression was detected using an enhanced chemiluminescence detection reagent. RIPK1 antibody was purchased from Santa Cruz.

The activities of exemplary compounds as described herein, measured in terms of the degradation concentration (DC₅₀ representing the concentration at which 50% of the target, in this case RIPK1 kinase, has been degraded), are provided in Table 11. In Table 11, a DC₅₀ value less than 1 μM is indicated as an “A;” a DC₅₀ value from 1 μM to 10 μM is indicated as a “B” and a DC₅₀ value of greater than 10 μM is indicated as a “C.”

TABLE 11

| Compound | Degradation | Compound | Degradation | Compound | Degradation |
|---|---|---|---|---|---|
| ID | Activity | ID | Activity | ID | Activity |
| 1 | B | 30 | A | 59 | A |
| 2 | B | 31 | A | 60 | A |
| 3 | A | 32 | A | 61 | A |
| 4 | A | 33 | A | 62 | A |
| 5 | A | 34 | A | 63 | A |
| 6 | A | 35 | A | 64 | A |
| 7 | A | 36 | A | 65 | A |
| 8 | B | 37 | A | 66 | A |
| 9 | B | 38 | B | 67 | A |
| 10 | B | 39 | A | 68 | A |
| 11 | C | 40 | A | 69 | B |
| 12 | C | 41 | A | 70 | B |
| 13 | C | 42 | A | 71 | A |
| 14 | C | 43 | A | 72 | A |
| 15 | B | 44 | A | 73 | A |
| 16 | B | 45 | A | 74 | C |
| 17 | B | 46 | A | 75 | C |
| 18 | B | 47 | C | 76 | C |
| 19 | B | 48 | A | 77 | C |
| 20 | B | 49 | A | 78 | A |
| 21 | C | 50 | B | 79 | A |
| 22 | C | 51 | A | 80 | A |
| 23 | C | 52 | A | 81 | A |
| 24 | C | 53 | C | 82 | B |
| 25 | A | 54 | A | 83 | B |
| 26 | A | 55 | A | 84 | C |
| 27 | A | 56 | B | 85 | C |
| 28 | A | 57 | A | 86 | B |
| 29 | A | 58 | A | 87 | B |
| 88 | B | 89 | B | 90 | A |
| 91 | A | 92 | B | | |

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A compound of the following formula:
TPM-L-ELM
or a pharmaceutically acceptable salt, co-crystal, tautomer, stereoisomer, solvate, hydrate, polymorph, isotopically enriched derivative, or prodrug thereof, wherein:
TPM is a targeting protein binding moiety;
L is a bond or a chemical linker group; and
ELM is an E3 ubiquitin ligase binding moiety,
wherein L is covalently bonded to the TPM and the ELM.

2. The compound of claim 1, wherein the TPM has the following formula TPM-1: 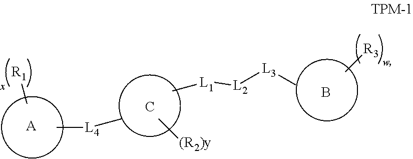
wherein:
Ring A, Ring B, and Ring C are each independently selected from an aryl ring, a heteroaryl ring, a cycloalkyl, or a heterocycloalkyl ring;
L₁ and L₃ are each independently a bond, —N(R₁)— or —CH(R₁)—;
L₂ is —S(O)—, —S(O)₂—, or —C(O)—;
L₄ is a bond or a C₁₋₃ alkyl;
R₁, R₂, and R₃ are each independently hydrogen, halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, —R₄, tetrazole, aryl, aryl substituted with from one to three substituents independently selected from halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, and —R₄, heterocycle, heterocycle substituted with from one to three substituents independently selected from halogen, —OH, —OR₄, —NH₂, —N₄R₅, —NR₄COR₅, —CN, —COOH, —COOR₄, —CONH₂, —CONR₄R₅, —CF₃, —OCF₃, —SO₃H, —SO₃R₄, and —R₄;
R₄ and R₅ are each independently C₁₋₆ alkyl, wherein one or more carbon is optionally replaced with halo, —CO—, —CONH—, —O—, —S—, —SO—, —SO₂—, —N—, —NHCO—, or a heterocycle; and
x, y, and w are each independently an integer from 0 to 4.

3. The compound of claim 2, wherein Ring A is selected from the group consisting of:

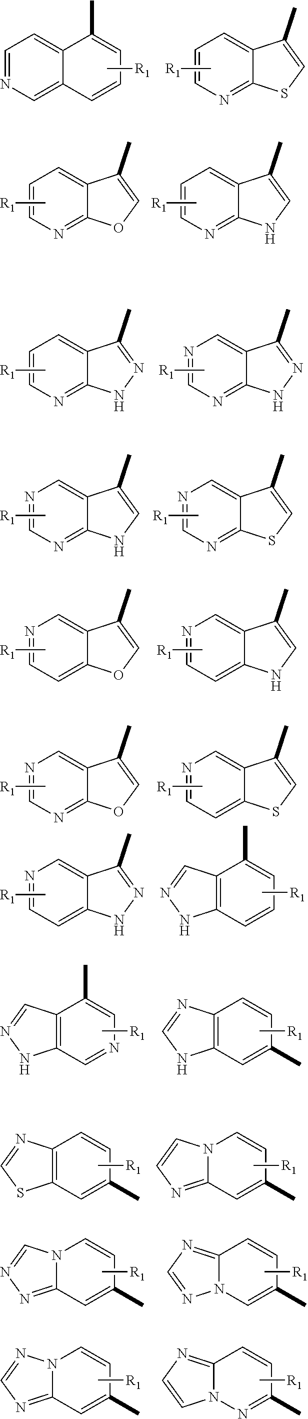

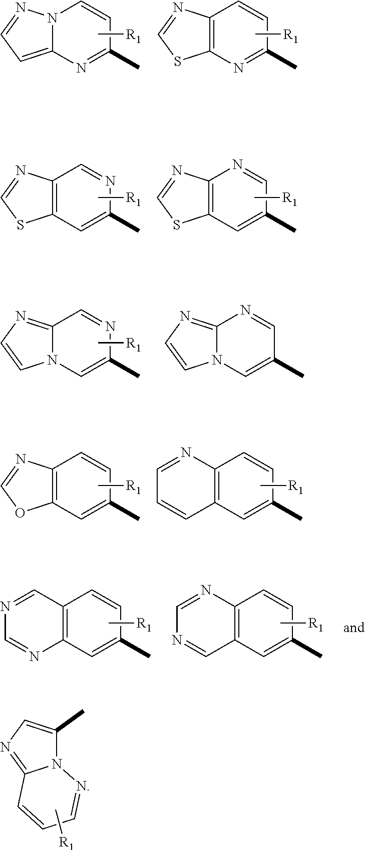

4. The compound of claim 2, wherein Ring B and Ring C are each independently phenyl or a 6-10 membered heterocycle ring.

5. The compound of claim 2, wherein L₁ is —N(R₁)—.

6. The compound of claim 2, wherein L₂ is —S(O)—, —S(O)₂— or —C(O)—.

7. The compound of claim 2 wherein L₃ is —N(R₁)— or —CH(R₁)—.

8. The compound of claim 2, wherein each R₁ is independently selected from hydrogen, halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, —R₄, tetrazole, aryl, aryl substituted with from one to three substituents independently selected from halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, —R₄, heterocycle, heterocycle substituted with from one to three substituents independently selected from halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, and —R₄.

9. The compound of claim 2, wherein R₂ and R₃ are each independently hydrogen, halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, and —R₄.

10. The compound of claim 2 wherein x, y and w are each independently an integer from 0 to 3.

11. The compound of claim 2, wherein the TPM-1 is selected from the group consisting of

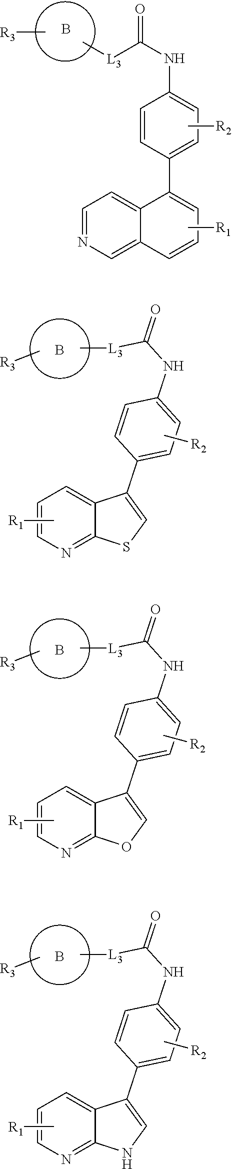

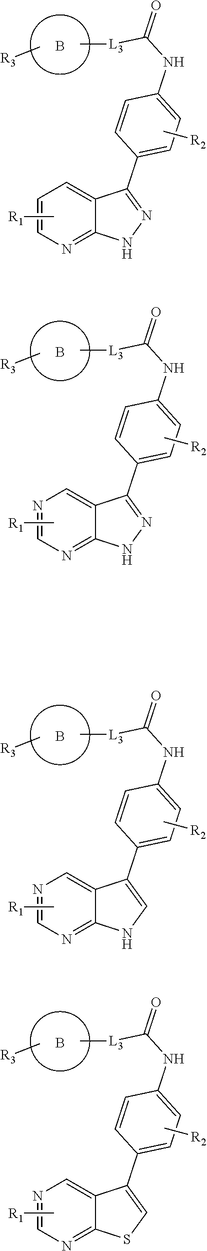

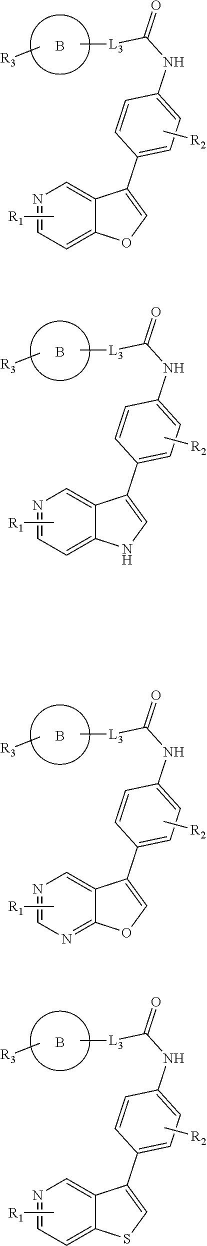

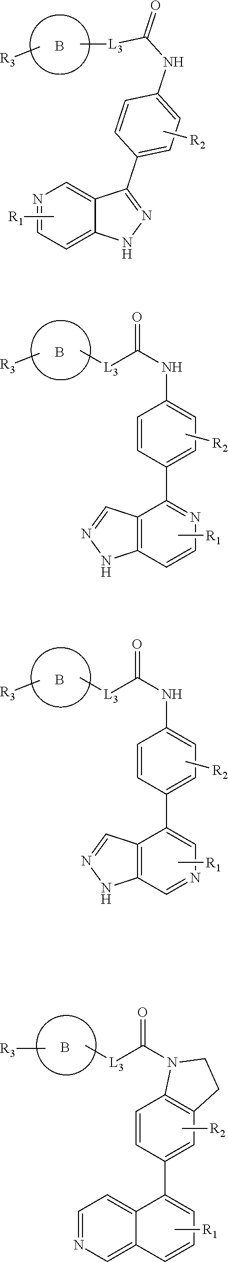

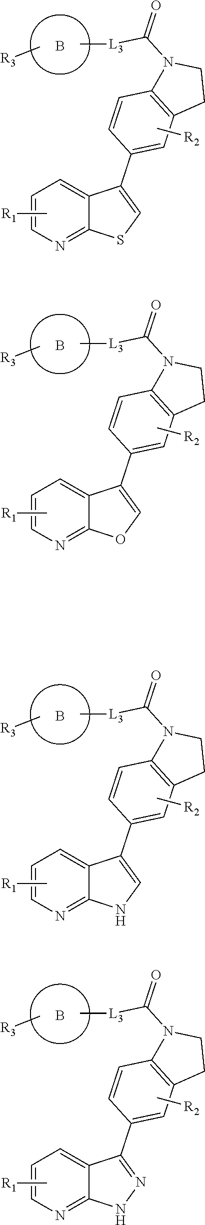

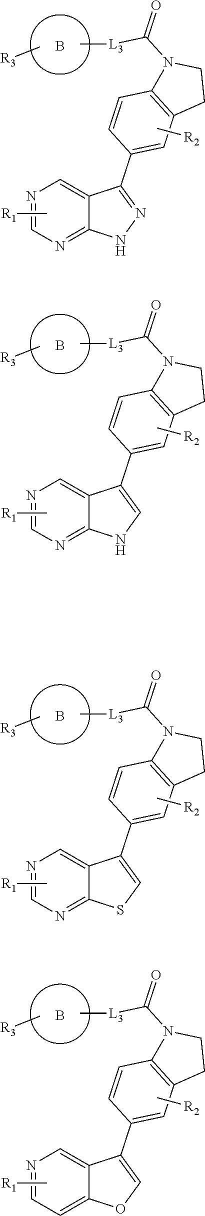

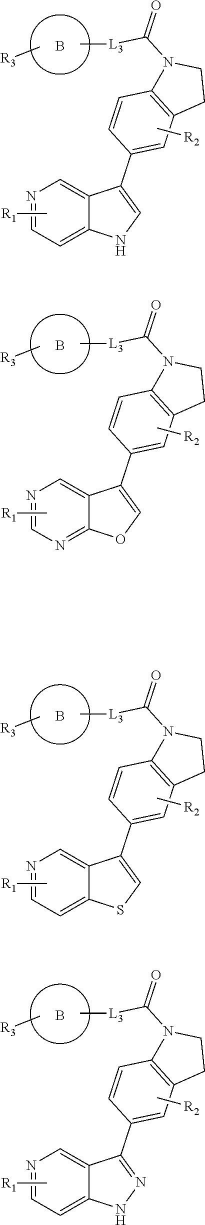

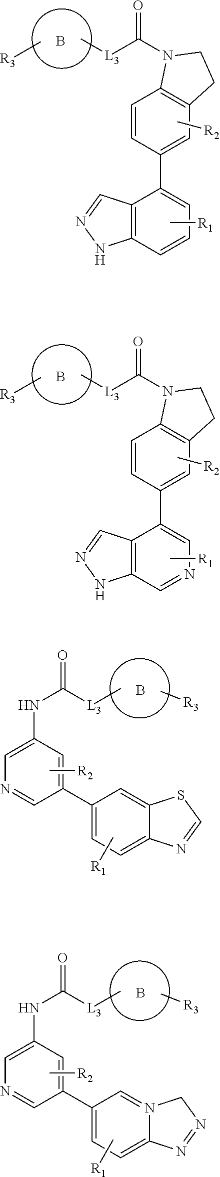

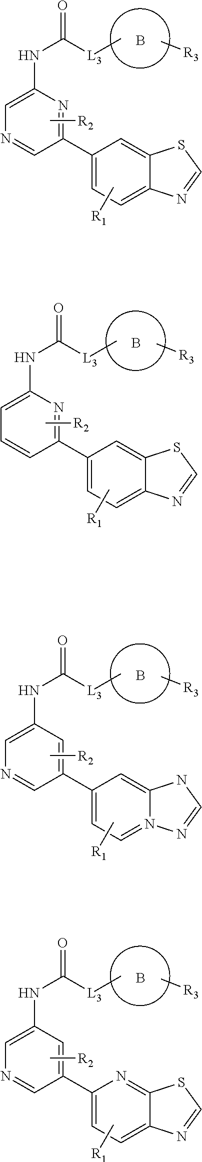

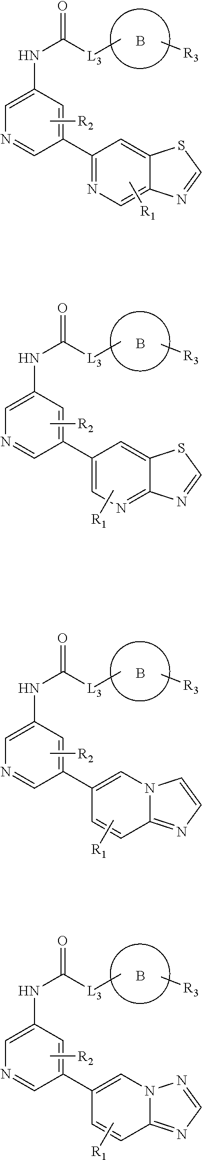

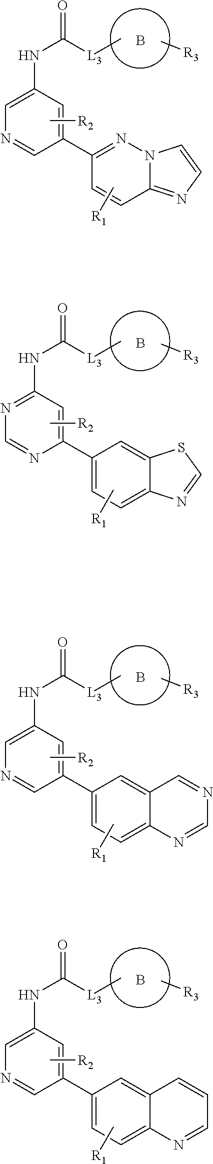

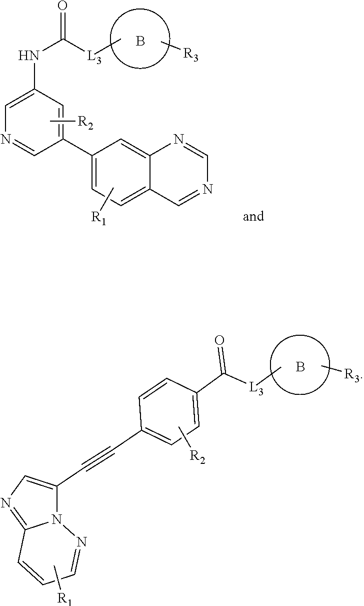

12. The compound of claim 1, wherein the TPM has the following formula TPM-2: 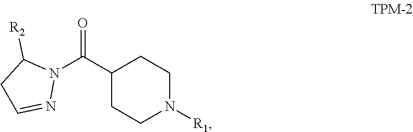
wherein:
R₁ is selected from R₃, 6-10 membered aryl, 6-10 membered aryl substituted by one or two R₃, 5-10 membered heteroaryl, or 5-10 membered heteroaryl substituted by one or two R₃;
R₂ is selected from phenyl, phenyl substituted by one to three substituents selected from halogen, —CN, and R₄, 5-6 membered heteroaryl, 5-6 membered heteroaryl substituted by one to three substituents selected from halogen, —CN, and R₄, 5-6 membered saturated ring, and 5-6 membered saturated ring substituted by one to three substituents selected from halogen, —CN, and R₄;
each R₃ is independently hydrogen, halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, R₄, tetrazole, aryl, aryl substituted with from one to three substituents independently selected from halogen, —OH, —OR₄, NH₂, —NR₄R₅, —NR₄COR₅, —CN, —COOH, —COOR₄, —CONH₂, —CONR₄R₅, —CF₃, —OCF₃, —SO₃H, —SO₃R₄, and R₄, heterocycle, and heterocycle substituted with from one to three substituents independently selected from halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, and R₄; and
R₄ and R₅ are independently C₁₋₆ alkyl, wherein one or more carbon is optionally replaced with halo, CO, CONH, O, S, SO, SO₂, N, NHCO, or heterocycle.

13. The compound of claim 12, wherein R₁ is selected from R₃, phenyl, phenyl substituted by one or two R₃, 5-6 membered heteroaryl, 5-6 membered heteroaryl substituted by one or two substituents R₃; 5-6 membered saturated ring, 5-6 membered saturated ring substituted by one to three substituents selected from halogen, —CN, and R₄.

14. The compound of claim 12, wherein R₂ is selected from phenyl, phenyl substituted by one to three substituents selected from halogen, —CN, or 5-6 membered heteroaryl, 5-6 membered heteroaryl substituted by one to three substituents selected from halogen, and —CN.

15. The compound of claim 12, wherein R₃ is independently selected from the group consisting of hydrogen, halogen, —OH, —NH₂, —NHCOR₄, —CN, —COOH, —COOR₄, —CONH₂, —CONH₂R₄, —CF₃, —OCF₃, —SO₃H, R₄, tetrazole, aryl, aryl substituted with from one to three substituents independently selected from halo, —OH, —NH₂, —CN, —COOH, —CONH₂, —CONH₂R₄, —CF₃, —OCF₃, —SO₃H, R₄, heterocycle, and heterocycle substituted with from one to three substituents independently selected from halo, —OH, —NH₂, —CN, —COOH, —CONH₂, —CONH₂R₄, —CF₃, —OCF₃, —SO₃H, and R₄.

16. The compound of claim 12, wherein the TPM-2 is selected from the group consisting of

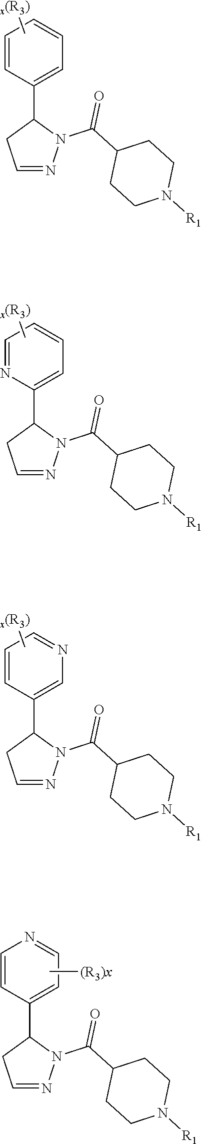

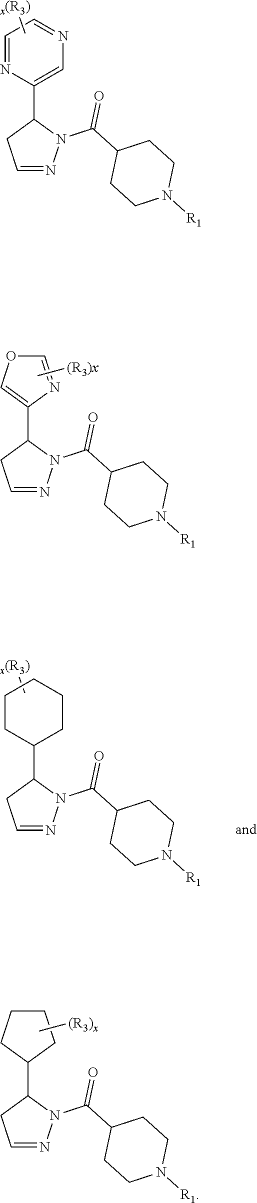

17. The compound of claim 1, wherein the ELM is selected from the group consisting of a cereblon ligase-binding moiety (CLM), a VHL ligase-binding moiety (VLM), and a MDM2 ligase-binding moiety (MLM).

18. The compound of claim 17, wherein the ELM is a CLM selected from the group consisting of: 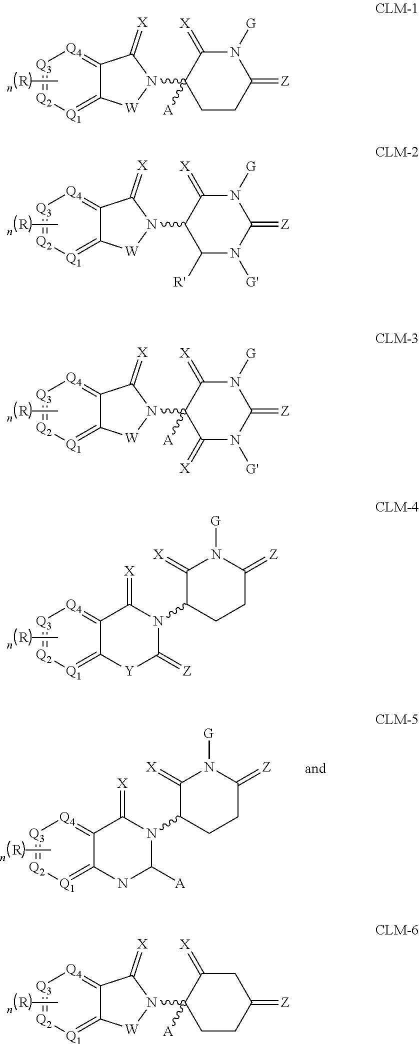
wherein:
W is independently selected from the group consisting of CH₂, CHR, C═O, SO₂, NH, and N-alkyl;
X is independently selected from the group consisting of O, S and H₂;
Y is independently selected from the group consisting of CH₂, —CR′, NH, N-alkyl, N-aryl, N-heteroaryl, N-cycloalkyl, N-heterocyclyl, O, S, and H₂;
Z is independently selected from the group consisting of O, S, and H₂;
G and G′ are independently selected from the group consisting of H, alkyl (linear, branched, optionally substituted with R′), OH, R′OCOOR, ROCONRR″, CH₂-heterocyclyl optionally substituted with R′, and benzyl optionally substituted with R′;
Q₁, Q₂, Q₃ and Q₄ represent a carbon C substituted with a group independently selected from R′, N or N—O;
A is independently selected from the group consisting of H, alkyl, cycloalkyl, Cl and F;
R is selected from the group consisting of halogen, —CF₃, —CN, —CONR′R″, —OR′, —NR′R″, —SR′, —SO₂R′, —SO₂NR′R″, —CR′R″—, —CR′NR′R″—, -aryl, -heteroaryl, -alkyl (linear, branched, optionally substituted), -cycloalkyl, -heterocyclyl, —P(O)(OR′)R″, —OP(O)(OR′)R″, —OP(O)R′R″, —NR′SO₂NR′R″, —NR′CONR′R″—, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —CO₂R′, —C(C═N—OR′)R″, —CR′═CR′R″, —CCR′, —S(C═O)(C═N—R′)R″, —SF₅ or —OCF₃;
R′ and R″ are independently selected from the group consisting of a bond, H, N, N—O, alkyl (linear, branched), cycloalkyl, aryl, heteroaryl, heterocyclic, —C(═O)R, or heterocyclyl, each of which is optionally substituted;
n represents an integer from 1 to 4; [symbol]
represents a bond that may be stereospecific ((R) or (S)) or non-stereospecific; and
Rn comprises 1-4 independent functional groups or atoms, and optionally, one of which is modified to be covalently joined to a chemical linker group (L).

19. The compound of claim 18, wherein the CLM has the following formula: 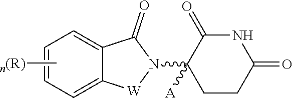
wherein:
W is independently selected from the group CH₂, C═O, NH, and N-alkyl;
A is independently selected from a H, methyl, or optionally substituted linear or branched alkyl;
each R is independently selected from a H, O, OH, N, NH, NH₂, methyl, optionally substituted linear or branched alkyl, optionally substituted C₁₋₆ alkoxy, optionally substituted heterocyclyl, optionally substituted -alkyl-aryl, optionally substituted aryl, optionally substituted heteroaryl aryl, amine, amide, or carboxy;
n represent an integer from 1 to 4; and [symbol]
represents a bond that may be stereospecific ((R) or (S)) or non-stereospecific.

20. The compound of claim 19, wherein the CLM is selected from the group consisting of:

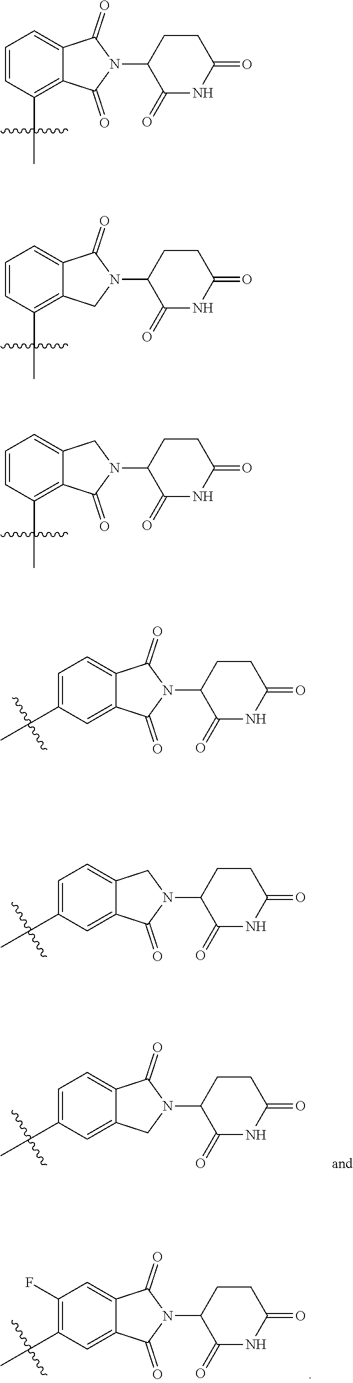

21. The compound of claim 17, wherein the ELM is a VLM having the following structure: 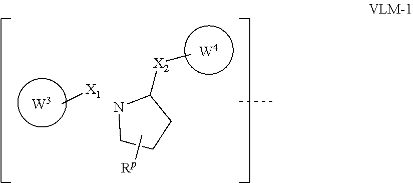
wherein:
the dashed line indicates the attachment a chemical linker moiety coupling at least one TPM;
X¹ and X² are independently selected from the group of a bond, O, NR^{Y3}, CR_{Y3}R_{Y4}, C═O, C═S, SO, and SO₂;
R^{Y3} and R^{Y4} are each independently selected from the group of hydrogen, linear or branched C₁₋₆ alkyl, optionally substituted by one or more halo, optionally substituted C₁₋₆ alkoxyl, wherein the R^{Y3} and R^{Y4} groups are optionally substituted by 0-3 R^{p} groups, wherein R^{p} is one to three groups, each independently selected from the group hydrogen, halogen, —OH, C₁₋₃ alkyl, C═O;
W³ is selected from the group of an optionally substituted T, an optionally substituted -TN(R^{1a}R^{1b})X³, optionally substituted -T-N(R^{1a}R^{1b}), optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR^{X}-T-Aryl, an optionally substituted —NR′-T-Heteroaryl or an optionally substituted —NR′-T-Heterocycle;
X³ is C═O, R¹, R^{1a}, R^{1b};
R¹, R^{1a}, and R_{1b} are each independently selected from the group consisting of hydrogen, linear or branched C₁₋₆ alkyl group optionally substituted by one or more halogen or —OH groups, R^{Y3}C═O, R^{Y3}C═S, R^{Y3}SO, R^{Y3}SO₂, N(R^{Y3}R^{Y4}) C═O, N(R^{Y3}R^{Y4})C═S, N(R^{Y3}R^{Y4})SO, and N(R^{Y3}R^{Y4})SO₂;
T is selected from the group of an optionally substituted alkyl, —(CH₂)ₙ— group, wherein each one of the methylene groups is optionally substituted with one or two substituents selected from the group consisting of halogen, methyl, optionally substituted alkoxy, a linear or branched C1-C6 alkyl group optionally substituted by 1 or more halogen, C(O)NR^{x}R^{1a}, or NR^{x}R^{1a} or R¹ and R^{1a} are joined to form an optionally substituted heterocycle, or —OH groups or an amino acid side chain optionally substituted;
n is 0 to 6, e.g., 0, 1, 2, or 3, preferably 0 or 1;
W⁴ is an optionally substituted —NR¹-T-Aryl wherein the aryl group may be optionally substituted with an optionally substituted 5-6 membered heteroaryl or aryl, an optionally substituted —NR1-T-Heteroaryl group or an optionally substituted —NR¹-T-Heterocycle, where NR¹ is covalently bonded to X² and R¹ is H or CH₃, preferably H;
T is selected from the group consisting of an optionally substituted alkyl, —(CH₂)ₙ— group, wherein each one of the methylene groups is optionally substituted with one or two substituents selected from the group of halogen, methyl, optionally substituted alkoxy, a linear or branched C₁₋₆ alkyl group optionally substituted by 1 or more halogen, C(O) NRₓR^{1a}, or NRₓR^{1a} or R¹ and R^{1a} are joined to form an optionally substituted heterocycle, or —OH groups or an amino acid side chain optionally substituted; and
n is 0 to 6.

22. The compound of claim 21, wherein W⁴ of Formula VLM-1 is selected from the group consisting of: 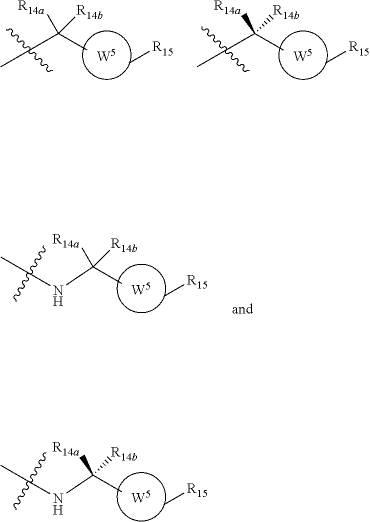
wherein R₁₄ₐ, R_{14b}, are independently selected from the group of H, haloalkyl, or optionally substituted alkyl
W⁵ is an optionally substituted phenyl, an optionally substituted napthyl, or an optionally substituted 5-10 membered heteroaryl;
R₁₅ is selected from the group of H, halogen, CN, OH, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl.

23. The compound of claim 17, wherein the ELM is a VLM having the following structure: 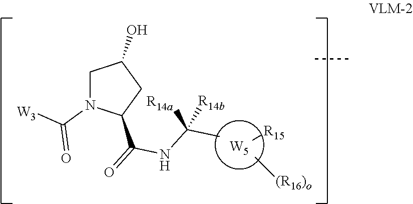
wherein:
the dashed line indicates the attachment a chemical linker moiety coupling at least one TPM;
W³ group is selected from the group consisting of an optionally substituted aryl, optionally substituted heteroaryl, and 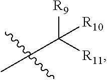
wherein R₉ and R₁₀ are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl; and R₁₁ is selected from the group of an optionally substituted heterocyclic, optionally substituted alkoxy, optionally substituted heteroaryl, optionally substituted aryl, 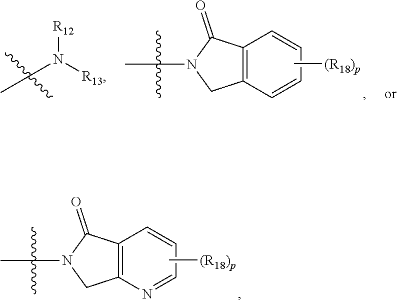
wherein R₁₂ is selected from the group of H or optionally substituted alkyl and R₁₃ is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ and R_{14b} are each independently selected from the group of H, haloalkyl, or optionally substituted alkyl;
W₅ is selected from the group of an optionally substituted phenyl or an optionally substituted 5-10 membered heteroaryl;
R₁₅ is selected from the group of H, halogen, CN, OH, NO2, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl;
each R₁₆ is independently selected from the group of H, CN, halogen, optionally substituted alkyl, optionally substituted haloalkyl, hydroxy, or optionally substituted haloalkoxy;
o is 0, 1, 2, 3, or 4;
R₁₈ is independently selected from the group of H, halogen, optionally substituted alkoxy, cyano, optionally substituted alkyl, haloalkyl, haloalkoxy or a linker;
p is 0, 1, 2, 3, or 4; and
the dashed line indicates the site of attachment of a chemical linker moiety coupling at least one TPM.

24. The compound of claim 17, wherein the ELM is a VLM selected from the group consisting of: 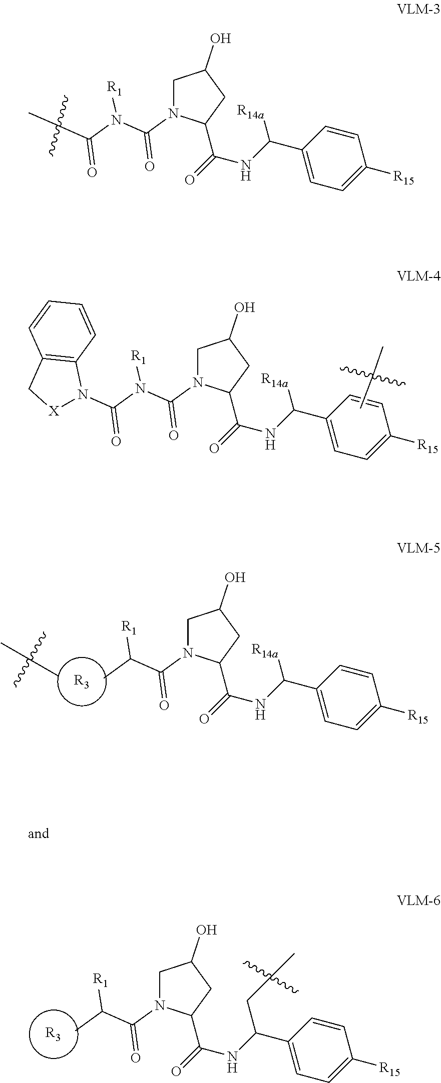
wherein:
R₁ is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, OH, NO2, optionally substituted heteroaryl, optionally substituted aryl, optionally substituted alkyl, optionally substituted haloalkyl, optionally substituted haloalkoxy, optionally substituted cycloalkyl, or optionally substituted cycloheteroalkyl;
X is C, CH₂, or C═O;
R₃ is absent or an optionally substituted 5 or 6 membered heteroaryl; and
the dashed line indicates the site of attachment of a chemical linker moiety coupling at least one TPM.

25. The compound of claim 17, wherein the ELM is a VLM having the following structure: 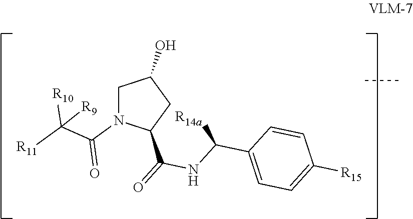
wherein:
R₁₅ₐ is H, haloalkyl, optionally substituted alkyl, methyl, fluoromethyl, hydroxymethyl, ethyl, isopropyl, or cyclopropyl;
R₉ is H;
R₁₀ is H, ethyl, isopropyl, tert-butyl, sec-butyl, cyclopropyl, cyclobutyl, cyclopentyl, or cyclohexyl;
R₁₁ is 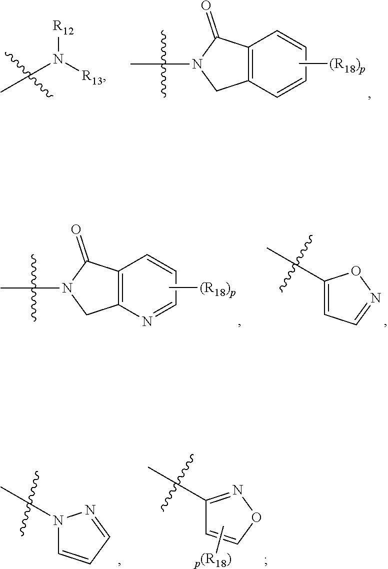
or optionally substituted heteroaryl;
p is 0, 1, 2, 3, or 4;
each R₁₈ is independently halo, optionally substituted alkoxy, cyano, optionally substituted alkyl, haloalkyl, haloalkoxy or a linker;
R₁₂ is H or C═O;
R₁₃ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₅ is selected from the group consisting of H, halogen, Cl, CN, OH, NO₂, optionally substituted heteroaryl, and optionally substituted aryl: 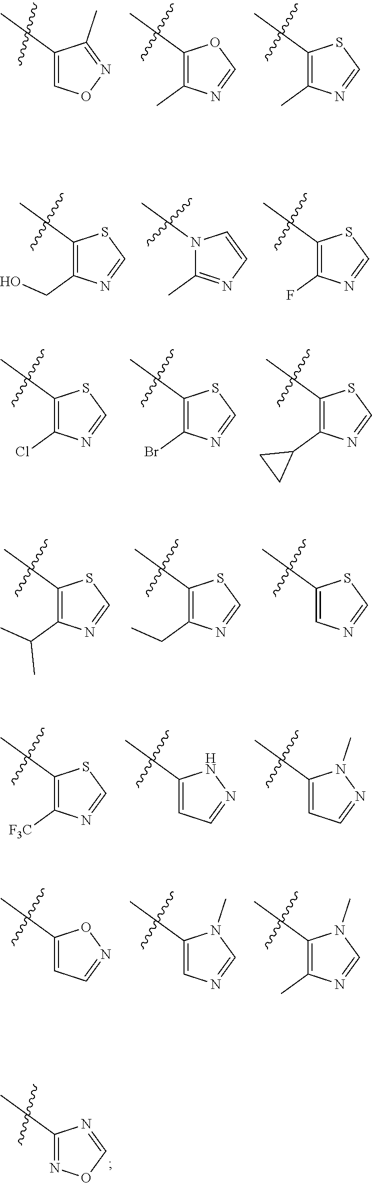
and
the dashed line indicates the site of attachment of a chemical linker moiety coupling at least one TPM.

26. The compound of claim 17, wherein the ELM is a VLM selected from the group consisting of:

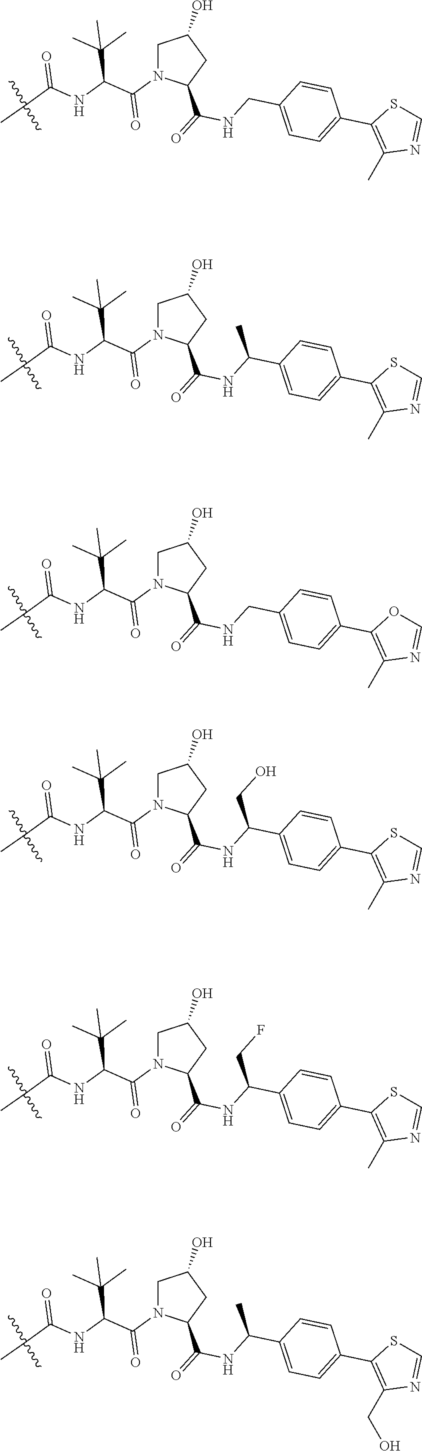 [SMILES: *C(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C ; *C(=O)N[C@H](C(=O)N1C[C@H](O)C[C@H]1C(=O)NCc1ccc(-c2ocnc2C)cc1)C(C)(C)C ; *C(=O)N[C@H](C(=O)N1C[C@H](O)C[C@H]1C(=O)N[C@@H](CO)c1ccc(-c2scnc2C)cc1)C(C)(C)C ; *C(=O)N[C@H](C(=O)N1C[C@H](O)C[C@H]1C(=O)N[C@@H](CF)c1ccc(-c2scnc2C)cc1)C(C)(C)C ; *C(=O)N[C@H](C(=O)N1C[C@H](O)C[C@H]1C(=O)N[C@@H](C)c1ccc(-c2scnc2CO)cc1)C(C)(C)C]

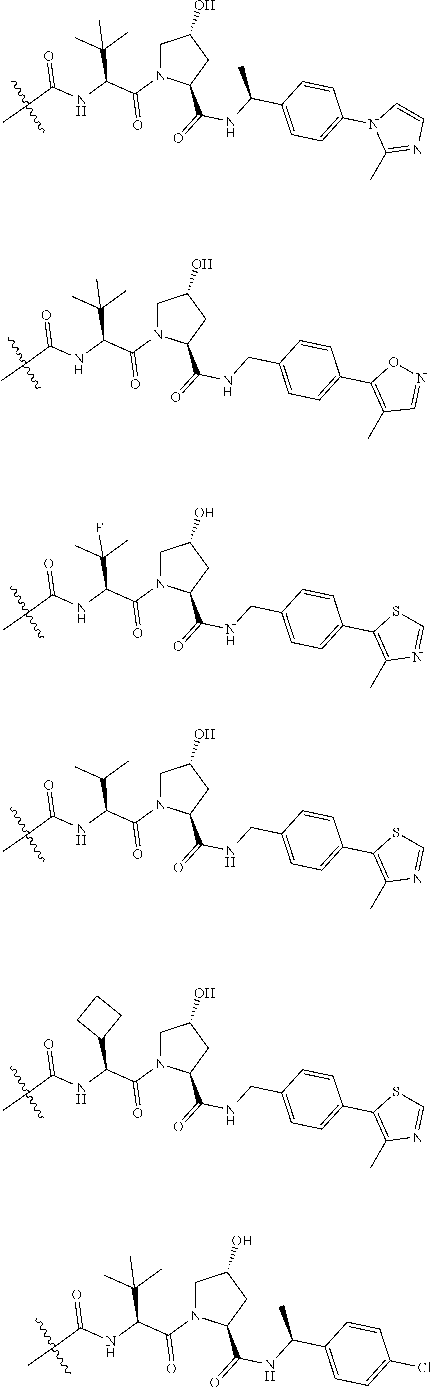 [SMILES: *C(=O)N[C@H](C(=O)N1C[C@H](O)C[C@H]1C(=O)N[C@@H](C)c1ccc(-n2ccnc2C)cc1)C(C)(C)C ; *C(=O)N[C@H](C(=O)N1C[C@H](O)C[C@H]1C(=O)NCc1ccc(-c2oncc2C)cc1)C(C)(C)C ; *C(=O)N[C@H](C(=O)N1C[C@H](O)C[C@H]1C(=O)NCc1ccc(-c2scnc2C)cc1)C(C)(C)F ; *C(=O)N[C@H](C(=O)N1C[C@H](O)C[C@H]1C(=O)NCc1ccc(-c2scnc2C)cc1)C(C)C ; *C(=O)N[C@H](C(=O)N1C[C@H](O)C[C@H]1C(=O)NCc1ccc(-c2scnc2C)cc1)C1CCC1 ; *C(=O)N[C@H](C(=O)N1C[C@H](O)C[C@H]1C(=O)N[C@@H](C)c1ccc(Cl)cc1)C(C)(C)C]

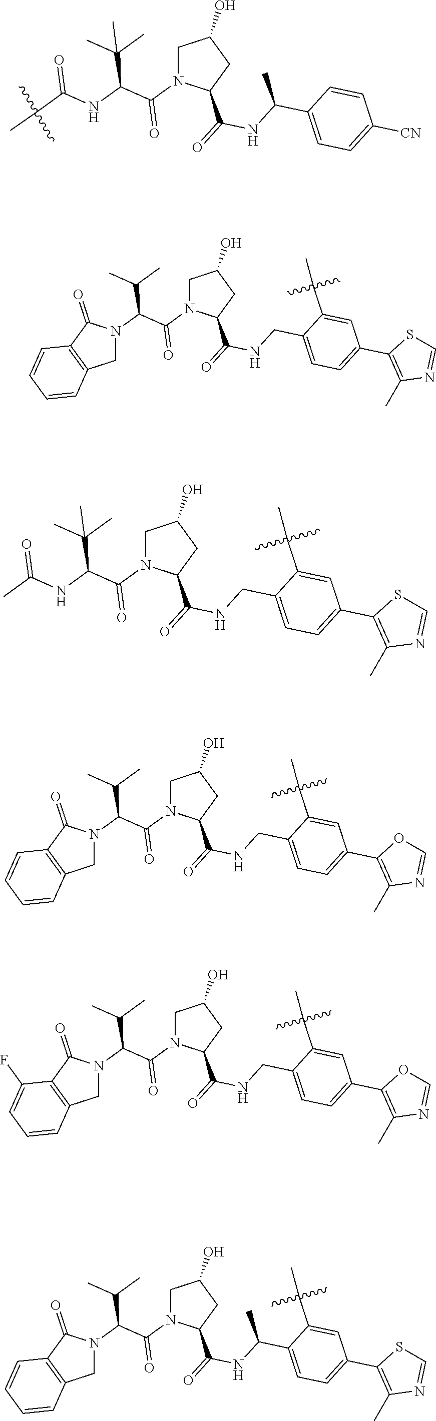 [SMILES: *C(=O)N[C@H](C(=O)N1C[C@H](O)C[C@H]1C(=O)N[C@@H](C)c1ccc(C#N)cc1)C(C)(C)C ; *c1cc(-c2scnc2C)ccc1CNC(=O)[C@@H]1C[C@@H](O)CN1C(=O)[C@H](C(C)C)N1Cc2ccccc2C1=O ; *c1cc(-c2scnc2C)ccc1CNC(=O)[C@@H]1C[C@@H](O)CN1C(=O)[C@@H](NC(C)=O)C(C)(C)C ; *c1cc(-c2ocnc2C)ccc1CNC(=O)[C@@H]1C[C@@H](O)CN1C(=O)[C@H](C(C)C)N1Cc2ccccc2C1=O ; *c1cc(-c2ocnc2C)ccc1CNC(=O)[C@@H]1C[C@@H](O)CN1C(=O)[C@H](C(C)C)N1Cc2cccc(F)c2C1=O ; *c1cc(-c2scnc2C)ccc1[C@H](C)NC(=O)[C@@H]1C[C@@H](O)CN1C(=O)[C@H](C(C)C)N1Cc2ccccc2C1=O]

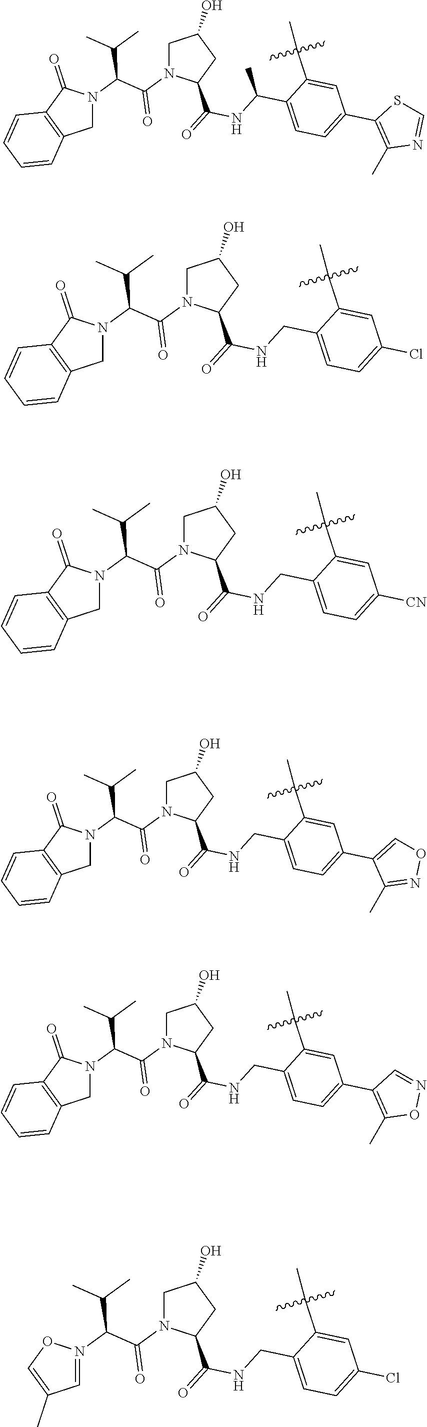

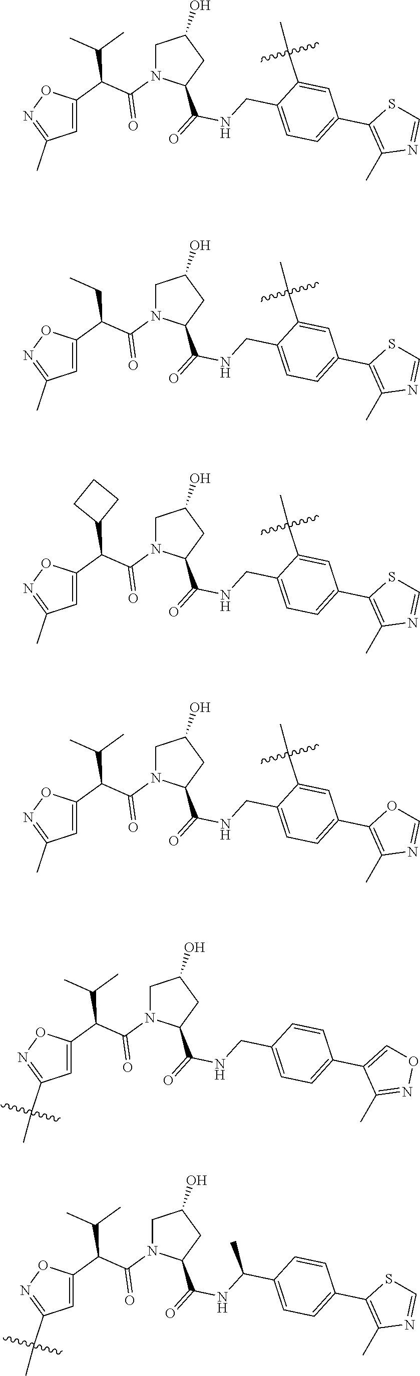 [SMILES: *c1cc(-c2scnc2C)ccc1CNC(=O)[C@@H]1C[C@@H](O)CN1C(=O)[C@@H](c1cc(C)no1)C(C)C ; *c1cc(-c2scnc2C)ccc1CNC(=O)[C@@H]1C[C@@H](O)CN1C(=O)[C@H](CC)c1cc(C)no1 ; *c1cc(-c2scnc2C)ccc1CNC(=O)[C@@H]1C[C@@H](O)CN1C(=O)[C@@H](c1cc(C)no1)C1CCC1 ; *c1cc(-c2ocnc2C)ccc1CNC(=O)[C@@H]1C[C@@H](O)CN1C(=O)[C@@H](c1cc(C)no1)C(C)C ; *c1cc([C@H](C(=O)N2C[C@H](O)C[C@H]2C(=O)NCc2ccc(-c3conc3C)cc2)C(C)C)on1 ; *c1cc([C@H](C(=O)N2C[C@H](O)C[C@H]2C(=O)N[C@@H](C)c2ccc(-c3scnc3C)cc2)C(C)C)on1]

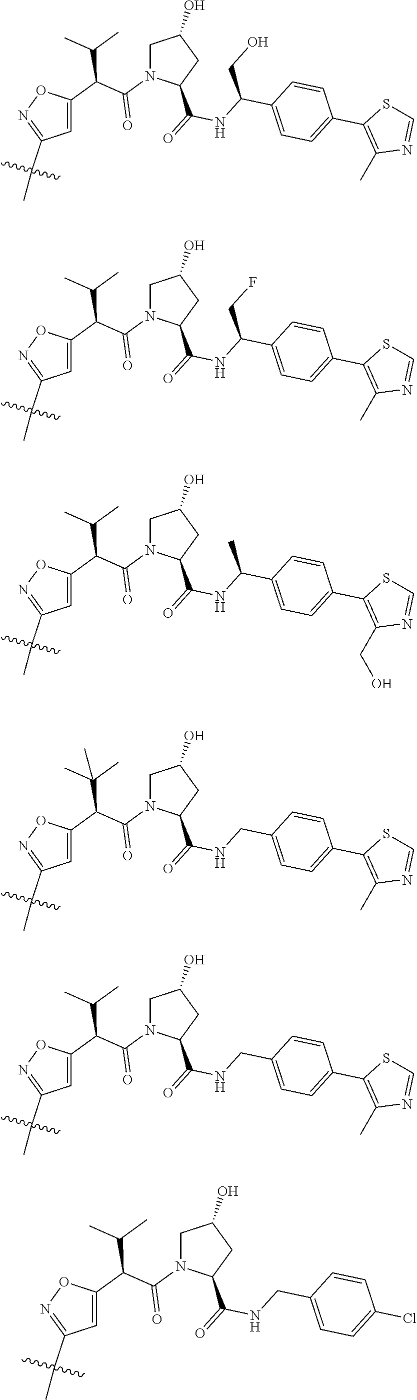 [SMILES: *c1cc([C@H](C(=O)N2C[C@H](O)C[C@H]2C(=O)N[C@@H](CO)c2ccc(-c3scnc3C)cc2)C(C)C)on1 ; *c1cc([C@H](C(=O)N2C[C@H](O)C[C@H]2C(=O)N[C@@H](CF)c2ccc(-c3scnc3C)cc2)C(C)C)on1 ; *c1cc([C@H](C(=O)N2C[C@H](O)C[C@H]2C(=O)N[C@@H](C)c2ccc(-c3scnc3CO)cc2)C(C)C)on1 ; *c1cc([C@H](C(=O)N2C[C@H](O)C[C@H]2C(=O)NCc2ccc(-c3scnc3C)cc2)C(C)(C)C)on1 ; *c1cc([C@H](C(=O)N2C[C@H](O)C[C@H]2C(=O)NCc2ccc(-c3scnc3C)cc2)C(C)C)on1 ; *c1cc([C@H](C(=O)N2C[C@H](O)C[C@H]2C(=O)NCc2ccc(Cl)cc2)C(C)C)on1]

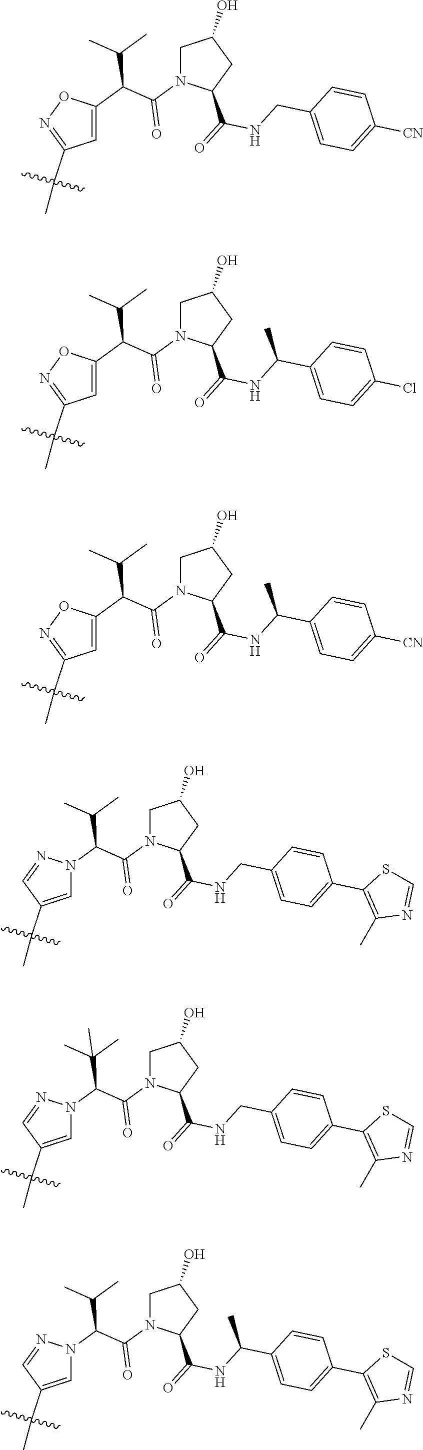 [SMILES: *c1cc([C@H](C(=O)N2C[C@H](O)C[C@H]2C(=O)NCc2ccc(C#N)cc2)C(C)C)on1 ; *c1cc([C@H](C(=O)N2C[C@H](O)C[C@H]2C(=O)N[C@@H](C)c2ccc(Cl)cc2)C(C)C)on1 ; *c1cc([C@H](C(=O)N2C[C@H](O)C[C@H]2C(=O)N[C@@H](C)c2ccc(C#N)cc2)C(C)C)on1 ; *c1cnn([C@H](C(=O)N2C[C@H](O)C[C@H]2C(=O)NCc2ccc(-c3scnc3C)cc2)C(C)C)c1 ; *c1cnn([C@H](C(=O)N2C[C@H](O)C[C@H]2C(=O)NCc2ccc(-c3scnc3C)cc2)C(C)(C)C)c1 ; *c1cnn([C@H](C(=O)N2C[C@H](O)C[C@H]2C(=O)N[C@@H](C)c2ccc(-c3scnc3C)cc2)C(C)C)c1]

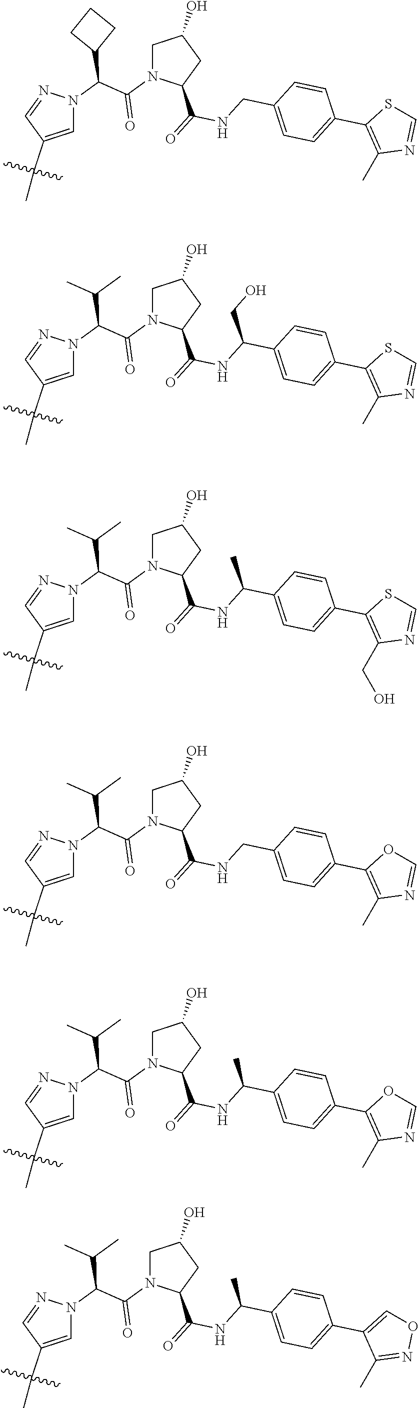 [SMILES: *c1cnn([C@H](C(=O)N2C[C@H](O)C[C@H]2C(=O)NCc2ccc(-c3scnc3C)cc2)C2CCC2)c1 ; *c1cnn([C@H](C(=O)N2C[C@H](O)C[C@H]2C(=O)N[C@@H](CO)c2ccc(-c3scnc3C)cc2)C(C)C)c1 ; *c1cnn([C@H](C(=O)N2C[C@H](O)C[C@H]2C(=O)N[C@@H](C)c2ccc(-c3scnc3CO)cc2)C(C)C)c1 ; *c1cnn([C@H](C(=O)N2C[C@H](O)C[C@H]2C(=O)NCc2ccc(-c3ocnc3C)cc2)C(C)C)c1 ; *c1cnn([C@H](C(=O)N2C[C@H](O)C[C@H]2C(=O)N[C@@H](C)c2ccc(-c3ocnc3C)cc2)C(C)C)c1 ; *c1cnn([C@H](C(=O)N2C[C@H](O)C[C@H]2C(=O)N[C@@H](C)c2ccc(-c3conc3C)cc2)C(C)C)c1]

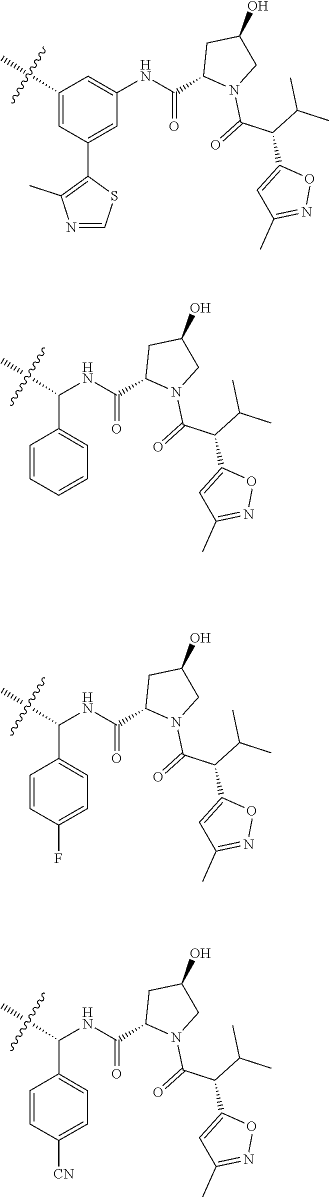

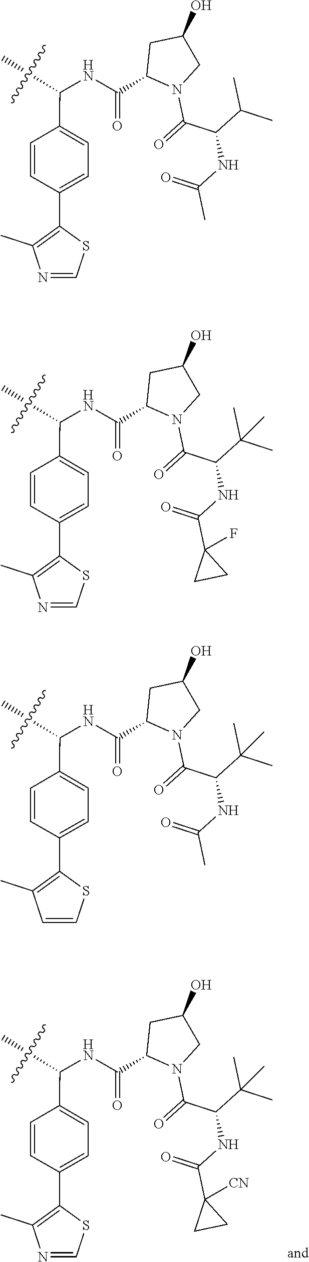

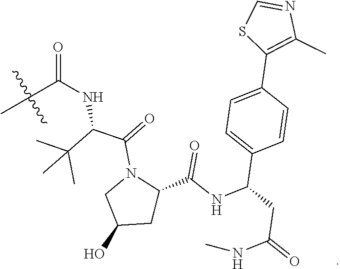 [SMILES: *C(=O)N[C@H](C(=O)N1C[C@H](O)C[C@H]1C(=O)N[C@@H](CC(=O)NC)c1ccc(-c2scnc2C)cc1)C(C)(C)C]

27. The compound of claim 17, wherein the ELM is a MLM is selected from the group consisting of: 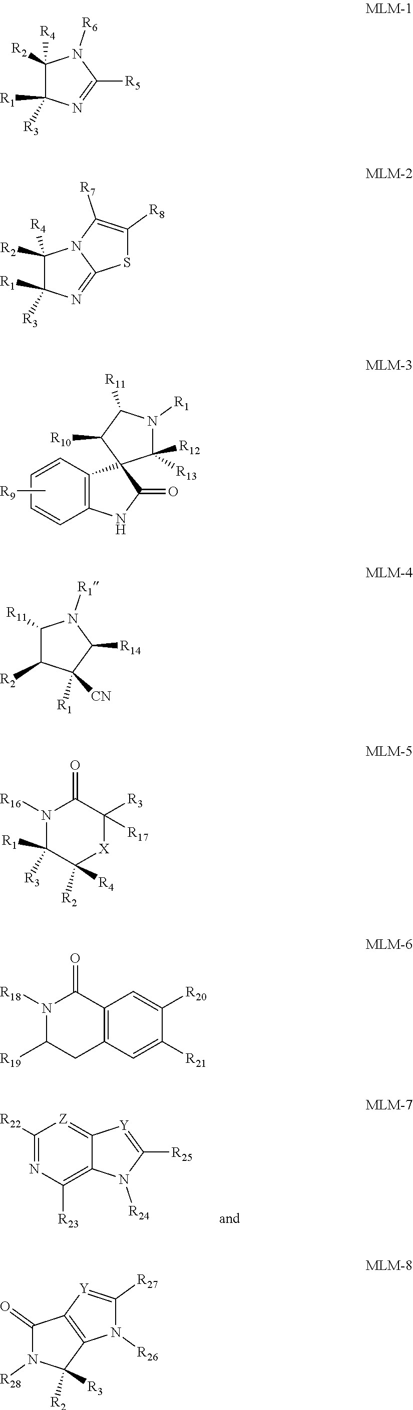
wherein:
X is selected from the group consisting of carbon, oxygen, sulfur, sulfoxide, sulfone, and N—R^{a}, wherein R^{a} is independently H or C₁₋₆ alkyl;
Y and Z are independently C or N;
R₁ and R₂ are independently selected from the group consisting of an aryl or heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of: halogen, —CN, C₁₋₆ alkyl group, C₃₋₆ cycloalkyl, —OH, unsubstituted or fluorine substituted C₁₋₆ alkoxy, C₁₋₆ sulfoxide, C₁₋₆ sulfone, C₂₋₆ ketone, C₂₋₆ amides, and di-C₂₋₆ alkyl amine;
R₃ and R₄ are independently selected from the group consisting of H, methyl and C₁₋₆ alkyl;
R₅ is selected from the group consisting of an aryl group, a heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of: halogen, —CN, C₁₋₆ alkyl, C₃₋₆ cycloalkyl, —OH, unsubstituted or fluorine substituted C₁₋₆ alkoxy, C₁₋₆ sulfoxide, C₁₋₆ sulfone, C₂₋₆ ketone, C₂₋₆ amides, di-C₂₋₆ alkyl amine, morpholinyl, C₃₋₆ alkyl ester, and C₃₋₆ alkyl cyanide;
R₆ is H or —C(═O)R^{b}, wherein Rb is selected from the group consisting of alkyl, cycloalkyl, mono-, di- or tri-substituted aryl or heteroaryl, 4-morpholinyl, 1-(3-oxopiperazinyl), 1-piperidinyl, 4-N—R^{c}-morpholinyl, 4-R^{c}-1-piperidinyl, and 3-R^{c}-1-piperidinyl, wherein R^{c} is selected from the group consisting of alkyl, fluorine substituted alkyl, cyano alkyl, hydroxyl-substituted alkyl, cycloalkyl, alkoxyalkyl, amide alkyl, alkyl sulfone, alkyl sulfoxide, alkyl amide, aryl, heteroaryl, mono-, bis- and tri-substituted aryl or heteroaryl, CH₂CH₂R^{d}, and CH₂CH₂CH₂R^{d}, wherein R^{d} is selected from the group consisting of alkoxy, alkyl sulfone, alkyl sulfoxide, N-substituted carboxamide, —NHC(O)-alkyl, —NH—SO₂-alkyl, aryl, substituted aryl, heteroaryl, substituted heteroaryl;
R₇ is selected from the group consisting of H, C₁₋₆ alkyl, cyclic alkyl, fluorine substituted alkyl, cyano substituted alkyl, 5- or 6-membered hetero aryl or aryl, substituted 5- or 6-membered hetero aryl or aryl;
R₈ is selected from the group consisting of —Rₑ—C(O)—R^{f}, —R^{e}-alkoxy, —R^{e}-aryl, —R^{e}-heteroaryl, and —R^{e}—C(O)—R^{f}—C(O)—R^{g}, wherein: R^{e} is an C₁₋₆ alkylene, or a bond; R^{f} and R^{g} are independently substituted pyrrolidine, substituted piperidine, substituted piperazine;
R₉ is selected from the group consisting of a mono-, bis- or tri-substituent on the fused bicyclic aromatic ring in MLM-3, wherein the substitutents are independently selected from the group consisting of halogen, alkene, alkyne, alkyl, unsubstituted or substituted with Cl or F;
R₁₀ is selected from the group consisting of an aryl or heteroaryl group, wherein the heteroaryl group can optionally contain one or two heteroatoms as sulfur or nitrogen, aryl or heteroaryl group can be mono-cyclic or bi-cyclic, the aryl or heteroaryl group can be unsubstituted or substituted with one to three substituents, including a halogen, F, Cl, —CN, alkene, alkyne, C₁₋₆ alkyl, C₃₋₆ cycloalkyl, —OH, unsubstituted or fluorine substituted C₁₋₆ alkoxy, C₁₋₆ sulfoxide, C₁₋₆ sulfone, C₂₋₆ ketone;
R₁₁ is —C(O)—N(R^{h})(Rⁱ), wherein R^{h} and Rⁱ are selected from groups consisting of the following: H; optionally substituted linear or branched C₁₋₆ alkyl; alkoxy substituted alkyl; mono- and di-hydroxy substituted alkyl, sulfone substituted alkyl; optionally substituted aryl; optionally substituted heteroaryl; mono-, bis- or tri-substituted aryl or heteroaryl; phenyl-4-carboxylic acid; substituted phenyl-4-carboxylic acid, alkyl carboxylic acid; optionally substituted heteroaryl carboxylic acid; alkyl carboxylic acid; fluorine substituted alkyl carboxylic acid; optionally substituted cycloalkyl, 3-hydroxycyclobutane, 4-hydroxycyclohehexane, aryl substituted cycloalkyl; heteroaryl substituted cycloalkyl; or R^{h} and Rⁱ taken together form a ring;
R₁₂ and R₁₃ are independently selected from H, C₁₋₆ alkyl, C₂₋₆ alkenyl, C₂₋₆ alkynyl, C₄₋₆ cycloalkyl, substituted cycloalkyl, cycloalkenyl, substituted cycloalkenyl, 5- and 6-membered aryl and heteroaryl, R₁₂ and R₁₃ can be connected to form a 5- and 6-membered ring with or without substitution on the ring;
R₁₄ is selected from the group consisting of alkyl, substituted alkyl, alkenyl, substituted alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₁₆ is selected from the group consisting of C₁₋₆ alkyl, C₁₋₆ cycloalkyl, C₂₋₆ alkenyl, C₁₋₆ alkyl or C₃₋₆ cycloalkyl with one or multiple hydrogens replaced by fluorine, alkyl or cycloalkyl with one CH₂ replaced by SO, —S, or —SO₂, alkyl or cycloalkyl with terminal CH₃ replaced by SO₂N(alkyl)(alkyl), —CON(alkyl)(alkyl), —N(alkyl)SO₂(alkyl), —CO₂(alkyl), —O(alkyl), C₁₋₆ alkyl or alkyl-cycloalkyl with hydrogen replaced by hydroxyl group, a 3 to 7 membered cycloalkyl or heterocycloalkyl, optionally containing a —CO— group, or a 5 to 6 membered aryl or heteroaryl group, which heterocycloalkyl or heteroaryl group can contain from one to three heteroatoms independently selected from O, N or S, and the cycloalkyl, heterocycloalkyl, aryl or heteroaryl group can be unsubstituted or substituted with from one to three substituents independently selected from halogen, C₁₋₆ alkyl, hydroxylated C₁₋₆ alkyl, C₁₋₆ alkyl containing thioether, ether, sulfone, sulfoxide, fluorine substituted ether or cyano group;
R₁₇ is selected from the group consisting of (CH₂)ₙC(O)NR^{k}R^{l}, wherein R^{k} and R^{l} are independently selected from H, C₁₋₆ alkyl, hydroxylated C₁₋₆ alkyl, C₁₋₆ alkoxy alkyl, C₁₋₆ alkyl with one or multiple hydrogens replaced by fluorine, C₁₋₆ alkyl with one carbon replaced by SO, SO₂, C₁₋₆ alkoxyalkyl with one or multiple hydrogens replaced by fluorine, C₁₋₆ alkyl with hydrogen replaced by a cyano group, 5 and 6 membered aryl or heteroaryl, alkyl aryl with alkyl group containing 1-6 carbons, and alkyl heteroaryl with C₁₋₆ alkyl, wherein the aryl or heteroaryl group can be further substituted;
R₁₈ is selected from the group consisting of substituted aryl, heteroaryl, alkyl, cycloalkyl, the substitution is preferably —N(C₁₋₄ alkyl)(cycloalkyl), —N(C₁₋₄ alkyl)alkyl-cycloalkyl, and —N(C₁₋₄ alkyl)[(alkyl)-(heterocycle-substituted)-cycloalkyl];
R₁₉ is selected from the group consisting of aryl, heteroaryl, bicyclic heteroaryl, and these aryl or heteroaryl groups can be substituted with halogen, C₁₋₆ alkyl, C₁₋₆ cycloalkyl, CF₃, F, CN, alkyne, alkyl sulfone, the halogen substitution can be mon-bis- or tri-substituted;
R₂₀ and R₂₁ are independently selected from C₁₋₆ alkyl, C₁₋₆ cycloalkyl, C₁₋₆ alkoxy, hydroxylated C₁₋₆ alkoxy, and fluorine substituted C₁₋₆ alkoxy, wherein R₂₀ and R₂₁ can further be connected to form a 5, 6 and 7-membered cyclic or heterocyclic ring, which can further be substituted;
R₂₂ is selected from the group consisting of H, C₁₋₆ alkyl, C₁₋₆ cycloalkyl, carboxylic acid, carboxylic acid ester, amide, reverse amide, sulfonamide, reverse sulfonamide, N-acyl urea, nitrogen-containing 5-membered heterocycle, the 5-membered heterocycles can be further substituted with C₁₋₆ alkyl, alkoxy, fluorine-substituted alkyl, CN, and alkylsulfone;
R₂₃ is selected from aryl, heteroaryl, —O-aryl, —O— heteroaryl, —O-alkyl, —O-alkyl-cycloalkyl, —NH-alkyl, —NH-alkyl-cycloalkyl, —N(H)-aryl, —N(H)-heteroaryl, —N(alkyl)-aryl, —N(alkyl)-heteroaryl, the aryl or heteroaryl groups can be substituted with halogen, C₁₋₆ alkyl, hydroxylated C₁₋₆ alkyl, cycloalkyl, fluorine -substituted C₁₋₆ alkyl, CN, alkoxy, alkyl sulfone, amide and sulfonamide;
R₂₄ is selected from the group consisting of —CH₂—C₁₋₆ alkyl, —CH₂-cycloalkyl, —CH₂-aryl, —CH₂-heteroaryl, where alkyl, cycloalkyl, aryl and heteroaryl can be substituted with halogen, alkoxy, hydroxylated alkyl, cyano-substituted alkyl, cycloalkyl and substituted cycloalkyl;
R₂₅ is selected from the group consisting of C₁₋₆ alkyl, C₁₋₆ alkyl-cycloalkyl, alkoxy-substituted alkyl, hydroxylated alkyl, aryl, heteroaryl, substituted aryl or heteroaryl, 5, 6, and 7-membered nitrogen-containing saturated heterocycles, 5,6-fused and 6,6-fused nitrogen-containing saturated heterocycles and these saturated heterocycles can be substituted with C₁₋₆ alkyl, fluorine-substituted C₁₋₆ alkyl, alkoxy, aryl and heteroaryl group;
R₂₆ is selected from the group consisting of C₁₋₆ alkyl, C₃₋₆ cycloalkyl, the alkyl or cycloalkyl can be substituted with —OH, alkoxy, fluorine-substituted alkoxy, fluorine-substituted alkyl, —NH₂, —NH-alkyl, NH—C(O)alkyl, —NH—SO₂— alkyl, and —SO₂-alkyl;
R₂₇ is selected from the group consisting of aryl, heteroaryl, bicyclic heteroaryl, wherein the aryl or heteroaryl groups can be substituted with C₁₋₆ alkyl, alkoxy, NH₂, NH-alkyl, halogen, or —CN, and the substitution can be independently mono-, bis- and tri-substitution;
R₂₈ is selected from the group consisting of aryl, 5 and 6-membered heteroaryl, bicyclic heteroaryl, cycloalkyl, saturated heterocycle such as piperidine, piperidinone, tetrahydropyran, N-acyl-piperidine, wherein the cycloalkyl, saturated heterocycle, aryl or heteroaryl can be further substituted with —OH, alkoxy, mono-, bis- or tri-substitution including halogen, —CN, alkyl sulfone, and fluorine substituted alkyl groups; and
R₁″ is selected from the group consisting of H, alkyl, aryl substituted alkyl, alkoxy substituted alkyl, cycloalkyl, aryl-substituted cycloalkyl, and alkoxy substituted cycloalkyl.

28. The compound of claim 1, wherein L is selected from the group consisting of substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, a bond, —O—, —NH(R^{A})—, —S—, —CO—, —COO—, —CONR^{A}—, —NR^{A}CO, —NR^{A}COR^{A}—, and —COR^{A}—, wherein R^{A} is C₁₋₆ alkyl, wherein one or more carbon is optionally replaced with halo, CO, CONH, O, S, SO, SO₂, N, NHCO, or heterocycle.

29. The compound of claim 28, wherein L is represented by the formula -(A^{L})_{q}-, wherein A^{L} is a chemical moiety and q is greater than or equal to 0.

30. The compound of claim 29, wherein each A^{L} is independently selected from the group consisting of a bond, CR^{L1}R^{L2}, O, S, SO, SO₂, NR^{L3}, SO₂NR^{L3}, SONR^{L3}, CONR^{L3}, NR^{L3}CONR^{W}, NR^{L3}SO₂NR_{W}, CO, CR^{L1}═CR^{L2}, C═C, SiR^{L1}R^{L2}, P(O)R^{L1}, P(O)OR^{L1}, NR^{L3}C(═NCN)NR^{W}, NR^{L3}C(═NCN), NR^{L3}C(═CNO₂)NR^{L4}, C₃₋₁₁ cycloalkyl optionally substituted with 0-6 R^{L1} and/or R^{L2} groups, C₅₋₁₃ spirocycloalkyl optionally substituted with 0-9 R^{L1} and/or R^{L2} groups, C3-diheterocyclyl optionally substituted with 0-6 R^{L1} and/or R^{L2} groups, C5-13 spiroheterocycloalkyl optionally substituted with 0-8 R_{L1} and/or R_{L2} groups, aryl optionally substituted with 0-6 R^{L1} and/or R^{L2} groups, heteroaryl optionally substituted with 0-6 R_{L1} and/or R^{L2} groups, where R^{L1} or R^{L2}, each independently are optionally linked to other groups to form cycloalkyl and/or heterocyclyl moiety, optionally substituted with 0-4 R¹⁵ groups; and R^{L1}, R^{L2}, R^{L3}, R^{L4} and R^{L5} are, each independently, H, halo, C₁₋₈ alkyl, OC₁₋₈ alkyl, SC₁₋₈ alkyl, NHC₁₋₈ alkyl, N(C₁₋₈ alkyl)₂, C₃₋₁₁cycloalkyl, aryl, heteroaryl, C₃₋₁₁ heterocyclyl, OC₁₋₈ cycloalkyl, SC₁₋₈ cycloalkyl, NHC₁₋₈ cycloalkyl, N(C₁₋₈ cycloalkyl)₂, N(C₁₋₈ cycloalkyl)(C₁₋₈alkyl), OH, NH₂, SH, SO₂C₁₋₈ alkyl, P(O)(OC₁₋₈ alkyl)(C₁₋₈alkyl), P(O)(OC₁₋₈alkyl)₂, CC—C₁₋₈alkyl, CCH, CH═H(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NHSO₂NH(C₁₋₈alkyl), NHSO₂N(C₁₋₈alkyl)₂, NHSO₂NH₂.

31. The compound of claim 29, wherein q is greater than or equal to 1.

32. The compound of claim 29, wherein q is 1 to 100.

33. The compound of claim 29, wherein each A^{L} is independently selected from the group consisting of: —NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(hetero cycloalkyl)-, —NR(CH₂CH₂O)ₙ-(lower alkyl)-O—CH₂—, —NR(CH₂CH₂O)ₙ-(hetero cycloalkyl)-O—CH₂—, —NR(CH₂CH₂O)ₙ-Aryl-O—CH₂—, NR(CH₂CH₂O)ₙ-(hetero aryl)-O—CH₂—, —NR(CH₂CH₂O)ₙ-(cycloalkyl)-O-(hetero aryl)-O—CH₂—, —NR(CH₂CH₂O)ₙ-(cycloalkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)l-, —NR(CH₂CH₂)ₙ-(cycloalkyl)-O-(heterocycle)-CH₂, —NR(CH₂CH₂)ₙ-(heterocycle)-(heterocycle)-CH₂, N(R₁R₂)-(heterocycle)-CH₂; wherein:
n of the linker can be 0 to 10;
R of the linker can be H, lower alkyl; and
R₁ and R₂ of the linker can form a ring with the connecting N.

34. The compound of claim 29, wherein A^{L} is selected from the group consisting of: 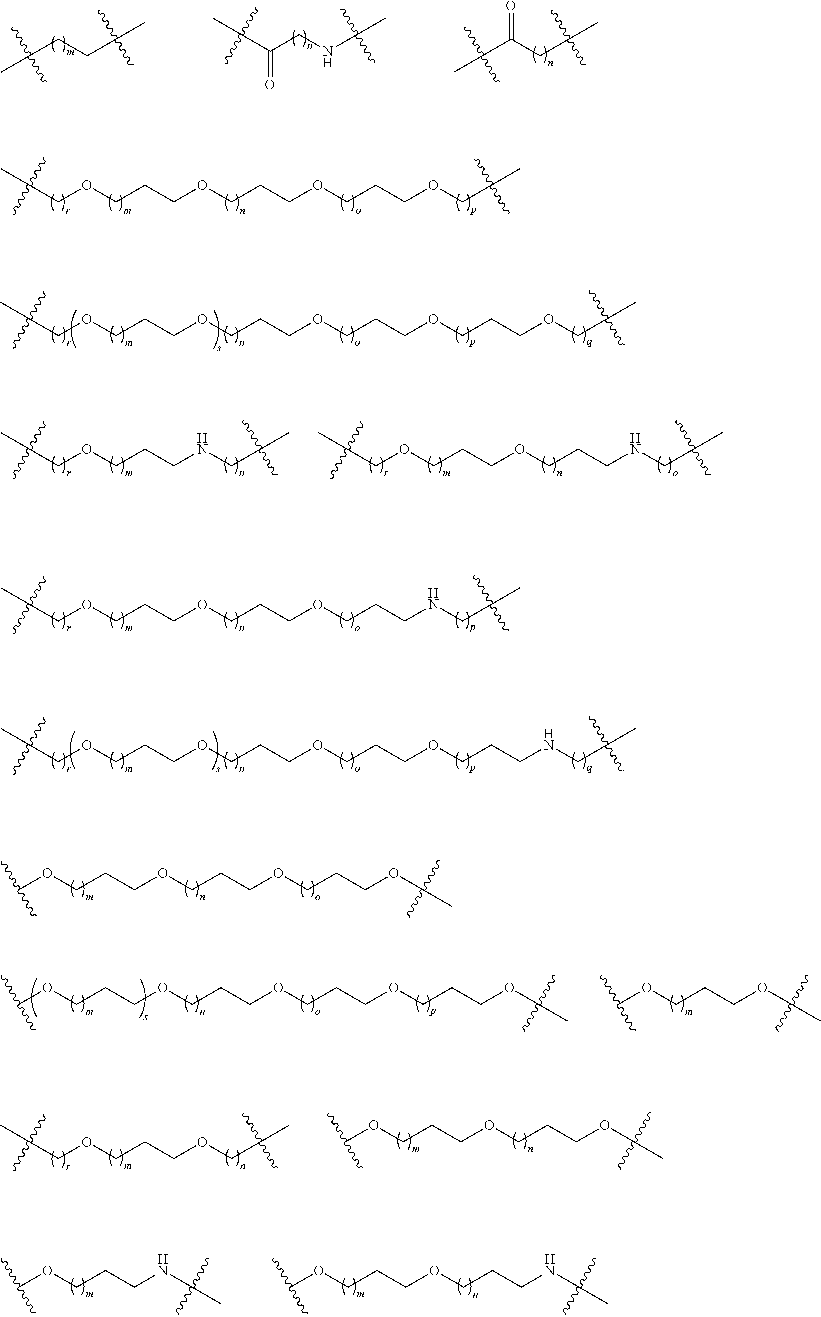 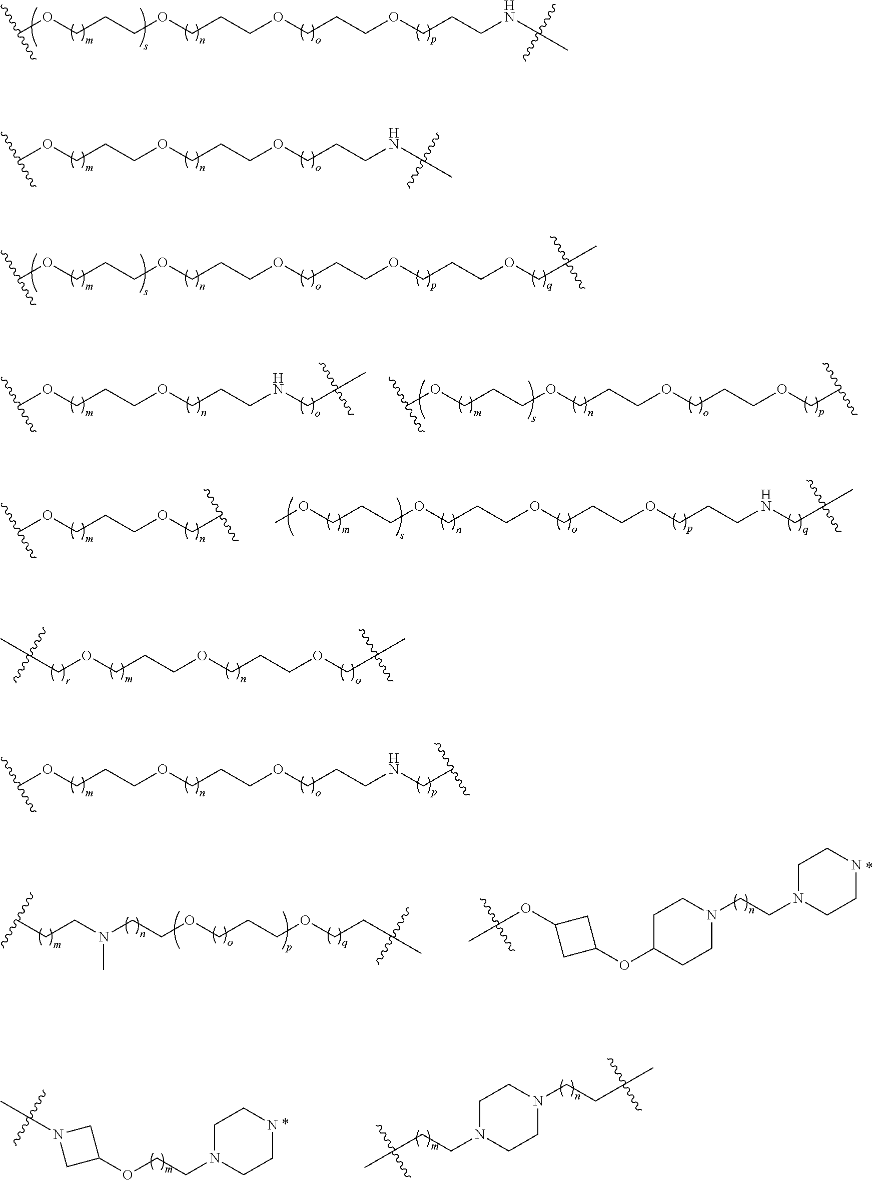 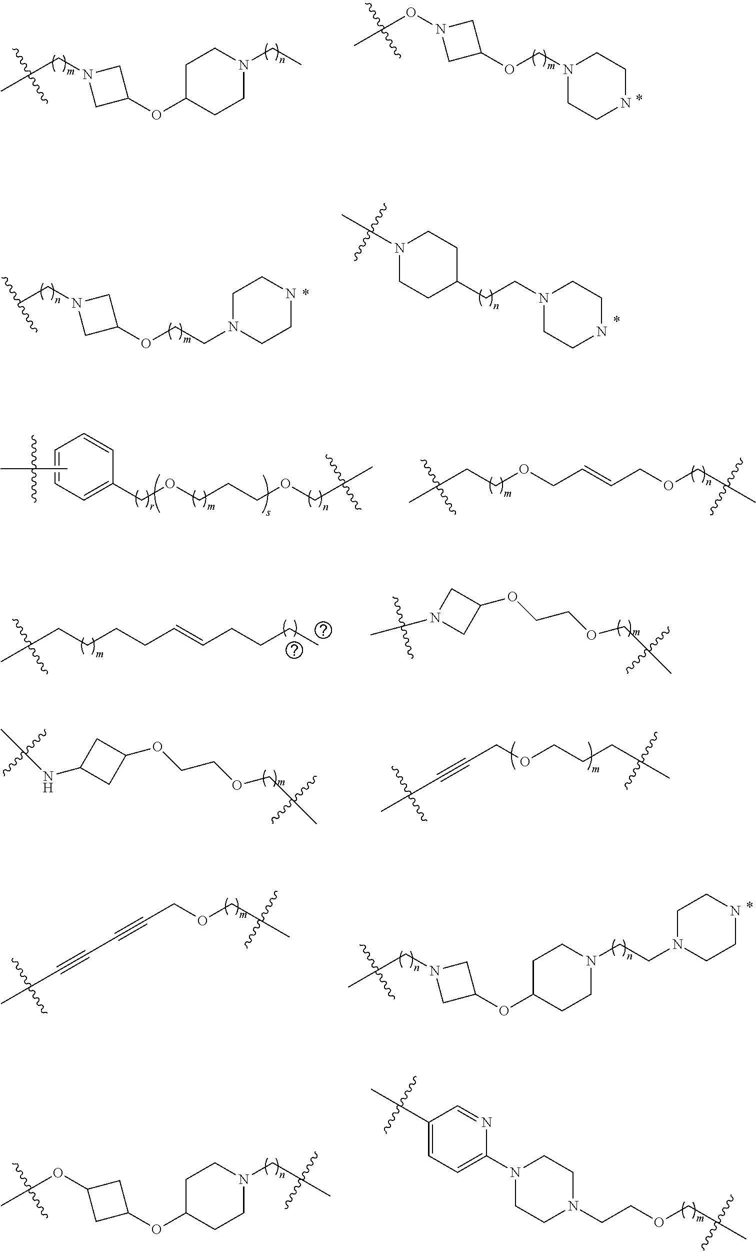 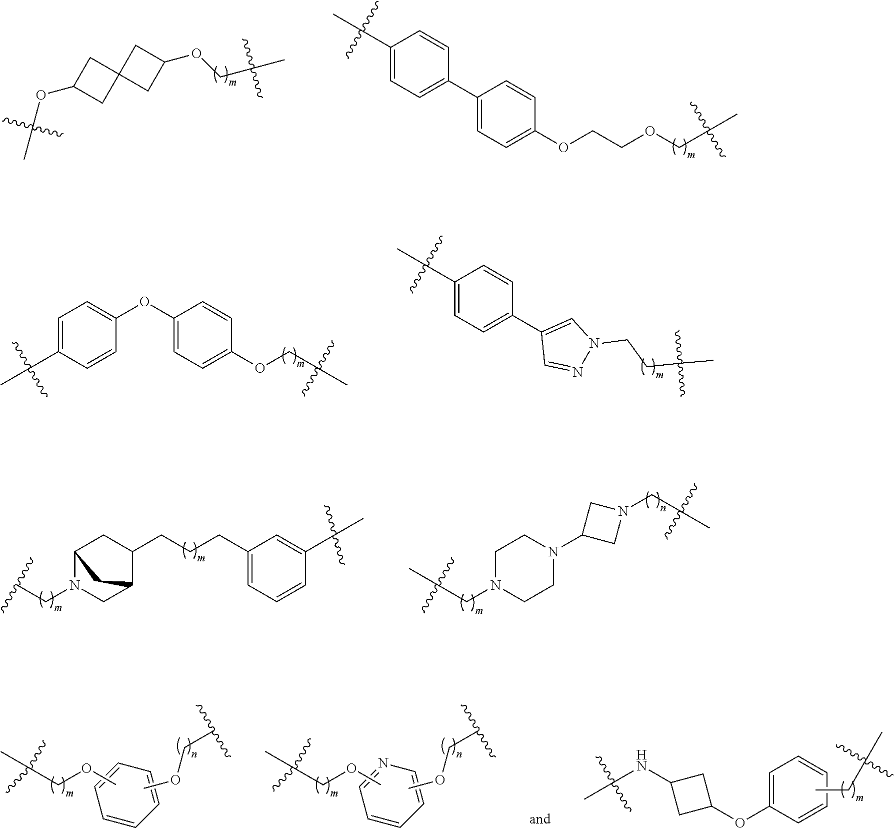
wherein:
each of m, n, o, p, q, r, and s are independently selected from 0-10, and N* of the heterocycloalkyl is shared with the TPM or the ELM or is linked to the TPM or the ELM via a bond.

35. The compound of claim 1, wherein the compound has the following formula: 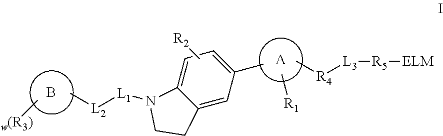
or a pharmaceutically acceptable salt, co-crystal, tautomer, stereoisomer, solvate, hydrate, polymorph, isotopically enriched derivative, or prodrug thereof, wherein:
ELM is an E3 ubiquitin ligase binding moiety;
Ring A and Ring B are each independently selected from a 5-10 membered aryl ring or a heterocycle ring;
L₁ is —S(O)—, —S(O)₂—, or —C(O)—;
L₂ is a bond, —N(R₁)— or —CH(R₁)—;
L₃ is —(CH₂)ₙ— or —(OCH₂CH₂)ₙ—;
R₁, R₂, R₃ are each independently hydrogen, halogen, —OH, —OR₄, —NH₂, —NR₄R₅, —NR₄COR₅, —CN, —COOH, —COOR₄, —CONH₂, —CONR₄R₅, —CF₃, —OCF₃, —SO₃H, —SO₃R₄, R₄, tetrazole, aryl, aryl substituted with from one to three substituents independently selected from halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, R₄, heterocycle, heterocycle substituted with from one to three substituents independently selected from halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, and R₄;
R₄ and R₅ are independently a bond or C₁-12 alkyl, wherein one or more carbons are optionally replaced with halo, CO, CONH, O, S, SO, SO₂, N, NHCO, heterocycle;
n is independently an integer from 0 to 15; and
w is independently an integer from 0 to 4.

36. The compound of claim 35, wherein:
Ring A is selected from the following bicycloheteroaryls: 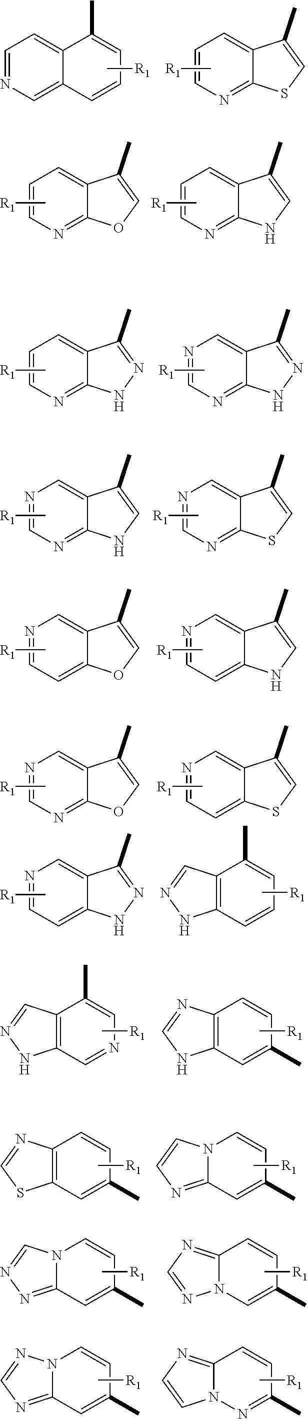 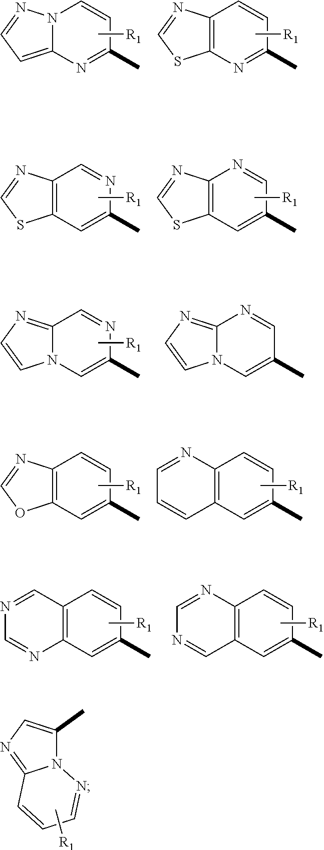
Ring B is independently phenyl or 6 membered heterocycle ring;
L₁ is —C(O)—;
L₂ is —N(R₁)— or —CH(R₁)—;
L₃ is —(CH₂)ₙ— or —(OCH₂CH₂)ₙ—;
R₁ is selected from hydrogen, halogen, —OH, —OR₄, —NH₂, —NR₄R₅, —NR₄COR₅, —CN, —CONH₂, —CONR₄R₅, —CF₃, —OCF₃, tetrazole, aryl, heterocycle;
R₂ and R₃ are independently hydrogen, halogen, —OH, —OR₄, —NH₂, —NR₄R₅, —NR₄COR₅, —CN, —CONR₄R₅, —CF₃, —OCF₃;
R₄ and R₅ are independently a bond or C₁₋₆ alkyl, wherein one or more carbon is optionally replaced with halo, CO, CONH, O, SO₂, N, NHCO;
n is independently an integer from 0 to 10; and
w is independently an integer from 0 to 3.

37. The compound of claim 35, wherein:
Ring A is selected from the following bicycloheteroaryls: 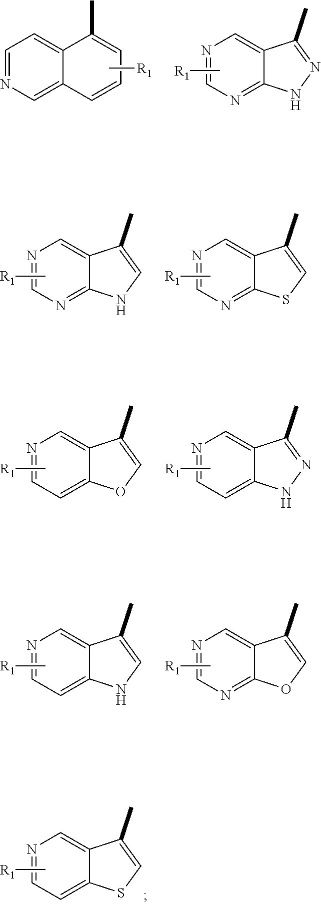
Ring B is independently phenyl, pyridine, pyrimidine, pyridazine, pyrazine;
L₁ is —C(O)—;
L₂ is —N(R₁)-or —CH(R₁)—;
L₃ is —(CH₂)ₙ— or —(OCH₂CH₂)ₙ—;
R₁ is selected from hydrogen, halogen —OR₄, —NH₂, —NR₄R₅, —NR₄COR₅, —CONR₄R₅, —CF₃, —OCF₃;
R₂, R₃ are independently hydrogen, halogen, —OH, —OMe, —NH₂, —CN, —CONH₂, —CF₃, —OCF₃;
R₄ and R₅ are independently a bond or C₁₋₆ alkyl, wherein one or more carbon is optionally replaced with halo, CO, CONH, O, SO₂, N, NHCO;
n is independently an integer from 0 to 18; and
w is independently an integer from 0 to 3.

38. The compound of claim 1, wherein the compound has the following formula: 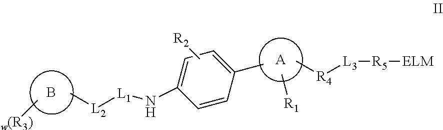
or a pharmaceutically acceptable salt, co-crystal, tautomer, stereoisomer, solvate, hydrate, polymorph, isotopically enriched derivative, or prodrug thereof, wherein:
ELM is an E3 ubiquitin ligase binding moiety;
Ring A and Ring B are independently selected from 5-10 membered aryl ring or heterocycle ring;
L₁ is —S(O)—, —S(O)₂—, or —C(O)—;
L₂ is a bond, —N(R₁)— or —CH(R₁)—;
L₃ is —(CH₂)ₙ— or —(OCH₂CH₂)ₙ—;
each of R₁, R₂, R₃ are independently hydrogen, halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, R₄, tetrazole, aryl, aryl substituted with from one to three substituents independently selected from halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, R₄, heterocycle, heterocycle substituted with from one to three substituents independently selected from halogen, —OH, —OR₄, —NH₂, —NR₄R₅, —NR₄COR₅, —CN, —COOH, —COOR₃, —CONH₂, —CONR₄R₅, —CF₃, —OCF₃, —SO₃H, —SO₃R₄, R₄;
R₄ and R₅ are independently a bond or C₁-12 alkyl, wherein one or more carbon is optionally replaced with halo, CO, CONH, O, S, SO, SO₂, N, NHCO, or heterocycle;
n is independently an integer from 0 to 15; and
w is independently an integer from 0 to 4.

39. The compound of claim 38, wherein
Ring A is selected from the following bicycloheteroaryls: 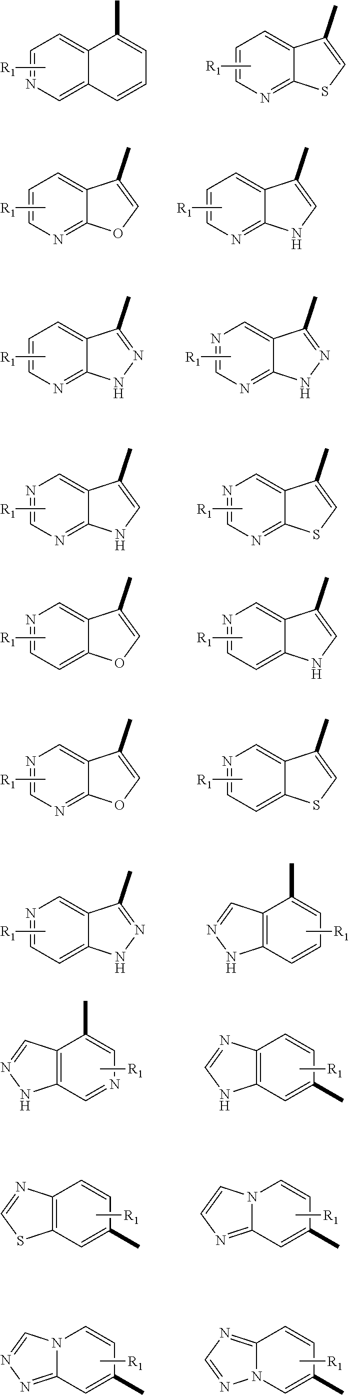 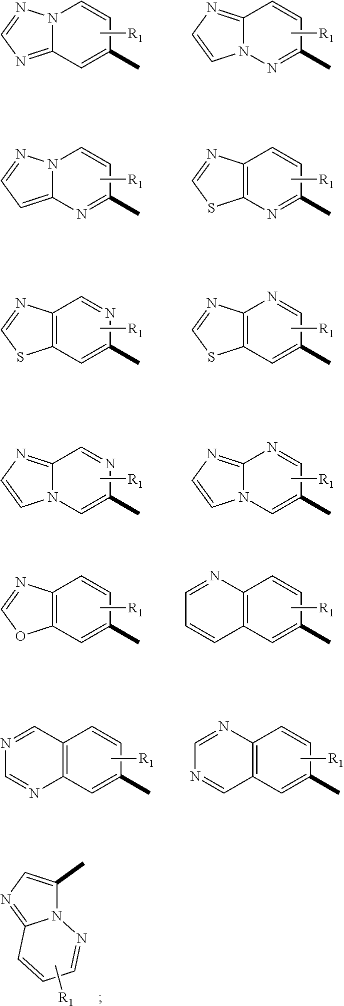
Ring B is independently phenyl or 6 membered heterocycle ring;
L₁ is —C(O)—;
L₂ is —N(R₁)-or —CH(R₁)—;
L₃ is —(CH₂)ₙ— or —(OCH₂CH₂)ₙ—;
R₁ is selected from hydrogen, halogen, —OH, —OR₄, —NH₂, —NR₄R₅, —NR₄COR₅, —CN, —CONH₂, —CONR₄R₅, —CF₃, —OCF₃, tetrazole, aryl, heterocycle;
R₂ and R₃ are independently hydrogen, halogen, —OH, —OR₄, —NH₂, —NR₄R₅, —NR₄COR₅, —CN, —CONR₄R₅, —CF₃, —OCF₃;
R₄ and R₅ are independently a bond or C₁₋₆ alkyl, wherein one or more carbon is optionally replaced with halo, CO, CONH, O, SO₂, N, NHCO;
n is independently an integer from 0 to 10; and
w is independently an integer from 0 to 3.

40. The compound of claim 38, wherein:
Ring A is selected from the following bicycloheteroaryls: 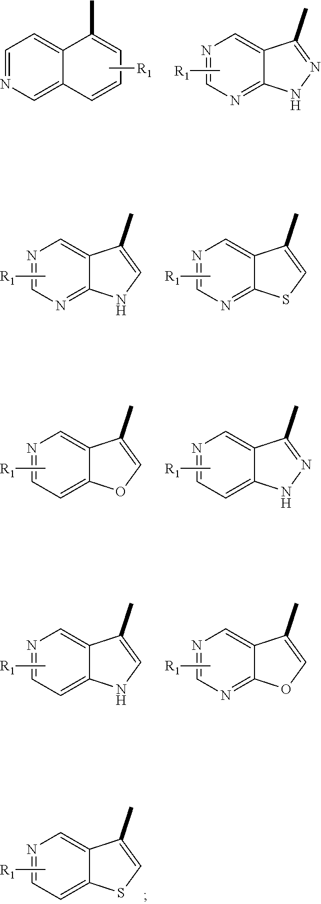
Ring B is independently phenyl, pyridine, pyrimidine, pyridazine, pyrazine;
L₁ is —C(O)—;
L₂ is —N(R₁)-or —CH(R₁)—;
L₃ is —(CH₂)ₙ— or —(OCH₂CH₂)ₙ—;
R₁ is selected from hydrogen, halogen —OR₄, —NH₂, —NR₄R₅, —NR₄COR₅, —CONR₄R₅, —CF₃, —OCF₃;
R₂ and R₃ are independently hydrogen, halogen, —OH, —OMe, —NH₂, —CN, —CONH₂, —CF₃, —OCF₃;
R₄ and R₅ are independently a bond or C₁₋₆ alkyl, wherein one or more carbon is optionally replaced with halo, CO, CONH, O, SO₂, N, NHCO;
n is independently an integer from 0 to 18; and
w is independently an integer from 0 to 3.

41. The compound of claim 1, wherein the compound has the following formula: 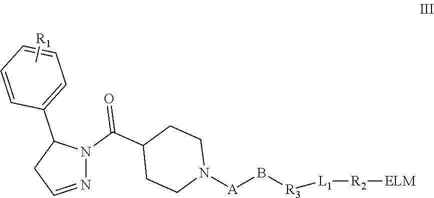
or a pharmaceutically acceptable salt, co-crystal, tautomer, stereoisomer, solvate, hydrate, polymorph, isotopically enriched derivative, or prodrug thereof, wherein:
ELM is an E3 ubiquitin ligase binding moiety;
A is —CO—, one or two R₁ substituted 5-10 membered aryl ring or one or two R₁ substituted heteroaryl ring;
B is a bond, one or two R₁ substituted 5-10 membered aryl ring, or one or two R₁ substituted heteroaryl ring;
R₁ is independently hydrogen, halogen, —OH, —OR₂, —NH₃, —NR₂R₃, —NR₂COR₃, —CN, —COOH, —COOR₃, —CONH₂, —CONR₂R₃, —CF₃, —OCF₃, —SO₃H, —SO₃R₃, R₃;
R₂ and R₃ are independently a bond or C₁₋₆ alkyl, wherein one or more carbon is optionally replaced with halo, CO, CONH, O, S, SO, SO₂, N, NHCO;
L₁ is —(CH₂)ₙ— or —(OCH₂CH₂)ₙ—; and
n is independently an integer from 0 to 20.

42. The compound of claim 41, wherein:
R₁ is independently hydrogen, halogen, —OH, —OMe, —NH₃, —CN, —CONH₂, —CONR₂R₃, —CF₃, —OCF₃;
R₂ and R₃ are independently C₁₋₆ alkyl, wherein one or more carbon is optionally replaced with halo, CO, CONH, O, S, SO, SO₂, N, NHCO;
L₁ is —(CH₂)ₙ— or —(OCH₂CH₂)ₙ—; and
n is independently an integer from 0 to 10.

43. The compound of claim 1, wherein the compound is selected from the group consisting of: 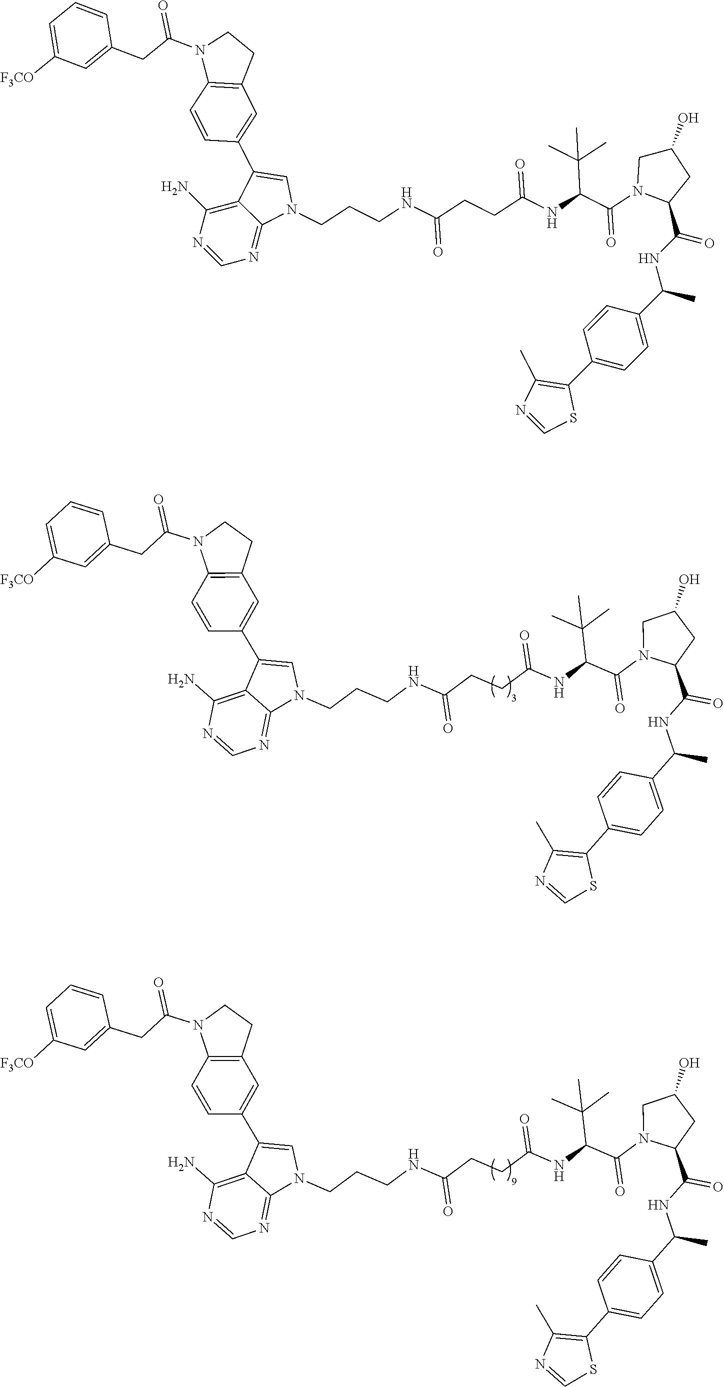 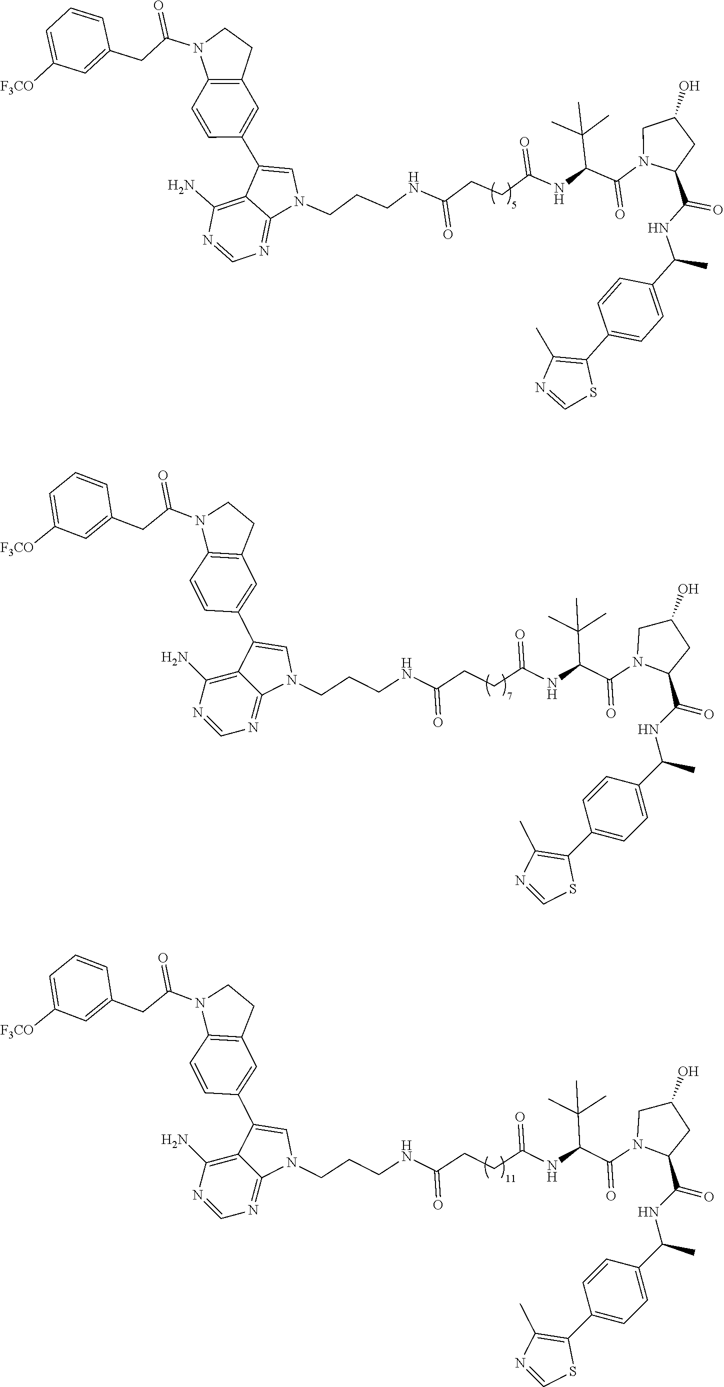 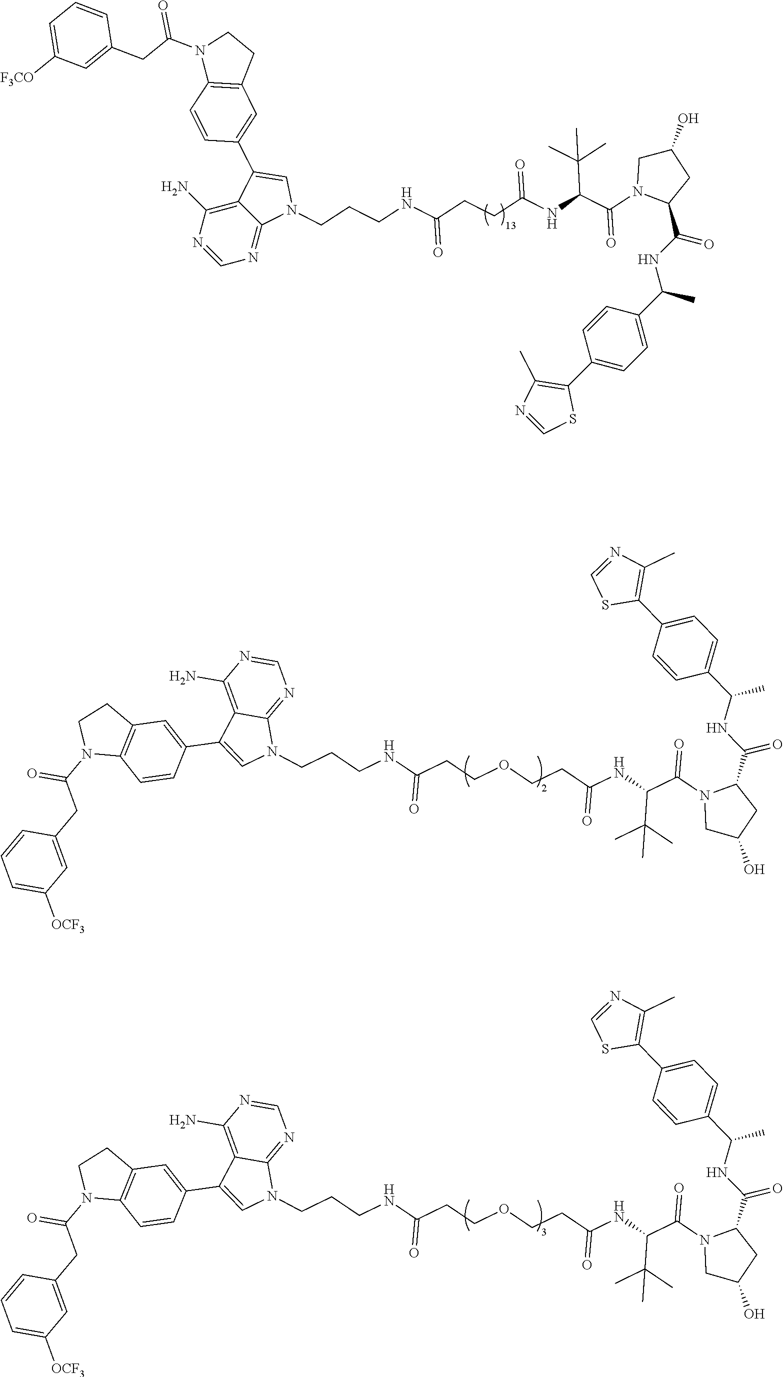 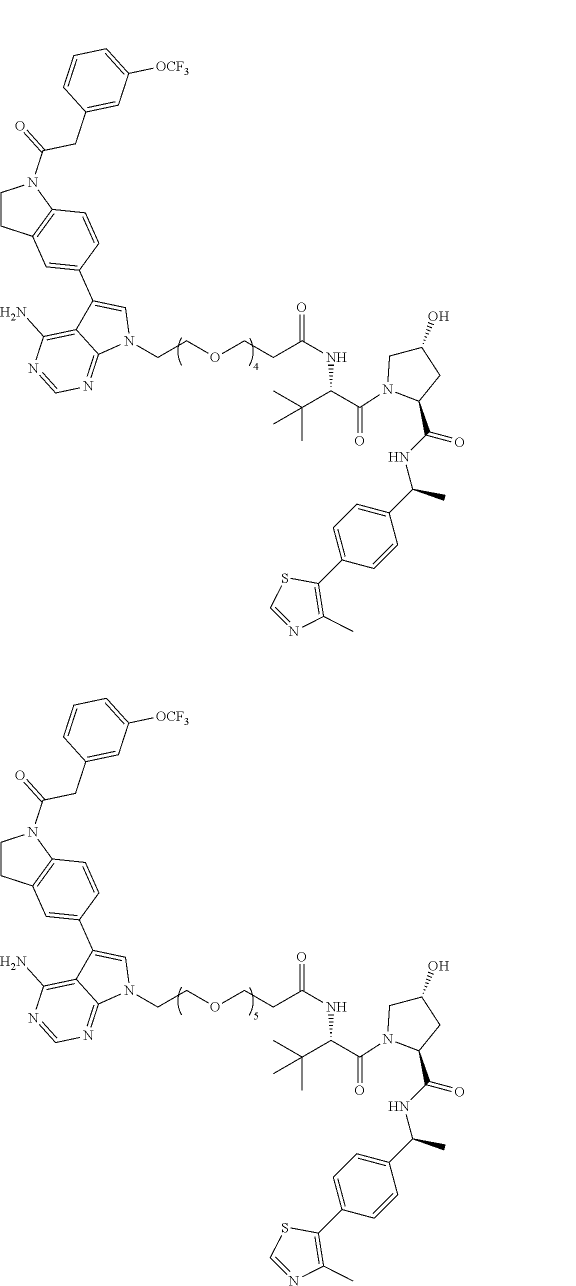 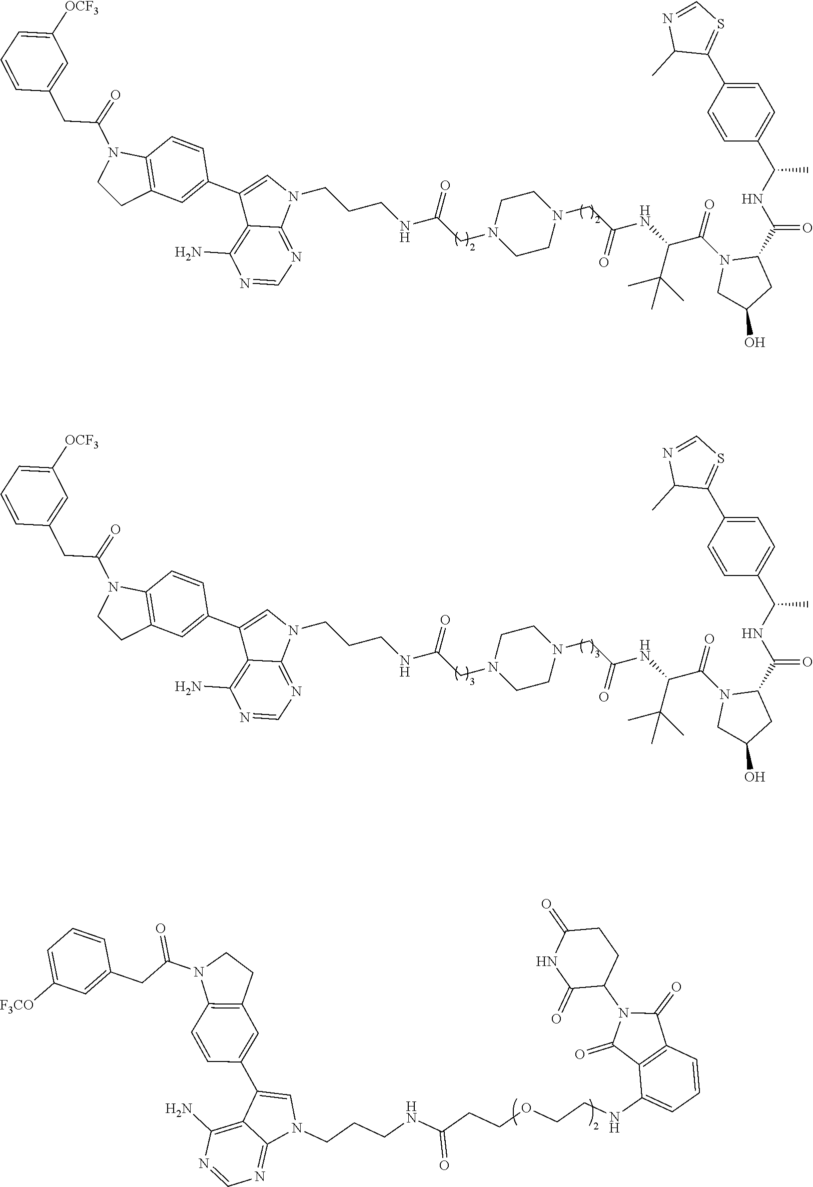 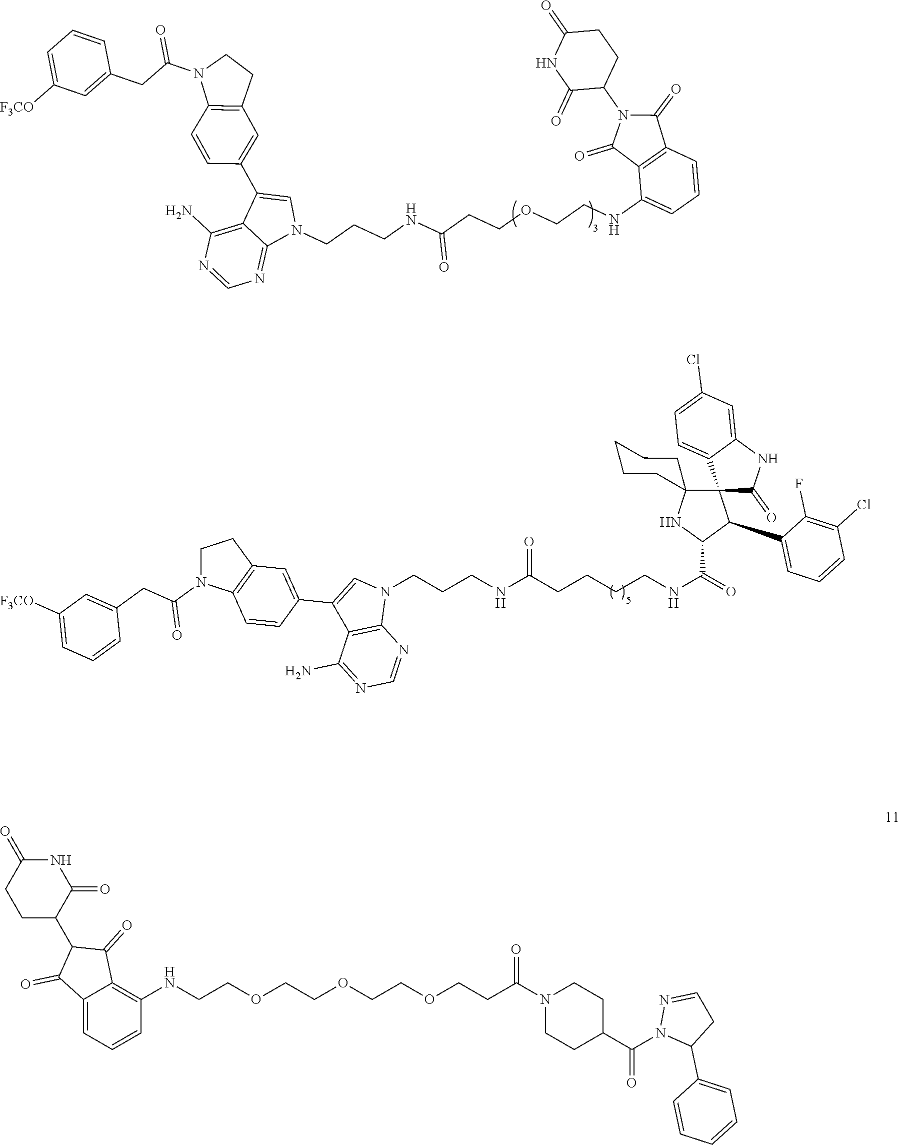 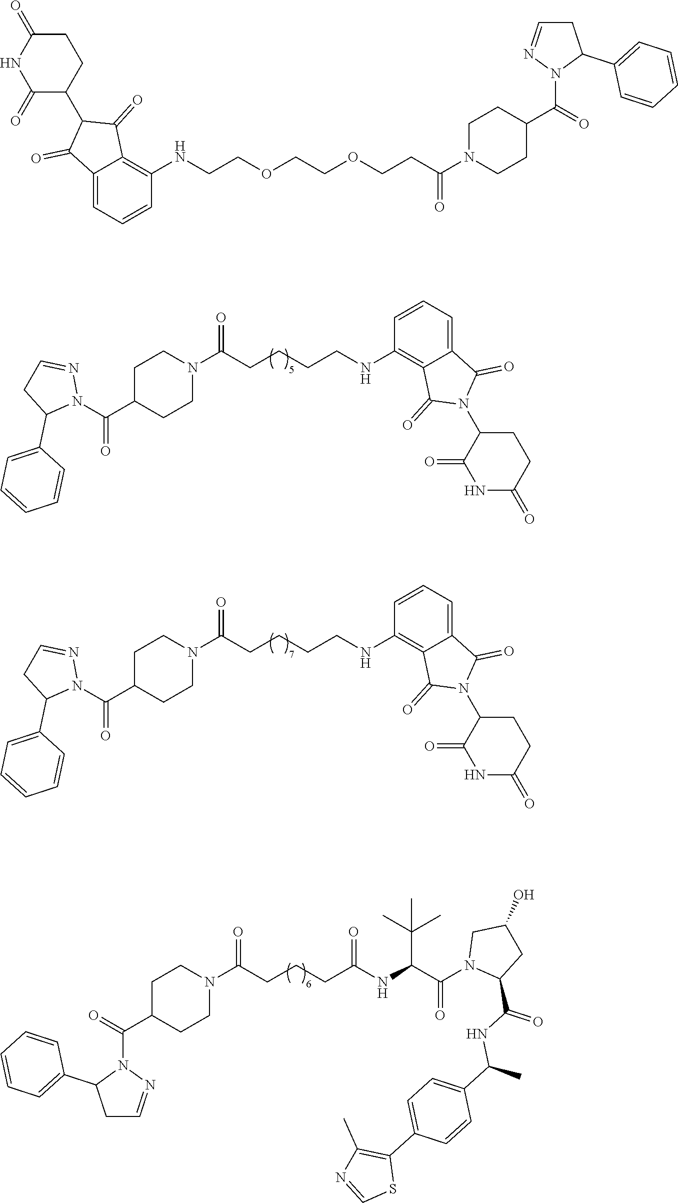 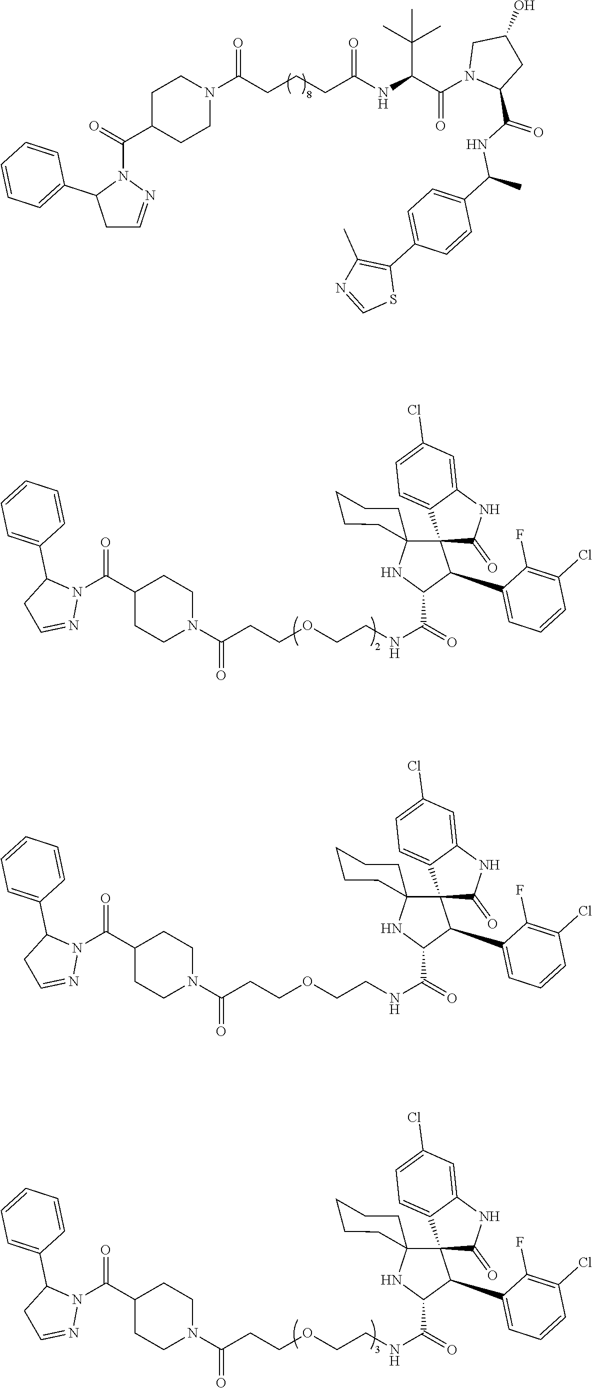 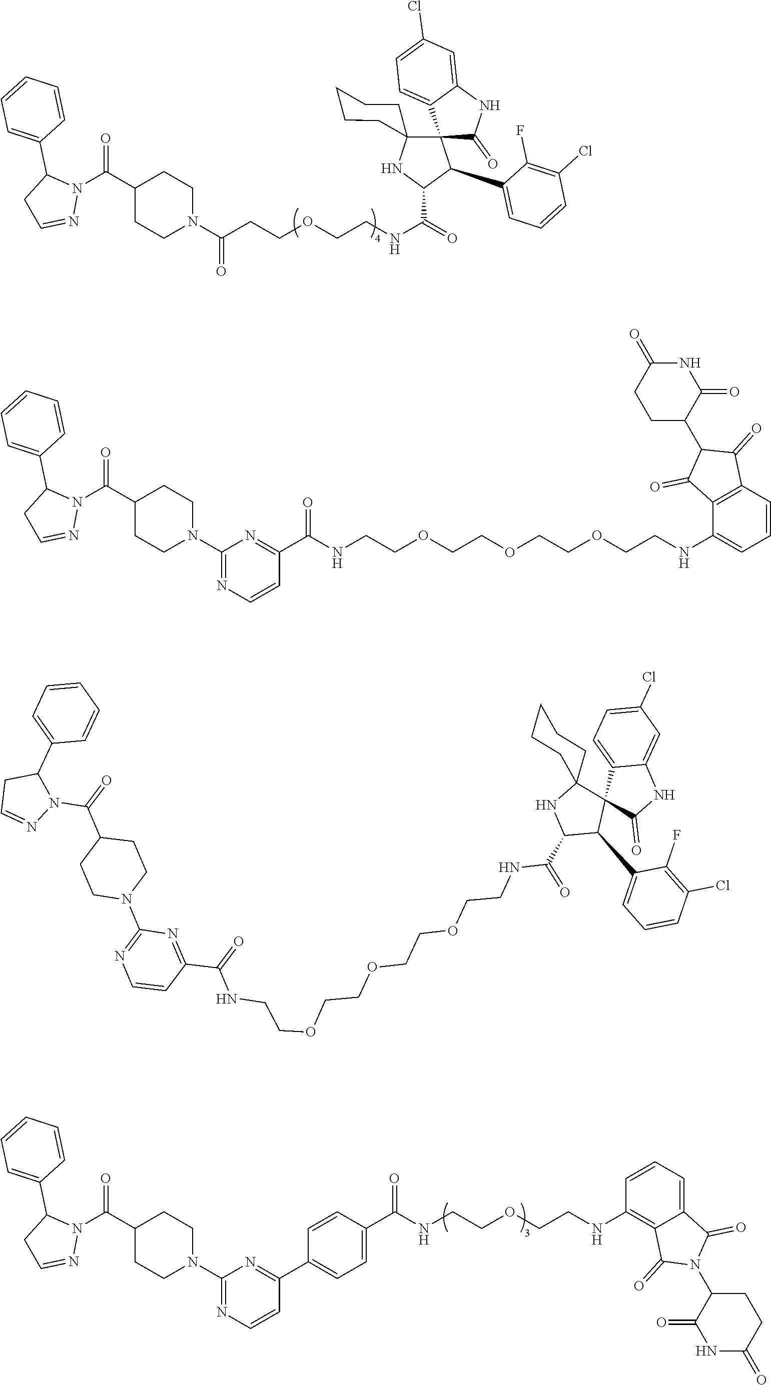 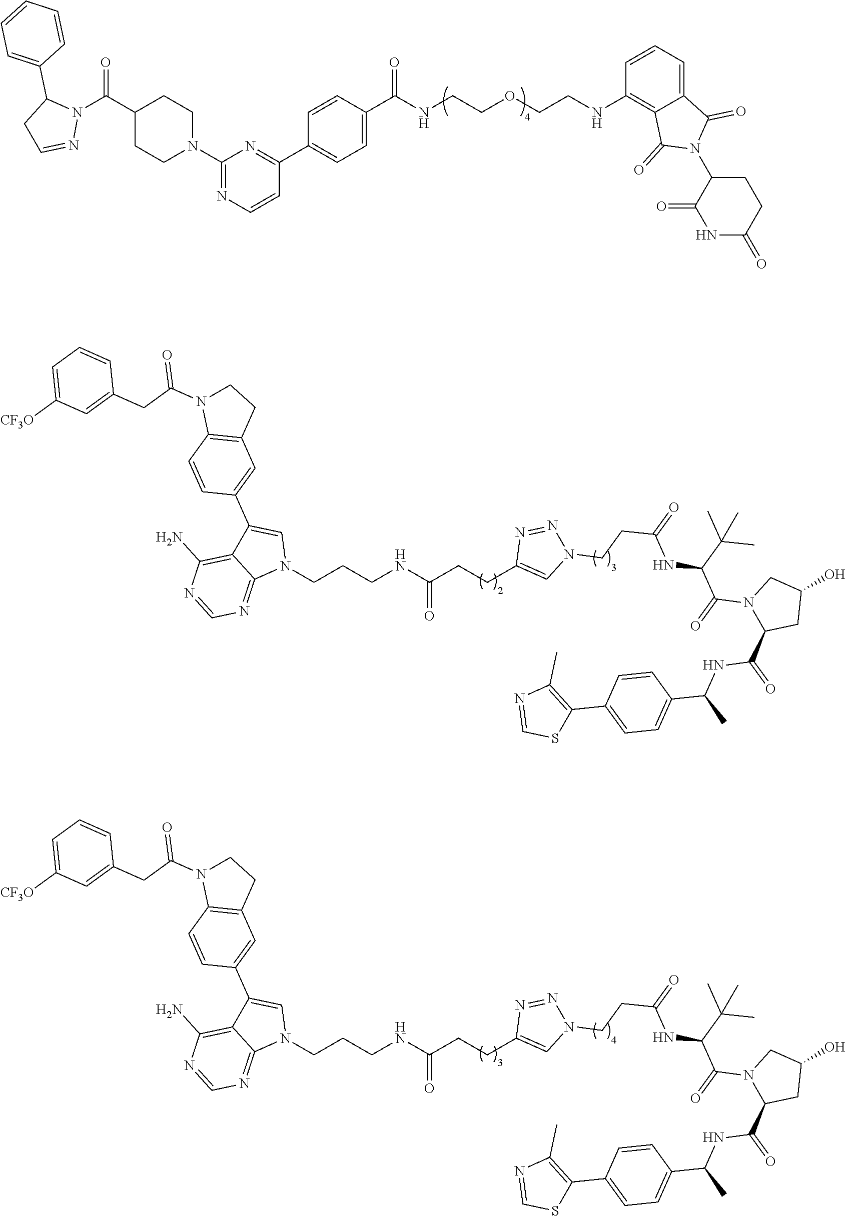 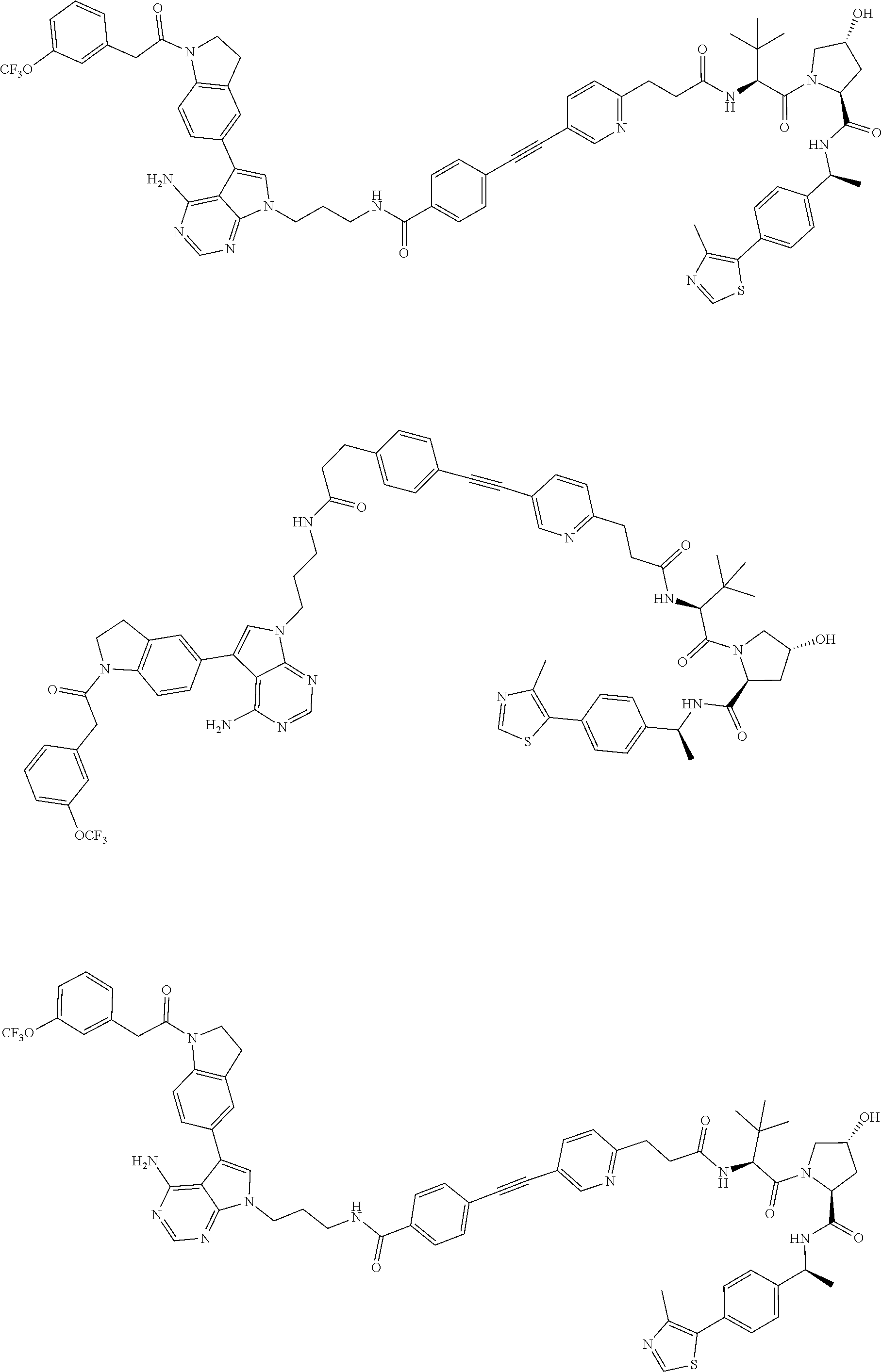 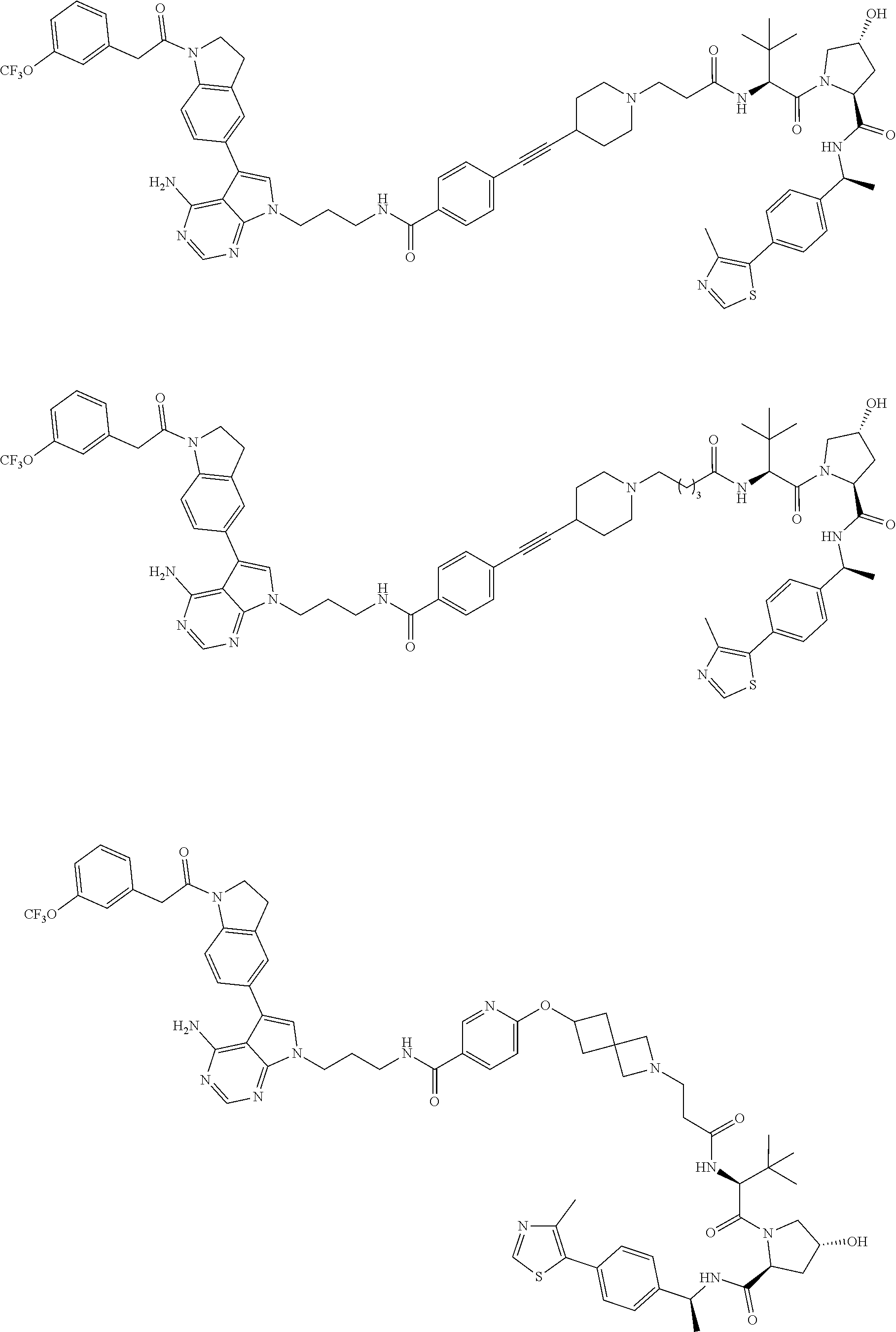 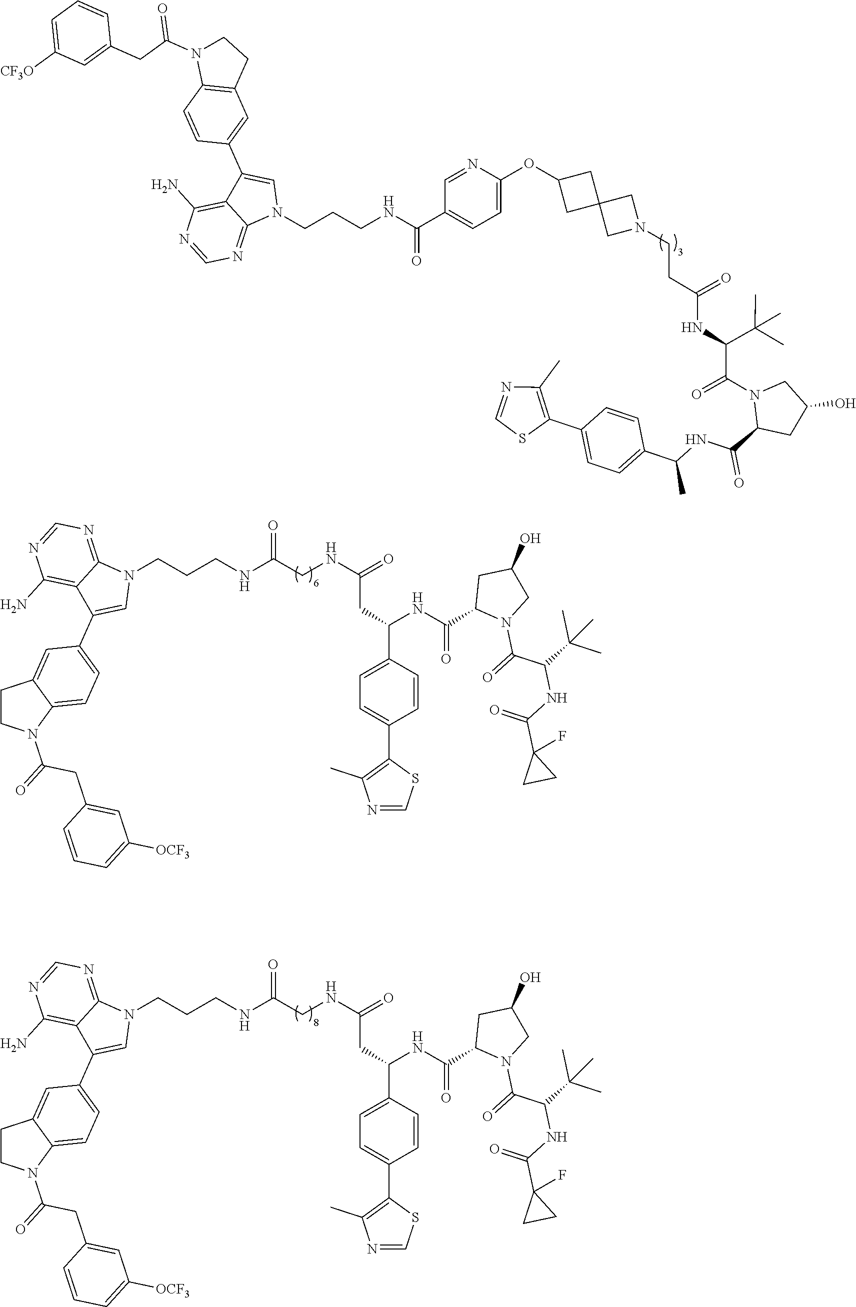 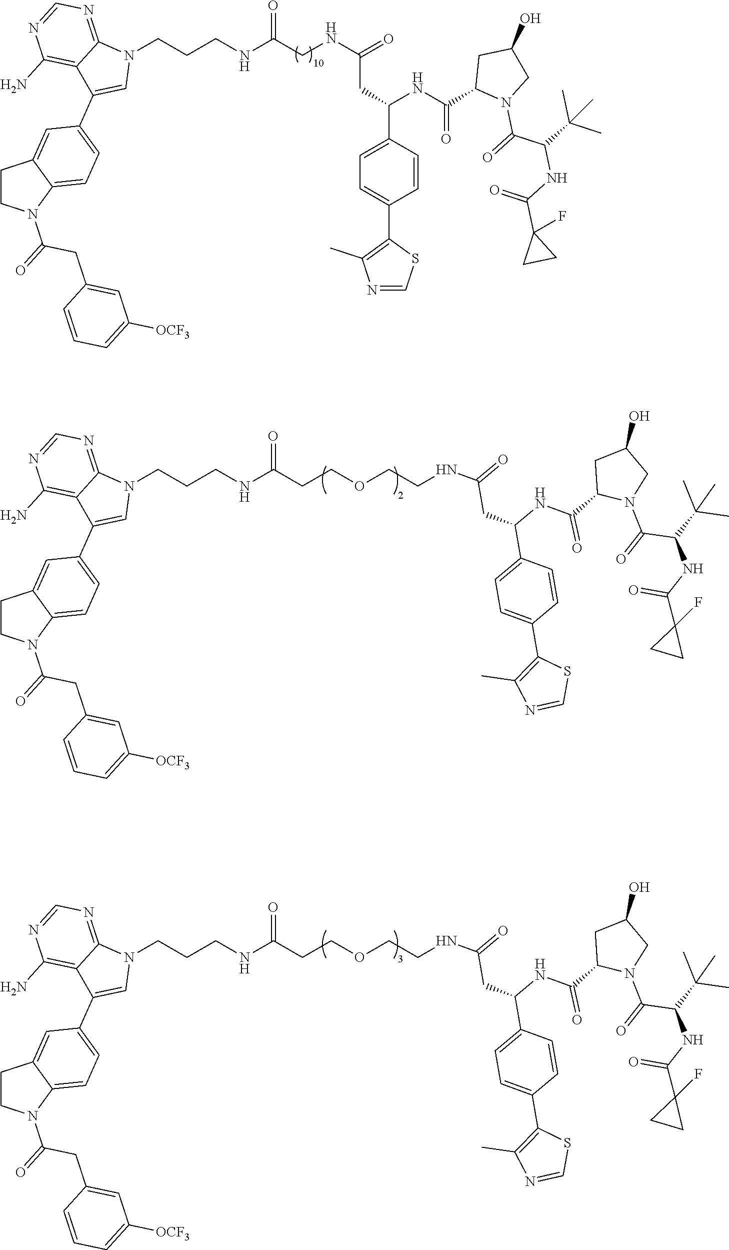 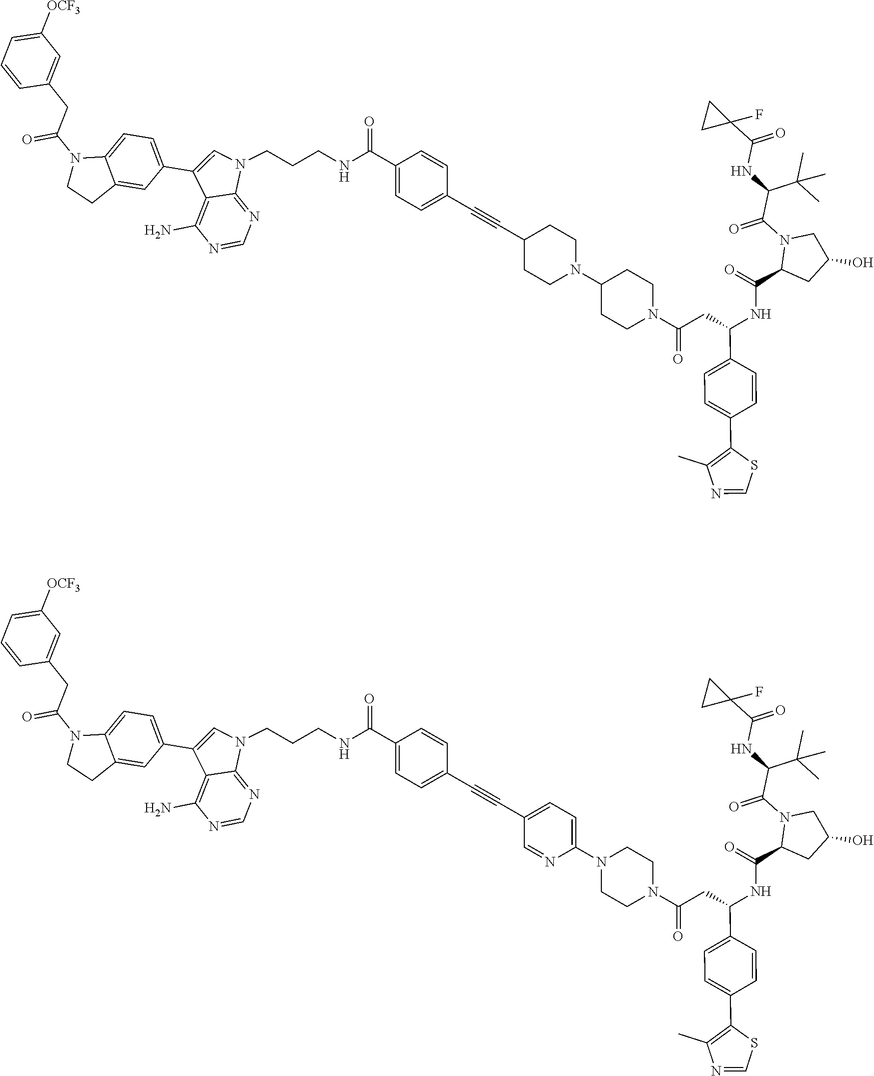 [SMILES: Cc1ncsc1-c1ccc([C@H](CC(=O)N2CCC(N3CCC(C#Cc4ccc(C(=O)NCCCn5cc(-c6ccc7c(c6)CCN7C(=O)Cc6cccc(OC(F)(F)F)c6)c6c(N)ncnc65)cc4)CC3)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)N2CCN(c3ccc(C#Cc4ccc(C(=O)NCCCn5cc(-c6ccc7c(c6)CCN7C(=O)Cc6cccc(OC(F)(F)F)c6)c6c(N)ncnc65)cc4)cn3)CC2)NC(=O)[C@@H]2C[C@@H](O)CN2C(=O)[C@@H](NC(=O)C2(F)CC2)C(C)(C)C)cc1] 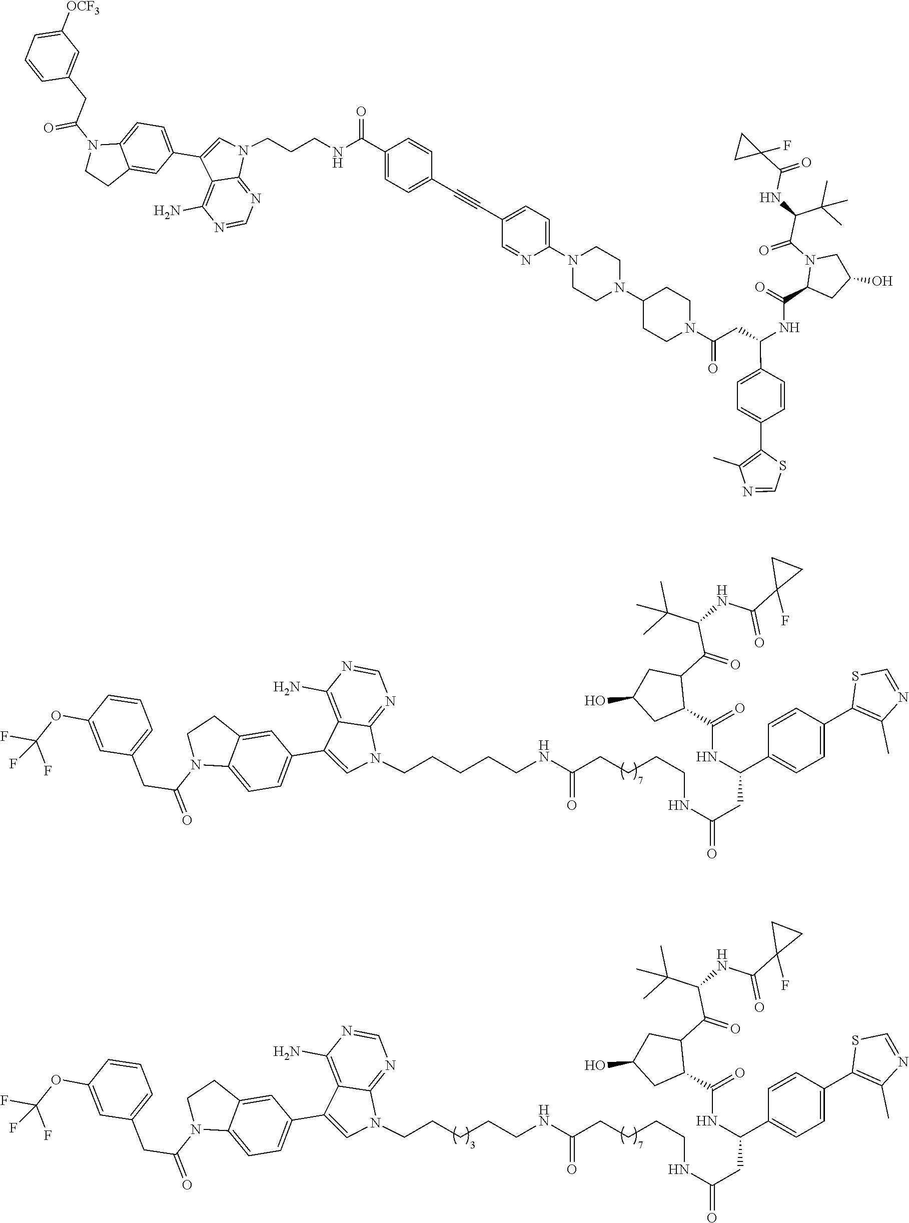 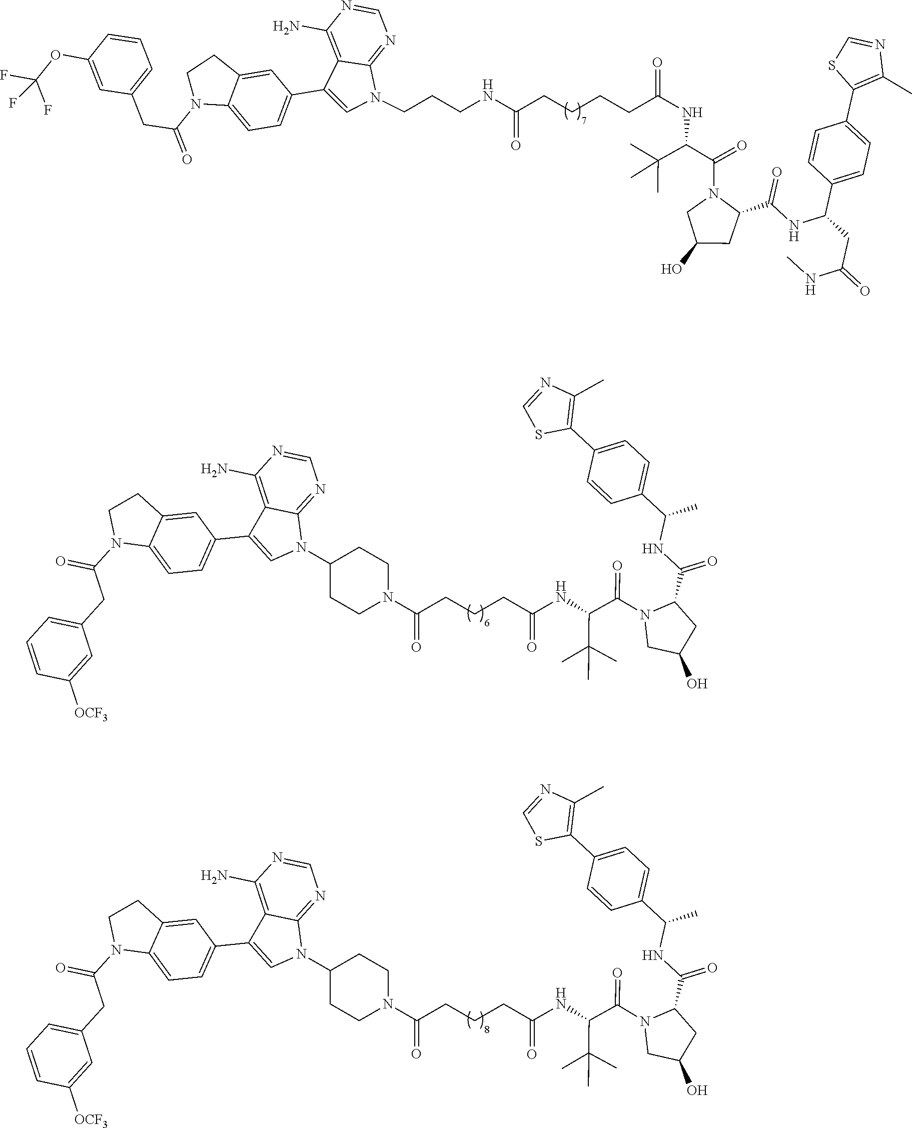 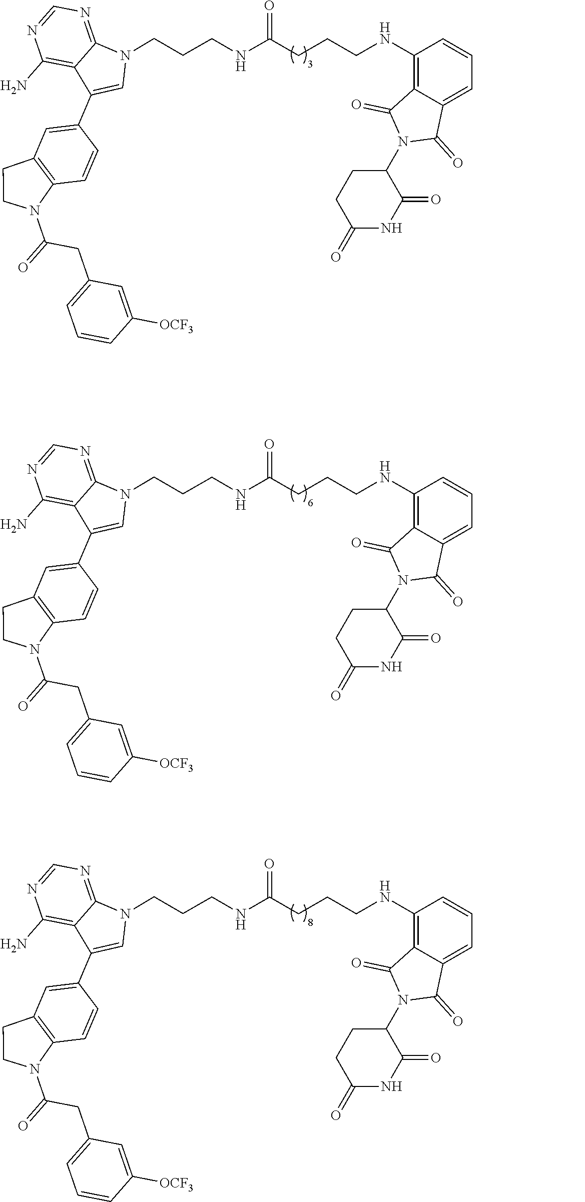 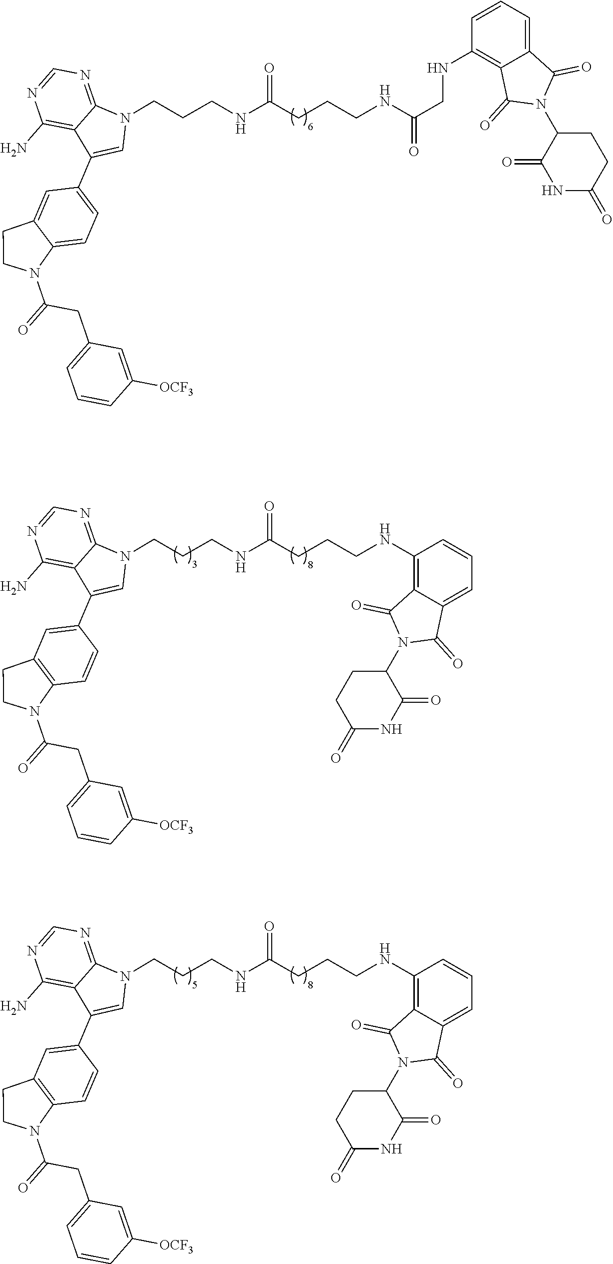 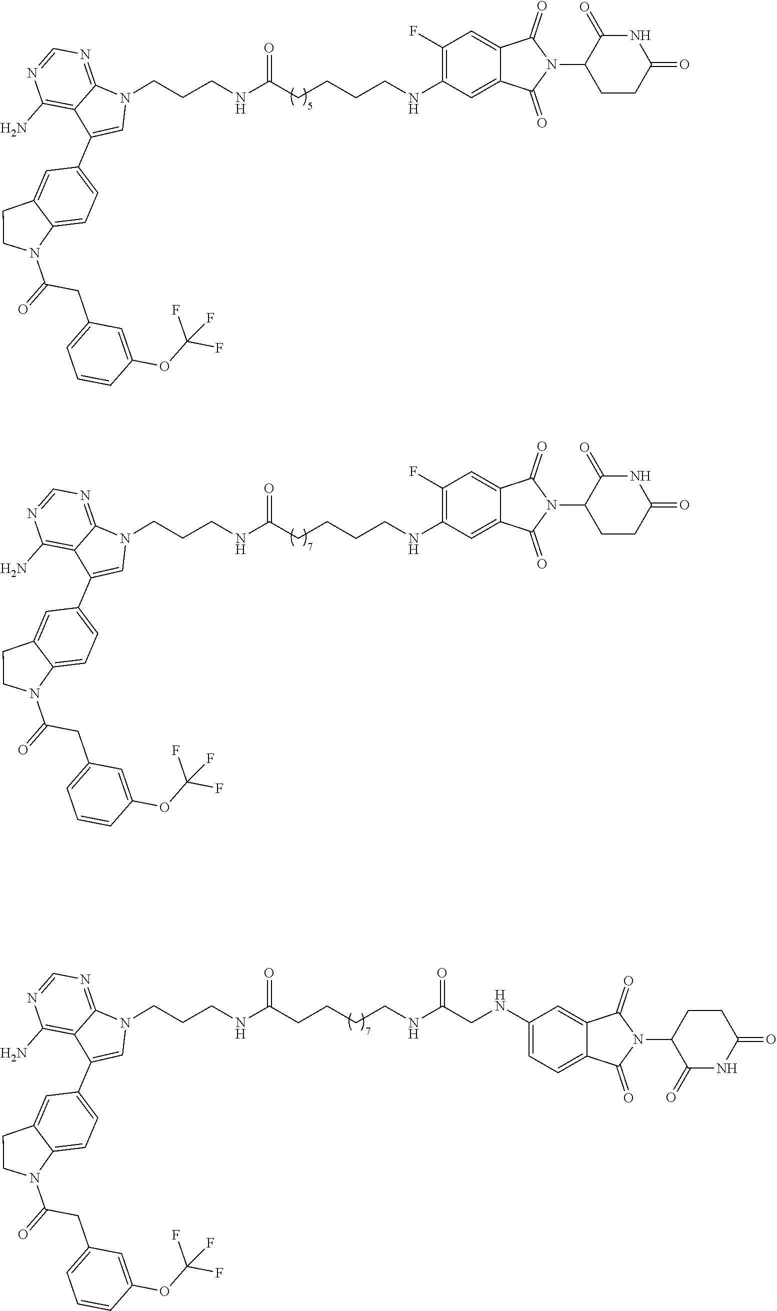 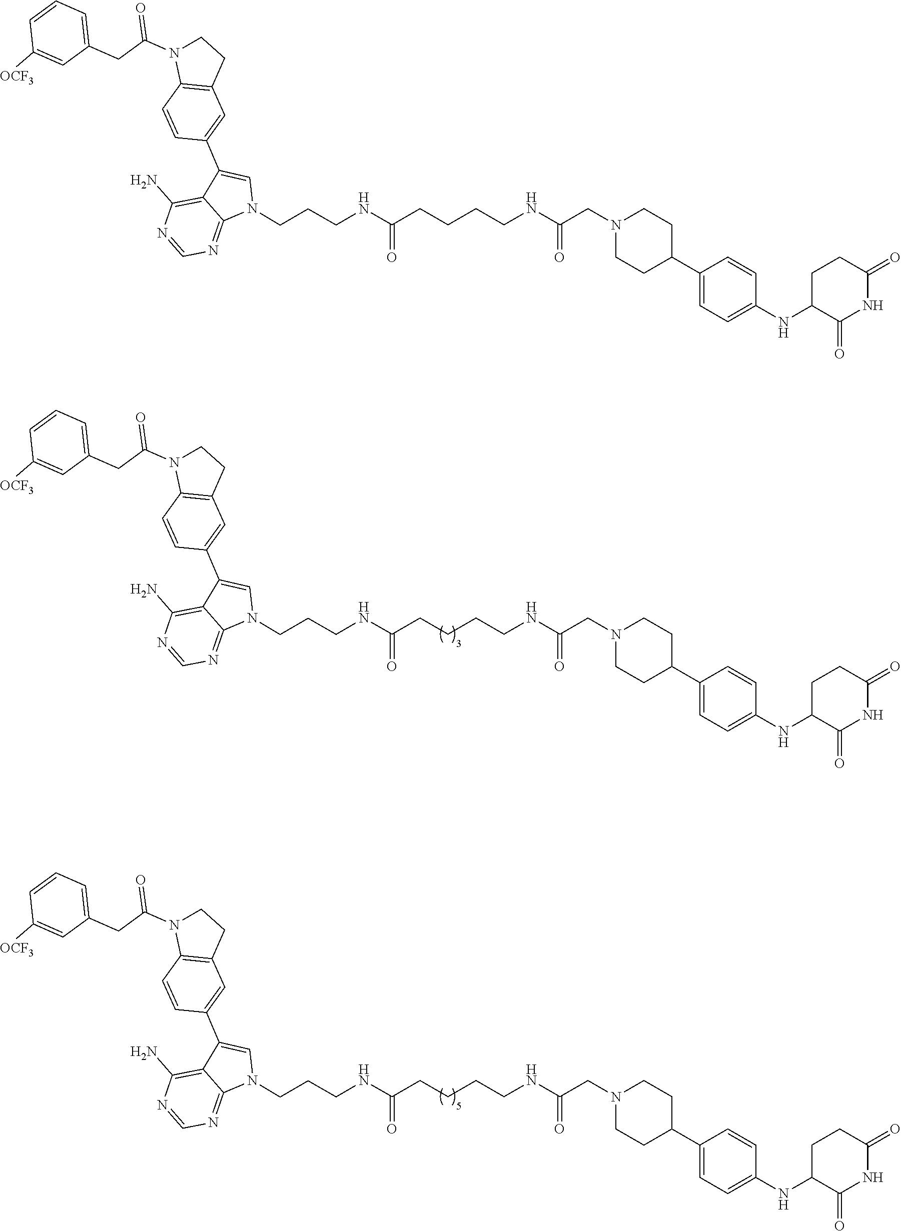 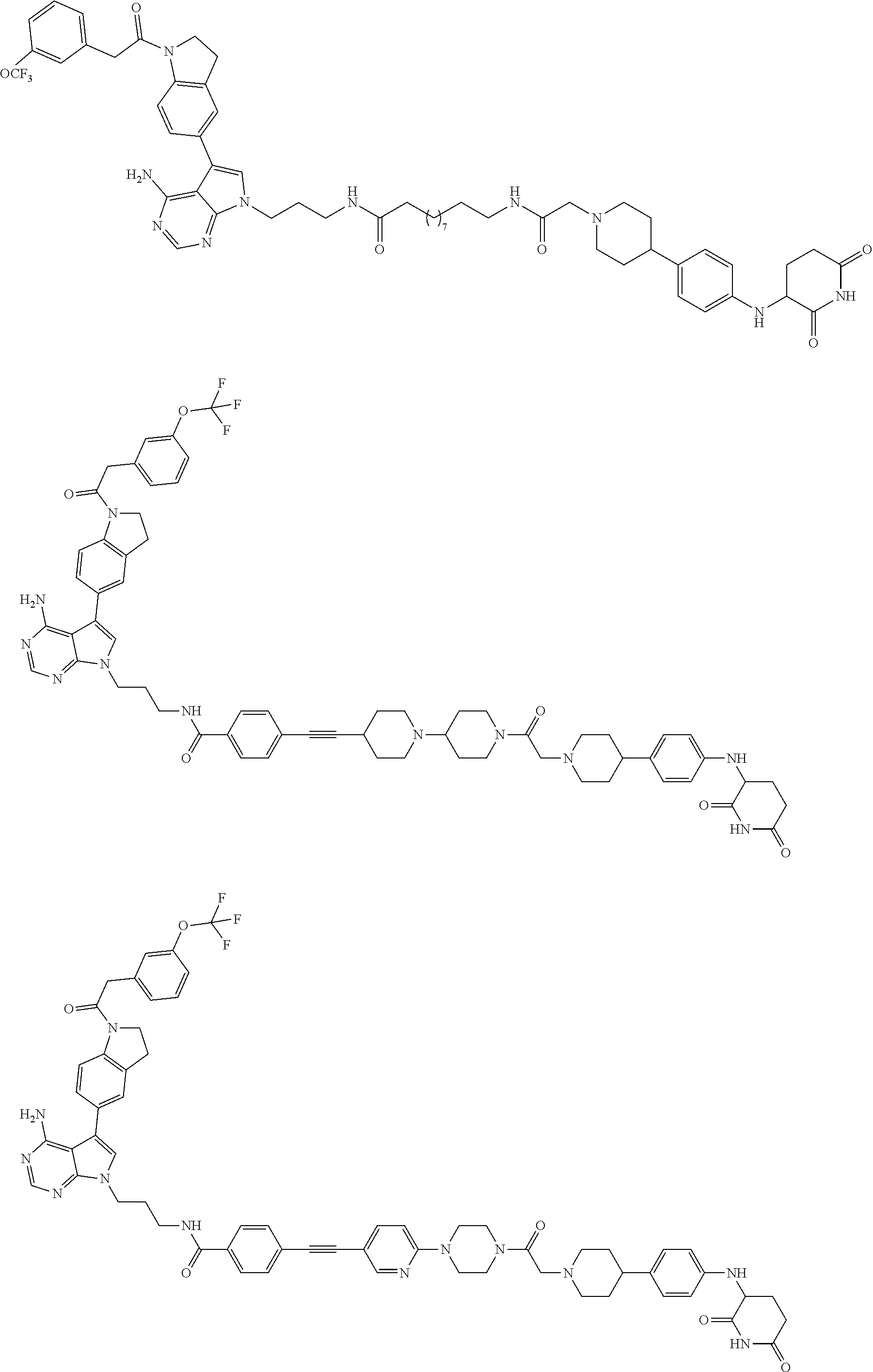 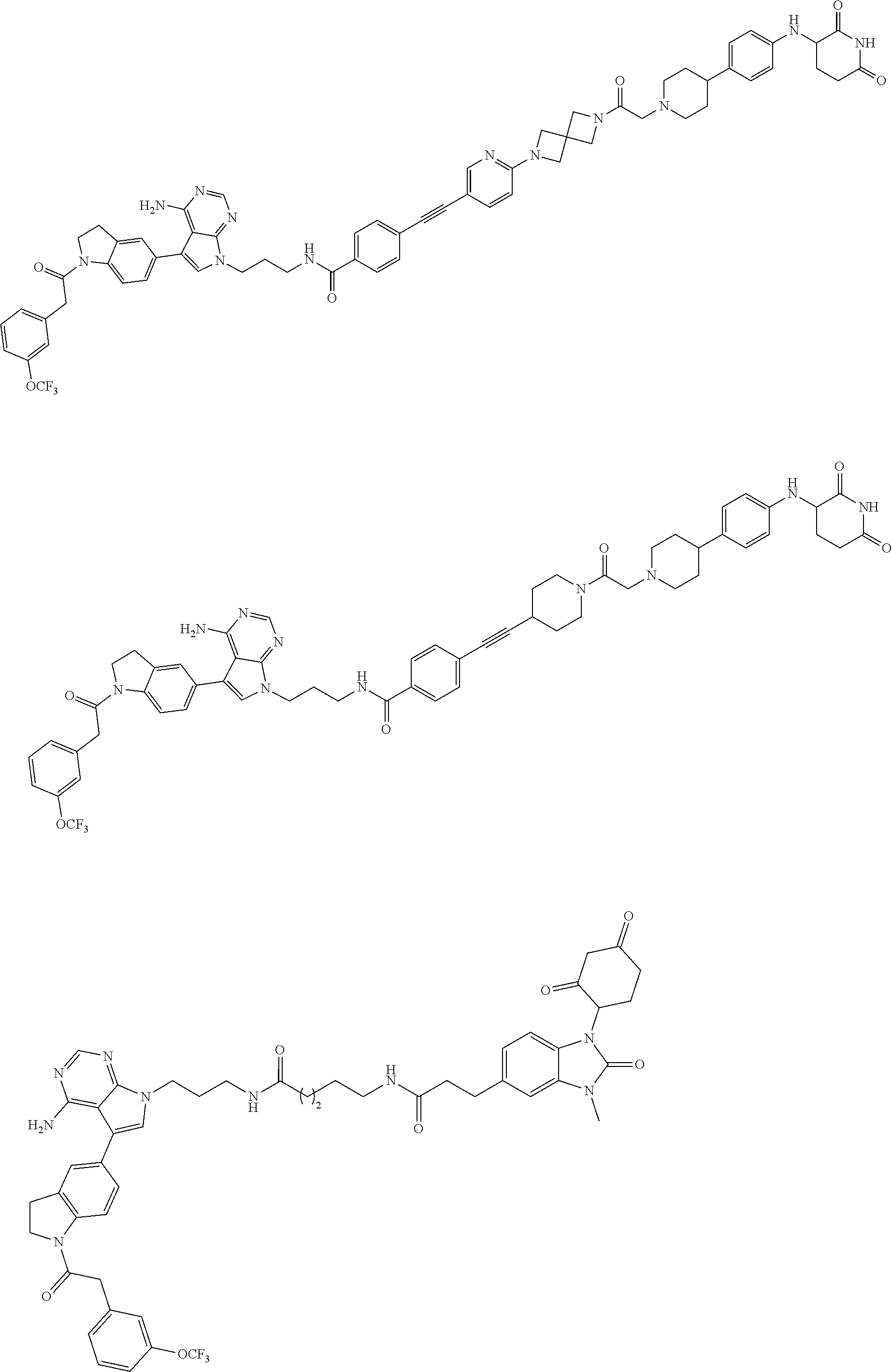 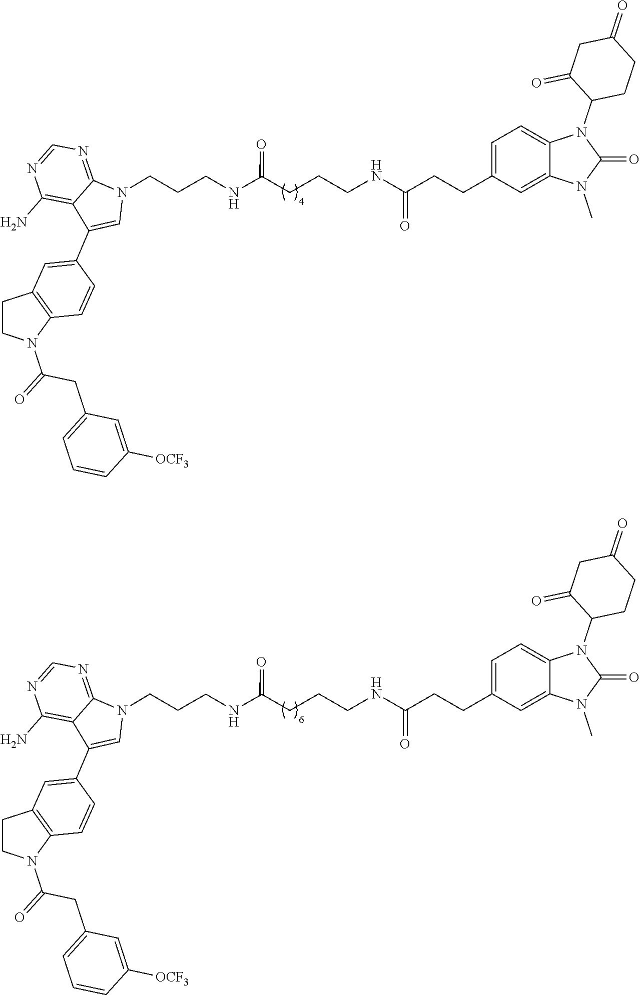 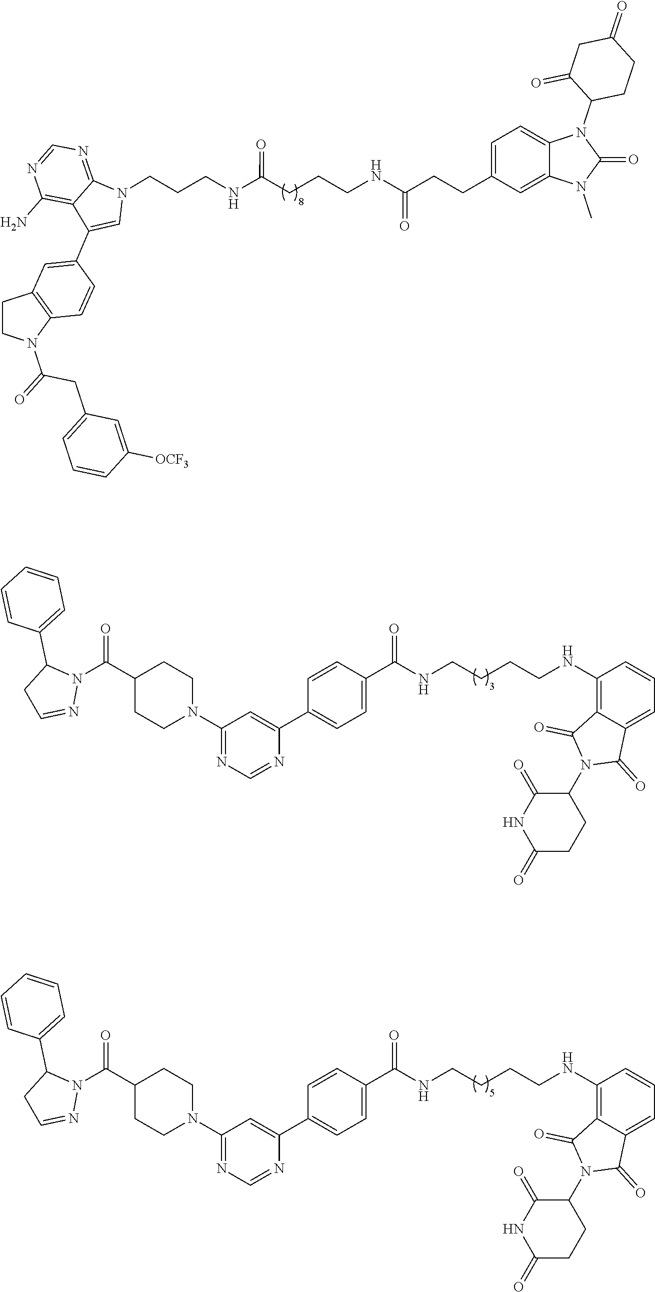 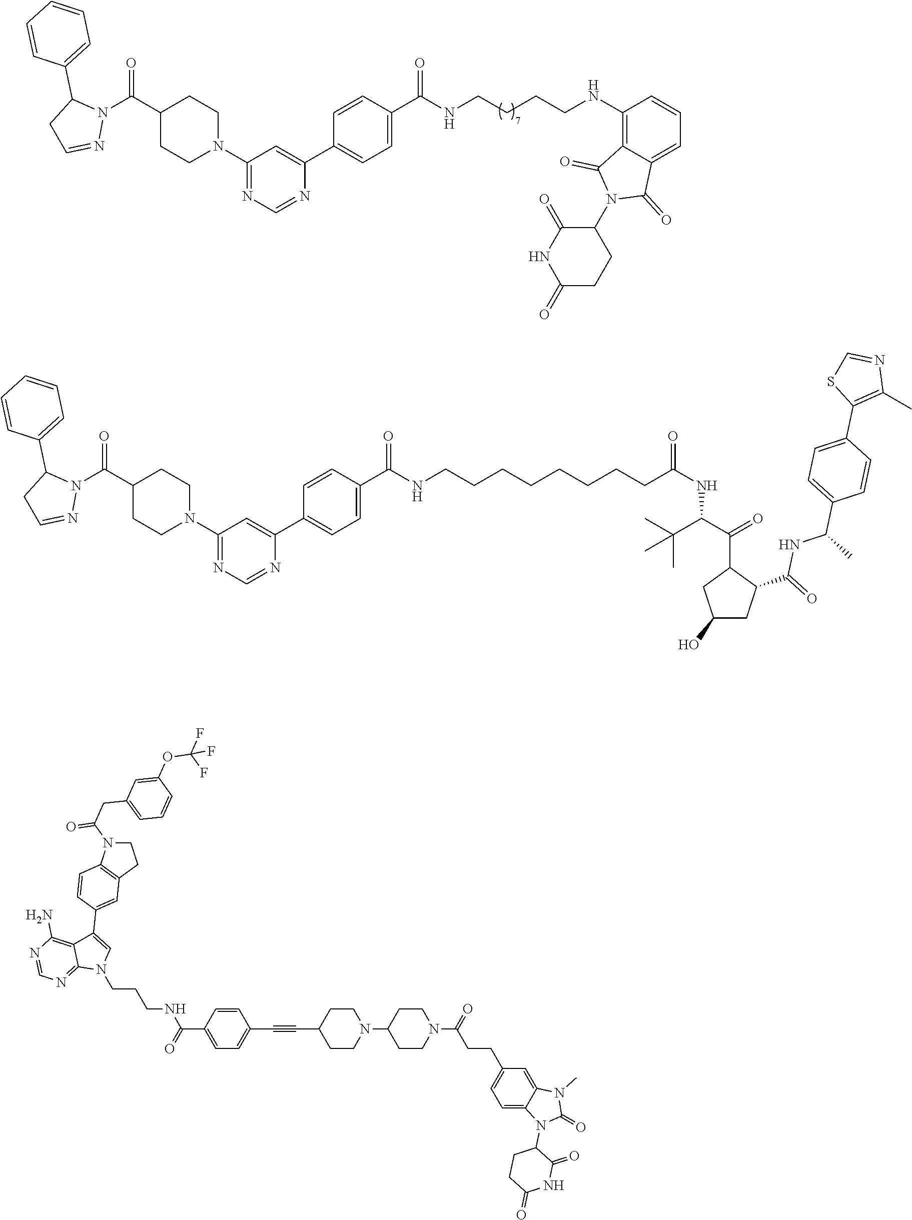 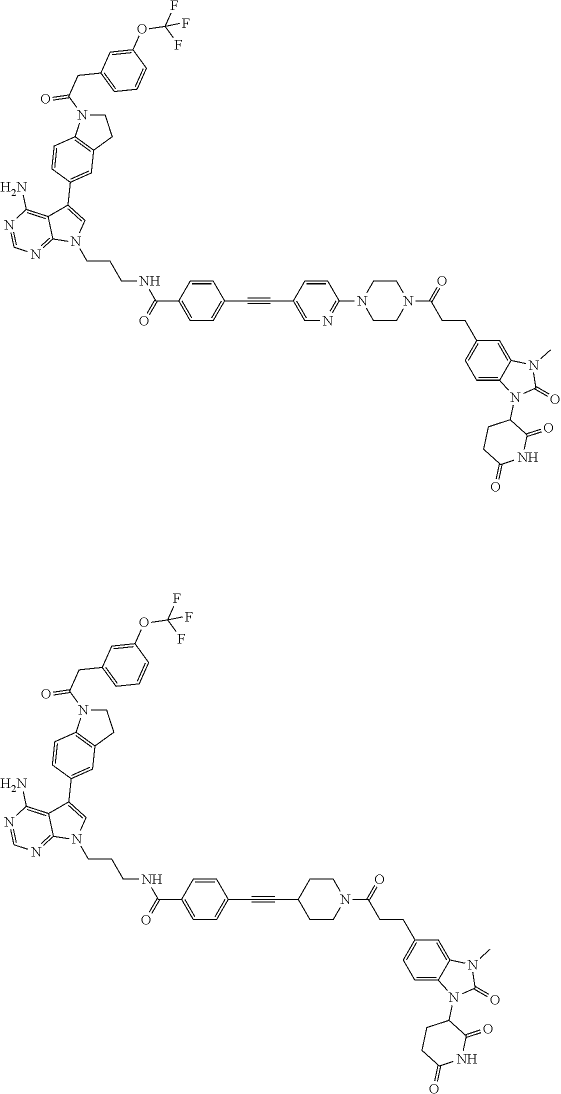 [SMILES: Cn1c(=O)n(C2CCC(=O)NC2=O)c2ccc(CCC(=O)N3CCN(c4ccc(C#Cc5ccc(C(=O)NCCCn6cc(-c7ccc8c(c7)CCN8C(=O)Cc7cccc(OC(F)(F)F)c7)c7c(N)ncnc76)cc5)cn4)CC3)cc21 ; Cn1c(=O)n(C2CCC(=O)NC2=O)c2ccc(CCC(=O)N3CCC(C#Cc4ccc(C(=O)NCCCn5cc(-c6ccc7c(c6)CCN7C(=O)Cc6cccc(OC(F)(F)F)c6)c6c(N)ncnc65)cc4)CC3)cc21] 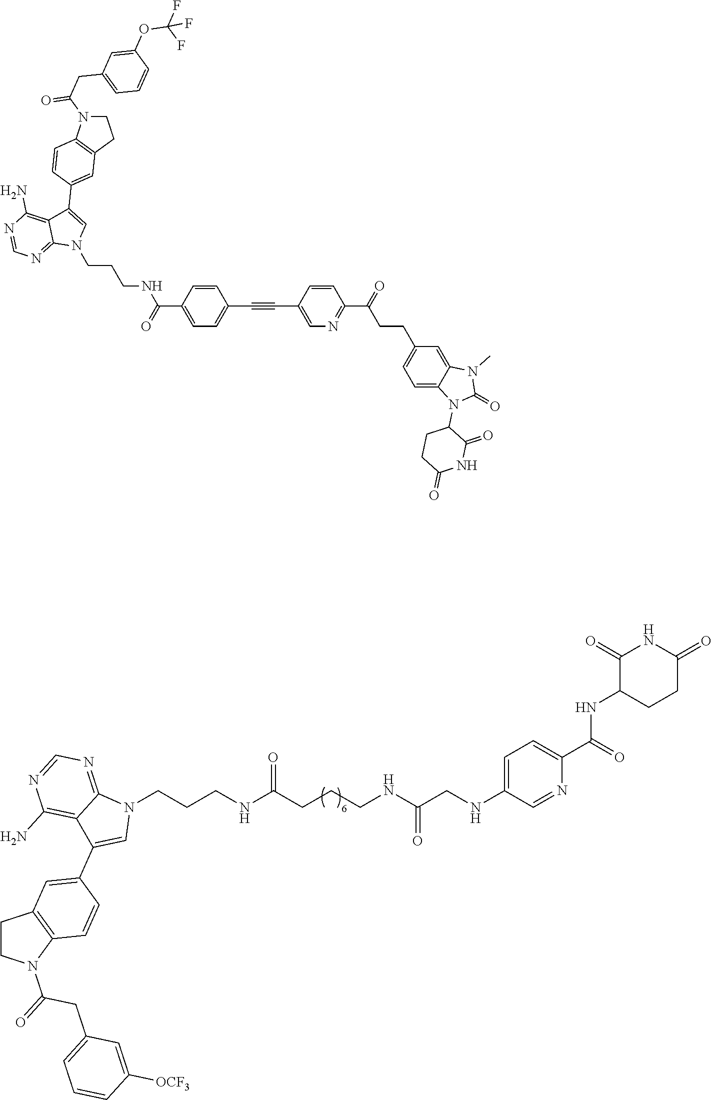 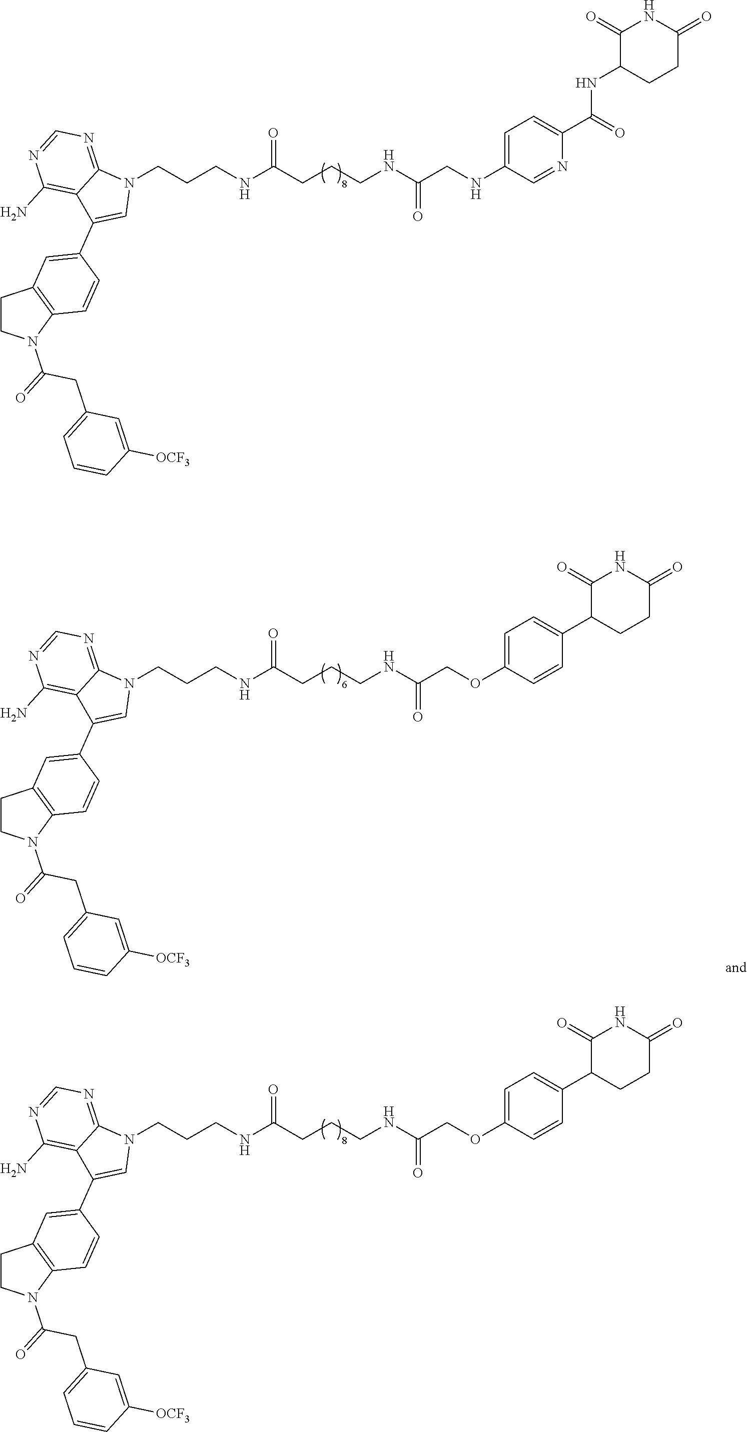
or a pharmaceutically acceptable salt, co-crystal, tautomer, stereoisomer, solvate, hydrate, polymorph, isotopically enriched derivative, or prodrug thereof.

44. A pharmaceutical composition comprising a compound of claim 1 and a pharmaceutically acceptable carrier.

45. A kit comprising a compound of claim 1.

46. A method of treating or preventing a RIPK1 kinase-related disease in a subject, comprising:
administering to the subject an effective amount of a compound of claim 1.

47. The method of claim 46, wherein the RIPK1 kinase-related disease is cancer.

48. The method of claim 47, wherein the cancer is bladder cancer, blood cancer, a bone marrow cancer, brain cancer, breast cancer, bronchus cancer, colorectal cancer, cervical cancer, chondrosarcoma, endometrial cancer, gastrointestinal cancer, gastric cancer, genitourinary cancer, head and neck cancer, hepatic cancer, hepatocellular carcinoma, leukemia, liver cancer, lung cancer, lymphoma, melanoma of the skin, ovarian cancer, pancreatic cancer, prostate cancer, renal cancer, skin cancer, testicular cancer, thyroid cancer, or uterine cancer.

49. The method of claim 46, wherein the RIPK1 kinase-related disease is an inflammatory disease.

50. The method of claim 49, wherein the inflammatory disease is neuroinflammation, asthma, chronic obstructive pulmonary disorder (COPD), chronic bronchitis, cystic fibrosis, atherosclerosis, post-angioplasty, restenosis, coronary artery diseases, angina, rheumatoid arthritis, osteoarthritis, dermatitis, eczematous dermatitis, psoriasis, post transplantation late and chronic solid organ rejection, systemic lupus erythematosis, dermatomyositis, polymyositis, Sjogren's syndrome, polymyalgia rheumatica, temporal arteritis, Behcet's disease, Guillain Barre syndrome, Wegener's granulomatosus, polyarteritis nodose, an inflammatory neuropathy, vasculitis, an inflammatory disorder of adipose tissue, Kaposi's sarcoma, or a smooth muscle cell proliferative disorder.

51. The method of claim 46, wherein the RIPK1 kinase-related disease is a neurodegenerative disorder.

52. The method of claim 51, wherein the neurodegenerative disorder is Alexander disease, Alper's disease, Alzheimer's disease, amyotrophic lateral sclerosis, ataxia telangiectasia, Batten disease, Canavan disease, Cockayne syndrome, corticobasal degeneration, Creutzfeldt-Jakob disease, Huntington's disease, Kennedy's disease, Krabbe disease, Lewy body dementia, Machado-Joseph disease, spinocerebellar ataxia type 3, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinocerebellar ataxia, spinal muscular atrophy, Steele-Richardson-Olszewski disease, Tay-Sachs, transmissible spongiform encephalopathies (TSE), or tabes dorsalis.

53. The method of claim 46, further comprising administering a second compound, biomolecule, or composition.

54. The method of claim 53, wherein the second compound, biomolecule, or composition is an anti-inflammatory agent.

55. The method of claim 46, wherein the step of administering the compound or the pharmaceutical composition is performed orally, intraperitoneally, sublingually, subcutaneously, intravenously, or any clinically acceptable administration route.

56. A method of degrading or inhibiting an RIPK1 kinase in a cell, comprising:
contacting the cell with an effective amount of a compound of claim 1.

57. The method of claim 56, wherein the contacting is performed in vitro or in vivo.
